(19) United States
(12) Patent Application Publication
(10) Pub. No.: US 2005/0196412 A1
(43) Pub. Date: Sep. 8, 2005

(54) Non-pathogenic strains of HIV-1

(76) Inventors: Nicholas John Deacon, Balwyn (AU); Jennifer Catherine Learmont, Patongo (AU); Dale Alan McPhee, Fitzroy (AU); Suzanne Crome, South Yarra (AU); David Cooper, Bellevue Hill (AU)

Correspondence Address: SCULLY SCOTT MURPHY & PRESSER, PC, 400 GARDEN CITY PLAZA, SUITE 300, GARDEN CITY, NY, 11530, US

(21) Appl. No.: 11/035,899

(22) Filed: Jan. 14, 2005

Related U.S. Application Data
(63) Continuation of application No. 09/146,783, filed on Sep. 3, 1998, now abandoned.
Application No. 09/146,783 is a continuation of application No. 08/477,464, filed on Jun. 7, 1995, now abandoned.
Application No. 08/477,464 is a continuation-in-part of application No. 08/388,353, filed on Feb. 14, 1995, now Pat. No. 6,010,895.

(30) Foreign Application Priority Data
May 17, 1995 (AU) PN3021/95

(51) Int. Cl. A61K 39/21

(52) U.S. Cl. 424/208.1

(57) ABSTRACT

The present invention relates to non-pathogenic strains of HIV-1 and to components, parts, fragments and derivatives thereof and to genetic sequences derived therefrom and their use in the development of diagnostic and therapeutic compositions for the treatment and prophylaxis of AIDS and AIDS-related disorders. The present invention also relates to a method for attenuating pathogenic strains of HIV-1 by mutagenizing particular regions of the HIV-1 genome.

### CROSS REFERENCE TO RELATED APPLICATION

This application is a continuation in-part application of U.S. Ser. No. 388,353 filed on 14 Feb., 1995.

### FIELD OF THE INVENTION

The present invention relates to non-pathogenic strains of HIV-1 and to components, parts, fragments and derivatives thereof and to genetic sequences derived therefrom and their use in the development of diagnostic and therapeutic compostions for the treatment and prophylaxis of AIDS and AIDS-related disorders. The present invention also relates to a method for attenuating pathogenic strains of HIV-1 by mutagenizing particular regions of the HIV-1 genome. A particularly useful aspect of the present invention is a method for determining the likelihood or otherwise of an individual who is seropositive for HIV-1 developing AIDS or AIDS-related ms. Another aspect of the present invention is directed to strains of HIV-1 capable of synthesizing a modified Nef protein or having a wild-type Nef protein modified after thereby rendering those streams of HIV-1 substantially non-pathogenic.

### BACKGROUND OF THE INVENTION

Bibliographic details of the publications referred to in this specification are collected at the end of the description. Sequence Identity Numbers (SEQ ID NOs.) for the nucleotide and amino acid sequences referred to in the specification are defined following the bibliography.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. Genomic nucleotide sequences of HIV-1 strains referred to here are represented by their corresponding DNA sequence.

Exemplary viral isolates referred to herein as “C18” and “C99” were at the PHLS Centre for Applied Microbiology and Research European Collection of Animal Cell Cultures (ECACC), Division of Biologies, Porton Down. Salisbury, Wiltshire SP4 OJG. C18 was deposited on 17 Oct. 1994 under Provisional Accession Number V94101706 and C98 was deposited on 31 October, 1994 under Provisional Accession Number V941031169. Viral isolate “C54” was deposited at ECACC on 10 Mar. 1995 under Provisional Accession Number V95031022.

A summary of particular deletion mutants of HIV-1 of the present invention refaced to herein is given in FIG. 11.

Acquired Immune Deficiency Syndrome (AIDS) and AIDS relay did arc the clinical result of infection by Human Immunodeficiency Virus type I (HIV-1) (Barre-Sinoussi at, 1983). Infection by HIV-1 is generally characterised by progressive immune system damage (T e t at, 1990; Clerici et al, 1989 leading to opportunistic infections malignancies or wasting syndrome that constitute clinically-defined AIDS (Busch et al, 1991; Klaslow et at, 1990).

The high mortality rate of individuals infected with HIV-1 together with the social and economic consequences of the continuing HIV-1 epidemic has created an urgent need for a safe and effective treatment and/or prophylaxis against the devastating effects of AIDS. However, despite over a decade of high level scientific into the pathogenesis of HIV-1 and the clinical manifestations of the disease, together with a detailed molecular analysis of the brink there has been little success in the development of an effective vaccine. To date, the most effective therapy is treatment with zidovudine (AZT) which delays the onset of full blown AIDS and alleviates to some extent the symptoms of HIV-1 infection. However, AZT is not an in s compound and AZT, metabolic products thereof or impurities therein can cause a number of side effects which limit long term treatment with the drug. Furthermore, AZT resistant isolates have been reported during s ent Clearly, therefore, a need exists to develop alternative strategies in preventing and treating HIV-1 infection.

The initial phases of HIV-I infection are summarised by Levy (1993) as involving attachment, fusion and nucleocapsid entry. These phases have been the traditional foci in research into development of antiviral strategies. The molecular cats at the virus genomic level have also been the subject of intense scientific research with an aim being the development of a live attenuated vaccine as a possible approach for the treatment or prophylaxis of HIV-1 infection.

There is a high variable rate of progression from initial HIV-1 on to AIDS which reflects a rapidly changing pathogen and variable immune response of the host to infection (Sheppard et al, 1993). With regards to the later, HIV-1 can be considered as a heterogenous group of viruses differing at the genetic level with concomitant variable pathogenicity. For example, HIV-1 strains can differ in their capacity to kill cells. Furthermore, it appears that HIV-1 strains evolve in a host after infection and that the evolution vanes depending on the tissues infected by the virus. The major sites in the genome apparently responsible for biological and pathological variation are the highly variable envelope region (Cheng-Mayer et al., 1991; shioda et al., 1992; Hwang, et al 1991; Sullivan et at, 1993; Groenink et al., 1993) and the viral regulatory regions such as tat (Leguern et at, 1993). The genetic complexity of the HIV-1 group of viruses together with their variable pathogenicity, are major difficulties in the development of live vaccines, genetic vaccines or component vaccines.

Notwithstanding the highly pathogenic nature of HIV-1, there are some reports of long term survival of subjects infected with the virus (Learmont et al, 1992; Levy, 1993; Sheppard et al, 1993; Lifson et al 1991). It is not always clear, however, whether a benign course following HIV-1 infection is due to host factors, viral factors, or other unknown factors. There are reports that most infected people have at least laboratory evidence of progressive immune system damage in the form of CD4+ cell loss (Lang et al, 1989) and defective immune responses (Clerici et al, 1989).

Although simian monkeys have been used as an in vivo model for HIV and Simian immunodeficiency Virus (SIV) infection, a major handicap in AIDS research has been the absence of suitable in vivo models to study the pathogens of the disease and, in particular, to study the viruses involved in benign infection The need for a suitable in vivo model is heightened by the fact that results obtained in vitro cannot necessarily be extrapolated to what occurs in vivo. This was clearly observed by Mosier et al (1993) where conflicting results were obtained in animals compared to cell cultures.

Despite the absence of suitable in vitro models, considerable scientific research has been directed to attenuating HIV-1 strains by mutagenesis of the virus genome Deletions in the nef gene have been implicated in attenuated strains of SIV and their use in providing protective effects in monkeys (Daniel et at, 1992). However, there are conflicting reports on the possible negative influence the nef gene product has on the rate or extent of virus replication (Terwilliger et at, 1986; Luciw et al., 1987; Niederman et al., 1989; Kim et al, 1989; Hammes et al, 1989). In fact, Kim et al (1989) found that nef did not affect HIV-1 replication or HIV-1 long terminal repeat (LTR)-driven CAT expression. Kestler III et al (1991) found that the nef gene is required for full pathogenic potential in SIV. However, such is the complexity of the HIV-1 group of and the variability of immune responses between individuals let alone different species, that it is far from clear whether nef deleted strains of HIV-1 would behave to nef deleted strains of SIV-I. There is a need, therefore, in order to investigate the possibility of nef deleted HIV-1 strain as a vaccine candidate, to identify individuals infected with such modified viruses.

Learmont et at (1992) that a cohort of five persons infected with blood products from a single HIV-1 infected donor have remained asymptomatic from up to about 10-14 years after infection. Subsequently, a sixth Non has been identified as being part of the cohort. Both the donor and recipients were HIV-1 seropositive but with no indications of clinical Symptoms of HIV-1 related disease and CD4+ cell number and β₂-microglobulin levels have remained in the normal range. The identification of this cohort of benignly infected individuals provides a unique in vitro model in which the pathogenesis of HIV-1 infection can be studied at the clinical and molecular biological levels.

However, it has not always been possible using conventional isolation procedures to routinely and reproducibly isolate viral strains from the above mentioned donor or recipients which has ated efforts to investigate the cause of the asymptomatic individuals. In accordance with the present invention, methods have now been established to isolate viruses from the above individuals. It has been determined, in accordance with the present invention, that individuals of the cohort are infected by non-pathogenic strains of HIV-1. Furthermore, the non-pathogenic strains of HIV-1 carry one or more nucleotide mutations. The non-pathogenic strains of the present invention enable the generation of arrange of therapeutic, diagnostic and targeting agents against HIV-1 infection. The present invention also enables the Dual of previously pathogenic strains of HIV-1. Additionally, an investigation of the immunological profiles of cohort individuals has revealed that a non-pathogenic strain of HIV-1 is indicated by a particular deletion in the coding region of a protein resulting in an altered immunological profile for the expressed protein. An example of an altered immunological profile results from a deletion of certain amino acids in the Nef protein.

### SUMMARY OF THE INVENTION

One aspect of the invention is directed to an isolated HIV-1 strain or a component, part, fragment or derivative thereof which is substantially non-pathogenic.

Another aspect of the invention is direction an isolated strain of HIV-1 or a biological source thereof, said HIV-1 having the following characteristics:
(i) is substantially non-pathogenic in human subjects; and
(ii) carries a modified nef gene which encodes a nef gene product substantially immunologically non-interactive with antibodies to amino acids 162 to 177 of Nef in wild-type HIV-1.

Yet another aspect of the invention is directed to an isolated non-pathogenic strain of HIV-1 comprising a genome which is substantially incapable of hybridizing under medium stringent conditions to a nucleic acid molecule comprising a sequence of nucleotides which encodes all or part of amino acids 162 to 177 of wild-type HIV-1 Nef.

Still another aspect of the invention provides a non-pathogenic HIV-1 isolate which:
(i) induces an immune response in a human or primate subject;
(ii) does not substantially produce a proliferative response or cytokine production to a mitogen, alloantigen and/or recall at relative to a healthy, non-infected subject, and
(iii) is substantially incapable of inducing an antibody to amino acids 162 to 177 of wild-type HIV-1 Nef protein.

Still yet another aspect of the invention contemplates a viral isolate which:
(i) is interactive to antibodies to a glycoprotein from HIV-1 selected form gp41-45, gp120 and gp160;
(ii) is substantially non-pathogenic in human subjects; and
(iii) carries a deletion mutation of at least ten nucleotides in a region corresponding to all or part of amino acids 162 to 177 encoded by the nef gene of a pathogenic so of HIV-1.

Another aspect of the invention provides an isolated strain of HIV-1 which is reactive to antibodies to a glycoprotein of HIV-1, is capable of inducing an immune response to at least one of gag, pol and/or env and which is incapable of directing of a nef gene product or a full length nef gene product.

A further aspect of the invention contemplates a method or inhibiting or reducing productive infection of an individual by a pathogenic strain of HIV-1, said method comprising administering to a subject a non-pathogenic isolate of HIV-1 in an amount effective to infect target cells and to generate target cells carrying DNA derived from said non-pathogenic HIV-1.

In yet another aspect of the invention there is contemplated a method for vaccinating an individual against the development of AIDS or AIDS related diseases, said method comprising administering to said individual a non-pathogenic isolate of HIV-1 in an amount effective to infect target cells and to generate target cells carrying DNA derived from mid non-pathogenic HIV-1.

In still yet another aspect of the invention there is provided a method for obtaining a preparation of non-pathogenic HIV-1 from a biological sample, said method comprising co-culturing PBMCs from said biological sample from an individual putatively infected with said non-pathogenic HIV-1 with HIV-1 seronegative donor PBMCs depleted for CD8+ cells, Gong the PBMCs and supernatant fluid every from about 5 to about 10 days a adding fresh medium with CD8+ depleted PBMCs with said fresh medium and isolating said virus from the supernatant fluid.

Another of the invention contemplate a method for obtaining preparation of non-pathogenic HIV-1 from a biological sample, said method comprising co-culturing monocytes from said biological sample from an individual putatively infected with said non-pathogenic HIV-1 with HIV-1 seronegative donor PBMCs depleted for CD8+ cells, harvesting the monocytes and PBMCs and supernatant fluid every from about 5 to about 10 days and adding fresh medium with CD8+ depleted PBMCs with said fresh medium and isolating said virus from the supernatant fluid.

In yet further aspect of the invention there is contemplated a method for identifying or screening for compounds capable of reducing or otherwise interfering with HIV-1 replication, said method comprising contacting a compound to be tested with a cell or cell extract containing or capable of containing a nef gene product fused to a reporter molecule capable of giving an identifiable signal and screening for a compound which inhibits said signal.

In still yet a further aspect of the invention there is provided a viral isolate which:
(i) is genetically or immunologically related to a pathogenic strain of HIV-1;
(ii) is substantially non-pathogenic in human subjects;
(iii) comprises a first nucleotide sequence constituting its genome which is capable of hybridising under medium stringency conditions to SEQ ID NO: 1 or a complementary form thereof; and
(iv) comprises a second nucleotide sequence within said first nucleotide sequence and which second nucleotide sequence direct expression of a mRNA molecule capable of inhibiting, reducing or otherwise down-regulating translation of a protein or polypeptide encoded by a pathogenic strain of non-protein HIV-1 or inhibit, reduce or otherwise down regulate operation of a non-protein encoding a region of a pathogenic strain of HIV-1.

Still yet another aspect of the invention is directed to a method for determining the pathogenicity of an HIV-1 strain after said HIV-1 strain infects cells of an individual, said method comprising determining the presence of a deletion mutation in the genome of said HIV-1 wherein said deletion mutation results in said genome being unable to synthesize a polypeptide or protein from a pathogenic strain of HIV-1 or directing the synthesis of a truncated form of said polypeptide or protein wherein the presence of a such a mutation is indicative of the presence of a non-pathogenic strain of HIV-1.

A further aspect of the invention contemplates a method for determining the pathogenicity of a strain of HIV-1 after said HIV-1 strain infects cells of an individual, said method comprising contacting a biological sample from said individual with a peptide corresponding to a deleted or truncated region of an HIV-1-derived protein and screening for the absence of antibody binding to said peptide, wherein the absence of antibody binding is indicative of a deletion or truncation in that protein and further indicative of the non-pathogenicity of said strain of HIV-1.

Another aspect of the invention is directed to a method for determining the pathogenicity of a stain of HIV-1 after said HIV-1 strain infects cells of an individual, said method comprising contacting a biological sample from said individual with an effective amount of a peptide having an amino acid sequence comprising or within amino acids 162-177 of wild-type HIV-1_{NL43} Nef, said contact being for a time and under conditions sufficient for an antibody if present in said biological sample to form a complex with said peptide and then detecting the presence of said complex wherein the absence of a complex in an individual seropositive for HIV-1 is indicative of that individual being infected with a non-pathogenic strain of HIV-1.

Still another aspect of the invention provides a method for determining the pathogenicity of an HIV-1 strain after said HIV-1 strain infects cells of an individual, said method comprising determining the presence of a mutation in the genome of said HIV-1 wherein the presence of the mutation results in a Nef protein subtly lacking amino acids 162 to 177 of Nef of a wild-type strain of HIV-1 and wherein said mutation is indicative of a non-pathogenic strain of HIV-1.

Still yet another aspect of the invention is directed to a synthetic peptide comprising a sequence of amino acids defined by SEQ ID NO:801 or a part or fragment thereof.

In yet a further aspect of the invention there is contemplated a method for determining the risk of an individual seropositive for HIV-1 developing symptoms of AIDS, said method comprising contacting antibodies from said patient with a q peptide defined by SEQ ID NO:801 or a part or derivative thereof and detecting non-binding of antibodies to said peptide wherein the substantial absence of antibodies to said peptide is indicative of a low risk of the individual developing AIDS.

### BRIEF DESCRIPTION OF THE FIGURES

FIG. 1 is a representation showing the alignment of the nucleotide sequences from donor D36 peripheral blood mononuclear cell (PBMC) [D36P] and non-pathogenic HIV-1 from recipient C18 HIV_{StV} [C18S], C18 HIV_{MBC} [C18M]and C98 HIV [C98H]and C54 PBMC [C54P]with the equivalent region of HIV-1_{NL43}. Sequences labelled PBMC are from patient PBMC, those labelled HIV are from virus isolated from patient PBMC and grown in culture. Numbering for HIV-1_{NL43} is as per Myer et al (1992; 1994) where nucleotide 1 is the first nucleotide of the complete proviral DNA sequence. D36P, C18S, C18M, C98H and C54P are numbered from the start of the region sequenced. Identity with NL43 sequence is shown by (*). Deleted nucleotides are shown by (−). Spaces introduced to maximise alignment are shown by (,). Features in HIV-1_{NL43} are marked by overlining the sequence, features in D36 and C18 are marked by underlining the sequence.

FIG. 2 shows the alignment of encoded amino acid sequence of (a) tat exon 3 and (b) rev exon 3 from HIV-1_{NL43}, D36 PBMC, C18 HIV_{StV} and C98 HIV. In-phase termination codons (*) and NL43 encoded amino acid numbers are shown.

FIG. 3 is a representation showing the alignment of C-terminal envelope glycoprotein gp41 amino acid sequences encoded by D36 PMBC, C18 HIV_{StV} , C18 HIV_{MBC} and C98 HIV. Numbering is that of the amino acid sequence of the mature envelope glycoprotein of HIV-1_{NL43}. Termination in shown by (*).

FIG. 4 is a representation showing alignment of amino acid sequences encoded by the nef genes of HIV-1_{NL43}, D36 PBMC, C18 HIV_{StV}, C18 HIV_{MBC} and C98 HIV. In phase termination codons are shown by (#). Identical amino acids are shown by (*). Residues underlined are those immediately before a deletion.

FIG. 5 shows a duplication of NFKB and Sp1 sequences in D36 PBMC, C18 HIV_{StV} , C18 HIV_{MBC} and C98 HIV demonstrated by alignment of their low homology region sequences with the NFKB-Sp1 region of HIV-1_{NL43}. Nucleotide numbering according to FIG. 1. Identity with NL43 sequence shown by (*) and NFKB and Sp1 sites in NL43 overlined. Position of nef/LTR region sequence deletion shown by (▴).

FIG. 6 is a graphical representation showing replication of C18 and C98 viral isolates and D36 PBMCs from asymptomatic patients in PHA-stimulated PBMCs.

| | |
|---|---|
| [symbol] | C18 |
| [symbol] | C98 |
| [symbol] | D36 |
| [symbol] | Mock |

FIG. 7 is a graphical representation showing replication of viral isolates from asymptomatic patients in non-PHA stimulated PBMCs.

| | |
|---|---|
| [symbol] | 228200 |
| [symbol] | C98 |
| [symbol] | C18 |
| [symbol] | Mock |

228200 is an Australian isolate of HIV-1 described by Kiernan, R. et al (1990). Its characteristics include being T cell trophic, with fast kinetics, high producer of HIV-1 and/or SI phenotype.

FIG. 8 is a graphical representation of cell surface receptor expression for syncytia-inducing (SI)/non-syncytia-inducing (NSI)/asymptomatic patient isolates.

| | |
|---|---|
| [symbol] | TIR |
| [symbol] | IL-2R |
| [symbol] | CD4 |

228220 is defined in the legend to FIG. 7. 243925 is a viral isolate of HIV-1 which is monocyte/macrophage trophic and exhibits NSI phenotype (Dr Karen Coats-Fryer PhD thesis entitled “Viral determinants of HIV-1 syncytium formation”, the University of Melbourne, Parkville, Victoria, Australia.

FIG. 9 is a representation of the nucleotide sequence of C18 HIV-1_{MBC} (SEQ ID NO: 800).

FIG. 10(a)-(g) are graphical representations showing clinical immunology of cohort; (a) CD3; (b)(i) CD4 (ii) CD4%; (c)(i) CD8; (ii) CD8%; (d) lymphocyte count; (e) CD4/CD8 ratio; (f) β-2-microglobulin; and (g) Kaplan-Meier estimates of time to disease progression (Cox & Oakes, 1989).

FIG. 11 is a schematic representation of the deletion mutants of the present invention.

FIG. 12 shows reactivity of sera from LTP individuals (1a); HIV-1-ve individuals (1bi, 1bii); individuals with autoimmune disease (A/HIV-1) (1biii); LTNP1 (1c) and LTNP2 (1d) with fill length Nef 27 derived from HIV-1_{NL43} (referred to heren as “Nef 27”). The term “LTNP” is an abbreviation for “Long Term Non-Progressor”. NTNP1 and LTNP2 arc defined in Example 16.

Wells of 96-well polystyrene microtitre plates coated with purified Nef 27 (100 ng/well) were incubated with sera (titrated from 1:100 to 1:10,000) o from LTP individuals, HIV-1-ve individuals, individuals with autoimmune disease, LTNP1 and LTNP2. The presence of antibodies in the sera which recognise Nef 27 were detected using a biotin-streptavidin HRP system with o-phenylenediamine as substrate. Absorbance was measured using a Titertek plate reader at wavelength of 630 and 450 nm.

FIG. 13a shows reactivity of sera from LTP individuals against Nef-derived peptides. Synthetic peptides corresponding to o acid residues 1 to 19 (i), 20 to 36 (ii), 44 to 65 (iii); 72 to 83 (iv), 89 to 97 (v9); 109 to 114 (vi), 164 to 186 (vii), 187 to 206 (viii), 121 to 135 (ix) and 162 to 177 (x) of HIV-1_{NL43} Nef 27 were coated onto wells of 96-well microtitre plates at a concentration of 500 ng/well. Sera (titrated from 1:300 to 1;100,000) from the LTP individuals were then incubated with the immobilised peptides and the presence of antibodies in the sera which on the Nef-derived peptide were detected using a biotin-streptavidin HRP system with o-phenylenediamine as substrate. Absorbance was measured using a Titertek plate reader at wavelengths of 630 and 450 nm.

FIG. 13b(i) shows reactivity of sera from HIV-1-ve individual against Nef-derived peptides. Synthetic peptides corresponding to amino acid residues 1 to 19(i), 20 to 36 (ii), 44 to 65 (iii), 72 to 83 (iv), 89 to 97 (v), 109 to 114 (vi) 164 to 186 (vii), 187 to 206 (viii), 121 to 135 (ix) and 162 to 177 (x) of HIV-1_{N43} Nef were coated onto wells of 96-well microtitre plates at a concentration of 500 ng/well Sera (titrated from 1:300 to 1:100,000) from A/HIV-1-ve individuals with autoimmune disease was then incubated with the immobilised peptides and the presence of antibodies in the sera which recognise the Nef-derived peptides were detected using a biotin-streptavidin HRP system with o-phenylenediamine as substrate. Absorbance was measured using a Titerek plate reader at wavelengths of 630 and 450 nm.

FIG. 13b(ii) shows reactivity of sera from autoimmune A/HIV-1-ve individuals against Nef-derived peptides. Synthetic peptides corresponding to amino acid residues 1 to 19 (i), 20 to 36 (i), 44 to 65 (ii), 72 to 83 (iv), 89 to 97 (v) 109 to 114 (vi), 164 to 186 (vii), 187 to 206 (viii), 121 to 135 (ix) ad 162 to 177 (x) of HIV-1_{NL43} Nef were coated onto wells of 96-well microtitre plates at a concentration of 500 ng/well. Sera (titrated from 1:300 to 1:100,000) from A/HIV-1-ve individuals with autoimmune disease was then incubated with the immobilised peptides and the presence of antibodies in the sera which recognise the Nef-derived peptides were using a biotin-streptavidin HRP system with o-phenylenediamine as substrate. Absorbance was measured using a Titerek plate reader at wavelengths of 630 and 450 nm.

FIG. 13c shows reactivity of Sera from LTNP1 individuals against Nef-derived peptides. Synthetic peptides corresponding to amino acid residues 1 to 19 (i), 20 to 36 (ii), 44 to 65 (iii), 72 to 83 (iv), 89 to 97 (v), 109 to 14 (vi), 164 to 136 (vi), 187 to 206 (viii), 121 to 135 (ix) and 162 to 177 (vi) of HIV-1_{NL43} Nef were coated onto wells of 96-well microtitre plates at a concentration of 500 ng/well. Sera (titrated from 1:300 to 1:100,000) from the LTNP1 individuals were then include with the immobilised peptides and the presence of antibodies in the sera which recognise the Nef-derived peptides were detected using a biotin-streptavidin HRP system with o-phenylenediamine as substrate. Absorbance was measured using a Titertek plate reader at wavelengths of 630 and 450 nm.

FIG. 13d shows reactivity of sera from LTNP2 individuals against Nef-derived peptides. Synthetic peptides corresponding to amino acid residues 1 to 19 (i), 20 to 36 (ii), 44 to 65 (iii), 72 to 83 (iv), 89 to 97 (v), 109 to 114 (vi), 164 to 186 (vii), 187 to 206 (viii), 121 to 135 (ix) and 162 to 177 (x) of HIV-1_{NL43} Nef were coated onto wells of 96-well microtitre plates at a concession of 500 ng/well. Sera (titrated from 1:300 to 1:100,000) from the LTNP2 individuals were then incubated with the immobilised peptides and the presence of antibodies in thc sera which recognise the Nef-derived peptides were detected using a biotin streptavidin HRP system with o-phenylenediamine as substrate. Absorbance was measure using a Titerek plate reader at wavelengths of 630 and 450 nm.

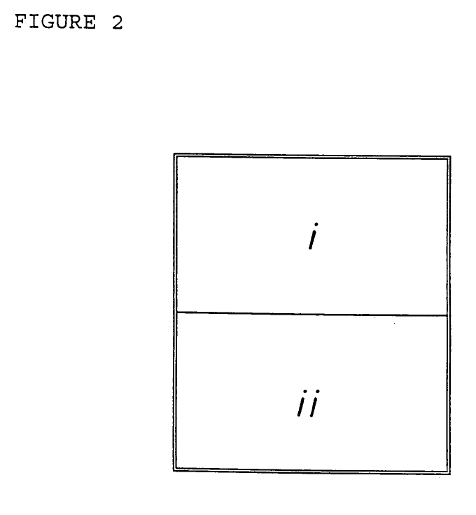

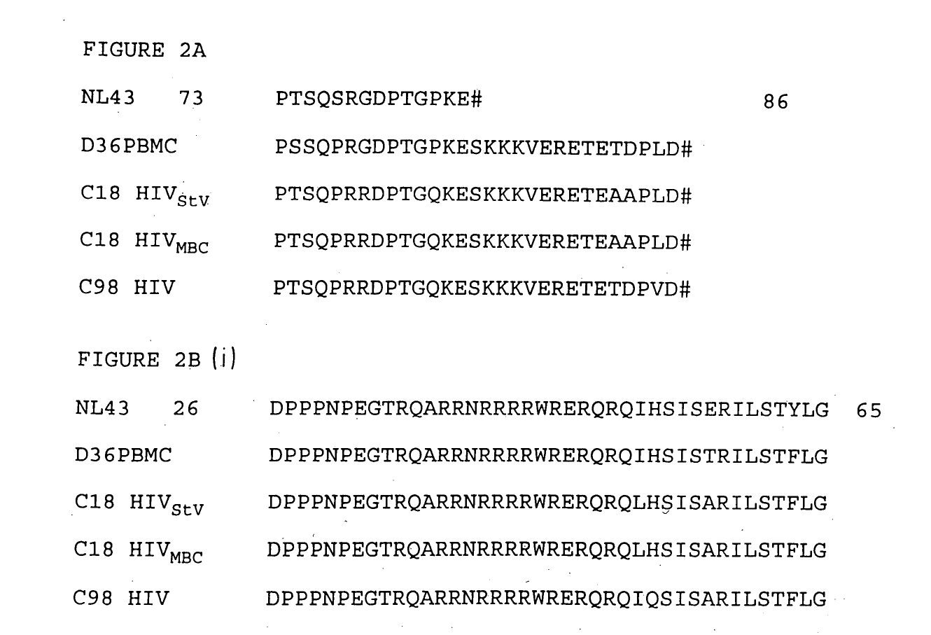

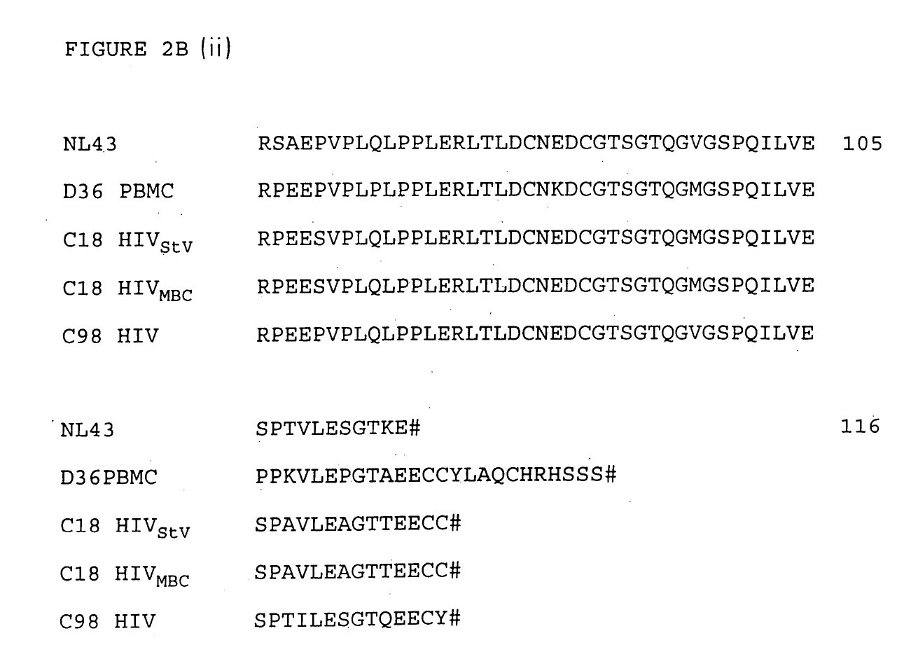

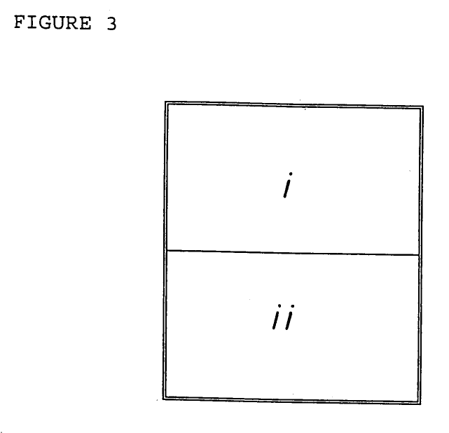

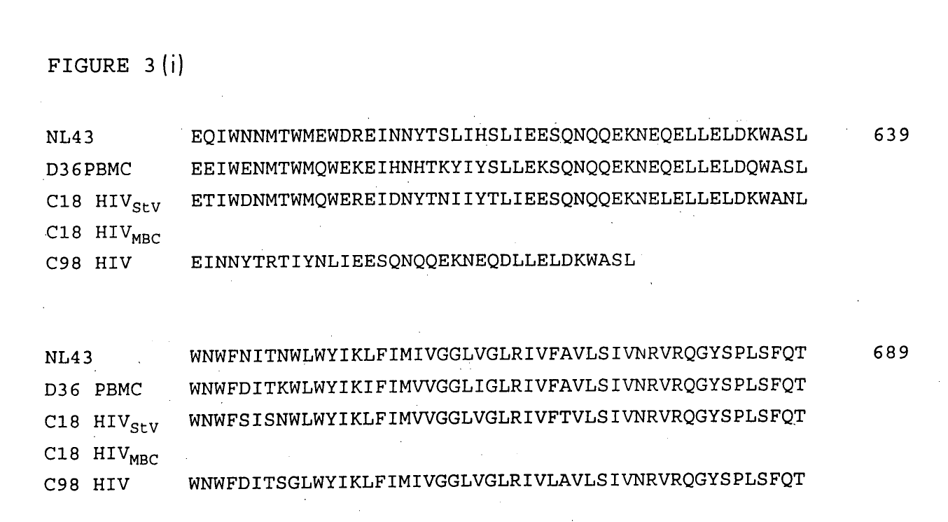

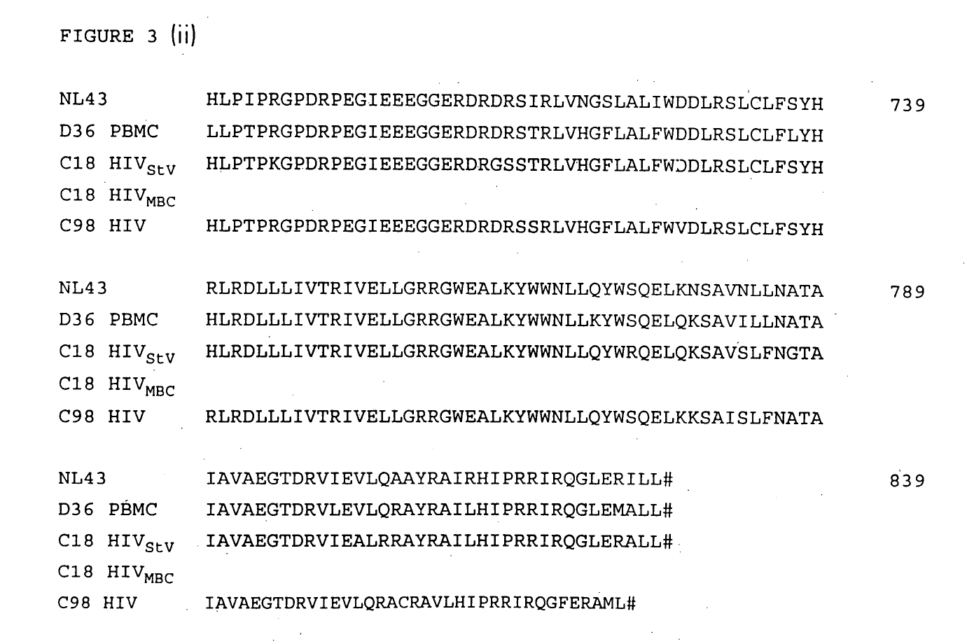

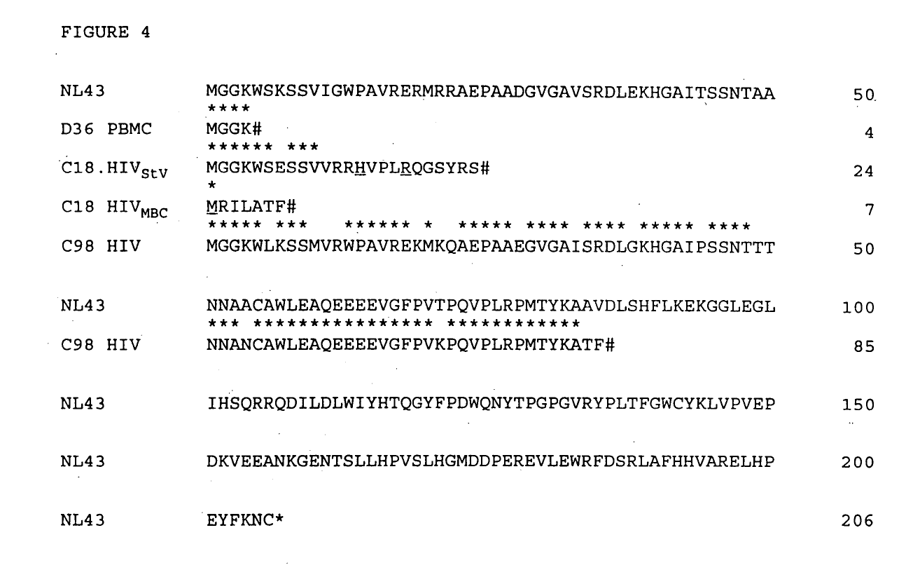

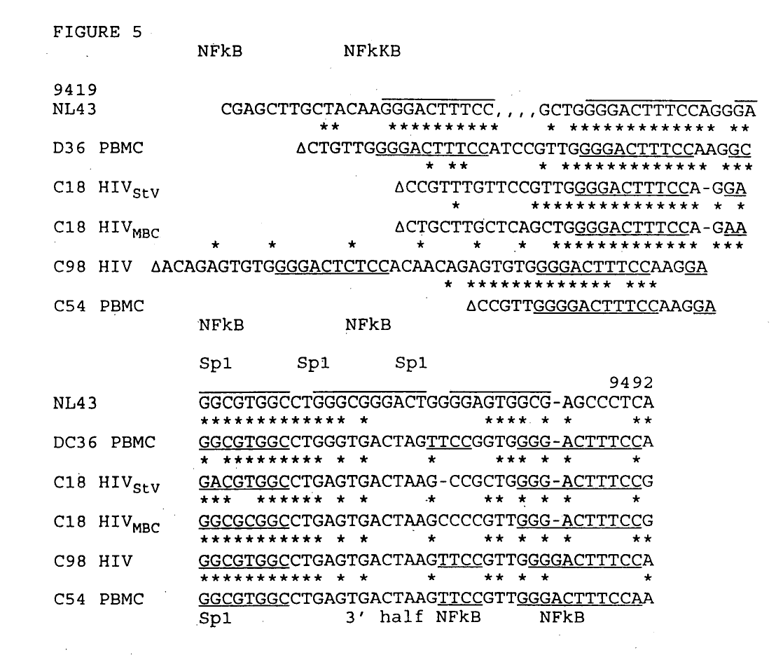

A summary of the SEQ ID Nos. used in the subject specification is shown below:

| SEQ ID NO: | DESCRIPTION |
|---|---|
| 1 | Nucleotide sequence of HIV-1_{NL43} genome |
| 2-613 | Decanucleotides of nef gene from HIV-1_{NL43} |
| 614 | Partial nucleotide sequence of D36 HIV-1 isolate |
| 615 | Partial nucleotide sequence of C18 HIV-1_{MBC} isolate |
| 616-625 | PCR primers shown in Table 1 |
| 626-633 | Sequence primers shown in Table 2 |
| 634 | Amino acid residues 15-27 of HIV-1_{NL43} nef protein |
| 635 | HIV-1_{NL43} tat exons (FIG. 2) |
| 636 | HIV-1 D36 tat exons (FIG. 2) |
| 637 | HIV-1 C18 tat exons (FIG. 2) |
| 638 | HIV-1_{NL43} rev exons (FIG. 2) |
| 639 | HIV-1 D36 rev exons (FIG. 2) |
| 640 | HIV-1 C18 rev exons (FIG. 2) |
| 641 | HIV-1_{NL43} C-terminal of gp41 (FIG. 3) |
| 642 | HIV-1 D36 C-terminal of gp41 (FIG. 3) |
| 643 | HIV-I C18 C-terminal of gp41 (FIG. 3) |
| 644 | HIV-1_{NL43} nef gene (FIG. 4) |
| 645 | HIV-1 D36 nef gene (FIG. 4) |
| 646 | HIV-1 C18 nef gene (FIG. 4) |
| 647 | HIV-1_{NL43} NFKB/SP1 sequence (FIG. 5) |
| 648 | HIV-1 D36 NFKB/SP1 sequence (FIG. 5) |
| 649 | HIV-1 C18 NFKB/SP1 sequence (FIG. 5) |
| 650 | Nucleotide sequence of nef gene from HIV-1_{NL43} |
| 651 | Nucleotide sequence of env and nef regions of HIV-1_{NL43} |
| 652-799 | Decanucleotides of LTR region HIV-1_{NL43} |
| 800 | Nucleotide sequence of C18 HIV-1_{MBC} |
| 801 | Amino acids 162 to 177 of wild-type HIV-1_{NL43} Nef |
| 802 | Nucleotide sequence encoding amino acids 162 to |
| | 177 of wild-type HIV-1_{NL43} Nef |
| 803-841 | Decanucleotide deletion of Nef gene covering |
| | amino acids 162 to 177 |

### DETAILED DESCRIPTION OF THE PREFERRED EMBODIMENTS

One aspect of the present invention contemplates a non-pathogenic isolate of HIV-1 or a component, part, fragment or derivative thereof.

In a related embodiment, there is provided a novel isolate of HIV-1 or a component, part, fragment or derivative thereof wherein said HIV-1 isolate is capable of stimulating in a human or primate subject an immune response such as a humoral immune response to at least one HIV-1 glycoprotein such as but not limited to gp41-45, gp120 and/or gp160 while not substantially redwing in said human or primate subject proliferative responses and cytokine production to a mitogen, alloantigen and/or recall antigen compared to a healthy, non-infected human or primate subject. Preferably, the cytokine is IL-2. Preferably, the mitogen is ConA or PHA and the recall antigen is influenza or tetanus toxoid. Preferably, the HIV-1 isolate is non-pathogenic.

More particularly, the present invention relates to an isolated HIV-1 strain which:
(i) is substantially nonpathogenic in human subjects; and
(ii) carries one or more mutations in its genome resulting in the inability to direct synthesis of at least one pathogenic HIV-1-derived polypeptide or protein.

Even, more particularly, the present invention provides an isolated HIV-1 strain which:
(i) is substantially non-pathogenic in human subjects; and
(ii) carries a mutation in the nef gene and/or a long terminal repeat (LTR) region or in a functionality equivalent location in the HIV-1 genome.

Still even more particularly, the present invention is directed to an isolated virus which:
(i) has a genome which is capable of hybridising under medium stringency conditions to complementary nucleic acid from a pathogenic strain of HIV-1;
(ii) is substantially non-pathogenic in human subjects;
(iii) carries one or more deletion mutations in a region of its genome corresponding to a nef gene in said pathogenic stain of HIV-1; and
(iv) optionally carries a mutation in one or both LTR regions.

In a related embodiment, there is provided an isolated virus which:
(i) has a genome which is capable of hybridising under medium stringency conditions to complementary nucleic acid from a pathogenic strain of HIV-1;
(ii) is substantially non-pathogenic in human subjects;
(iii) carries one or more deletion mutations in an LTR region of its genome; and
(iv) optionally carries a mutation in a region corresponding to a nef gene in said pathogenic strain of HIV-1.

In a further related embodiment, there is provided an isolated virus which:
(i) has a genome which is capable of hybridising under medium stringency conditions to complementary nucleic acid from a pathogenic strain of HIV-1;
(ii) is substantially non-pathogenic in human subjects; and
(iii) carries one or more deletion mutations in a region of its genome corresponding to a region which contains nef sequences and LTR nucleotide sequences.

Another aspect of the present invention is dire to an isolated strain of HIV-1 or a biological source thereof, wherein sad HIV-1 has the following characteristics:
(i) is substantially non-pathogenic in human subjects; and
(ii) caries a modified nef gene which encodes a nef gene product substantially immunologically non-interactive with antibodies to amino 1162 to 177 of Nef in wild-type HIV-1.

Amino acids of 162-177 of wild-type HIV-1_{NL43} strain (Myers et al, 1994) [hereinafter referred to as “HIV-1_{NL43}”]are as follows:
Thr Ser Leu His Pro Val Ser Leu His Gly Met Asp Asp Pro Glu [SEQ ID NO:801].

This aspect of the present invention relates in part to amino acid sequence SEQ ID NO:801 from HIV-1_{NL43} or from the functionality equivalent region of other pathogenic strains of HIV-1.

A further aspect of the present invention contemplates an isolated strain of HIV-1 or a biological source thereof, wherein said HIV-1 has the following characteristics:
(i) is substantially non-pathogenic in human subjects; and
(ii) encodes an Nef protein or portion thereof which is interactive with wild-type HIV-1 Nef antibodies but which is substantially non-interactive with Embodies to amino acids 162 to 177 of wild-type HIV-1 Nef protein.

Still another aspect of the present invention relates to an isolated strain of HIV-1 or a biological source thereof which is substantially non-pathogenic in human subjects and which is substantially incapable of directing synthesis of a Nef protein or portion thereof comprising amino acids 162 to 177 of wild-type HIV-1 Nef protein.

In still yet another aspect of the present invention, there is provided an isolated strain of HIV-1 or a biological source thereof, said HIV-1 being substantially non-pathogenic in humans and comprising a mutation in its genome corresponding to amino acids 162 to 177 of wild-type HIV-1 Nef such that these amino acids are substantially not represented in a Nef protein or derivative thereof produced by said isolated HIV-1 strain, or insufficient of the amino acid sequence is represented to induce an immune response to that region of Nef.

In a related embodiment, the genomic mutation in the non-pathogenic strain of HIV-1 is a mutation in one or more of nucleotides 9271 to 9317 relative to HIV-1_{NL43} or in a functionally equivalent region in another pathogenic strain of HIV-1.

In a related embodiment, there is provided a non-pathogenic strain of HIV-1 comprising a genome which is substantially incapable of hybridising under medium stringent conditions a nucleic acid molecule comprising that sequence of nucleotides which encodes all or part of amino acids 162 to 177 of wild-type HIV-1. Preferably, the nucleic acid molecule is a synthetic oligonucleotide.

In a particularly preferred embodiment, the present invention provides non-pathogenic HIV-1 isolate C18 deposited at the ECACC on 17 Oct. 1994 under Provisional Accession Number V94101706.

In a related embodiment, the present invention provides non-pathogenic HIV-1 isolate C98 deposited at the ECACC on 31 Oct. 1994 under Provisional Accession Number V941031169.

In another embodiment, tie present invention provides non-pathogenic HIV-1 isolate C54 deposited at ECACC on 10 Mar. 1995 under Provisional Accession No. V95031022.

Although pathogenicity is a relative term, it is used herein in relation to the capacity of a strain of HIV-1 to induce AIDS or AIDS-related disorders in an individual over time Accordingly, a “non-pathogenic” stain of HIV-1 is a stain which, at die clinical level, does not lead to th development of AIDS, at least within the median time of 6-10 years following infection with HIV-1. At the laboratory level, a non-pathogenic strain of HIV-1 is considered not to alter CD4+ cell counts or β₂-microglobulin concentrations. In addition, a non-pathogenic strain of HIV-1 may not alter CD8+ and CD3+ cell counts and would not alter lymphocyte counts. CD4+:CD8+ ratio also remain unchanged relative to normal non-infected individuals. Furthermore, generally, a non-pathogenic strain of HIV-1 does not induce p24 antigenaemia. A non-pathogenic HIV-1 of the present invention is generally still infectious but individuals infected with the virus remain free of symptoms for at least 6-10 years after infection.

A laboratory classified non-pathogenic strain of HIV-1 may be dec slat any time after infection. The term “non-pathogenic” is not to be considered as a strain that is never pathogenic under any conditions as this might depend on the host individual, the level of immune responsiveness in that individual and the extent or otherwise of other, for example, immune comprising disorders. Accordingly, a “non-pathogenic”HIV-1 isolate of the present invention may also be considered a “low virulent” strain of the virus. A non-pathogenic strain of HIV-1 as contemplated herein may be isolated from an asymptomatic individual or may be derived from a pathogenic strain by mutation. Although the present invention is not to be limited to any particular pathogenic strain of HIV-1, for reference purposes, an example of a pathogenic strain is HIV-1_{NL43} strain as described by Myers et al (1992; 1994).

The non-pathogenic nature of the HIV-1 of the present invention is conveniently evidenced by the cohort of seven individuals comprising one donor and six recipients which have remained free of symptoms or signs of HIV-1 infection for greater than the median time of 6-10 years. However, the individuals of the cohort are seropositive for HIV-1 following infection with the virus as determined by Western blot analysis and genetic analysis (e.g. using PCR techniques). A seropositive individual is one showing reactivity to at least one HIV-1 glycoprotein (such as but not limited gp 41-45, gp120, gp160) and at least three other virus-specific bands.

In accordance with the present invention, a non-pathogenic HIV-1 isolate is also a strain of HIV-1 which:
(i) induce an immune response in a human or primate subject; and
(ii) does not substantially reduce proliferative responses or cytokine production to a mitogen, alloantigen and/or recall antigen relative to a healthy, non-infected subject.

Preferably, the immune response such as to a glycoprotein, for example gp41-45, gp120 and/or gp160. Preferably, the cytokine monitored is an interleukin, such s IL-2. Preferably, the recall antigen is influenza or tetanus toxoid. A non-pathogenic HIV-1 isolate is also one which:
(iii) does not substantially alter proliferative responses or cytokine production to allorgeneic mononuclear cells.

Furthermore, a non-pathogenic strain of HIV-1 carries a deletion in an HIV-1-derived protein which results in an altered immunologic profile. In particularly preferred embodiment, the non-pathogenic strain of HIV-1 is substantially incapable of inducing an antibody response to amino acids 162 to 177 to wild-type HIV-1 Nef protein. According to this preferred aspect of the present invention, there is provided a non-pathogenic HIV-1 isolate which:
(i) induces an immune response in a human or primate subject;
(ii) does not substantially reduce proliferative responses or cytokine production to a mitogen, alloantigen and/or recall antigen relative to a healthy, non-infected subject; and
(iii) is substantially incapable of inducing an antibody response to amino acids 162 to 177 of wild-type HIV-1 Nef protein.

The genomes or complementary DNA therefrom of the non-pathogenic HIV-1 isolates of the present invention are capable of hybridising under medium stringency conditions to the corresponding genome or complementary DNA to a pathogenic strain of HIV-1 (e.g. HIV-1 strain HIV-1_{NL43}). The ability to hybridize to a pathogenic strain of HIV-1 only applies to a comparison of the entire genome/complementary DNA of a non-pathogenic strain or a fragment which includes genetic material corresponding to a region in the genome 3′ of the nef gene in a pathogenic strain of HIV-1.

Reference herein to “wild-type HIV-1” is meant to include reference to architypal pathogenic strain HIV-1_{NL43} (Myers et al, 1992; 1994). For the purposes of reference only, a suitable genomic nucleotide sequence from HIV-1_{NL43} is set forth in SEQ ID NO: 1 (Myers et al, 1992; 1994):

| | |
|---|---|
| 1 | TGGAAGGGCTAATTTGGTCCCAAAAAAGACAAGAGATCCTTGATCTGTGG |
| 51 | ATCTACCACACACAAGGCTACTTCCCTGATTGGCAGAACTACACACCAGG |
| 101 | GCCAGGGATCAGATATCCACTGACCTTTGGATGGTGCTTCAAGTTAGTAC |
| 151 | CAGTTGAACCAGAGCAAGTAGAAGAGGCCAAATAAGGAGAGAAGAACAGC |
| 201 | TTGTTACACCCTATGAGCCAGCATGGGATGGAGGACCCGGAGGGAGAAGT |
| 251 | ATTAGTGTGGAAGTTTGACAGCCTCCTAGCATTTCGTCACATGGCCCGAG |
| 301 | AGCTGCATCCGGAGTACTACAAAGACTGCTGACATCGAGCTTTCTACAAG |
| 351 | GGACTTTCCGCTGGGGACTTTCCAGGGAGGTGTGGCCTGGGCGGGACTGG |
| 401 | GGAGTGGCGAGCCCTCAGATGCTACATATAAGCAGCTGCTTTTTGCCTGT |
| 451 | ACTGGGTCTCTCTGGTTAGACCAGATCTGACCCTGGGAGCTCTCTGGCTA |
| 501 | ACTAGGGAACCCACTGCTTAAGCCTCAATAAAGCTTGCCTTGAGTGCTCA |
| 551 | AAGTAGTGTGTGCCCGTCTGTTGTGTGACTCTGGTAkCTAGAGATCCCTC |
| 601 | AGACCCTTTTAGTCAGTGTGGAAAATCTCTAGCAGTGGCGCCCGAACAGG |
| 651 | GACTTGAAAGCGAAAGTAAAGCCAGAGGAGATCTCTCGACGCAGGACTCG |
| 701 | GCTTGCTGAAGCGCGCACGGCAAGAGGCGAGGGGCGGCGACTGGTGAGTA |
| 751 | CGCCAAAAATTTTGACTAGCGGAGGCTAGAAGGAGAGAGATGGGTGCGAG |
| 801 | AGCGTCGGTATTAAGCGGGGGAGAATTAGATAAATGGGAAAAAATTCGGT |
| 851 | TAAGGCCAGGGGGAAAQAAACAATATAAACTAAAACATATAGTATGGGCA |
| 901 | AGCAGGGAGCTAGAACGATTCGCAGTTAATCCTGGCCTTTTAGAGACATC |
| 951 | AGAAGGCTGTAGACAAATACTGGGACAGCTACAACCATCCCTTCAGACAG |
| 1001 | GATCAGAAGAACTTAGATCATTATATAATACAATAGCAGTCCTCTATTGT |
| 1051 | GTGCATCAAAGGATAGATGTAAAAGACACCAAGGAAGCCTTAGATAAGAT |
| 1101 | AGAGGAAGAGCAAAACAAAAGTAAGAAAAAGGCACAGCAAGCAGCAGCTG |
| 1151 | ACACAGGAAACAACAGCCAGGTCAGCCAAAATTACCCTATAGTGCAGAAC |
| 1201 | CTCCAGGGGCAAATGGTACATCAGGCCATATCACCTAGAACTTTAAATGC |
| 1251 | ATGGGTAAAAGTAGTAGAAGAGAAGGCTTTCAGCCCAGAAGTAATACCCA |
| 1301 | TGTTTTCAGCATTATCAGAAGGAGCCACCCCACAAGATTTAAATACCATG |
| 1351 | CTAAACACAGTGGGGGGACATCAAGCAGCCATGCAAATGTTAAAAGAGAC |
| 1401 | CATCAATGAGGAAGCTGCAGAATGGGATAGATTGCATCCAGTGCATGCAG |
| 1451 | GGCCTATTGCACCAGGCCAGATGAGAGAACCAAGGGGAAGTGACATAGCA |
| 1501 | GGAACTACTAGTACCCTTCAGGAACAAATAGGATGGATGACACATAATCC |
| 1551 | ACCTATCCCAGTAGGAGAAATCTATAAAAGATGGATAATCCTGGGATTAA |
| 1601 | ATAAAATAGTAAGAATGTATAGCCCTACCAGCATTCTGGACATAAGACAA |
| 1651 | GGACCAAAGGAACCCTTTAGAGACTATGTAGACCGATTCTATAAAACTCT |
| 1701 | AAGAGCCGAGCAAGCTTCACAAGAGGTAAAAAATTGGATGACAGAAACCT |
| 1751 | TGTTGGTCCAAAATGCGAACCCAGATTGTAAGACTATTTTAAAAGCATTG |
| 1801 | GGACCAGGAGCGACACTAGAAGAAATGATGACAGCATGTCAGGGAGTGGG |
| 1851 | GGGACCCGGCCATAAAGCAAGAGTTTTGGCTGAAGCAATGAGCCAAGTAA |
| 1901 | CAAATCCAGCTACCATAATGATACAGAAAGGCAATTTTAGGAACCAAAGA |
| 1951 | AAGACTGTTAAGTGTTTCAATTGTGGCAAAGAAGGGCACATAGCCAAAAA |
| 2001 | TTGCAGGGCCCCTAGGAAAAAGGGCTGTTGGAAATGTGGAAAGGAAGGAC |
| 2051 | ACCAAATGAAAGATTGTACTGAGAGACAGGCTAATTTTTTAGGGAAGATC |
| 2101 | TGGCCTTCCCACAAGGGAAGGCCAGGGAATTTTCTTCAGAGCAGACCAGA |
| 2151 | GCCAACAGCCCCACCGAAGAAGAGCTTCAGGTTTGGGGAAGAGACAACAA |
| 2201 | CTCCCTCTCAGAAGCAGGAGCCGATAGACAAGGAACTGTATCCTTTAGCT |
| 2251 | TCCCTCAGATCACTCTTTGGCAGCGACCCCTCGTCACAATAAAGATAGGG |
| 2301 | GGGCAATTAAAGGAAGCTCTATTAGATACAGGAGCAGATGATACAGTATT |
| 2351 | AGAAGAAATGAATTTGCCAGGAAGATGGAAACCAAAAATGATAGGGGGAA |
| 2401 | TTGGAGGTTTTATCAAAGTAGGACAGTATGATCAGATACTCATAGAAATC |
| 2451 | TGCGGACATAAAGCTATAGGTACAGTATTAGTAGGACCTACACCTGTCAA |
| 2501 | CATAATTGGAAGAAATCTGTTGACTCAGATTGGCTGCACTTTAAATTTTC |
| 2551 | CCATTAGTCCTATTGAGACTGTACCAGTAAAATTAAAGCCAGGAATGGAT |
| 2601 | GGCCCAAAAGTTAAACAATGGCCATTGACAGAAGAAAAAATAAAAGCATT |
| 2651 | AGTAGAAATTTGTACAGAAATGGAAAAGGAAGGAAAAATTTCAAAAATTG |
| 2701 | GGCCTGAAAATCCATACAATACTCCAGTATTTGCCATAAAGAAAAAAGAC |
| 2751 | AGTACTAAATGGAGAAAATTAGTAGATTTCAGAGAACTTAATAAGAGAAC |
| 2801 | TCAAGATTTCTGGGAAGTTCAATTAGGAATACCACATCCTGCAGGGTTAA |
| 2851 | AACAGAAAAAATCAGTAACAGTACTGGATGTGGGCGATGCATATTTTTCA |
| 2901 | GTTCCCTTAGATAAAGACTTCAGGAAGTATACTGCATTTACCATACCTAG |
| 2951 | TATAAACAATGAGACACCAGGGATTAGATATCAGTACAATGTGCTTCCAC |
| 3001 | AGGGATGGAAAGGATCACCAGCAATATTCCAGTGTAGCATGACAAAAATC |
| 3051 | TTAGAGCCTTTTAGAAAACAAAATCCAGACATAGTCATCTATCAATACAT |
| 3101 | GGATGATTTGTATGTAGGATCTGACTTAGAAATAGGGCAGCATAGAACAA |
| 3151 | AAATAGAGGACTGAGACAACATCTGTTGAGGTGGGGGATTTACCACACCA |
| 3201 | GACAAAAAACATCAGAAAGAACCTCCATTCCTTTGGATGGGTTATGAACT |
| 3251 | CCATCCTGATAAATGGACAGTACAGCCTATAGTGCTGCCAGAAAAGGACA |
| 3301 | GCTGGACTGTCAATGACATACAGAAATTAGTGGGAAAATTGAATTGGGCA |
| 3351 | AGTCAGATTTATGCAGGGATTAAAAGTAAGGCAATATGTAAACTTCTTAG |
| 3401 | GGGAACCAAAGCACTAACAGAAGTAGTACCACTAACAGAAGAAGCAGAGC |
| 3451 | TAGAACTGGCAGAAAACAGGGAGATTCTAAAAGAACCGGTACATGGAGTG |
| 3501 | TATTATGACCCATCAAAAGACTTAATAGCAGAAATACAGAAGCAGGGGCA |
| 3551 | AGGCCAATGGACATATCAAATTTATCAAGAGCCATTTAAAAATCTGAAAA |
| 3601 | CAGGAAAATATGCAAGAATGAAGGGTGCCCACACTAATGATGTGAAACAA |
| 3651 | TTAACAGAGGCAGTACAAAAAATAGCCACAGAAAGCATAGTAATATGGGG |
| 3701 | AAAGACTCCTAAATTTAAATTACCCATACAAAAGGAAACATGGGAAGCAT |
| 3751 | GGTGGACAGAGTATTGGCAAGCCACCTGGATGTCCTGAGTGGAGTTTGTC |
| 3801 | AATACCCCTCCCTTAGTGAAGTTATGGTACCAGTTAGAGAAAGAACCCAT |
| 3851 | AATAGGAGCAGAAACTTTCTATGTAGATGGGGCAGCCAATAGGGAAACTA |
| 3901 | AATTAGGAAAAGCAGGATATGTAACTGACAGAGGAAGACAAAAAGTTGTC |
| 3951 | CCCCTAACGGACACAACAAATCAGAAGACTGAGTTACAAGCAATTCATCT |
| 4001 | AGCTTTGCAGGATTCGGGATTAGAAGTAAACATAGTGACAGACTCACAAT |
| 4051 | ATGCATTGGGAATCATTCAAGCACAACCAGATAAGAGTGAATCAGAGTTA |
| 4101 | GTCAGTCAAATAATAGAGCAGTTAATAAAAAAGGAAAAAGTCTACCTGGC |
| 4151 | ATGGGTACCAGCACACAAAGGAATTGGAGGAAATGAACAAGTAGATGGGT |
| 4201 | TGGTCAGTGCTGGAATCAGGAAAGTACTATTTTTAGATGGAATAGATAAG |
| 4251 | GCCCAAGAAGAACATGAGAAATATCACAGTAATTGGAGAGCAATGGCTAG |
| 4301 | TGATTTTAACCTACCACCTGTAGTAGCAAAAGAAATAGTAGCCAGCTGTG |
| 4351 | ATAAATGTCAGCTAAAAGGGGAAGCCATGCATGGACAAGTAGACTGTAGC |
| 4401 | CCAGGAATATGGCAGCTAGATTGTACACATTTAGAAGGAAAAGTTATCTT |
| 4451 | GGTAGCAGTTCATGTAGCCAGTGGATATATAGAAGCAGAAGTAATTCCAG |
| 4501 | CAGAGACAGGGCAAGAAACAGCATACTTCCTCTTAAAATTAGCAGGAAGA |
| 4551 | TGGCCAGTAAAAACAGTACATACAGACAATGGCAGCAATTTCACCAGTAC |
| 4601 | TACAGTTAAGGCCGCCTGTTGGTGGGCGGGGATCAAGCAGGAATTTGGCA |
| 4651 | TTCCCTACAATCCCCAAAGTCAAGGAGTAATAGAATCTATGAATAAAGAA |
| 4701 | TTAAAGAAAATTATAGGACAGGTAAGAGATCAGGCTGAACATCTTAAGAC |
| 4751 | AGCAGTACAAATGGCAGTATTCATCCACAATTTTAAAAGAAAAGGGGGGA |
| 4801 | TTGGGGGGTACAGTGCAGGGGAAAGAATAGTAGACATAATAGCAACAGAC |
| 4851 | ATACAAACTAAAGAATTACAAAAACAAATTACAAAAATTCAAAATTTTCG |
| 4901 | GGTTTATTACAGGGACAGCAGAGATCCAGTTTGGAAAGGACCAGCAAAGC |
| 4951 | TCCTCTGGAAAGGTGAAGGGGCAGTAGTAATACAAGATAATAGTGACATA |
| 5001 | AAAGTAGTGCCAAGAAGAAAAGCAAAGATCATCAGGGATTATGGAAAACA |
| 5051 | GATGGCAGGTGATGATTGTGTGGCAAGTAGACAGGATGAGGATTAACACA |
| 5101 | TGGAAAAGATTAGTAAAACACCATATGTATATTTCAAGGAAAGCTAAGGA |
| 5151 | CTGGTTTTATAGACATCACTATGAAAGTACTAATCCAAAAATAAGTTCAG |
| 5201 | AAGTACACATCCCACTAGGGGATGCTAAATTAGTAATAACAACATATTGG |
| 5251 | GGTCTGCATACAGGAGAAAGAGACTGGCATTTGGGTCAGGGAGTCTCCAT |
| 5301 | AGAATGGAGGAAAAAGAGATATAGCACACAAGTAGACCCTGACCTAGCAG |
| 5351 | ACCAACTAATTCATCTGCACTATTTTGATTGTTTTTCAGAATCTGCTATA |
| 5401 | AGAAATACCATATTAGGACGTATAGTTAGTCCTAGGTGTGAATATCAAGC |
| 5451 | AGGACATAACAAGGTAGGATCTCTACAGTACTTGGCACTAGCAGCATTAA |
| 5501 | TAAAACCAAAACAGATAAAGCCACCTTTGCCTAGTGTTAGGAAACTGACA |
| 5551 | GAGGACAGATGGAACAAGCCCCAGAAGACCAAGGGCCACAGAGGGAGCCA |
| 5601 | TACAATGAATGGACACTAGAGCTTTTAGAGGAACTTAAGAGTGAAGCTGT |
| 5651 | TAGACATTTTCCTAGGATATGGCTCCATAACTTAGGACAACATATCTATG |
| 5701 | AAACTTACGGGGATACTTGGGCAGGAGTGGAAGCCATAATAAGAATTCTG |
| 5751 | CAACAACTGCTGTTTATCCATTTCAGAATTGGGTGTCGACATAGCAGAAT |
| 5801 | AGGCGTTACTCGACAGAGGAGAGCAAGAAATGGAGCCAGTAGATCCTAGA |
| 5851 | CTAGAGCCCTGGAAGCATCCAGGAAGTCAGCCTAAAACTGCTTGTACCAA |
| 5901 | TTGCTATTGTAAAAAGTGTTGCTTTCATTGCCAAGTTTGTTTCATGACAA |
| 5951 | AAGCCTTAGGCATCTCCTATGGCAGGAAGAAGCGGAGACAGCGACGAAGA |
| 6001 | GCTCATCAGAACAGTCAGACTCATCAAGCTTCTCTATCAAAGCAGTAAGT |
| 6051 | AGTACATGTAATGCAACCTATAATAGTAGCAATAGTAGCATTAGTAGTAG |
| 6101 | CAATAATAATAGCAATAGTTGTGTGGTCCATAGTAATCATAGAATATAGG |
| 6151 | AAAATATTAAGACAAAGAAAAATAGACAGGTTAATTGATAGACTAATAGA |
| 6201 | AAGAGCAGAAGACAGTGGCAATGAGAGTGAAGGAGAAGTATCAGCACTTG |
| 6251 | TGGAGATGGGGGTGGAAATGGGGCACCATGCTCCTTGGGATATTGATGAT |
| 6301 | CTGTAGTGCTACAGAAAAATTGTGGGTCACAGTCTATTATGGGGTACCTG |
| 6351 | TGTGGAAGGAAGCAACCACCACTCTATTTTGTGCATCAGATGCTAAAGCA |
| 6401 | TATGATACAGAGGTACATAATGTTTGGGCCACACATGCCTGTGTACCCAC |
| 6451 | AGACCCCAACCCACAAGAAGTAGTATTGGTAAATGTGACAGAAAATTTTA |
| 6501 | ACATGTGGAAAAATGACATGGTAGAACAGATGCATGAGGATATAATCAGT |
| 6551 | TTATGGGATCAAAGCCTAAAGCCATGTGTAAAATTAACCCCACTCTGTGT |
| 6601 | TAGTTTAAAGTGCACTGATTTGAAGAATGATACTAATACCAATAGTAGTA |
| 6651 | GCGGGAGAATGATAATGGAGAAAGGAGAGATAAAAAACTGCTCTTTCAAT |
| 6701 | ATCAGCACAAGCATAAGAGATAAGGTGCAGAAAGAATATGCATTCTTTTA |
| 6751 | TAAACTTGATATAGTACCAATAGATAATACCAGCTATAGGTTGATAAGTT |
| 6801 | GTAACACCTCAGTCATTACACAGGCCTGTCCAAAGGTATCCTTTGAGCCA |
| 6851 | ATTCCCATACATTATTGTGCCCCGGCTGGTTTTGCGATTCTAAAATGTAA |
| 6901 | TAATAAGACGTTCAATGGAACAGGACCATGTACAAATGTCAGCACAGTAC |
| 6951 | AATGTACACATGGAATCAGGCCAGTAGTATCAACTCAACTGCTGTTAAAT |
| 7001 | GGCAGTCTAGCAGAAGAAGATGTAGTAATTAGATCTGCCAATTTCACAGA |
| 7051 | CAATGCTAAAACCATAATAGTACAGCTGAACACATCTGTAGAAATTAATT |
| 7101 | GTACAAGACCCAACAACAATACAAGAAAAAGTATCCGTATCCAGAGGGGA |
| 7151 | CCAGGGAGAGCATTTGTTACAATAGGAAAAATAGGAAATATGAGACAAGC |
| 7201 | ACATTGTAACATTAGTAGAGCAAAATGGAATGCCACTTTAAAACAGATAG |
| 7251 | CTAGCAAATTAAGAGAACAATTTGGAAATAATAAAACAATAATCTTTAAG |
| 7301 | CAATCCTCAGGAGGGGACCCAGAAATTGTAACGCACAGTTTTAATTGTGG |
| 7351 | AGGGGAATTTTTCTACTGTAATTCAACACAACTGTTTAATAGTACTTGGT |
| 7401 | TTAATAGTACTTGGAGTACTGAAGGGTCAAATAACACTGAAGGAAGTGAC |
| 7451 | ACAATCACACTCCCATGCAGAATAAAACAATTTATAAACATGTGGCAGGA |
| 7501 | AGTAGGAAAAGCAATGTATGCCCCTCCCATCAGTGGACAAATTAGATGTT |
| 7551 | CATCAAATATTACTGGGCTGCTATTAACAAGAGATGGTGGTAATAACAAC |
| 7601 | AATGGGTCCGAGATCTTCAGACCTGGAGGAGGCGATATGAGGGACAATTG |
| 7651 | GAGAAGTGAATTATATAAATATAAAGTAGTAAAAATTGAACCATTAGGAG |
| 7701 | TAGCACCCACCAAGGCAAAGAGAAGAGTGGTGCAGAGAGAAAAAAGAGCA |
| 7751 | GTGGGAATAGGAGCTTTGTTCCTTGGGTTCTTGGGAGCAGCAGGAAGCAC |
| 7801 | TATGGGCTGCACGTCAATGACGCTGACGGTACAGGCCAGACAATTATTGT |
| 7851 | CTGATATAGTGCAGCAGCAGAACAATTTGCTGAGGGCTATTGAGGCGCAA |
| 7901 | CAGCATCTGTTGCAACTCACAGTCTGGGGCATCAAACAGCTCCAGGCAAG |
| 7951 | AATCCTGGCTGTGGAAAGATACCTAAAGGATCAACAGCTCCTGGGGATTT |
| 8001 | GGGGTTGCTCTGGAAAACTCATTTGCACCACTGCTGTGCCTTGGAATGCT |
| 8051 | AGTTGGAGTAATAAATCTCTGGAACAGATTTGGAATAACATGACCTGGAT |
| 8101 | GGAGTGGGACAGAGAAATTAACAATTACACAAGCTTAATACACTCCTTAA |
| 8151 | TTGAAGAATCGCAAAACCAGCAAGAAAAGAATGAACAAGAATTATTGGAA |
| 8201 | TTAGATAAATGGGCAAGTTTGTGGAATTGGTTTAACATAACAAATTGGCT |
| 8251 | GTGGTATATAAATTATTCATAATGATAGTAGGATGGCTTGGTAGGTTTAA |
| 8301 | GAATAGTTTTTGCTGTACTTTCTATAGTGAATAGAGTTAGGCAGGGATAT |
| 8351 | TCACCATTATCGTTTCAGACCCACCTCCCAATCCCGAGGGGACCCGACAG |
| 8401 | GCCCGAAGGAATAGAAGAAGAAGGTGGAGAGAGAGACAGAGACAGATCCG |
| 8451 | TTCGATTAGTGAACGGATCCTTAGCACTTATCTGGGACGATCTGCGGAGC |
| 8501 | CTGTGCCTCTTCAGCTACCACCGCTTGAGAGACTTACTCTTGATTGTAAC |
| 8551 | GAGGATTGTGGAACTTCTGGGACGCAGGGGGTGGGAAGCCCTCAAATATT |
| 8601 | GGTGGAATCTCCTACAGTATTGGAGTCAGGAACTAAAGAATAGTGCTGTT |
| 8651 | AACTTGCTCAATGCCACAGCCATAGCAGTAGCTGAGGGGACAGATAGGGT |
| 8701 | TATAGAAGTATTACAAGCAGCTTATAGAGCTATTCGCCACATACCTAGAA |
| 8751 | GAATAAGACAGGGCTTGGAAAGGATTTTGCTATAAGATGGGTGGCAAGTG |
| 8801 | GTCAAAAAGTAGTGTGATTGGATGGCCTGCTGTAAGGGAAAGAATGAGAC |
| 8851 | GAGCTGAGCCAGCAGCAGATGGGGTGGGAGCAGTATCTCGAGACCTAGAA |
| 8901 | AAACATGGAGCAATCACAAGTAGCAATACAGCAGCTAACAATGCTGCTTG |
| 8951 | TGCCTGGCTAGAAGCACAAGAGGAGGAAGAGGTGGGTTTTCCAGTCACAC |
| 9001 | CTCAGGTACCTTTAAGACCAATGACTTACAAGGCAGCTGTAGATCTTAGC |
| 9051 | CACTTTTTAAAAGAAAAGGGGGGACTGGAAGGGCTAATTCACTCCCAAAG |
| 9101 | AAGACAAGATATCCTTGATCTGTGGATCTACCACACACAAGGCTACTTCC |
| 9151 | CTGATTGGCAGAACTACACACCAGGGCCAGGGGTCAGATATCCACTGACC |
| 9201 | TTTGGATGGTGCTACAAGCTAGTACCAGTTGAGCCAGATAAGGTAGAAGA |
| 9251 | GGCCAATAAAGGAGAGAACACCAGCTTGTTACACCCTGTGAGCCTGCATG |
| 9301 | GAATGGATGACCCTGAGAGAGAAGTGTTAGAGTGGAGGTTTGACAGCCGC |
| 9351 | CTAGCATTTCATCACGTGGCCCGAGAGCTGCATCCGGAGTACTTCAAGAA |
| 9401 | CTGCTGACATCGAGCTTGCTACAAGGGACTTTCCGCTGGGGACTTTCCAG |
| 9451 | GGAGGCGTGGCCTGGGCGGGACTGGGGAGTGGCGAGCCCTCAGATGCTGC |
| 9501 | ATATAAGCAGCTGCTTTTTGCCTGTACTGGGTCTCTCTGGTTAGACCAGA |
| 9551 | TCTGAGCCTGGGAGCTCTCTGGCTAACTAGGGAACCCACTGCTTAAGCCT |
| 9601 | CAATAAAGCTTGCCTTGAGTGCTTCAAGTGATGTGTGCCCGTCTGTTGTG |
| 9651 | TGACTCTGGTAACTAGAGATCCCTCAGACCCTTTTAGTCAGTGTGGAAAA |
| 9701 | TCTCTAGCA |

However, for the purposes of comparing the nucleotide sequences of non-pathogenic HIV-1 strains including the ability to hybridize to a reference strain, the present invention extends to a genomic nucleotide sequence from any pathogenic strain of HIV-1.

Reference to a biological source includes blood or blood-related products or components such as lymphocytes, plasma, tissue fluid and tissue extract.

Accordingly, in a particularly preferred embodiment, there is provided a viral isolate which:
(i) carries a genome which is capable of hybridising under medium stringency conditions to SEQ ID NO: 1 or a complementary form thereof or an analogous sequence from another pathogenic strain of HIV-1; and
(ii) caries a deletion mutation in a region corresponding to the nef gene and/or in an LTR region. Generally, such an HIV-1 isolate is non-pathogenic as hereinbefore defined.

In a related embodiment, there is provided an isolated virus which:
(i) has a genome which is capable of hybridising under medium stringency conditions to complementary nucleic acid from a pathogenic strain of HIV-1; and
(ii) caries one or more deletion mutations in a region of its genome corresponding to a region which contains nef coding sequences and LTR nucleotide sequences.

For the purposes of defining the level of stringency, reference can conveniently be made to Maniatis et al (1982) at pages 387-389 which is herein incorporated by reference where the washing steps disclosed arm considered high stringency. A low stringency is defined herein as being in 1-3×SSC/0.1-0.5% w/v SDS at 37-50° C. or 2-3 hours. Depending on the source and concentration of nucleic acid involved in the hybridisation, alternative conditions of stringency may be employed such as medium stringent conditions which are considered herein to be 0.1-1×SSC/0.25-0.5% w/v SDS at ≧45° C. for 2-3 hours or high stringent conditions considered herein to be 0.1-1×SSC/0.1% w/v SDS at 60° C. for 1-3 hours.

In a particularly preferred embodiment of the present invention, the non-pathogenic strain of HIV-1 carries a mutation in the nef gene and/or LTR legion of the genome. In an even more preferred embodiment, the non-pathogenic strain of HIV-1 carries a mutation in the nef gene such that a Nef protein is not produced or a modified Nef protein is produced substantially not carrying amino acids 162 to 177 or a portion thereof from wild-type Nef.

A “mutation” is considered herein to include a single or multiple nucleotide substitution, deletion and/or addition Most preferred mutations are single or multiple deletions of at least one, most preferably at least ten and even more preferably at least twenty contiguous nucleotides from a region corresponding to the nef gene and/or the LTR region. When the non-pathogenic virus carries a mutation in the LTR region, this generally occurs 5′ of the Sp1 sites. A particularly preferred deletion is from a region within the nef gene corresponding to amino acids 162 to 177 of the Nef protein.

According to a preferred aspect of the present invention, there is provided a viral isolate which:
(i) is reactive to antibodies to a glycoprotein from HIV-1 such as at least one of gp41-45, gp120 and/or gp160;
(ii) is substantially non-pathogenic in human subjects; and
(iii) cares a deletion mutation of at least ten nucleotides in a region corresponding to the nef gene an/or LTR region of a pathogenic strain of HIV-1.

In another embodiment, there is provided a viral isolate which:
(i) is capable of inducing an immune response to at least one of gag, pol and/or env;
(ii) is substantially nonpathogenic in human subjects; and
(iii) carries a deletion mutation of at left ten nucleotides in a region corresponding to the nef gene and/or LTR region of a pathogenic strain of HIV-1.

Preferably, in respect of the latter embodiment, the immune response is an antibody or a cell mediated response. In a most preferred embodiment, the immune response is a humoral immune response.

In another aspect of the present invention, there is provided a viral isolate which:
(i) is interactive to antibodies toga glycoprotein from HIV-1 as at least one of gp41-45, gp120 and/or gp160;
(ii) is substantially non-pathogenic in human subjects; and
(iii) carries a deletion mutation of at least ten nucleotides in a corresponding to all or part of amino acids 162 to 177 encoded by the nef gene of a pathogenic strain of HIV-1.

The nucleotide sequence of the nef gene in HIV-1_{NL43} is defined in SEQ ID NO: 650:

ATGGGTGGCAAGTGGTCAAAAAGTAGTGTGATTGGATGGCCTGCTGTAAGGGAAAGAAT
GAGACGAGCTGAGCCAGCAGCAGATGGGGTGGGAGCAGTATCTCGAGACCTAGAAAAAC
ATGGAGCAATCACAAGTAGCAATACAGCAGCTAACAATGCTGCTTGTGCCTGGCTAGAA
GCACAAGAGGAGGAAGAGGTGGGTTTTCCAGTCACACCTCAGGTACCTTTAAGACCAAT
GACTTACAAGGCAGCTGTAGATCTTAGCCACTTTTTAAAAGAAAAGGGGGGACTGGAAG
GGCTAATTCACTCCCAAAGAAGACAAGATATCCTTGATCTGTGGATCTACCACACACAA
GGCTACTTCCCTGATTGGCAGAACTACACACCAGGGCCAGGGGTCAGATATCCACTGAC
CTTTGGATGGTGCTACAAGCTAGTACCAGTTGAGCCAGATAAGGTAGAAGAGGCCAATA
AAGGAGAGAACACCAGCTTGTTACACCCTGTGAGCCTGCATGGAATGGATGACCCTGAG
AGAGAAGTGTTAGAGTGGAGGTTTGACAGCCGCCTAGCATTTCATCACGTGGCCCGAGA
GCTGCATCCGGAGTACTTCAAGAACTGCTGA

The nucleotide sequence encoding amino acids 162-177 of wild-type HIV-1_{NL43} Nef is as follows:

[SEQ ID NO:802]
ACCAGCTTGTTACACCCTGTGAGCCTGCATGGAATGGATGACCCTGAG.

The present invention extends to any or all single or multiple nucleotide deletions to a contiguous series of at least ten nucleotides from the nef gene which render the strain avirulent. The deletions may encompass the entire gene or parts thereof and may represent a single deletion or two or more deletions. Put in alternative terms, the non-pathogenic HIV-1 isolates of the present invention comprise a nucleotide sequence at the corresponding nef gene region non-identifiable to SEQ ID NO: 650, non-identity comprising at least 5%, more preferably at least 100% and even more preferably at least 20% variation thereon. The present invention particularly extends to any or all single or multiple nucleotide deletions to a contiguous series of at least ten nucleotides from the region of the nef gene corresponding to all or part of amino acids 162 to 177 and which render the strain avirulent. Although this aspect of the present invention is exemplified by specific reference to amino acids 162 to 177, the present invention also extends to a functionally analogous sequence where, for example, acids are substituted by structurally or functionally similar amino acids. In addition, the present invention extends to any deletion or other mutation in an HIV-1 derived protein which renders that strain non-pathogenic.

In a preferred embodiment, therefore, the present invention contemplates a viral isolate which:
(i) is reactive to antibodies to a glycoprotein from HIV-1 such as at leas one of gp41-45, gp120 and/or gp160;
(ii) carries a genome or a part or fragment thereof capable of hybridising under medium stringency conditions to a nucleotide sequence as set forth in SEQ ID NO: 1 or a complementary form thereof or an analogous sequence from another pathogenic strain of HIV-1;
(iii) carries a deletion of at least ten nucleotides in a region corresponding to the nef gene in HIV-1_{NL43}; and

wherein said deletion encompass one or more of the following decanucleotides from the nef gene of HIV-1_{NL43} or corresponding sequences from another pathogenic strain of HIV-1:

| | |
|---|---|
| ATGGGTGGCA; | (SEQ ID NO: 2) |
| TGGGTGGCAA; | (SEQ ID NO: 3) |
| GGGTGGCAAG; | (SEQ ID NO: 4) |
| GGTGGCAAGT; | (SEQ ID NO: 5) |
| GTGGCAAGTG; | (SEQ ID NO: 6) |
| TGGCAAGTGG; | (SEQ ID NO: 7) |
| GGCAAGTGGT; | (SEQ ID NO: 8) |
| GCAAGTGGTC; | (SEQ ID NO: 9) |
| CAAGTGGTCA; | (SEQ ID NO: 10) |
| AAGTGGTCAA; | (SEQ ID NO: 11) |
| AGTGGTCAAA; | (SEQ ID NO: 12) |
| GTGGTCAAAA; | (S3Q ID NO: 13) |
| TGGTCAAAAA; | (SEQ ID NO: 14) |
| GGTCAAAAAG; | (SEQ ID NO; 15) |
| GTCAAAAAGT; | (SEQ ID NO: 16) |
| TCAAAAAGTA; | (SEQ ID NO: 17) |
| CAAAAAGTAG; | (SEQ ID NO: 16) |
| AAAAAGTAGT; | (SEQ ID NO: 19) |
| AAAAGTAGTG; | (SEQ ID NO: 20) |
| AAAGTAGTGT; | (SEQ ID NO: 21) |
| AAGTAGTGTG; | (SEQ ID NO: 22) |
| AGTAGTGTGA; | (SEQ ID NO: 23) |
| GTAGTGTGAT; | (SEQ ID NO: 24) |
| TAGTGTGATT; | (SEQ ID NO: 25) |
| AGTGTGATTG; | (SEQ ID NO: 26) |
| GTGTGATTGG; | (SEQ ID NO: 27) |
| TGTGATTGGA; | (SEQ ID NO: 28) |
| GTGATTCGAT; | (SEQ ID NO: 29) |
| TGATTGGATG; | (SEQ ID NO: 30) |
| GATTGGATGG; | (SEQ ID NO: 31) |
| ATTGGATGGC; | (SEQ ID NO: 32) |
| TTGGATGGCC; | (SEQ ID NO: 33) |
| TGGATGGCCT; | (SEQ ID NO: 34) |
| GGATGGCCTG; | (SEQ ID NO: 35) |
| GATGGCCTGC; | (SEQ ID NO: 36) |
| ATGGCCTGCT; | (SEQ ID NO: 37) |
| TGGCCTGCTG; | (SEQ ID NO: 38) |
| GGCCTGCTGT; | (SEQ ID NO: 39) |
| GCCTGCTGTA; | (SEQ ID NO: 40) |
| CCTGCTGTAA; | (SEQ ID NO: 41) |
| CTGCTGTAAG; | (SEQ ID NO: 42) |
| TGCTGTAAGG; | (SEQ ID NO: 43) |
| GCTGTAAGGG; | (SEQ ID NO: 44) |
| CTGTAAGGGA; | (SEQ ID NO: 45) |
| TGTAAGGGAA; | (SEQ ID NO: 46) |
| GTAAGGGAAA; | (SEQ ID NO: 47) |
| TAAGGGAAAG; | (SEQ ID NO: 48) |
| AAGGGAAAGA; | (SEQ ID NO: 49) |
| AGGGAAAGAA; | (SEQ ID NO: 50) |
| GGGAAAGAAT; | (SEQ ID NO: 51) |
| GGAAAGAATG; | (SEQ ID NO: 52) |
| GAAAGAATGA; | (SEQ ID NO: 53) |
| AAAGAATGAG; | (SEQ ID NO: 54) |
| AAGAATGAGA; | (SEQ ID NO: 55) |
| AGAATGAGAC; | (SEQ ID NO: 56) |
| GAATGAGACG; | (SEQ ID NO: 57) |
| AATGAGACGA; | (SEQ ID NO: 58) |
| ATGAGACGAG; | (SEQ ID NO: 59) |
| TGAGACGAGC; | (SEQ ID NO: 60) |
| GAGACGAGCT; | (SEQ ID NO: 61) |
| AGACGAGCTG; | (SEQ ID NO: 62) |
| GACGAGCTGA; | (SEQ ID NO: 63) |
| ACGAGCTGAG; | (SEQ ID NO: 64) |
| CGAGCTGAGC; | (SEQ ID NO: 65) |
| GAGCTGAGCC; | (SEQ ID NO: 66) |
| AGCTCAGCCA; | (SEQ ID NO: 67) |
| GCTGAGCCAG; | (SEQ ID NO: 68) |
| CTGAGCCAGC; | (SEQ ID NO: 69) |
| TGAGCCAGCA; | (SEQ ID NO: 70) |
| GAGCCAGCAG; | (SEQ ID NO: 71) |
| AGCCAGCAGC; | (SEQ ID NO: 72) |
| GCCAGCAGCA; | (SEQ ID NO: 73) |
| CCAGCAGCAG; | (SEQ ID NO: 74) |
| CAGCAGCAGA; | (SEG ID NO: 75) |
| AGCAGCAGAT; | (SEQ ID NO: 76) |
| GCAGCAGATG; | (SEQ ID NO: 77) |
| CAGCAGATGG; | (SEQ ID NO: 78) |
| AGCAGATGGG; | (SEQ ID NO: 79) |
| GCAGATGGGG; | (SEQ ID NO: 80) |
| CAGATGGGGT; | (SEQ ID NO: 81) |
| AGATGGGGTG; | (SEQ ID NO: 82) |
| GATGGGGTGG; | (SEQ ID NO: 83) |
| ATGGGGTGGG; | (SEQ ID NO; 84) |
| TGGGGTGGGA; | (SEQ ID NO: 85) |
| GGGGTGGGAG; | (SEQ ID NO: 86) |
| GGGTGGGAGC; | (SRG ID NO: 87) |
| GGTGGGAGCA; | (SEQ ID NO: 88) |
| GTGGGAGCAG; | (SEQ ID NO: 89) |
| TGGGAGCAGT; | (SEQ ID NO: 90) |
| GGGAGCAGTA; | (SEQ ID NO: 91) |
| GGAGCAGTAT; | (SEQ ID NO: 92) |
| GAGCAGTATC; | (SEQ ID NO: 93) |
| AGCAGTATCT; | (SEQ ID NO: 94) |
| GCAGTATCTC; | (SEQ ID NO: 95) |
| CAGTATCTCG; | (SEQ ID NO: 96) |
| AGTATCTCGA; | (SEQ ID NO: 97) |
| GTATCTCGAG; | (SEQ ID NO: 98) |
| TATCTCGAGA; | (SEQ ID NO: 99) |
| ATCTCGAGAC; | (SEQ ID NO: 100) |
| TCTCGAGACC; | (SEQ ID NO: 101) |
| CTCGAGACCT; | (SEQ ID NO: 102) |
| TCGAGACCTA; | (SEQ ID NO: 103) |
| CGAGACCTAG; | (SEQ ID NO: 104) |
| GAGACCTAGA; | (SEQ ID NO: 105) |
| AGACCTAGAA; | (SEQ ID NO: 106) |
| GACCTAGAAA; | (SEQ ID NO: 107) |
| ACCTAGAAAA; | (SEQ ID NO: 108) |
| CCTAGAAAAA; | (SEQ ID NO: 109) |
| CTAGAAAAAC; | (SEQ ID NO: 110) |
| TAGAAAAACA; | (SEQ ID NO: 111) |
| AGAAAAACAT; | (SEQ ID NO: 112) |
| GAAAAACATG; | (SEQ ID NO: 113) |
| AAAAACATGG; | (SEQ ID NO: 114) |
| AAAACATGGA; | (SEQ ID NO: 115) |
| AAACATGGAG; | (SEQ ID NO: 116) |
| AACATGGAGC; | (SEQ ID NO: 117) |
| ACATGGAGCA; | (SEQ ID NO: 118) |
| CATGGAGCAA; | (SEQ ID NO: 119) |
| ATGGAGCAAT; | (SEQ ID NO: 120) |
| TGGAGCAATC; | (SEQ ID NO: 121) |
| GGAGCAATCA; | (SEQ ID NO: 122) |
| GAGCAATCAC; | (SEQ ID NO: 123) |
| AGCAATCACA; | (SEQ ID NO: 124) |
| GCAATCACAA; | (SEQ ID NO: 125) |
| CAATCACAAG; | (SEQ ID NO: 126) |
| AATCACAAGT; | (SEQ ID NO: 127) |
| ATCACAAGTA; | (SEQ ID NO: 128) |
| TCACAAGTAG; | (SEQ ID NO: 129) |
| CACAAGTAGC; | (SEQ ID NO: 130) |
| ACAAGTAGCA; | (SEQ ID NO: 131) |
| CAAGTAGCAA; | (SEQ ID NO: 132) |
| AAGTAGCAAT; | (SEQ ID NO: 133) |
| AGTAGCAATA; | (SEQ ID NO: 134) |
| GTAGCAATAC; | (SEQ ID NO: 135) |
| TAGCAATACA; | (SEQ ID NO: 136) |
| AGcAATACAG; | (SEQ ID NO: 137) |
| GCAATACAGC; | (SEQ ID NO: 138) |
| CAATACAGCA; | (SEQ ID NO: 139) |
| AATACAGCAG; | (SEQ ID NO: 140) |
| ATACAGCAGC; | (SEQ ID NO: 141) |
| TACAGCAGCT; | (SEQ ID NO: 142) |
| ACAGCAGCTA; | (SEQ ID NO: 143) |
| CAGCAGCTAA; | (SEQ ID NO: 144) |
| AGCAGCTAAC; | (SEQ ID NO: 145) |
| GCAGCTAACA; | (SEQ ID NO: 146) |
| CAGCTAACAA; | (SEQ ID NO: 147) |
| AGCTAACAAT; | (SEQ ID NO: 148) |
| GCTAACAATG; | (SEQ ID NO: 149) |
| CTAACAATGC; | (SEQ ID NO: 150) |
| TAACAATGCT; | (SEQ ID NO: 151) |
| AACAATGCTG; | (SEQ ID NO: 152) |
| ACAATGCTGC; | (SEQ ID NO: 153) |
| CAATGCTGCT; | (SEQ ID NO: 154) |
| AATGCTGCTT; | (SEQ ID NO: 155) |
| ATGCTGCTTG; | (SEQ ID NO: 156) |
| TGCTGCTTGT; | (SEQ ID NO: 157) |
| GCTGCTTGTG; | (SEQ ID NO: 158) |
| CTGCTTGTGC; | (SEQ ID NO: 159) |
| TGCTTGTGCC; | (SEQ ID NO: 160) |
| GCTTGTGCCT; | (SEQ ID NO: 161) |
| CTTGTGCCTG; | (SEQ ID NO: 162) |
| TTGTGCCTGG; | (SEQ ID NO: 163) |
| TGTGCCTGGC; | (SEQ ID NO: 164) |
| GTGCCTGGCT; | (SEQ ID NO: 165) |
| TGCCTGGCTA; | (SEQ ID NO: 166) |
| GCCTGGCTAG; | (SEQ ID NO: 167) |
| CCTGGCTAGA; | (SEQ ID NO: 168) |
| CTGGCTAGAA; | (SEQ ID NO: 169) |
| TGGCTAGAAG; | (SEQ ID NO: 170) |
| GGCTAGAAGC; | (SEQ ID NO: 171) |
| GCTAGAAGCA; | (SEQ ID NO: 172) |
| CTAGAAGCAC; | (SEQ ID NO: 173) |
| TAGAAGCACA; | (SEQ ID NO: 174) |
| AGAAGCACAA; | (SEQ ID NO: 175) |
| GAAGCACAAG; | (SEQ ID NO: 176) |
| AAGCACAAGA; | (SEQ ID NO: 177) |
| AGCACAAGAG; | (SEQ ID NO: 178) |
| GCACAAGAGG; | (SEQ ID NO: 179) |
| CACAAGAGGA; | (SEQ ID NO: 180) |
| ACAAGAGGAG; | (SEQ ID NO: 181) |
| CAAGAGGAGG; | (SEQ ID NO: 182) |
| AAGAGGAGGA; | (SEQ ID NO: 183) |
| AGAGGAGGAA; | (SEQ ID NO: 184) |
| GAGGAGGAAG; | (SEQ ID NO: 185) |
| AGGAGGAAGA; | (SEQ ID NO: 186) |
| GGAGGAAGAG; | (SEQ ID NO: 187) |
| GAGGAAGAGG; | (SEQ ID NO: 188) |
| AGGAAGAGGT; | (SEQ ID NO: 189) |
| GGAAGAGGTG; | (SEQ ID NO: 190) |
| GAAGAGGTGG; | (SEQ ID NO: 191) |
| AAGAGGTGGG; | (SEQ ID NO: 192) |
| AGAGGTGGGT; | (SEQ ID NO: 193) |
| GAGGTGGGTT; | (SEQ ID NO: 194) |
| AGGTGGGTTT; | (SEQ ID NO: 195) |
| GGTGGGTTTT; | (SEQ ID NO: 196) |
| GTGGGTTTTC; | (SEQ ID NO: 197) |
| TGGGTTTTCC; | (SEQ ID NO: 198) |
| GGGTITTCCA; | (SEQ ID NO: 199) |
| GGTTTTCCAG; | (SEQ ID NO: 200) |
| GTTTTCCAGT; | (SEQ ID NO: 201) |
| TTTTCCAGTC; | (SEQ ID NO: 202) |
| TTTCCAGTCA; | (SEQ ID NO: 203) |
| TTCCAGTCAC; | (SEQ ID NO: 204) |
| TCCAGTCACA; | (SEQ ID NO: 205) |
| CCAGTCACAC; | (SEQ ID NO: 206) |
| CAGTCACACC; | (SEQ ID NO: 207) |
| AGTCACACCT; | (SEQ ID NO: 208) |
| GTCACACCTC; | (SEQ ID NO: 209) |
| TCACACCTCA; | (SEQ ID NO: 210) |
| CACACCTCAG; | (SEQ ID NO: 211) |
| ACACCTCAGG; | (SEQ ID NO: 212) |
| CACCTCAGGT; | (SEQ ID NO: 213) |
| ACCTCAGGTA; | (SEQ ID NO: 214) |
| CCTCAGGTAC; | (SEQ ID NO: 215) |
| CTCAGGTACC; | (SEQ ID NO: 216) |
| TCAGGTACCT; | (SEQ ID NO: 217) |
| CAGGTACCTT; | (SEQ ID NO: 218) |
| AGGTACCTTT; | (SEQ ID NO: 219) |
| GGTACCTTTA; | (SEQ ID NO: 220) |
| GTACCTITAA; | (SEQ ID NO: 221) |
| TACCTTTAAG; | (SEQ ID NO: 222) |
| ACCTTTAAGA; | (SEQ ID NO: 223) |
| CCTTTAAGAC; | (SEQ ID NO: 224) |
| CTTTAAGACC; | (SEQ ID NO: 225) |
| TTTAAGACCA; | (SEQ ID NO: 226) |
| TTAAGACCAA; | (SEQ ID NO: 227) |
| TAAGACCAAT; | (SEQ ID NO: 228) |
| AAGACCAATG; | (SEQ ID NO: 229) |
| AGACCAATGA; | (SEQ ID NO: 230) |
| GACCAATGAC; | (SEQ ID NO: 231) |
| ACCAATGACT; | (SEQ ID NO: 232) |
| CCAATGACTT; | (SEQ ID NO: 233) |
| CAATGACTTA; | (SEQ ID NO: 234) |
| AATGACTTAC; | (SEQ ID NO: 235) |
| ATGACTAACA; | (SEQ ID NO: 236) |
| TGACTTACAA; | (SEQ ID NO: 237) |
| GACTTACAAG; | (SEQ ID NO: 238) |
| ACTTACAAGG; | (SEQ ID NO: 239) |
| CTTACAAGGC; | (SEQ ID NO: 240) |
| TTACAAGGCA; | (SEQ ID NO: 241) |
| TACAAGGCAG; | (SEQ ID NO: 242) |
| ACAAGGCAGC; | (SEQ ID NO: 243) |
| CAAGGCAGCT; | (SEQ ID NO: 244) |
| AAGGCAGCTG; | (SEQ ID NO: 245) |
| AGGCAGCTGT; | (SEQ ID NO: 246) |
| GGCAGCTGTA; | (SEQ ID NO: 247) |
| GCAGCTGTAG; | (SEQ ID NO: 248) |
| CAGCTGTAGA; | (SEQ ID NO: 249) |
| AGCTGTAGAT; | (SEQ ID NO: 250) |
| GCTGTAGATC; | (SEQ ID NO: 251) |
| CTGTAGATCT; | (SEQ ID NO: 252) |
| TGTAGATCTT; | (SEQ ID NO: 253) |
| GTAGATCTTA; | (SEQ ID NO: 254) |
| TAGATCTTAG; | (SEQ ID NO: 255) |
| AGATCTTAGC; | (SEQ ID NO: 256) |
| GATCTTAGCC; | (SEQ ID NO: 257) |
| ATCTTAGCCA; | (SEQ ID NO: 258) |
| TCTTAGCCAC; | (SEQ ID NO: 259) |
| CTTAGCCACT; | (SEQ ID NO: 260) |
| TTAGCCACTT; | (SEQ ID NO: 261) |
| TAGCCACTTT; | (SEQ ID NO: 262) |
| AGCCACTTTT; | (SEQ ID NO: 263) |
| GCCACTTTTT; | (SEQ ID NO: 264) |
| CCACTTTTTA; | (SEQ ID NO: 265) |
| CACTTTTTAA; | (SEQ ID NO: 266) |
| ACTTTTTAAA; | (SEQ ID NO: 267) |
| CTTTTTAAAA; | (SEQ ID NO: 268) |
| TTTTTAAAAG; | (SEQ ID NO: 269) |
| TTTTAAAAGA; | (SEQ ID NO: 270) |
| TTTAAAAGAA; | (SEQ ID NO; 271) |
| TTAAAAGAAA; | (SEQ ID NO: 272) |
| TAAAAGAAAA; | (SEQ ID NO: 273) |
| AAAAGAAAAG; | (SEQ ID NO: 274) |
| AAAGAAAAGG; | (SEQ ID NO: 275) |
| AAGAAAAGGG; | (SEQ ID NO: 276) |
| AGAAAAGGGG; | (SEQ ID NO: 277) |
| GAAAAGGGGG; | (SEQ ID NO: 278) |
| AAAAGGGGGG; | (SEQ ID NO: 279) |
| AAAGGGGGGA; | (SEQ ID NO: 280) |
| AAGGGGGGAC; | (SEQ ID NO: 281) |
| AGGGGGGACT; | (SEQ ID NO: 282) |
| GGGGGGACTG; | (SEQ ID NO: 283) |
| GGGGGACTGG; | (SEQ ID NO: 284) |
| GGGGACTGGA; | (SEQ ID NO: 285) |
| GGGACTGGAA; | (SEQ ID NO: 286) |
| GGACTGGAAG; | (SEQ ID NO: 287) |
| GACTGGAAGG; | (SEQ ID NO: 288) |
| ACTGGAAGGG; | (SEQ ID NO: 289) |
| CTGGAAGGGC; | (SEQ ID NO: 290) |
| TGGAAGGGCT; | (SEQ ID NO: 291) |
| GGAAGGGCTA; | (SEQ ID NO: 292) |
| GAAGGGCTAA; | (SEQ ID NO: 293) |
| AAGGGCTAAT; | (SEQ ID NO: 294) |
| AGGGCTAATT; | (SEQ ID NO: 295) |
| GGGCTAATTC; | (SEQ ID NO: 296) |
| GGCTAATTCA; | (SEQ ID NO: 297) |
| GCTAATTCAC; | (SEQ ID NO: 298) |
| CTAATTCACT; | (SEQ ID NO: 299) |
| TAATTCACTC; | (SEQ ID NO: 300) |
| AATTCACTCC; | (SEQ ID NO: 301) |
| ATTCACTCCC; | (SEQ ID NO: 302) |
| TTCACTCCCA; | (SEQ ID NO: 303) |
| TCACTCCCAA; | (SEQ ID NO: 304) |
| CACTCCCAAA; | (SEQ ID NO: 305) |
| ACTCCCAAAG; | (SEQ ID NO: 306) |
| CTCCCAAAGA; | (SEQ ID NO: 307) |
| TCCCAAAGAA; | (SEQ ID NO: 308) |
| CCCAAAGAAG; | (SEQ ID NO: 309) |
| CCAAAGAAGA; | (SEQ ID NO: 310) |
| CAAAGAAGAC; | (SEQ ID NO: 311) |
| AAAGAAGACA; | (SEQ ID NO: 312) |
| AAGAAGACAA; | (SEQ ID NO: 313) |
| AGAAGACAAG; | (SEQ ID NO: 314) |
| GAAGACAAGA; | (SEQ ID NO: 315) |
| AAGACAAGAT; | (SEQ ID NO: 316) |
| AGACAAGATA; | (SEQ ID NO: 317) |
| GACAAGATAT; | (SEQ ID NO: 318) |
| ACAAGATATC; | (SEQ ID NO: 319) |
| CAAGATATCC; | (SEQ ID NO: 320) |
| AAGATATCCT; | (SEQ ID NO: 321) |
| AGATATCCTT; | (SEQ ID NO: 322) |
| GATATCCTTG; | (SEQ ID NO: 323) |
| ATATCCTTGA; | (SEQ ID NO: 324) |
| TATCCTTGAT; | (SEQ ID NO: 325) |
| ATCCTTGATC; | (SEQ ID NO: 326) |
| TCCTTGATCT; | (SEQ ID NO: 327) |
| CCTTGATCTG; | (SEQ ID NO: 328) |
| CTTGATCTGT; | (SEQ ID NO: 329) |
| TTGATCTGTG; | (SEQ ID NO: 330) |
| TGATCTGTGG; | (SEQ ID NO: 331) |
| GATCTGTGGA; | (SEQ ID NO: 332) |
| ATCTGTGGAT; | (SEQ ID NO: 333) |
| TCTGTGGATC; | (SEQ ID NO: 334) |
| CTGTGGATCT; | (SEQ ID NO: 335) |
| TGTGGATCTA; | (SEQ ID NO: 336) |
| GTGGATCTAC; | (SEQ ID NO: 337) |
| TGGATCTACC; | (SEQ ID NO: 338) |
| GGATCTACCA; | (SEQ ID NO: 339) |
| GATCTACCAC; | (SEQ ID NO: 340) |
| ATCTACCACA; | (SEQ ID NO: 341) |
| TCTACCACAC; | (SEQ ID NO: 342) |
| CTACCACACA; | (SEQ ID NO: 343) |
| TACCACACAC; | (SEQ ID NO: 344) |
| ACCACACACA; | (SEQ ID NO: 345) |
| CCACACACAA; | (SEQ ID NO: 346) |
| CACACACAAG; | (SEQ ID NO: 347) |
| ACACACAAGG; | (SEQ ID NO: 348) |
| CACACAAGGC; | (SEQ ID NO: 349) |
| ACACAAGGCT; | (SEQ ID NO: 350) |
| CACAAGGCTA; | (SEQ ID NO: 351) |
| ACAAGGCTAC; | (SEQ ID NO: 352) |
| CAAGGCTACT; | (SEQ ID NO: 353) |
| AAGGCTACTT; | (SEQ ID NO: 354) |
| AGGCTACTTC; | (SEQ ID NO: 355) |
| GGCTACTTCC; | (SEQ ID NO: 356) |
| GCTACTTCCC; | (SEQ ID NO: 357) |
| CTACTTCCCT; | (SEQ ID NO: 358) |
| TACTTCCCTG; | (SEQ ID NO: 359) |
| ACTTCCCTGA; | (SEQ ID NO: 360) |
| CTTCCCTGAT; | (SEQ ID NO: 361) |
| TTCCCTGATT; | (SEQ ID NO: 362) |
| TCCCTGATTG; | (SEQ ID NO: 363) |
| CCCTGATTGG; | (SEQ ID NO: 364) |
| CCTGATTGGC; | (SEQ ID NO: 365) |
| CTGATTGGCA; | (SEQ ID NO: 366) |
| TGATTGGCAG; | (SEQ ID NO: 367) |
| GATTGGCAGA; | (SEQ ID NO: 368) |
| ATTGGCAGAA; | (SEQ ID NO: 369) |
| TTGGCAGAAC; | (SEQ ID NO: 370) |
| TGGCAGAACT; | (SEQ ID NO; 371) |
| GGCAGAACTA; | (SEQ ID NO: 372) |
| GCAGAACTAC; | (SEQ ID NO: 373) |
| CAGAACTACA; | (SEQ ID NO: 374) |
| AGAACTACAC; | (SEQ ID NO: 375) |
| GAACTACACA; | (SEQ ID NO: 376) |
| AACTACACAC; | (SEQ ID NO; 377) |
| ACTACACACC; | (SEQ ID NO: 378) |
| CTACACACCA; | (SEQ ID NO; 379) |
| TACACACCAG; | (SEQ ID NO: 380) |
| ACACACCAGG; | (SEQ ID NO: 381) |
| CACACCAGGG; | (SEQ ID NO: 382) |
| ACACCAGGGC; | (SEQ ID NO: 383) |
| CACCAGGGCC; | (SEQ ID NO: 384) |
| ACCAGGGCCA; | (SEQ ID NO: 385) |
| CCAGGGCCAG; | (SEQ ID NO: 386) |
| CAGGGCCAGG; | (SEQ ID NO: 387) |
| AGGGCCAGGG; | (SEQ ID NO: 388) |
| GGGCCAGGGG; | (SEQ ID NO: 389) |
| GGCCAGGGGT; | (SEQ ID NO: 390) |
| GCCAGGGGTC; | (SEQ ID NO: 391) |
| CCAGGGGTCA; | (SEQ ID NO: 392) |
| CAGGGGTCAG; | (SEQ ID NO; 393) |
| AGGGGTCAGA; | (SEQ ID NO: 394) |
| GGGGTCAGAT; | (SEQ ID NO: 395) |
| GGGTCAGATA; | (SEQ ID NO: 396) |
| GGTCAGATAT; | (SEQ ID NO: 397) |
| GTCAGATATC; | (SEQ ID NO: 398) |
| TCAGATATCC; | (SEQ ID NO: 399) |
| CAGATATCCA; | (SEQ ID NO: 400) |
| AGATATCCAC; | (SEQ ID NO: 401) |
| GATATCCACT; | (SEQ ID NO: 402) |
| ATATCCACTG; | (SEQ ID NO: 403) |
| TATCCACTGA; | (SEQ ID NO: 404) |
| ATCCACTGAC; | (SEQ ID NO: 405) |
| TCCACTGACC; | (SEQ ID NO: 406) |
| CCACTGACCT; | (SEQ ID NO: 407) |
| CACTGACCTT; | (SEQ ID NO: 408) |
| ACTGACCTTT; | (SEQ ID NO: 409) |
| CTGACCTTTG; | (SEQ ID NO: 410) |
| TGACCTTTGG; | (SEQ ID NO: 411) |
| GACCTTTGGA; | (SEQ ID NO: 412) |
| ACCTTTGGAT; | (SEQ ID NO: 413) |
| CCTTTGGATG; | (SEQ ID NO: 414) |
| CTTTGGATGG; | (SEQ ID NO: 415) |
| TTTGGATGGT; | (SEQ ID NO: 416) |
| TTGGATGGTG; | (SEQ ID NO: 417) |
| TGGATGGTGC; | (SEQ ID NO: 418) |
| GGATGGTGCT; | (SEQ ID NO: 419) |
| GATGGTGCTA; | (SEQ ID NO: 420) |
| ATGGTGCTAC; | (SEQ ID NO: 421) |
| TGGTGCTACA; | (SEQ ID NO: 422) |
| GGTGCTACAA; | (SEQ ID NO: 423) |
| GTGCTACAAG; | (SEQ ID NO: 424) |
| TGCTACAAGC; | (SEQ ID NO: 425) |
| GCTACAAGCT; | (SEQ ID NO: 426) |
| CTACAAGCTA; | (SEQ ID NO: 427) |
| TACAAGCTAG; | (SEQ ID NO: 428) |
| ACAAGCTAGT; | (SEQ ID NO: 429) |
| CAAGCTAGTA; | (SEQ ID NO: 430) |
| AAGCTAGTAC; | (SEQ ID NO: 431) |
| AGCTAGTACC; | (SEQ ID NO: 432) |
| GCTAGTACCA; | (SEQ ID NO: 433) |
| CTAGTACCAG; | (SEQ ID NO: 434) |
| TAGTACCAGT; | (SEQ ID NO: 435) |
| AGTACCAGTT; | (SEQ ID NO: 436) |
| GTACCAGTTG; | (SEQ ID NO: 437) |
| TACCAGTTGA; | (SEQ ID NO: 438) |
| ACCAGTTGAG; | (SEQ ID NO: 439) |
| CCAGTTGAGC; | (SEQ ID NO: 440) |
| CAGTTGAGCC; | (SEQ ID NO: 441) |
| AGTTGAGCCA; | (SEQ ID NO: 442) |
| GTTGAGCCAG; | (SEQ ID NO: 443) |
| TTGAGCCAGA; | (SEQ ID NO: 444) |
| TGAGCCAGAT; | (SEQ ID NO: 445) |
| GAGCCAGATA; | (SEQ ID NO: 446) |
| AGCCAGATAA; | (SEQ ID NO: 447) |
| GCCAGATAAG; | (SEQ ID NO: 448) |
| CCAGATAAGG; | (SEQ ID NO: 449) |
| CAGATAAGGT; | (SEQ ID NO: 450) |
| AGATAAGGTA; | (SEQ ID NO: 451) |
| GATAAGGTAG; | (SEQ ID NO: 452) |
| ATAAGGTAGA; | (SEQ ID NO: 453) |
| TAAGGTAGAA; | (SEQ ID NO: 454) |
| AAGGTAGAAG; | (SEQ ID NO: 455) |
| AGGTAGAAGA; | (SEQ ID NO: 456) |
| GGTAGAAGAG; | (SEQ ID NO: 457) |
| GTAGAAGAGG; | (SEQ ID NO: 458) |
| TAGAAGAGGC; | (SEQ ID NO: 459) |
| AGAAGAGGCC; | (SEQ ID NO: 460) |
| GAAGAGGCCA; | (SEQ ID NO: 461) |
| AAGAGGCCAA; | (SEQ ID NO: 462) |
| AGAGGCCAAT; | (SEQ ID NO: 463) |
| GAGGCCAATA; | (SEQ ID NO: 464) |
| AGGCCAATAA; | (SEQ ID NO: 465) |
| GGCCAATAAA; | (SEQ ID NO: 466) |
| GCCAATAAAG; | (SEQ ID NO: 467) |
| CCAATAAAGG; | (SEQ ID NO: 468) |
| CAATAAACGA; | (SEQ ID NO: 469) |
| AATAAAGGAG; | (SEQ ID NO: 470) |
| ATAAAGGAGA; | (SEQ ID NO: 471) |
| TAAAGGAGAG; | (SEQ ID NO: 472) |
| AAAGGAGAGA; | (SEQ ID NO: 473) |
| AAGGAGAGAA; | (SEQ ID NO: 474) |
| AGGAGAGAAC; | (SEQ ID NO: 475) |
| GGAGAGAACA; | (SEQ ID NO: 476) |
| GAGAGAACAC; | (SEQ ID NO: 477) |
| AGAGAACACC; | (SEQ ID NO: 478) |
| GAGAACACCA; | (SEQ ID NO: 479) |
| AGAACACCAG; | (SEQ ID NO: 480) |
| GAACACCAGC; | (SEQ ID NO: 481) |
| AACACCAGCT; | (SEQ ID NO: 482) |
| ACACCAGCTT; | (SEQ ID NO: 483) |
| CACCAGCTTG; | (SEQ ID NO: 484) |
| ACCAGCTTGT; | (SEQ ID NO: 485) |
| CCAGCTTGTT; | (SEQ ID NO: 486) |
| CAGCTTGTTA; | (SEQ ID NO: 487) |
| AGCTTGTTAC; | (SEQ ID NO: 488) |
| GCTTGTTACA; | (SEQ ID NO: 489) |
| CTTGTTACAC; | (SEQ ID NO: 490) |
| TTGTTACACC; | (SEQ ID NO: 491) |
| TGTTACACCC; | (SEQ ID NO: 492) |
| GTTACACCCT; | (SEQ ID NO: 493) |
| TTACACCCTG; | (SEQ ID NO; 494) |
| TACACCCTGT; | (SEQ ID NO: 495) |
| ACACCCTGTG; | (SEQ ID NO: 496) |
| CACCCTGTGA; | (SEQ ID NO: 497) |
| ACCCTGTGAG; | (SEQ ID NO: 498) |
| CCCTGTGAGC; | (SEQ ID NO: 499) |
| CCTGTGAGCC; | (SEQ ID NO: 500) |
| CTGTGAGCCT; | (SEQ ID NO: 501) |
| TGTGAGCCTG; | (SEQ ID NO: 502) |
| GTGAGCCTGC; | (SEQ ID NO: 503) |
| TGAGCCTGCA; | (SEQ ID NO: 504) |
| GAGCCTGCAT; | (SEQ ID NO: 505) |
| AGCCTGCATG; | (SEQ ID NO: 506) |
| GCCTGCATGG; | (SEQ ID NO: 507) |
| CCTGCATGGA; | (SEQ ID NO: 508) |
| CTGCATGGAA; | (SEQ ID NO: 509) |
| TGCATGGAAT; | (SEQ ID NO: 510) |
| GCATGGAATG; | (SEQ ID NO: 511) |
| CATGGAATGG; | (SEQ ID NO: 512) |
| ATGGAATGGA; | (SEQ ID NO: 513) |
| TGGAATGGAT; | (SEQ ID NO: 514) |
| GGAATGGATG; | (SEQ ID NO: 515) |
| GAATGGATGA; | (SEQ ID NO: 516) |
| AATGGATGAC; | (SEQ ID NO: 517) |
| ATGGATGACC; | (SEQ ID NO: 518) |
| TGGATGACCC; | (SEQ ID NO; 519) |
| GGATGACCCT; | (SEQ ID NO: 520) |
| GATGACCCTG; | (SEQ ID NO: 521) |
| ATGACCCTGA; | (SEQ ID NO: 522) |
| TGACCCTGAG; | (SEQ ID NO: 523) |
| GACCCTGAGA; | (SEQ ID NO: 524) |
| ACCCTGAGAG; | (SEQ ID NO: 525) |
| CCCTGAGAGA; | (SEQ ID NO: 526) |
| CCTGAGAGAG; | (SEQ ID NO: 527) |
| CTGAGAGAGA; | (SEQ ID NO: 525) |
| TGAGAGAGAA; | (SEQ ID NO: 529) |
| GAGAGAGAAG; | (SEQ ID NO: 530) |
| AGAGAGAAGT; | (SEQ ID NO: 531) |
| GAGAGAAGTG; | (SEQ ID NO: 532) |
| AGAGAAGTGT; | (SEQ ID NO: 533) |
| GAGAAGTGTT; | (SEQ ID NO: 534) |
| AGAAGTGTTA; | (SEQ ID NO: 535) |
| GAAGTGTTAG; | (SEQ ID NO: 536) |
| AAGTGTTAGA; | (SEQ ID NO: 537) |
| AGTGTTAGAG; | (SEQ ID NO: 538) |
| GTGTTAGAGT; | (SEQ ID NO: 539) |
| TGTTAGAGTG; | (SEQ ID NO: 540) |
| GTTAGAGTGG; | (SEQ ID NO: 541) |
| TTAGAGTGGA; | (SEQ ID NO: 542) |
| TAGAGTGGAG; | (SEQ ID NO: 543) |
| AGAGTGGAGG; | (SEQ ID NO: 544) |
| GAGTGGAGGT; | (SEQ ID NO: 545) |
| AGTGGAGGTT; | (SEQ ID NO: 546) |
| GTGGAGGTTT; | (SEQ ID NO: 547) |
| TGGAGGTTTG; | (SEQ ID NO: 548) |
| GGAGGTTTGA; | (SEQ ID NO: 549) |
| GAGGTTTGAC; | (SEQ ID NO: 550) |
| AGGTTTGACA; | (SEQ ID NO: 551) |
| GGTTTGACAG; | (SEQ ID NO: 552) |
| GTTTGACAGC; | (SEQ ID NO: 553) |
| TTTGACAGCC; | (SEQ ID NO: 554) |
| TTGACAGCCG; | (SEQ ID NO: 555) |
| TGACAGCCGC; | (SEQ ID NO: 556) |
| GACAGCCGCC; | (SEQ ID NO: 557) |
| ACAGCCGCCT; | (SEQ ID NO: 558) |
| CAGCCGCCTA; | (SEQ ID NO: 559) |
| AGCCGCCTAG; | (SEQ ID NO: 560) |
| GCCGCCTAGC; | (SEQ ID NO: 561) |
| CCGCCTAGCA; | (SEQ ID NO: 562) |
| CGCCTAGCAT; | (SEQ ID NO: 563) |
| GCCTAGCATT; | (SEQ ID NO: 564) |
| CCTAGCATTT; | (SEQ ID NO: 565) |
| CTAGCATTTC; | (SEQ ID NO: 566) |
| TAGCATTTCA; | (SEQ ID NO: 567) |
| AGCATTTCAT; | (SEQ ID NO: 568) |
| GCATTTCATC; | (SEQ ID NO: 569) |
| CATTTCATCA; | (SEQ ID NO: 570) |
| ATTTCATCAC; | (SEQ ID NO: 571) |
| TTTCATCACG; | (SEQ ID NO: 572) |
| TTCATCACGT; | (SEQ ID NO: 573) |
| TCATCACGTG; | (SEQ ID NO: 574) |
| CATCACGTGG; | (SEQ ID NO: 575) |
| ATCACGTGGC; | (SEQ ID NO: 576) |
| TCACGTGGCC; | (SEQ ID NO: 577) |
| CACGTGGCCC; | (SEQ ID NO: 578) |
| ACGTGGCCCG; | (SEQ ID NO: 579) |
| CGTGGCCCGA; | (SEQ ID NO; 580) |
| GTGGCCCGAG; | (SEQ ID NO: 581) |
| TGGCCCGAGA; | (SEQ ID NO: 582) |
| GGCCCGAGAG; | (SEQ ID NO: 583) |
| GCCCGAGAGC; | (SEQ ID NO: 584) |
| CCCGAGAGCT; | (SEQ ID NO: 585) |
| CCGAGAGCTG; | (SEQ ID NO: 586) |
| CGAGAGCTGC; | (SEQ ID NO: 587) |
| GAGAGCTGCA; | (SEQ ID NO: 588) |
| AGAGCTGCAT; | (SEQ ID NO: 589) |
| GAGCTGCATC; | (SEQ ID NO: 590) |
| AGCTGCATCC; | (SEQ ID NO: 591) |
| GCTGCATCCG; | (SEQ ID NO: 592) |
| CTGCATCCGG; | (SEQ ID NO: 593) |
| TGCATCCGGA; | (SEQ ID NO: 594) |
| GCATCCGGAG; | (SEQ ID NO: 595) |
| CATCCGGAGT; | (SEQ ID NO: 596) |
| ATCCGGAGTA; | (SEQ ID NO: 597) |
| TCCGGAGTAC; | (SEQ ID NO: 598) |
| CCCGAGTACT; | (SEQ ID NO: 599) |
| CGGAGTACTT; | (SEQ ID NO: 600) |
| GGAGTACTTC; | (SEQ ID NO: 601) |
| GAGTACTTCA; | (SEQ ID NO: 602) |
| AGTACTTCAA; | (SEQ ID NO: 603) |
| GTACTTCAAG; | (SEQ ID NO: 604) |
| TACTTCAAGA; | (SEQ ID NO: 605) |
| ACTTCAAGAA; | (SEQ ID NO: 606) |
| CTTCAAGAAC; | (SEQ ID NO: 607) |
| TTCAAGAACT; | (SEQ ID NO: 608) |
| TCAAGAACTG; | (SEQ ID NO: 609) |
| CAAGAACTGC; | (SEQ ID NO: 610) |
| AAGAACTGCT; | (SEQ ID NO: 611) |
| AGAACTGCTG; | (SEQ ID NO: 612) |
| GAACTGCTGA. | (SEQ ID NO: 613) |

In a preferred embodiment the present invention contemplates a viral isolate which:
(i) is reactive to antibodies to a glycoprotein from HIV-1 such as at least one of gp41-45, gp120 and/or gp160;
(ii) carries a deletion of at least ten nucleotides in a region corresponding to the nef gene in HIV-1_{NL43}; and

wherein said deletion encompasses one or more decanucleotides from the nef gene of HIV-1_{NL43} or corresponding sequences from another pathogenic strain of HIV-1 defined in (or substantially analogous to) SEQ ID NOs:803 to 841:

| | |
|---|---|
| ACCAGCTTGT | [SEQ ID NO:803] |
| CCAGCTTGTT | [SEQ ID NO:804] |
| CAGCTTGTTA | [SEQ ID NO:805] |
| AGCTTGTTAC | [SEQ ID NO:806] |
| GCTTGTTACA | [SEQ ID NO:807] |
| CTTGTTACAC | [SEQ ID NO:808] |
| TTGT1ACACC | [SEQ ID NO:809] |
| TGTTACACCC | [SEQ ID NO:810] |
| GTTACACCCT | [SEQ ID NO:811] |
| TTACACCCTG | [SEQ ID NO:812] |
| TACACCCTGT | [SEQ ID NO:813] |
| ACACCCTGTG | [SEQ ID NO:814] |
| CACCCTGTGA | [SEQ ID NO:815] |
| ACCCTGTGAG | [SEQ ID NO:816] |
| CCCTGTGAGC | [SEQ ID NO:817] |
| CCTGTGAGCC | [SEQ ID NO:818] |
| CTGTGAGCCT | [SEQ ID NO:819] |
| TGTGAGCCTG | [SEQ ID NO:820] |
| GTGAGCCTGC | [SEQ ID NO:821] |
| TGAGCCTGCA | [SEQ ID NO:822] |
| GAGCCTGCAT | [SEQ ID NO:823] |
| AGCCTGCATG | [SEQ ID NO:824] |
| GCCTGCATCG | [SEQ ID NO:825] |
| CCTGCATGGA | [SEQ ID Nd:826] |
| CTGCATGGAA | [SEQ ID NO:827] |
| TGCATGGAAT | [SEQ ID NO:828] |
| GCATGGAATG | [SEQ ID NO:829] |
| CATGGAATGG | [SEQ ID NO:830] |
| ATGGAATGGA | [SEQ ID NO:831] |
| TGGAATGGAT | [SEQ ID NO:832] |
| GGAATGGATG | [SEQ ID NO:833] |
| GAATGGATGA | [SEQ ID NO:834] |
| AATGGATGAC | [SEQ ID NO:835] |
| ATGGATGACC | [SEQ ID NO 836] |
| TGGATGACCC | [SEQ ID NO:837] |
| GGATGACCCT | [SEQ ID NO:838] |
| GATGACCCTG | [SEQ ID NO:839] |
| ATGACCCTGA | [SEQ ID NO:840] |
| TGACCCTGAG | [SEQ ID NO:841] |

Generally, the subject HIV-1 isolate is non-pathogenic as hereinbefore defined. Additionally, reference herein to “a deletion” includes reference to a contiguous or non-contiguous series of two or more deletions.

The non-pathogenic isolate may carry a single decanucleotide deletion or may carry more than one decanucleotide deletion. Where it carries multiple deletions these may all correspond to a contiguous sequence or be from different parts of the nef gene. Furthermore, the terminal end portions of a deletion may lie within a decanucleotide as defined above. It is emphasised that the present invention extends to analogous sequences from other pathogenic strains of HIV-1 which might carry nef genes with a slightly altered sequence relative to HIV-1_{NL43}.

In a most preferred embodiment of the present invention, there is provided a non-pathogenic strain of HIV-1 carrying a nucleotide sequence in its genome as set forth

SEQ ID No. 614:
GAAGAGATTTGGGAGAACATGACCTGGATGCAGTGGGAAAAAGAAATTCACAATCACAC
AAAATACATATACTCCTTACTTGAAAAATCGCAGAACCAACAAGAAAAGAATGAACAAG
AACTATTGGAATTGGATCAATGGGCAAGTTTGTGGAATTGGTTTGACATAACAAAATGG
CTGTGGTATATAAAAATATTCATAATGGTAGTAGGAGGCTTGATAGGTTTAAGAATAGT
TTTTGCTGTACTTTCTATAGTGAATAGAGTTAGGCAGGGATACTCACCATTGTCGTTTC
AGACCCTCCTCCCAACCCCGAGGGGACCCGACAGGCCCGAAGGAATCGAAGAAGAAGGT
GGAGAGAGAGACAGAGACAGATCCACTCGATTAGTACACGGATTCTTAGCACTTTTCTG
GGACGACCTGAGGAGCCTGTGCCTCTTCCTCTACCACCACTTGAGAGACTTACTCTTGA
TTGTAACAAGGATTGTGGAACTTCTGGGACGCAGGGGATGGGAAGCCCTCAAATATTGG
TGGAACCTCCTAAAGTATTGGAGCCAGGAACTGCAGAAGAGTGCTGTTATCTTGCTCAA
TGCCACCGCCATAGCAGTAGCTGAGGGGACAGATAGAGTTTTAGAAGTATTACAAAGAG
CTTATAGAGCTATCCTCCACATACCTAGAAGAATAAGACAGGGCCTCGAAATGGCTTTG
CTATAAAATGGGTGGCAAGTGAGCAAAAAGTAGTGTAGTCAGATAGCATGCATCATAAG
GGGTGGGGGCCAACAACTAACAATGCTGATCGTGCCTGGCTAGAAGCACAAGAGAAGGA
AGAAGCGGGTTTTCCAGTCAAACCTCAGGTAGCTGTAGATCTTAGCCACTTTTTAAAAG
AAAAGGGGGGACTGGAAGGGCTAATTCACTCCCAAAGAAGACAAGATACACAGTGCTGC
AAACTATTACCAGTGGAGTCAGCGAAGATAGAAGAGGCCAATGGAGGAGAAAACCACAG
ATTGTTCTGTTGGGGACTTTCCATCCGTTGGGGACTTTCCAAGGCGGCGTGGCCTGGGT
GACTAGTTCCGGTGGGGACTTTCCAAGAAGGCGCGGCCTGGGCGGGACTGGGGAGTGGC
GAGCCCTCAGATGCTGCATATAAGCAGCTGCTTTCTGCTGTTACTGGGTCTCTCGGGTT
AGACCAGATCTGAGCCTGGGAGCTCTCTGGCTAACTAGGGAACCCACTGCTTAAGCCTC
AATAAAGCTTGCCTTGAGTGCTTCAAGTAGTGTGTGCCCGTCTGTTGTGTGACTCTGGT
ATCTAGA;
and/or
SEQ ID NO: 615:
GAAACAATTTGGGATAACATGACCTGGATGCAGTGGGAAAGAGAAATTGACAATTACAC
AAACATAATATACACCTTAATTGAAGAATCGCAGAACCAACAAGAAAAAAATGAACTAG
AATTATTGGAATTGGATAAATGGGCAAATTTGTGGAATTGGTTTAGTATATCAAACTGG
CTATGGTATATAAAATTATTCATAATGGTAGTAGGAGGCTTGGTAGGTTTAAGAATAGT
TTTTACTGTACTTTCTATAGTGAATAGAGTTAGGCAGGGATACTCACCATTGTCGTTTC
AGACCCACCTCCCAACCCCGAAGGGACCCGACAGGCCAGAAGGAATCGAAGAAGAAGGT
GGAGAGAGAGACAGAGGCAGCTCCACTCGATTAGTGCACGGATTCTTAGCACTTTTCTG
GGACGACCTGAGGAGTCTGTGCCTCTTCAGCTACCACCACTTGAGAGACTTACTCTTGA
TTGTAACGAGGATTGTGGAACTTCTGGGACGCAGGGGATGGGAAGCCCTCAAATACTGG
TGGAATCTCCTGCAGTATTGGAGGCAGGAACTACAGAAGAGTGCTGTTAGCTTGTTCAA
TGGCACGGCCATAGCAGTAGCTGAGGGGACAGATAGAGTTATAGAAGCTTTACGAAGGG
CTTATAGAGCTATTCTCCACATACCTAGAAGAATAAGACAGGGCTTAGAAAGGGCTTTG
CTATAAAATGGGTGGCAAGTGGTCAGAAAGTAGTGTGGTTAGAAGGCATGTACCTTTAA
GACAAGGCAGCTATAGATCTTAGCCGCTTTTTAAAAGAAAAGGGGGGACTGGAAGGGCT
AATTCACTCACAGAGAAGATCAGTTGAACCAGAAGAAGATAGAAGAGGCCATGAAGAAG
AAAACAACAGATTGTTCCGTTTGTTCCGTTGGGGACTTTCCAGGAGACGTGGCCTGAGT
GATAAGCCGCTGGGGACTTTCCGAAGAGGCGTGACGGGACTTTCCAAGGCGACGTGGCC
TGGGCGGGACTGGGGAGTGGCGAGCCCTCAGATGCTGCATATAAGCAGCTGCTTTCTGC
CTGTACTGGGTCTCTCTGGTTAGACCAGATCTGAGCCTGGGAGCTCTCTGGCTAACTAG
GGAACCCACTGCTTAAGCCTCAATAAAGCTTGCCTTGAGTGCTTCAAGTAGTGTGTGCC
CGTCTGTTGTGTGACTCTGGTATCTAGA.

The present invention, however, extends to HIV-1 isolates which ar non-pathogenic; carry genomes capable of hybridising under low stringency conditions to SEQ ID NO: 614 or SEQ ID NO: 615; and which do not direct the synthesis of a full length nef gene product.

In a further embodiment the present invention contemplates a viral isolate which:
(i) is reactive to antibodies to a glycoprotein from HIV-1 such as at least one of gp41-45, gp120 and/or gp160;
(ii) carries a genome or a part or fragment thereof capable of hybridising under medium stringency conditions to a nucleotide sequences set forth in SEQ ID NO: 1 or a complementary form thereof or an analogous sequence from another pathogenic strain of HIV-1;
(iii) carries a deletion of at lest ten nucleotides in a region corresponding to the LTR region in HIV-1_{NL43}; and

wherein said deletion encompasses one or more of the following decanucleotides from the LTR region of HIV-1_{NL43} or corresponding sequences from another pathogenic strain of HIV-1:

| | |
|---|---|
| GCTTTTTGCC; | (SEQ ID NO: 652) |
| CTTTTTGCCT; | (SEQ ID NO: 653) |
| TTTTTGCCTG; | (SEQ ID NO: 654) |
| TTTTGCCTGT; | (SEQ ID NO: 655) |
| TTTGCCTGTA; | (SEQ ID NO: 656) |
| TTGCCTGTAC; | (SEQ ID NO: 657) |
| TGCCTGTACT; | (SEQ ID NO: 658) |
| GCCTGTACTG; | (SEQ ID NO: 659) |
| CCTGTACTGG; | (SEQ ID NO: 660) |
| CTGTACTGGG; | (SEQ ID NO: 661) |
| TGTACTGGGT; | (SEQ ID NO: 662) |
| GTACTGGGTC; | (SEQ ID NO: 663) |
| TACTGGGTCT; | (SEQ ID NO: 664) |
| ACTGGGTCTC; | (SEQ ID NO: 665) |
| CTGGGTCTCT; | (SEQ ID NO: 666) |
| TGGGTCTCTC; | (SEQ ID NO: 667) |
| GGGTCTCTCT; | (SEQ ID NO: 668) |
| GGTCTCTCTG; | (SEQ ID NO: 669) |
| GTCTCTCTGG; | (SEQ ID NO: 670) |
| TCTCTCTGGT; | (SEQ ID NO: 671) |
| CTCTCTGGTT; | (SEQ ID NO: 672) |
| TCTCTGGTTA; | (SEQ ID NO: 673) |
| CTCTGGTTAG; | (SEQ ID NO: 674) |
| TCTCTGGTTA; | (SEQ ID NO: 675) |
| CTGGTTAGAC; | (SEQ ID NO: 676) |
| TGGTTAGACC; | (SEQ ID NO: 677) |
| GGTTAGACCA; | (SEQ ID NO: 678) |
| GTTAGACCAG; | (SEQ ID NO: 679) |
| TTAGACCAGA; | (SEQ ID NO: 680) |
| TAGACCAGAT; | (SEQ ID NO: 681) |
| AGACCAGATC; | (SEQ ID NO: 682) |
| GACCAGATCT; | (SEQ ID NO: 683) |
| ACCAGATCTG; | (SEQ ID NO: 684) |
| CCAGATCTGA; | (SEQ ID NO: 685) |
| CAGATCTGAG; | (SEQ ID NO: 686) |
| AGATCTGAGC; | (SEQ ID NO: 687) |
| GATCTGAGCC; | (SEQ ID NO: 688) |
| ATCTGAGCCT; | (SEQ ID NO: 689) |
| TCTGAGCCTG; | (SEQ ID NO: 690) |
| CTGAGCCTGG; | (SEQ ID NO: 691) |
| TGAGCCTGGG; | (SEQ ID NO: 692) |
| GAGCCTGGGA; | (SEQ ID NO: 693) |
| AGCCTGGGAG; | (SEQ ID NO: 694) |
| GCCTGGGAGC; | (SEQ ID NO: 695) |
| CCTGGGAGCT; | (SEQ ID NO: 696) |
| CTGGGAGCTC; | (SEQ ID NO: 697) |
| TGGGAGCTCT; | (SEQ ID NO: 698) |
| GGGAGCTCTC; | (SEQ ID NO: 699) |
| GGAGCTCTCT; | (SEQ ID NO: 700) |
| GAGCTCTCTG; | (SEQ ID NO: 701) |
| AGCTCTCTGG; | (SEQ ID NO: 702) |
| GCTCTCTGGC; | (SEQ ID NO: 703) |
| CTCTCTGGCT; | (SEQ ID NO: 704) |
| TCTCTGGCTA; | (SEQ ID NO: 705) |
| CTCTGGCTAA; | (SEQ ID NO: 706) |
| TCTGGCTAAC; | (SEQ ID NO: 707) |
| CTGGCTAACT; | (SEQ ID NO: 708) |
| TGGCTAACTA; | (SEQ ID NO: 709) |
| GGCTAACTAG; | (SEQ ID NO: 710) |
| GCTAACTAGG; | (SEQ ID NO: 711) |
| CTAACTAGGG; | (SEQ ID NO: 712) |
| TAACTAGGGA; | (SEQ ID NO: 713) |
| AACTAGGGAA; | (SEQ ID NO: 714) |
| ACTAGGGAAC; | (SEQ ID NO: 715) |
| CTAGGGAACC; | (SEQ ID NO: 716) |
| TAGGGAACCC; | (SEQ ID NO: 717) |
| AGGGAACCCA; | (SEQ ID NO: 718) |
| GGGAACCCAC; | (SEQ ID NO: 719) |
| GGAACCCACT; | (SEQ ID NO: 720) |
| GAACCCACTG; | (SEQ ID NO: 721) |
| AACCCACTGC; | (SEQ ID NO: 722) |
| ACCCACTGCT; | (SEQ ID NO: 723) |
| CCCACTGCTT; | (SEQ ID NO: 724) |
| CCACTGCTTA; | (SEQ ID NO: 725) |
| CACTGCTCAA; | (SEQ ID NO: 726) |
| ACTGCTTAAG; | (SEQ ID NO: 727) |
| CTGCTTAAGC; | (SEQ ID NO: 728) |
| TGCTTAAGCC; | (SEQ ID NO: 729) |
| GCTTAAGCCT; | (SEQ ID NO: 730) |
| CTTAAGCCTC; | (SEQ ID NO: 731) |
| TTAAGCCTCA; | (SEQ ID NO: 732) |
| TAAGCCTCAA; | (SEQ ID NO: 733) |
| AAGCCTCAAT; | (SEQ ID NO: 734) |
| AGCCTCAATA; | (SEQ ID NO: 735) |
| GCCTCAATAA; | (SEQ ID NO: 736) |
| CCTCAATAAA; | (SEQ ID NO: 737) |
| CTCAATAAAG; | (SEQ ID NO: 738) |
| TCAATAAAGC; | (SEQ ID NO: 739) |
| CAATAAAGCT; | (SEQ ID NO: 740) |
| AATAAAGCTT; | (SEQ ID NO: 741) |
| ATAAAGCTTG; | (SEQ ID NO: 742) |
| TAAAGCTTGC; | (SEQ ID NO: 743) |
| AAAGCTTGCC; | (SEQ ID NO: 744) |
| AAGCTTGCCT; | (SEQ ID NO: 745) |
| AGCTTGCCTT; | (SEQ ID NO: 746) |
| GCTTGCCTTG; | (SEQ ID NO: 747) |
| CTTGCCTTGA; | (SEQ ID NO: 748) |
| TTGCCTTGAG; | (SEQ ID NO: 749) |
| TGCCTTGAGT; | (SEQ ID NO: 750) |
| GCCTTGAGTG; | (SEQ ID NO: 751) |
| CCTTGAGTGC; | (SEQ ID NO: 752) |
| CTTGAGTGCT; | (SEQ ID NO: 753) |
| TTGAGTGCTT; | (SEQ ID NO: 754) |
| TGAGTGCTTC; | (SEQ ID NO: 755) |
| GAGTGCTTCA; | (SEQ ID NO: 756) |
| AGTGCTTCAA; | (SEQ ID NO: 757) |
| GTGCTTCAAG; | (SEQ ID NO: 758) |
| TGCTTCAAGT; | (SEQ ID NO: 759) |
| GCTTCAAGTA; | (SEQ ID NO: 760) |
| CTTCAAGTAG; | (SEQ ID NO: 761) |
| TTCAAGTAGT; | (SEQ ID NO: 762) |
| TCAAGTAGTG; | (SEQ ID NO: 763) |
| CAAGTAGTGT; | (SEQ ID NO: 764) |
| AAGTAGTGTG; | (SEQ ID NO: 765) |
| AGTAGTGTGT; | (SEQ ID NO: 766) |
| GTAGTGTGTG; | (SEQ ID NO: 767) |
| TAGTGTGTGC; | (SEQ ID NO: 768) |
| AGTGTGTGCC; | (SEQ ID NO: 769) |
| GTGTGTGCCC; | (SEQ ID NO: 770) |
| TGTGTGCCCG; | (SEQ ID NO: 771) |
| GTGTGCCCGT; | (SEQ ID NO: 772) |
| TGTGCCCGTC; | (SEQ ID NO: 773) |
| GTGCCCGTCT; | (SEQ ID NO: 774) |
| TGCCCGTCTG; | (SEQ ID NO: 775) |
| GCCCGTCTGT; | (SEQ ID NO: 776) |
| CCCGTCTGTT; | (SEQ ID NO: 777) |
| CCGTCTGTTG; | (SEQ ID NO: 778) |
| CGTCTGTTGT; | (SEQ ID NO: 779) |
| GTCTGTTGTG; | (SEQ ID NO: 780) |
| TCTGTTGTGT; | (SEQ ID NO: 781) |
| CTGTTGTGTG; | (SEQ ID NO: 782) |
| TGTTGTGTGA; | (SEQ ID NO: 783) |
| GTTGTGTGAC; | (SEQ ID NO: 784) |
| TTGTGTGACT; | (SEQ ID NO: 785) |
| TGTGTGACTC; | (SEQ ID NO: 786) |
| GTGTGACTCT; | (SEQ ID NO: 787) |
| TGTGTGACTC; | (SEQ ID NO: 788) |
| GTGTGACTCT; | (SEQ ID NO: 789) |
| TGTGACTCTG; | (SEQ ID NO: 790) |
| GTGACTCTGG; | (SEQ ID NO: 791) |
| TGACTCTGGT; | (SEQ ID NO: 792) |
| GACTCTGGTA; | (SEQ ID NO: 793) |
| ACTCTGGTAA; | (SEQ ID NO: 794) |
| CTCTGGTAAC; | (SEQ ID NO: 795) |
| TCTGGTAACT; | (SEQ ID NO: 796) |
| CTGGTAACTA; | (SEQ ID NO: 797) |
| TGGTAACTAG; | (SEQ ID NO: 798) |
| GGTAACTAGA. | (SEQ ID NO: 799) |

The non-pathogenic isolate may carry a single decanucleotide deletion in the LTR region or may carry multiple deletions in the same region or in the LTR region and another region such as the nef gene. In particular, the mutation may bc in the LTR/nef overlap region. Where it carries multiple deletions, these may correspond to a contiguous sequence or be from different parts of the LTR region and/or nef gene. Furthermore, the terminal end portions of a deletion may lie within a decanucleotide as defined above.

Yet another aspect of the present invention provides an infectious molecular clone comprising genetic sequences derived from the non-pathogenic HIV-I isolates as hereinbefore described and includes genetic sequences encoding major structural proteins such as gag; env and pol. Infectious molecular clones are particularly useful as genetic compositions capable of “infecting” host cells without need of viral coat The infectious molecular clones of the present invention may also be derived from pathogenic HIV-1 strains rendered non-pathogenic as hereindescribed.

According to this latter embodiment, there is contemplated a method of attenuating a pathogenic strain of HIV-1, said method comprising inducing a mutation in the nef gene and/or an LTR region to generate a non-pathogenic HIV-1 in as hereinbefore described. Preferred mutations are deletions of at least ten nucleotides such as one or more of the decanucleotides as hereinbefore described. Particularly preferred mutations result in modified Nef protein carrying a deletion substantially corresponding to amino acids 162 to 177 of Nef from wild-type HIV-1. The mutations may also constitute substitutions and/or insertions of heterologous nucleic acid molecules in the nef and/or LTR regions such as the incorporation of a sense (i.e. co-suppression) or antisense molecule.

In still yet another embodiment of the present invention, there is provided an isolated, non-pathogenic strain of HIV-1 comprising a deletion in its genome of at least 10 nucleotide within the region of nucleotide 8787 and 9709 and more particularly within the region 8800 and 9700 and even more preferably within the region 8800 and 9410, using the nucleotide numbering of HIV-1_{NL43}.

In one embodiment, the deletion of at least 10 nucleotides is from within a region selected from the list consisting of (using the nucleotide numbers of HIV-1_{NL43}):

| | | |
|---|---|---|
| nucleotide | (i) | 8830-8862; |
| | (ii) | 9009-9035; |
| | (iii) | 9019-9029; and |
| | (iv) | 9033-9049. |

In another embodiment, the deletion of at least 10 nucleotides is from within a region selected from the list consisting of (using the nucleotide numbers of HIV-1_{NL43}):

| | | |
|---|---|---|
| nucleotide | (v) | 9281-9371; |
| | (vi) | 9281-9362; |
| | (vii) | 9105-9224; and |
| | (viii) | 9271-9370. |

In yet another embodiment, the deletion of at least 10 nucleotides is from within a region selected from the list consisting of (using the nucleotide numbers of HIV-1_{NL43}):

| | | |
|---|---|---|
| nucleotide | (ix) | 8882-8928; |
| | (x) | 8850-9006; |
| | (xi) | 8792-9041; and |
| | (xii) | 9112-9204. |

In still yet another embodiment, the deletion of at least 10 nucleotides is from within a region selected from the list consisting of (using the nucleotide numbers of HIV-1_{NL43}):

| | | |
|---|---|---|
| nucleotide | (xiii) | 9105-9224; |
| | (xiv) | 9389-9395; and |
| | (xv) | 9281-9366. |

The above embodiments including any combinations thereof or functionality equivalents thereof. Most preferred variants of HIV-1 are defined in Table 3.

Reference herein to the non-pathogenic HIV-1 strains of the present invention includes reference to components, parts fragments and derivatives thereof including both genetic and non-genetic material. Furthermore, the non-pathogenic HIV-1 strains may be in isolated form or resident in peripheral blood mononuclear cells (PBMCs) or other like cells where the genome of the HIV-1 strains is integrated as DNA from said HIV-1 strains such as proviral DNA. In addition, the present invention extend to recombinant virus such as from (or resident in) prokaryotes or eukaryotes as well as in the form of infectious molecular clones.

Accordingly, the present invention provides for the non-pathogenic HIV-1 isolate, genomic material therefrom complementary proviral DNA, molecular infectious clones, recombinant viral particles or genetic sequences therefrom or cells expressing same or blood cells carrying proviral DNA or to any mutants, derivatives, components, fragments, parts, homologues or analogues of the foregoing.

Another aspect of the present invention contemplates a synthetic peptide comprising a sequence of amino acids as defined in SEQ ID NO:699 or a part or a fragment thereof. The synthetic peptide generally comprises at least four, preferably at east five, more preferably at least six and even more preferably at least seven or more of the amino acids as defined in SEQ ID NO:699. Furthermore, the synthetic peptides may comprise chemically modified amino acids or structurally, functionally and/or stereochemically equivalent substitute amino acids. Such synthetic peptides are useful in diagnostic protocols or in therapeutic regimums, such as in generating antibodies to that particular region of the Nef protein. By administering the synthetic peptides of this aspect of the present invention, higher titres of antibodies to a particular region of Nef may be obtained compared to what would be stimulative if a larger portion of Nef is used.

The non-pathogenic HIV-1 strain of the present invention are particularly useful in the development of therapeutic compositions, therapeutic molecules ad/or diagnostic reagents. With regards to the former, the non-pathogenic HIV-1 strain may be considered as a live attenuated vaccine where individuals carrying DNA derived from said non-pathogenic HIV-1 stain such as proviral DNA in target cells are protected from infection by a corresponding pathogenic strain. The term “vaccine” is used in its broadest sense as a therapeutic composition or molecule which prevents or reduces HIV-1 infection or risk of infection or which ameliorates the symptoms of infection. It may involve the stimulation of an immune response or may involve blocking HIV-1 cells receptors and/or the use of genetic compositions, for example, to introduce ribozymes or antisense molecules to HIV-1 directed genetic sequences or to prepare infectious molecular clones. For convenience, all such compositions are referred hereinafter to “therapeutic compositions”.

Accordingly, the present invention contemplates a method for inhibiting or reducing the risk of infection by a pathogenic strain of HIV-1, said method comprising administering to a subject a non-pathogenic HIV-1 as hereinbefore defined in an amount effective to infect target cells and to generate target cells carrying proviral DNA from said non-pathogenic HIV-1. More particularly, the present invention contemplates a method for inhibiting or reducing productive infection of an individual by a pathogenic strain of HIV-1, said method comprising administering to a subject a non-pathogenic isolate of HIV-1 in an amount effective to infect target cells and to generate target cells carrying proviral DNA from said non-pathogenic HIV-1. By “productive infection” as used in the specification and claims herein is meant the infection of a cell or cells by a pathogenic strain of HIV-1 which leads ultimately to the symptomology of AIDS or AIDS related diseases. A cell infected productively produces pathogenic virions. By definition, infection of an individual by a non-pathogenic s of HIV-1 would not lead to productive infection. Non-pathogenic HIV-1 stains generally replicate to a sufficient extent to protect cells against challenge by virulent or pathogenic drain The methods of the present invention are applicable prophylactically (i.e. to prevent de novo infection) or therapeutically (i.e. to reduce or slow disease progression).

The present invention further provides a method for vaccinating individual against the development of AIDS or AIDS-related diseases, aid method comprising administering to said individual a non-pathogenic isolate of HIV-1 in an amount effective to infect target cells and to generate target cells carrying proviral DNA from said non-pathogenic HIV-1. The term “vaccinating” should not be taken as limiting the invention to the prevention of HIV-I infection by solely immunological means. The term “vaccinating” includes any means of preventing productive Infection of an individual by pathogenic HIV-1. Particularly, preferred non-pathogenic strains of HIV-1 according to these aspects of the present invention arc generally defined as encoding a modified protein such as a modified Nef protein and in particular a modified Nef protein which is substantially non-interactive to antibodies to amino acids 162 to 177 of wild-type HIV-1 Nef.

As an alternative to the above methods, a therapeutic composition as hereinbefore defined is administered. The non-pathogenic isolate may be administered inter alia as an isolated viral preparation or via infected blood cells. Another aspect of the invention provides a therapeutic composition for inhibiting or reducing the risk of infection by a pathogenic strain of HIV-1 said therapeutic composition comprising a non-pathogenic strain of HIV-1 or genetic sequences derived therefrom as herein before described and optionally one or more pharmaceutically acceptable carriers and/or diluents. In a further embodiment, the therapeutic compositions comprise the synthetic peptides comprising an amino acid sequence as set forth in SEQ ID NO:699 or a pert, fragment or homologue thereof. Such a vaccine would generate high titre antibodies to a specific region of Nef protein.

The therapeutic composition of the present invention is generally suitable for intravenous, intraperitoneal, intramuscular, intramucosal (e.g. nasal spray, respiratory spray) or other forms of parenteral administration. The therapeutic composition might also be administered via an implant or rectally or orally. In addition to the mutations contemplated above, the non-pathogenic HIV-1 stain may also contain one or more other mutations to further reduce the risk of reversion to virulence and/or to insert a genetic sequence capable of providing directly or indirectly an identifiable signal; having further anti-HIV-1 properties and/or immunostimulatory or cell by properties. For example, the non pathogenic HIV-1 isolate in the therapeutic composition may comprise additional genetic material capable of directing expression of antisense nucleotide sequences to inhibit expression of one or more proteins encoded by a pathogenic strain of HIV-1. Alternatively, sense co-suppression may be employed Preferred sense or antisense molecules would reduce expression of the nef gene or affect normal functioning of the LTR region. In a particularly preferred embodiment, the nucleotide sequence encoding amino acids 162 to 177 is targetted by sense or antisense molecules.

According to his embodiment, the non-pathogenic HIV-1 strain may be considered as a targeting agent to introduce genetic constructs capable of reducing expression of one or more HIV-1 proteins or polypeptides In this embodiment there is provided a viral isolate which:
(i) is genetically or immunologically related to a pathogenic strain of HIV-1;
(ii) is substantially non-pathogenic in human subjects;
(iii) comprises a first nucleotide sequence constituting its genome which is capable of hybridising under medium stringency conditions to SEQ ID NO: 1 or a complementary from thereof; and
(iv) comprises a second nucleotide sequence within said first nucleotide sequence and which second nucleotide sequence directs expression of a mRNA molecule capable of inhibiting, reducing or otherwise down-regulating translation of a protein or polypeptide encoded by a pathogenic strain of HIV-1 or inhibit, reduce or otherwise down regulate operation of a non-protein encoding a region of a pathogenic strain of HIV-1.

Preferred proteins or polypeptides targeted for reduced expression are those encoded by one or more of the following: gag pot env, tat, rev, vpu, vpr, vif and/or nef genes. The most preferred protein or polypeptide targeted for reduced expression is the product of the nef gene. Alternatively, the target nucleotide sequence does not encode a polypeptide or protein but is required for other functions such a integration or excision from the human genome or expression of genes on the viral genome. An example of such a nucleotide sequence is the LTR region. Accordingly, the present invention extends to disruption to the function of such regions.

In a particularly preferred embodiment there is provided a viral isolate which:
(i) is genetically or immunologically related to a pathogenic strain of HIV-1;
(ii) is substantially non-pathogenic in human subjects, and
(iii) comprises a nucleotide sequence which directs expression of a mRNA molecule capable of inhibiting, reducing or otherwise down-regulating translation of Nef.

Preferably, the nucleotide sequence reduces levels of amino acids 162 to 177 in Nef. The above aspect relates to use of antisense technology. The present invention extends, however, to use of ribozymes and/or co-suppression to achieve the s results. In an alternative embodiment, or in addition to, the second (or optionally a third) nucleotide sequence encodes an antiviral agent (e.g. interferon) and/or an immune enhancing agent.

The identification of deletions inter alia in the nef gene and/or LTR region in asymptomatic subjects provides a unique opportunity to study the in vivo effects of attenuated HIV-1 strains carrying one or more mutations in selected genetic regions. In particular, the present invention provides a means for designing therapeutic compositions directed to inhibiting expression of a nef gene and/or LTR region in a pathogenic HIV-1 strain (such as contemplated above) as well as developing a therapeutic regimen aimed at inhibiting the activity of the nef gene product for the function of the LTR region. According to this latter embodiment, the present invention provides a therapeutic composition comprising a molecule capable of inhibiting the intracellular activity of the nef gene product and/or LTR region, said composition further comprising one or more pharmaceutically acceptable carriers and/or diluents. In one preferred embodiment, the inhibition effects an acids 162 to 177 of Nef.

The molecule contemplated by the above aspect of the subject invention may be a protein, polypeptide, peptide, chemical compound, sugar moiety or derivative of the nef gene product. The molecule will need to be capable of entering an infected cell. Where the molecule is a protein, polypeptide or peptide, it may be encoded by the second nucleotide sequence on the targeting vector as contemplated above. Alternatively, the molecule may be a nucleic acid molecule capable of targeting the nef gene or LTR region. Where the nef gene is targeted, the preferred region is the nucleotide sequence which encodes amino acids 162 to 177 of Nef.

The deletion mutants of the present invention may result in a modified nef gene product either having no readily discernable activity or having activity different to the naturally occurring nef protein. In any event, if a mutant nef gene product is produced it will generally have a lower molecular weight than the naturally occurring nef protein and will have a different overall amino acid sequence. Importantly, it will be immunologically distinguishable from wild-type Nef in hat it will be substantially non-interactive with antibodies to a particular region of Nef, such as amino acids 162 to 177 of wild-type Nef. This provides, therefore, for a means for diagnosing individuals with benign HIV-1 infection by, for example, assaying for a modified nef protein or screening for a modified nef gene sequence. Alternatively, benign HIV-1 infection may be detected by assaying for a modified LTR region such as an altered nucleotide sequence.

These aforementioned aspects of the present invention apply to screening deletion, truncation or other mutants of HIV-1 derived proteins where such mutations result in a strain of HIV-1 being substantially non-pathogenic. Although a variety of procedures are available to detect a modified HIV-1-derived protein, a particularly convenient approach is to screen HIV-1 infected individuals for the absence of antibodies to the deleted or truncated portion of a target protein.

According to one embodiment there is provided a method for determining the pathogenicity of a strain of HIV-1 after said HIV-1 strain infect cells of an individual said method comprising contacting a biological sample from said individual with a peptide corresponding to a deleted or truncated region of an HIV-1-derived protein and screening for the absence of antibody binding to said peptide, wherein the absence of antibody binding is indicative of a deletion or truncation in that protein and further indicative of the non-pathogenicity of said strain of HIV-1.

Although this general methodology is applicable to any protein encoded by HIV-1 which is critical for pathogenicity in a host, it is particularly useful in screening deletions in the Nef protein. An exemplary deletion in the Nef protein is all or part of the sequence of amino acids 162 to 177 of Nef Preferably, the assay would include a positive control such that where antibodies are present to Nef, such antibodies would bind to this positive control. When a Nef protein carries a deletion at or about amino acids 162 to 177, antibodies to this region would be absent in an individual infected by a non-pathogenic stain of HIV-1 and no, binding would be detected to a peptide covering this region.

According to a particularly preferred embodiment, there is contemplated a method for determining the pathogenicity of an HIV-I strain after said stain infects cells of an individual said method comprising contacting a biological sample from said individual with an effective amount of a peptide having an amino acid sequence comprising or within amino acids 162 to 177 of wild-type HIV-1_{NL43} Nef, said contact being for a time and under conditions sufficient for an antibody if preset in said biological sample to form a complex with said peptide and then detecting the presence of said complex wherein the absence of a complex is an individual seropositive for HIV-1 is indicative of that the individual being infected with a non-pathogenic strain of HIV-1. The method may also comprise contacting the biological sample with one or more peptides derived from other regions of Nef such as flanking regions to amino acids 162 to 177 where antibodies to such other regions may be expected even in non-pathogenic HIV-1 strains This would provide a suitable positive control. A biological sample according to this embodiment would be any source of antibodies such a: serum and whole blood. Preferably, the peptides are immobilized to a solid support as described below. This method of the present invention is also applicable for determining the risk of an individual seropositive for HIV-1 developing symptoms of AIDS. Again, substantial absence of antibodies to amino acids 162 to 177 of Nef in an HIV-1 seropositive individual would be indicative of a low risk of an individual developing AIDS.

Other methods of screening for the pathogenicity or otherwise of stains of HIV-1 readily become apparent as a result of the present invention. For example, antibodies may be first generated to modified Nef proteins from non-pathogenic strains of HIV-1 where such antibodies would not recognise wild-type Nef protein.

According to one embodiment, there is provided a method for determining the pathogenicity of an HIV-1 stain after said HIV-1 strain infects cells of an individual, said method comprising contacting a biological sample from said individual with an effective amount of an antibody specific to a nef protein from a non-pathogenic strain of HIV-1 (as hereinbefore defined) for a time and under conditions sufficient to form an antibody-modified nef protein complex and then detecting said complex. The presence of said complex is indicative of a modified nef gene product and of the non-pathogenicity of the strain of HIV-1. Alternatively, the isolated Nef could be tested against a panel of antibodies where certain antibodies are specific to a deleted region. The absence of antibody binding would be indicative of a deletion and, therefore, a modified Nef protein and in turn a putative non-pathogenic form of the virus. The biological sample is a sample likely to contain the modified nef gene product such as tissue extract or cell extract of an infected cell. However, where the modified nef gene product is capable of permeation or transport out of the cell, suitable biological fluid would include serum, whole blood, lymph and mucosal secretion amongst other fluids.

Many variations in the aforementioned assays are possible and am contemplated herein. For example, an assay could also be based on the inability for a nef specific antibody to bind to a modified nef protein. For the purposes of the present invention the term “contacting including mixing”.

The presence of a modified nef molecule, such as a molecule carrying a deletion, or other suitable HIV-1 derived protein in biological fluid can be detected using a wide range of immunoassay techniques such as those described in U.S. Pat. Nos. 4,016,043, 4,424,279 and 4,018,653. These include both single-site and two-site, or “sandwich”, assays of the non-competitive types, as well as in the traditional competitive binding assays and include ELISA and RIA techniques. Sandwich assays are among the most useful and commonly used assays and are favoured for use in the present invention. A number of variations of the sandwich assay technique exist, and all are intended to be encompassed by the present invention. Briefly, and by way of example only, in a typical forward assay, a modified nef product-specific antibody is immobilised onto a solid substrate to form a first complex and the sample to be tested for modified nef product brought into contact with the bound molecule. After a suitable period of incubation, for a period of time sufficient to allow formation of an antibody-modified nef product secondary complex, a second modified nef protein antibody, labelled with a reporter molecule capable of producing a detectable signal, is then added and incubated, allowing time sufficient for the formation of a tertiary complex of antibody-modified nef product-antibody. Any unreacted material is washed away, and the presence of the tertiary complex is determined by observation of a signal produced by the reporter molecule. The results may either be qualitative, by simple observation of the visible signal or may be quantitated by comparison with a control sample containing known amounts of hapten. Variations of the forward assay include a simultaneous assay, in which both sample and labelled antibody are added simultaneously to the bound antibody, or a reverse assay in which the labelled antibody and ample to be tested are first combined incubated and then added simultaneously to the bound body. These techniques are well known to those skilled in the art, and the possibility of minor variations will be readily apparent. The antibodies used above may be monoclonal or polyclonal. In a preferred embodiment, antibodies to Nef. In an individual's biological fluid are screened for using peptides derived from Nef. The absence of antibodies to specific regions of Nef such as amino acids 162 to 177 would be indicative of a non-pathogenic strain of HIV-1. All such assays and variations of such assays are encompassed by the present invention.

The solid substrate is typically glass or a polymer, the most commonly used polymers being cellulose, polyacrylamide, nylon, polystyrene, polyvinyl chloride or polypropylene. The solid supports may be in the form of tubes, beads, discs or microplates, or any other surface suitable for conducting an immunoassay. The binding processes are well-known in the art and generally consist of crosslinking covalently by or physically adsorbing the molecule to the insoluble carrier.

By “reporter molecule”, as used in the present specification, is meant a molecule which, by its chemical nature, produces an analytically identifiable signal which allows the detection of antigen-bound antibody. Detection may be either qualitative or quantitative. The most commonly used reporter molecules in this type of assay are enzymes, fluorophores or radionuclide containing molecules (i.e. radioisotopes). In the case of an enzyme immunoassay, an enzyme is conjugated to the second antibody, generally by means of glutaraldehyde or periodate. As will be readily recognised, however, a wide variety of different conjugation techniques exist which are readily available to one skilled in the art. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable colour change. It is also possible to employ fluorogenic substrates, which yield a fluorescent product.

Alternatively, fluorescent compounds, such as fluorescein and rhodamine, may be chemically coupled to antibodies without altering their binding capacity. When activated by illumination with light of a particular wavelength, the fluorochrome-labelled antibody adsorbs the light energy, inducing a state of excitability in the molecule, followed by emission of the light at a characteristic colour visually detectable with a light microscope. As in the EIA, the fluorescent labelled antibody is allowed to bind to the first antibody-hapten complex. After washing off the unbound reagent, the remaining complex is then exposed to the light of the appropriate wavelength, the fluorescene observed indicates the presence of the hapten of interest. Immunofluorescence and EIA techniques are both very well established in the art and are particularly preferred for the present method. However, other reporter molecules, such as radioisotope, chemiluminescent or bioluminescent molecules, may also be employed. It will be readily apparent to the skilled technician how to vary the procedure to suit the required purpose. It will also be apparent that the foregoing can be used to label a modified nef product and to use same directly in the detection of, for example, circulatory antibodies specific to said modified nef product.

Alternatively, genetic assays may be conducted to screen for abbreviations in the nef gene and/or LTR region. Such a genetic assay may be by Southern or Northern blot analysis, PCR analysis or the like using oligonucleotides specific to a deleted region of a nef gene and/or LTR region.

According to this embodiment there is provided a method for determining the pathogenicity of an HIV-1 strain after said HIV-1 strain infects cells of an individual, said method comprising determining directly or indirectly the presence of a deletion mutation in the genome of said HIV-1 wherein the presence of a such a mutation is indicative of the presence of a non-pathogenic strain of HIV-1. The deletion mutation may result in the genome being unable to synthesize a polypeptide or protein from a pathogenic strain of HIV-1 or may direct the synthesis of a truncated or deleted form of said polypeptide or protein For example, a Nef protein with amino acids 162 to 177 deleted therefrom. The mutation may also lead to altered expression of a polypeptide detectable by, for example, decreased synthesis of a particular protein, such as the nef gene product. Alternatively, the deletion mutation affects the LTR region or a regulatory region of the HIV-1 genome. In either case, affected viruses may also be detected by, for example, obey low viral copy number such a low viral load.

Preferably, said non-pathogenic HIV-1 carries a deletion in its genome of at least 10 nucleotides within the region from nucleotide 8787 to nucleotide 9709 using the nucleotide numbering of HIV-1_{NL43}.

Preferably, said non-pathogenic HIV-1 carries a deletion in its genome of at least 10 nucleotides from within a region selected from the list coning of:

| | | |
|---|---|---|
| nucleotide | (i) | 8830-8862; |
| | (ii) | 9009-9035; |
| | (iii) | 9019-9029; and |
| | (iv) | 9033-9049. |

Preferably, said non-pathogenic HIV-1 carries a deletion in its genome of at least 10 nucleotides from within a region selected from the list consisting of:

| | | |
|---|---|---|
| nucleotide | (v) | 9281-9371; |
| | (vi) | 9281-9362; |
| | (vii) | 9105-9224; and |
| | (viii) | 9271-9370. |

Preferably, said non-pathogenic HIV-1 carries a deletion in its genome of at least 10 nucleotides from within a region selected from the list consisting of:

| | | |
|---|---|---|
| nucleotide | (ix) | 8882-8928; |
| | (x) | 8850-9006; |
| | (xi) | 8792-9041; and |
| | (xii) | 9112-9204. |

Preferably, said non-pathogenic HIV-I carries a deletion in its genome of at least 10 nucleotides from within a region selected from the list consisting of:

| | | |
|---|---|---|
| nucleotide | (xiii) | 9105-9224; |
| | (xiv) | 9389-9395; and |
| | (xv) | 9281-9366. |

The above nucleotide numbers are based on the nucleotide numbering in the NL43 genome.

Particularly preferred oligonucleotides are based on the deleted regions of the nef gene and/or LTR region such as but not limited to one or more oligonucleotides based on SEQ ID NO: 2 to SEQ ID NO: 613 and/or SEQ ID NO: 652 to SEQ ID NO: 799.

Most preferred deletions include deletions of one or more of SEQ ID NO:803 to 841 which cover amino acids 162 to 177 of Nef. A particularly preferred genetic assay screens for this deletion.

The present invention further extends to kits for the diagnosis of infection by pathogenic strains of HIV-1 or for determining the pathogenicity of infecting virus. The kits would be in compartmental form each comprising one or more suitable reagents for conducting the assay.

The present invention is further described by the following non-limiting Examples.

### EXAMPLE 1

### Source Material

For the purposes of the following examples, non-pathogenic HIV-1 strains were isolated from recipients of HIV-1 infected blood. The recipients are designated “C18”, “C54”, “C98”, “C49”, “C64” and “C124”. The donor is identified herein as “D36”. The place of isolation may be indicated after the abbreviation of “HIV”. For example, St Vincents Hospital Sydney (HIV_{StV}) or Macfarlane Burnet Centre of Medical Research, Melbourne (HIV_{MBC}).

Exemplary viral isolates referred to herein as “C18” and “C98” war deposited at the PHLS Centre for Applied Microbiology and Research, European Collection of Animal Cell Culture (ECACC), Division of Biologies, Porton Down, Salisbury, Wiltshire SP4 OJG. C18 was deposited on 17 Oct. 1994 under Provisional Accession Number V94101706 and C98 was deposited on 31 Oct. 1994 under Provisional Accession Number V941031169. Another isolate “C54” was deposited at ECACC on 10 Mar. 1995 under Provisional Accession Number V95031022.

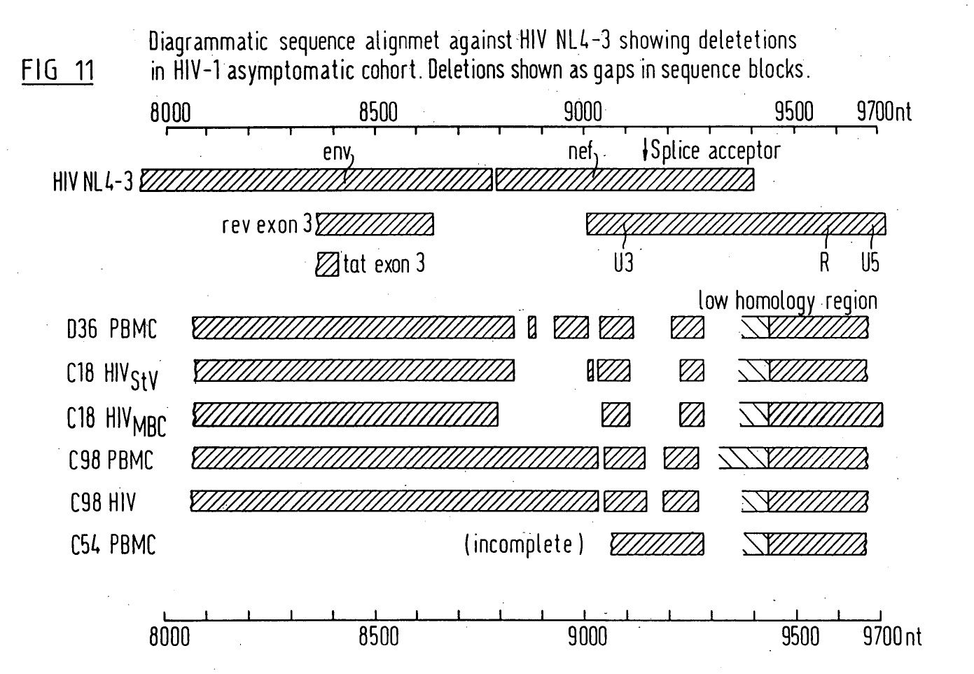

FIG. 11 is a summary of the deletion mutants of the present invention.

Viruses are isolatable by the following procedures:
1. Infected peripheral blood mononuclear cells (PBMCs) were co-cultured with HIV-1 seronegative donor PBMCs. A convenient source of seronegative donor PBMCs is a blood bank. The supernatants and cells ar harvest every 7 days and fresh medium added with CD8 depleted PBMCs. CD8 depletion promotes the ability to isolate HIV-1. The culture and procedure is continued for up to approximately 5 weeks;
2. The infected PBMCs are purified from whole blood and these cells are cultured alone for up to 5 weeks. PMBCs alone are used beaus the virus is more likely to be monocytotropic. Fresh medium is added weekly and supernatant fluid is harvested at this time;
3. Supernatant fluids are harvested every approximately 7 days, fresh medium and fresh HIV-1 seronegative CD8 depleted PBMCs are added at this time;
4. HIV-1 seronegative PBMCs are pretreated with with M-CSF for approximately 72 hours prior to the addition of infected PBMCs. M-CSF has been shown to enhance HIV-1 replication in monocytes (Gendelman at al, 1988); and
5. The supernatant fluid is harvested from the cultures of step 4 every approximately 7 days, fresh medium added together with HIV-1 seronegative stimulated CD8 depleted PBMCs. Te virus is isolated from the infected PBMCs.

A particularly preferred method of isolation is as follows:

HIV negative donor PBMC were stimulated by culture in RPMI 1640 containing 10% v/v fetal calf serum (FCS), 15 mM N-2-hydroxyethylpiperazine-N-2-ethanesulfonic acid, 0.1% w/v sodium bicarbonate with 100 IU/ml penicillin and 100 m streptomycin with the addition of 10 μg/ml PHA (Wellcome, Temple Hill, Dartford, England) for 72 h prior to co-culture. Fresh patient cells (10×10⁶ cells) were then co-cultured with the PHA-activated donor PBMC (10×10⁶ cells). Immediately on co-culture 2×10⁶ of the mixed cell population were UV irradiated (254 nm, 300 μW/cm², 15 sec), added back to the remaining cells and cultured for 29 days. After UV treatment cells were resuspended at 1×10⁶ cells/ml in RPMI 1640 containing 10% v/v FCS, 15 mm HEPES, 0.1% w/v sodium bicarbonate, 25 μg/ml glutamine, 100 IU/ml penicillin, 100 μg/ml streptomycin, 2 μml polybrene; 4 μg/ml hydrocortisone (Sigma, St Louis, Mo., USA). 20 U/ml interleukin 2 (Boehringer Mannheim Germany) and 120 nU/ml anti-interferon (ICN Biochemicals, Costa Mesa, Calif., USA). Cells were maintained by half medium changes every 3 to 4 days after PHA stimulation, with the addition of fresh stimulated donor PBMC on days 7, 14 and 21. Virus production was assayed for by cell-free reverse transcriptase activity (Neate et al, 1987) or p24 activity (Abbot Diagnostics assay).

### EXAMPLE 2

### DNA Preparation and PCR Amplification

Non-pathogenic HIV-1 (e.g. strain C18) infected peripheral blood mononuclear cells (PBMC) were harvested 4 days after infection of phytohaemagglutinin (PHA) stimulated HIV-1 negative donor PBMC cultured by the method of Ne et al (1987) and washed in phosphate buffered saline (PBS). PBMC from Donor D36 and Recipients C18, C54 and C98 were prepared by Ficoll isopaque centrifugation of buffy coat cells and washed with PBS.

Approximately 10⁷ cells were lysed in 1 ml lysis solution (0.45% v/v NP40, 0.45% v/v Tween 20, 10 mM Tris HCl pH 8.3, 40 mM KCl 2.5 mM MgCl₂) and digested with 60 μg/ml proteinase K (Boehringer Mannheim) at 55° C. for 1 hour followed by 100° C. for 10 minutes. Lysates were stored at −20° C.

All polymerase chain reaction (PCR) primers (Table 1) and sequencing primers (Table 2) were synthesized using an Applied Biosystems model 391 DNA synthesis using phosphoramidite chemistry.

Strict physical separation was maintained for sample, PCR reagent mix and PCR reaction preparations as well as amplification and analysis Final reaction mixes (50 μl) contained 2 μl neat or diluted cell lysate, 0.2 μM each primer, 200 mM dNTPs and 1.25 units Taq polymerase (Boehringer Mannheim) in PCR buffer, (10 mM Tris-HCl pH8.3, 50 mM KCl, 100 μg/ml gelatine) adjusted to the optimum MgCl₂ concentration for the primer pair (1.5-3.0 mM). Aliquoted reagent mix was overlaid with 50 μl oil prior to addition of DNA template lysate After template dc on at 94° C. for 3 min amplification was achieved with 30 cycles of 94° C., 1 min; 55° C., 1 min; 72° C., 2 mins. A final elongation reaction was conducted at 72° C. for 7 minutes. For double PCR amplification 2 μl of first round product was added to the second reagent mix and amplified as before.

PCR amplified DNA was checked for quality, quantity and fragment size by agarose gel electrophoresis in Tris-Acetate-EDTA buffer (Sambrook et al, 1989) stained in ethidium bromide and viewed by UV transillumination.

### EXAMPLE 3

### DNA Sequence Analysis

The DNA sequence of PCP amplified HIV-1 regions was determined by the dideoxynucleotide method (Sanger et al, 1997) using Sequenase T7 polymeras (United States Biochemicals).

PCR amplified DNA was purified by PCR Magic prep resin chromatography (Promega). Approximately 2 to 7 μg purified DNA plus long specific primer (Table 2) were denatured by boiling for 3 nuns and snap from to −20° C. Thc initial labelling reaction was for 3 minutes at 22° C. (room temperature) with ³⁵SdNTP (500 Cl/mmol; Dupont) followed by dideoxynucleotide termination reactions at 37° C. for 5 minutes. NP40, to 0.45% v/v, was included in denaturation and reaction mixes (Bachman et al, 1990). Sequencing reaction products were denatured in formamide and resolved on a 6% w/v polyacrylamide gel containing SM urea, fixed in 10/A v/v acetic acid, 10% v/v methanol and dried. Following autoradiography on XK1 film (Kodak) the gel were mead assembled, translated to protein and aligned using the PC/GENE ite of programs (IntelliGenetics, USA).

TABLE 1

PCR PRIMERS

| PRIMER | SEQUENCE^{1,3} | POSITION² |
|---|---|---|
| C1-1 | TGGAAGGGCTAATTTGGT(616) | 1-18 |
| C1-2 | ATCTTCCCTAAAAAATTAGCCTGTC(617) | 2099-2075 |
| LTR-3′ | AGGCTCAGATCTGGTCTAAC(618) | 9559-9540 |
| SK68 | AGCAGCAGGAAGCACTATGG(619) | 7786-7805 |
| C1-6 | TGCTAGAGATTTTCCACAC(620) | 9709-9691 |
| KS-2 | AGTGAATAGAGTTAGGCAGG(621) | 8326-8345 |
| RT5′-v3 | GTAAGACAGTATGATCAGATA(622) | 2418-2438 |
| RT3′-v2 | TTGTAGGGAATTCCAAATTCC(623) | 4660-4640 |
| RT5′-v2 | CAGGATCCTACACCTGTCAACATAAT(624) | 2487-2506 |
| RT3′-v1 | GGGAATTCCTTATTCCTGCTTG(625) | 4655-4634 |

¹Sequence is presented from 5′ to 3′ of the primer.
²Position is according to the numbering of HIV-1 in Myers et al (1992).
³SEQ ID NOs are given in parentheses.

TABLE 2

SEQUENCING PRIMERS

| PRIMER | SEQUENCE^{1,3} | POSITION² |
|---|---|---|
| KS3 | CCAGAAGTTCCACAATCC(626) | 8570-8553 |
| KS4 | TTCTTCTAGGTATGTGGA(627) | 8753-8735 |
| KS5 | AGTGAATTAGCCCTTCCAG(628) | 9093-9075 |
| KS6 | TGCTAGAGATTTTCCACAC(629) | 9709-9691 |
| SP2 | TGCTCTGGAAAACTCAT(630) | 8006-8022 |
| SP3 | CTTTCTATAGTGAATAGAG(631) | 8318-8336 |
| SP4 | TATTGGAGTCAGGAACT(632) | 8618-8634 |
| SPR | GGTCTAACCAGAGAGAC(633) | 9547-9531 |

¹Sequence is presented from 5′ to 3′ of the primer.
²Position is according to the numbering of HIV-1 in Myers et al (1992).
³SEQ ID NOs are given in parentheses.

### EXAMPLE 4

### Cells and Cell Culture

Peripheral blood was obtained from HIV-1 sero-negative volunteers and mononuclear cells prepared by centrifugation on a Ficoll/Hypaque density gradient (Peper et al, 1968). PBMC were activated with pytophemagglutin (PHA; 10 μg/10⁶ cells) for 48 h at 37° C. washed and then cultured in RPMI 1640 medium containing 10% v/v heat inactivated foetal calf serum, 15 mM HEPES, 0.1% v/v sodium bicarbonate, 25 μg/ml polybrene (Sigma), 10% v/v interleukin 2 (Boehringer Mannheim) and 1:1000 anti-interferon (Miles) (IL-2 medium). Non-PHA stimulated cells were prepared in a similar manner except they were cultured in medium lacking PHA and IL-2.

### EXAMPLE 5

### Antipeptide-Antisera

Antibodies specific for HIV-1 Nef were raised against a peptide corresponding to the predicted amino acid residues 15-27 (AVRERMRRAEPAA SEQ ID NO: 634) of Nef encoded by the HIV-1 clone pNL4.3 (Kemp et al, 1988). The peptide was conjugated to keyhole limpet hemocyanin (KLH; Calbiochem, Behring Diagnostics, Calif.) via glutaraldehyde and this complex used to immunise (0.5 mg peptide conjugate/sheep). Antibodies to the peptide were purified by affinity chromatography. Reactivity of the antibodies with recombinant HIV-1 Nef 25 d 27 was demonstrated by immunoblotting.

### EXAMPLE 6

Reactivity of anti-nef₍₁₅₋₂₇₎ with HIV C18-Infected Cells in Immunoblotting Seven days post-infection HIV-1 C18-infected PBMCs and mock-infected cells were washed in PBS then lysed (0.5% w/v NP-40, 0.5% w/v sodium deoxycholate, 50 mM NaCl, 25 mM Tris-Ha, 10 mM EDTA, 0.01% w/v sodium azide and 10 mM phenylmethylsulphonylfluoride). After nuclei were spun out lysates were electrophoresed in a 13% w/v SDS-polyacrylamide gel (SDS-PAGE) and subsequently transferred to Hybond-C nitrocellulose (Amersham, Buckinghamshire England) for 1 h at 100 V using a Bio-Rad protein transfer cell (Bio-Rad, Richmond, Calif.). Membranes were pre-incubated with 1% w/v BSA/PBS for 2 h at room temperature and then reacted with affinity purified sheep anti-Nef₍₁₅₋₂₇₎, diluted 1:100, overnight at room temperature. After three washes in 1% w/v BSA/PBS, the blots were incubated with donkey anti-sheep Ig conjugated to biotin (Amersham, diluted 1:500) fa 1 h at room temperature. After extensive washing as described above the membranes were incubated with streptavidin-conjugated horse radish peroxidase (Amersham; diluted 1:500 for 1 h at room temperature. All dilutions were made with 1% w/v BSA in PBS. After further washing the membrane was developed with phenylenediamine substrate (Dako, Dapopatts, Denmark). The antibody preparation used in the immunoblotting experiments was free of detectable antibodies to the immunogenic carrier protein and coupling reagent.

### EXAMPLE 7

### Analysis by Polymerase Chain Reaction Amplification

A 5′ fragment defined by primers Cl1 and Cl2 containing the 5′ LTR and part of the gag gene was amplified. DNA from HIV-1 C18 infected PBMC gave an amplified fragment (amplimer) of about 1.9 kb compared with 2.1 kb for pHXB2 control template, implying a deletion of about 200 bp from HIV C18. Further amplification of this fragment with primers defining the U3 region of the LTR (Cl-1 and LTR-3′) gave amplimers of about 300 bp for HIV-1 C18 infected PBMC DNA compared with 340 bp for C18 and D36 PBMC DNA and 484 bp for pHXB2 control. This implies the loss of approximately 140 to 180 bp from the U3 region of these proviral DNAs.

To analyse the nef-gene-3′ LTR region, the nested primer pairs SK68-Cl-6 and KS-2-LTR-3′ were used in a double PCR. Amplimers of approximately 830 bp were obtained for HIV-1 C18 infected PBMC DNA as well as for PBMC DNA from Donor D36 and Recipients C18, C54 and C98 compared with approximately 1230 bp for pHXB2 DNA. These results suggest that about 400 bp of DNA have been deleted from the Donor and Recipient proviral DNAs.

In comparison, amplification of the polymeras gene region by double PCR with the nested primer pairs RT5′-v3-RT3′-v2 and RT5′-v2.RT3′-v1 gave a fragment (approximately 2.1 kb) the same size as the molecular clone pHXB2 fragment for HIV-1 C18 infected PBMC DNA, suggesting that deletions from this region were unlikely.

### EXAMPLES 8

### Nucleotide Sequence of the nef-3′ LTR Region

PCR amplification cents indicated an approximately 200 bp nucleotide deletion from both the nef gene and LTR regions of Donor D36 PBMC and Recipient C18 HIV-1 proviral DNA. To further analyse these regions, the DNA sequence was determined for the PCR amplified nef-3′-LTR region of D36 PBMC, C18 isolates HIV_{MBC} and HIV_{StV} as well as isolate C₉₈ HIV infected PBMC proviral DNA. The 3′ region was amplified with outer primers (SK68-C16) and inner primers (SK68-LTR 3′ or KS2-C16) and sequenced directly using a number of internal sequencing primers based on the HIV-1_{NL43} nucleotide sequence (Table 2).

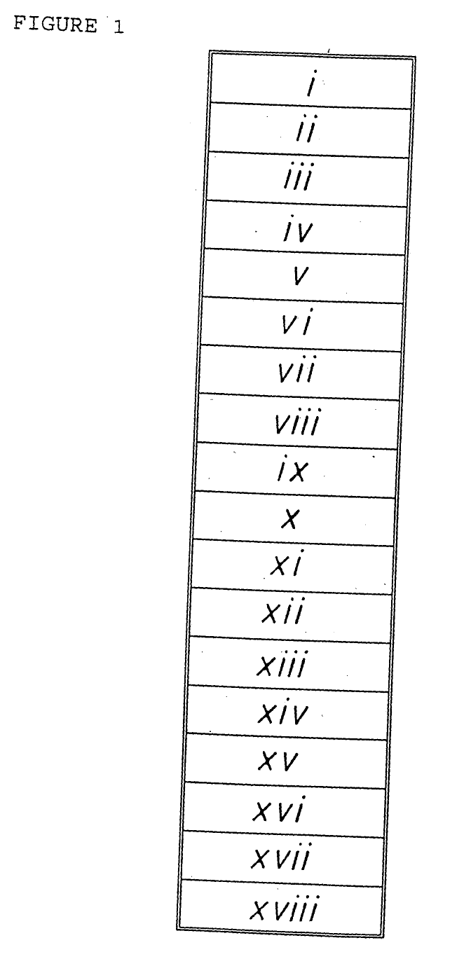

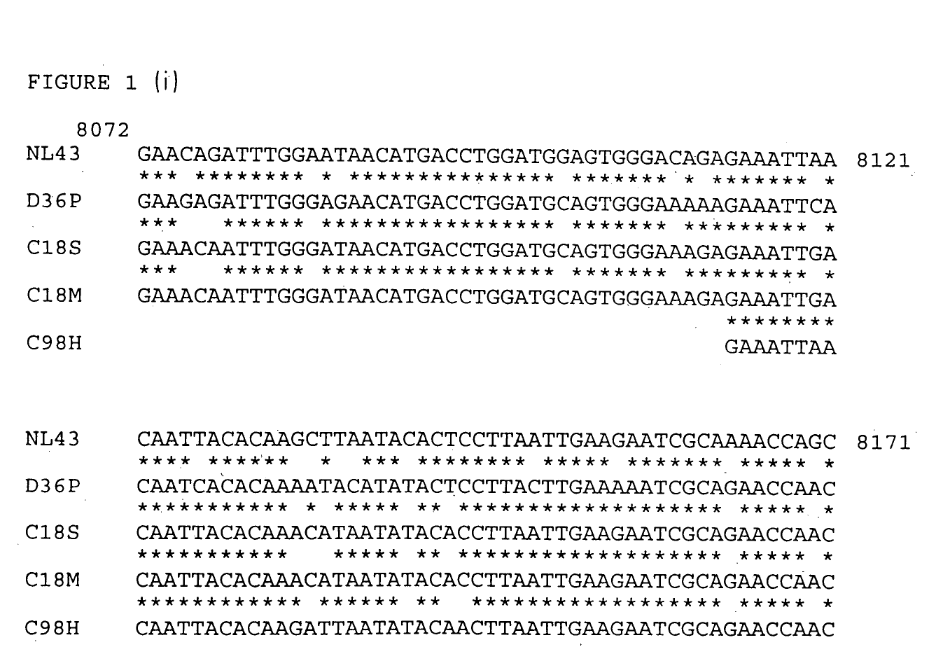

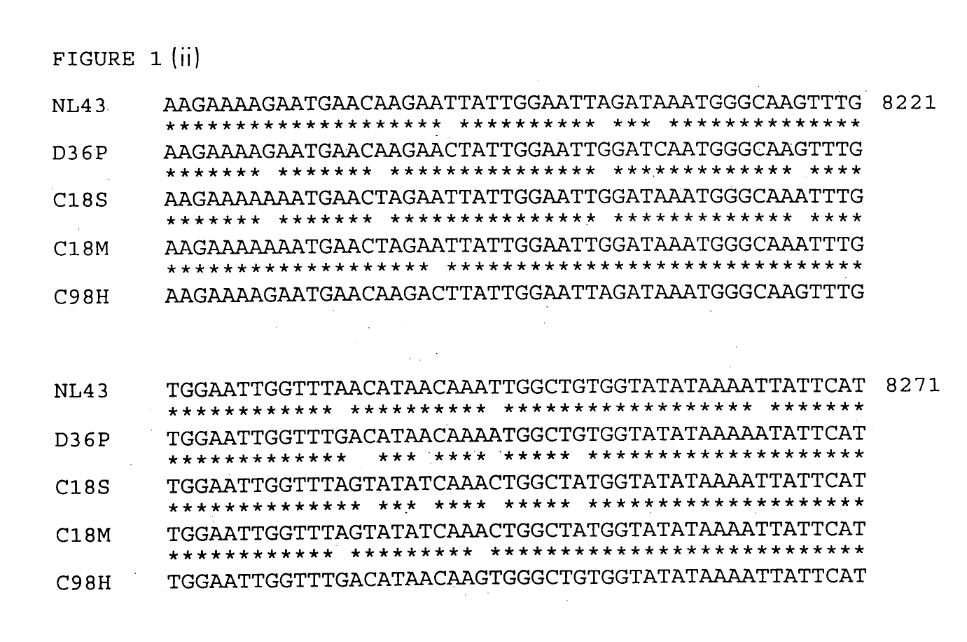

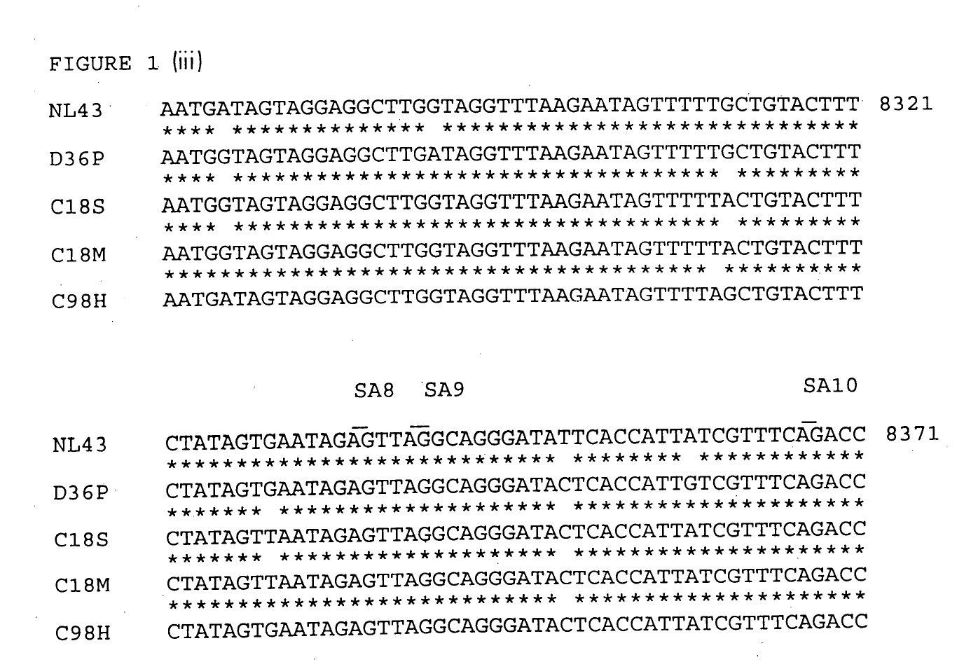

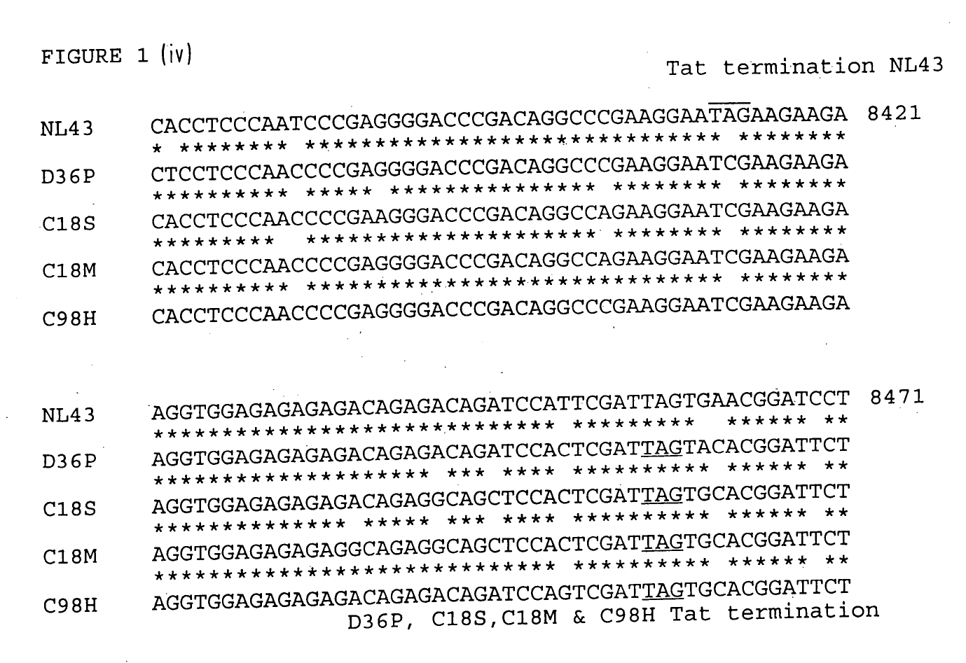

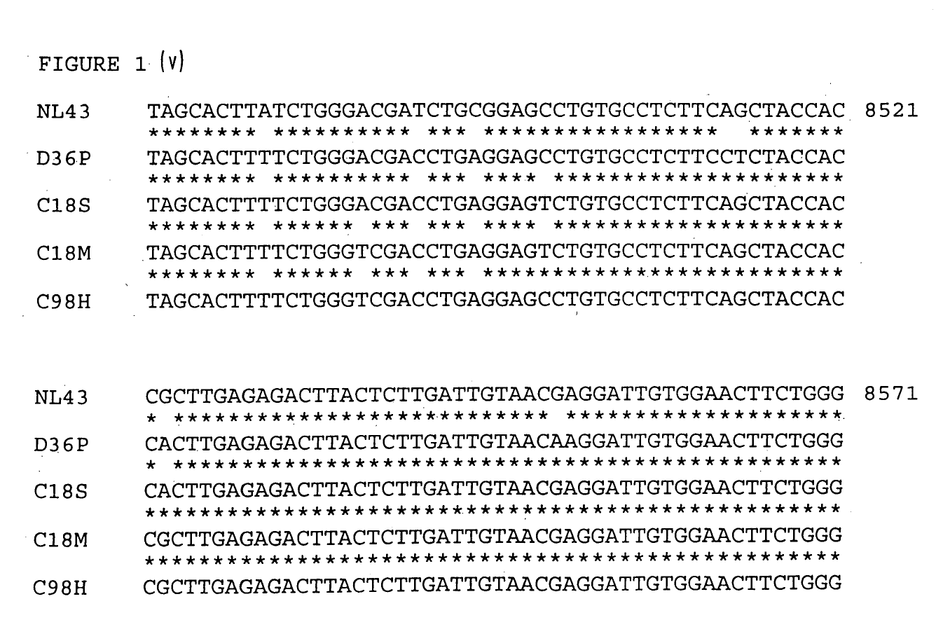

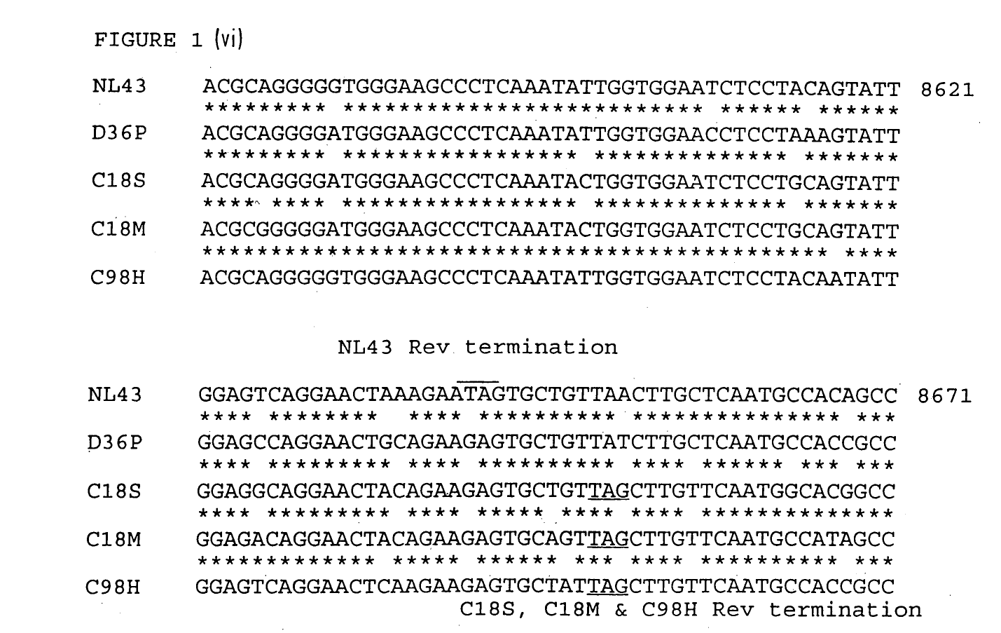

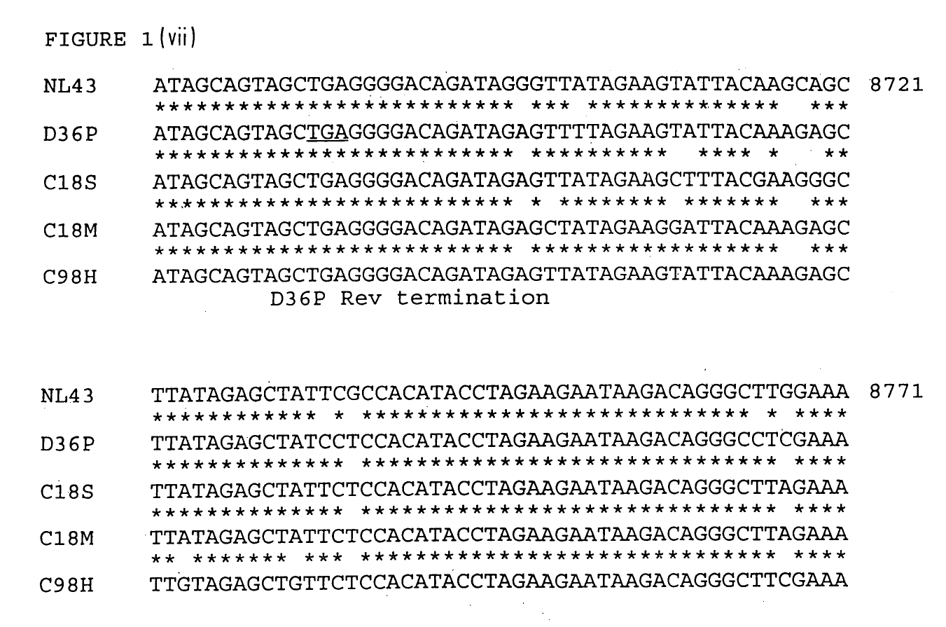

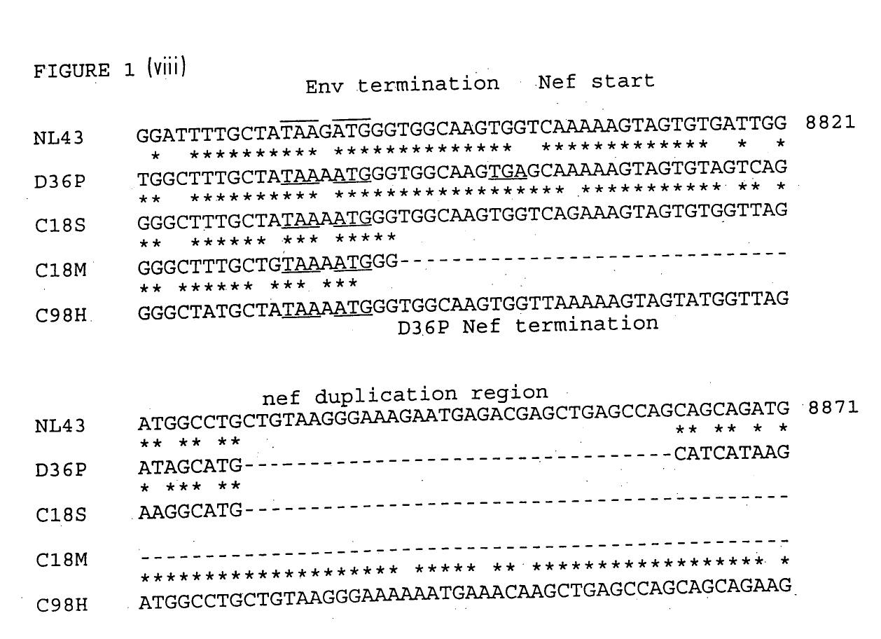

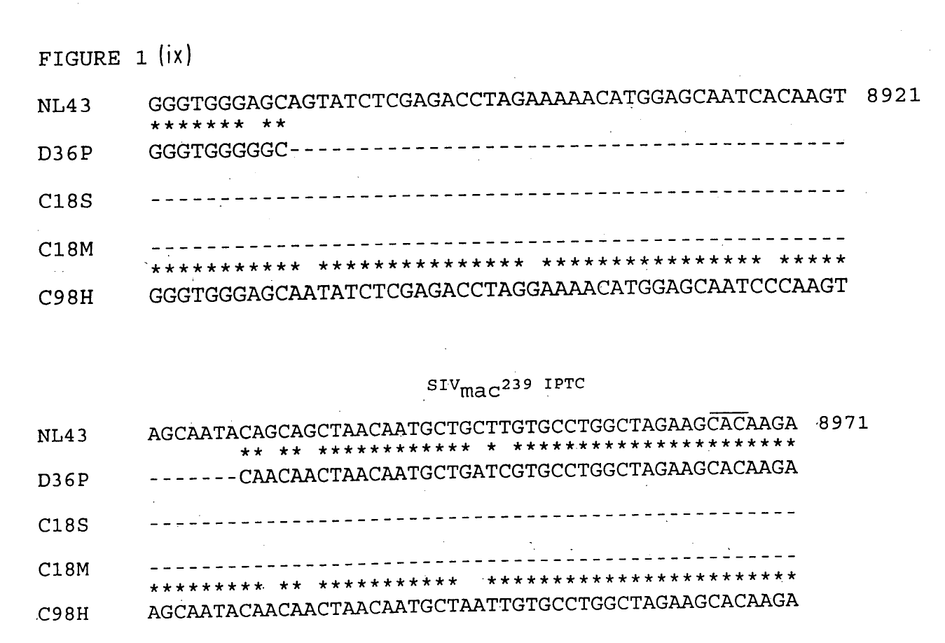

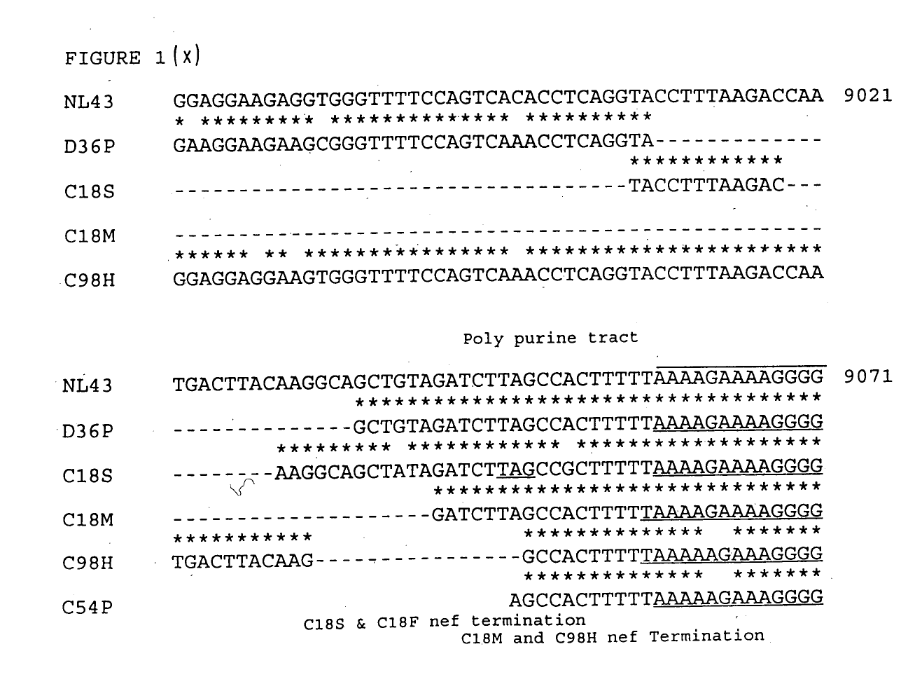

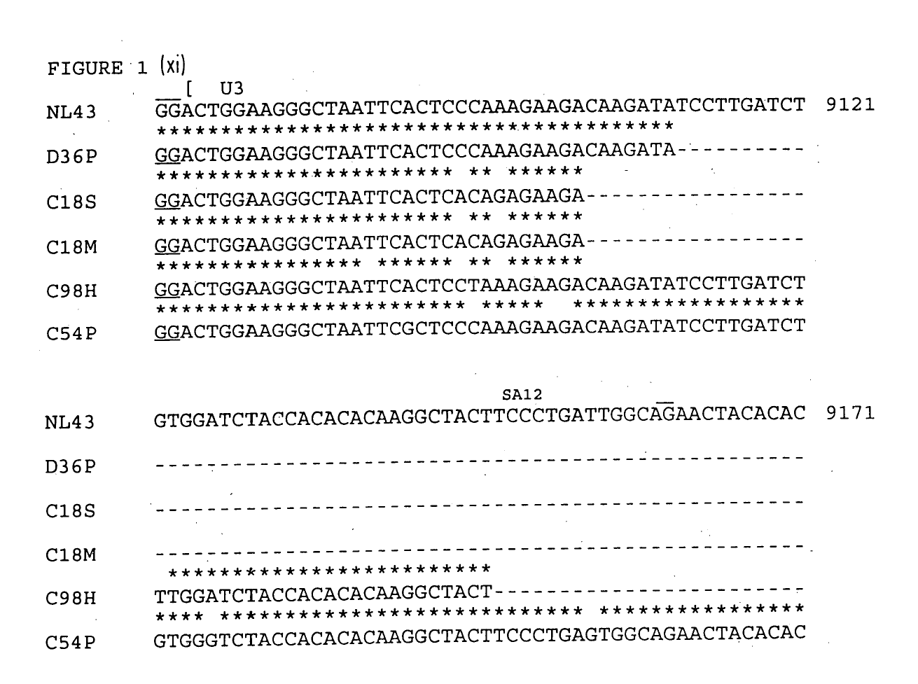

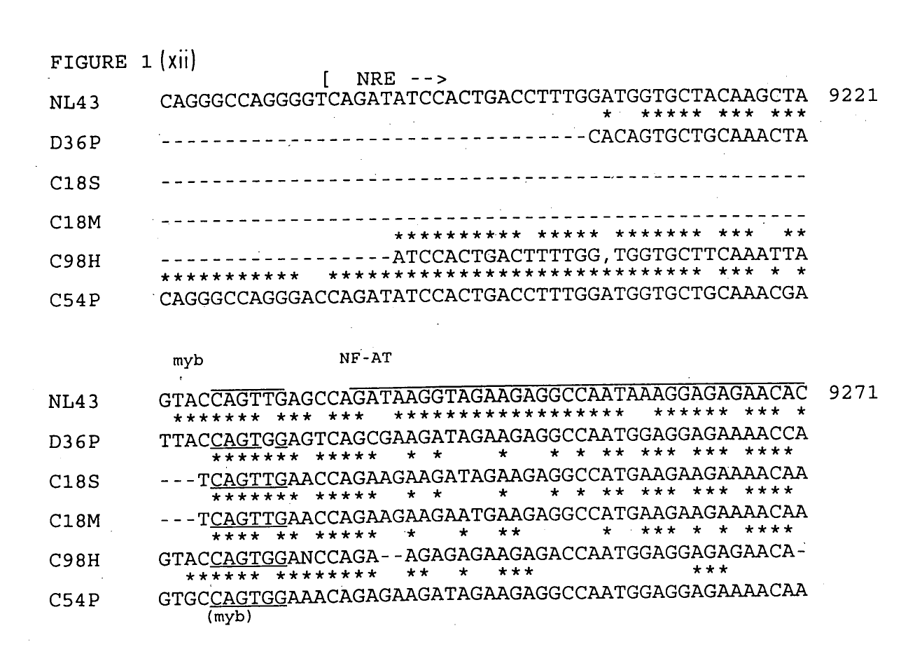

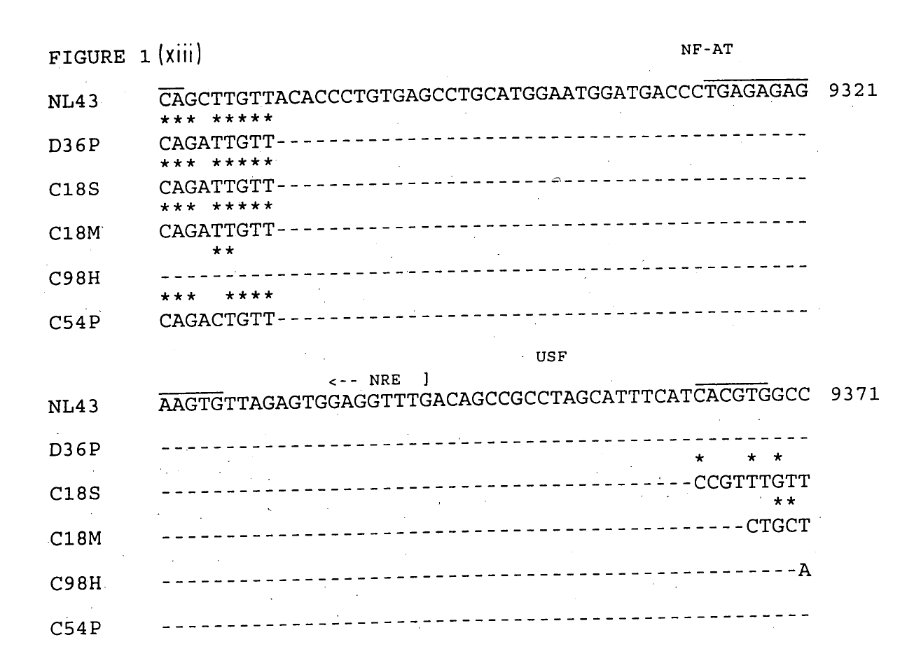

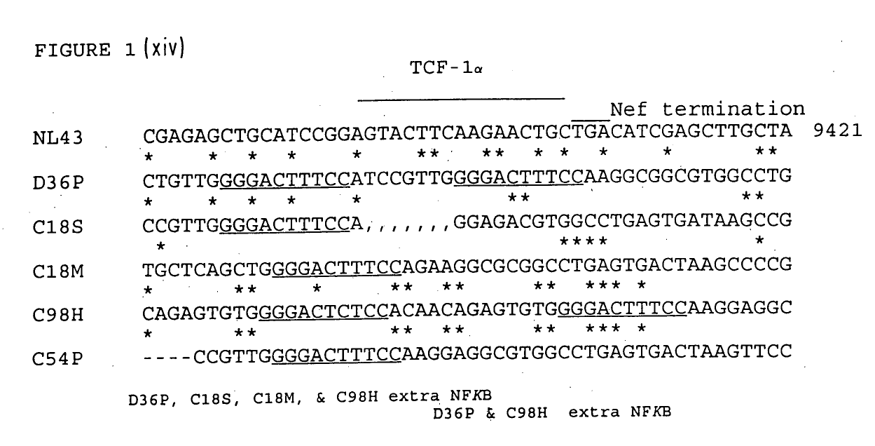

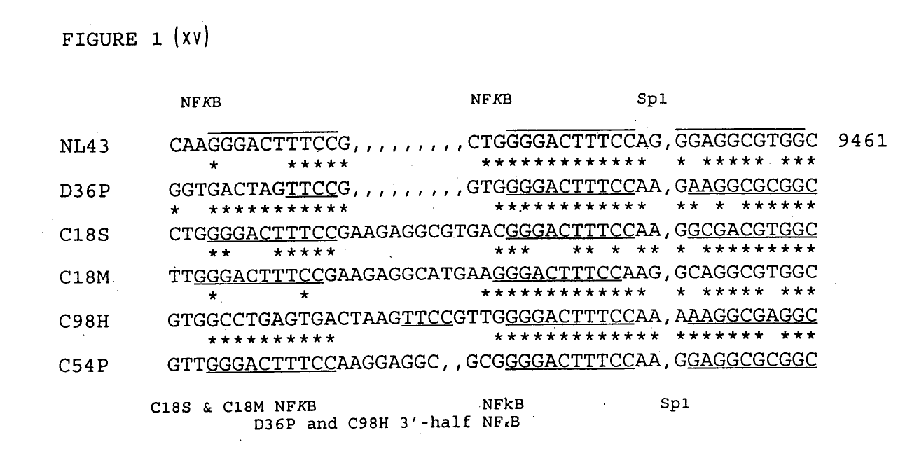

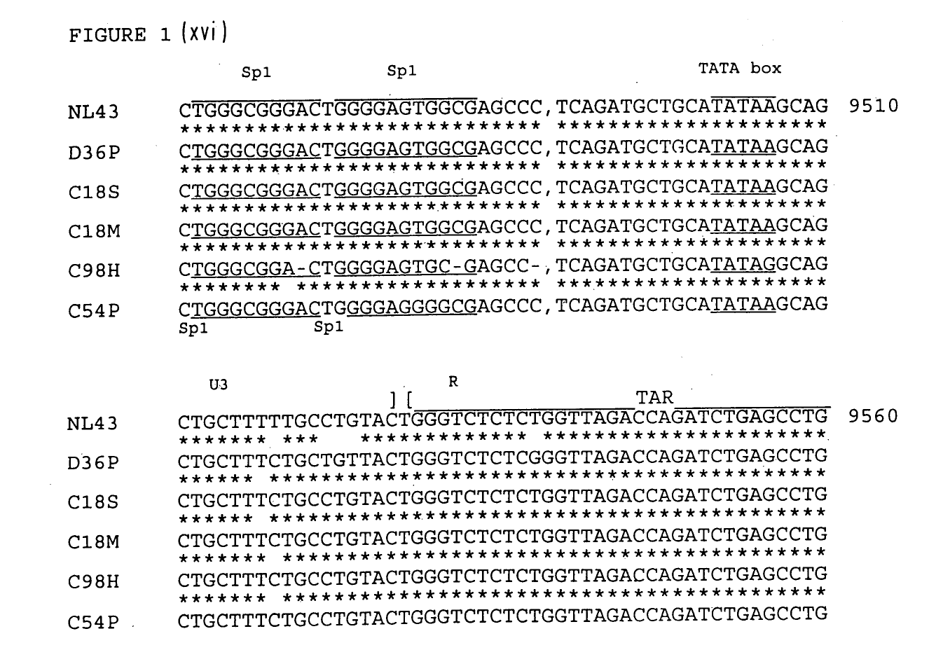

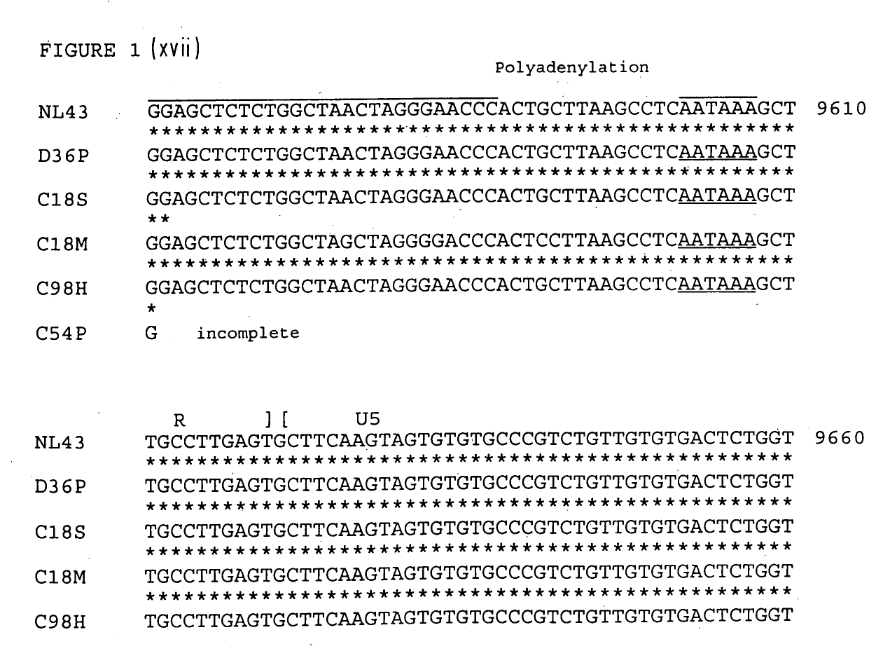

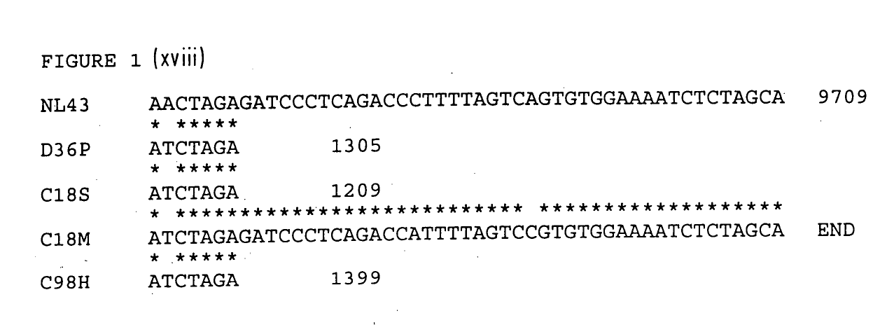

Alignment of the nucleotide sequences of the amplified 3′ region of donor D36 PBMC and recipient C18 isolates HIV_{MBC} and HIV_{StV} and C98 HIV (FIG. 1) showed a number of nucleotide sequences changes, including deletions, relative to the nucleotide sequence of wild-type infectious HIV-1 (HIV-1_{NL43}). In the region of alignment, D36 PBMC lacked 291 nucleotides, C18 HIV_{StV} differed in size by 388 nucleotides (comprising deletions of 397 nucleotides and an insertion of 9 nucleotides), C18 HIV_{MBC} differed by 456 nucleotides and C98 HIV lacked 158 nucleotides compared with HIV-1_{NL43}. The overall identity with HIV-1_{NL43} nucleotide sequence of D36 PBMC, C18 HIV_{StV}, HIV_{MBC} and C98 HIV nucleotide sequences, including deletions, was 73% (1157/1596), 67% (1459/1592), 62% (982/1592) and 79% (1105/1399), respectively.

The D36 PBMC sequence differed from HIV-1_{NL43} in a number of features. A change in the wild type tat termination codon from TAG to TCG (Ser) extended the third tat exon (which starts at splice acceptor 10) by a further 15 amino acids to terminate at a conserved TAG (FIG. 1). The resulting C-terminal peptide is rich in charged amino acids (8/15) (FIG. 2a). The wild type rev termination codon has also changed (TAG to GAG, Glu) and the third rev exon is extended for 14 codons to terminate at a conserved TAG (FIG. 2b). The encoded extra amino acids are mainly polar (11/14) and charged in nature (FIG. 2b). The sequence also encodes the C-terminal 237 amino acids of Env gp4 (FIG. 3) terminating at the normal termination codon. The D36 PBMC Env amino acid sequence has 85% identity with the HIV-1_{NL43} sequence, increasing to 89% if similarities are included.

There are significant differences from HIV-1_{NL43} downstream of the env (gp41) gene. A change in the fifth nef codon, from TGG (Trp) to TGA (FIG. 1), introduces an early termination in the D36 PBMC nef gene. The encoded Nef protein is identical to the N-terminal 4 amino acids of HIV-1_{NL43} Nef (FIG. 4). Following the early termination are deletions of 33, 47, 93 and 91 nucleotides and a region of low sequence homology, compared with HIV-1, prior to the wild type network on codon site (HIV-1 nts 9405-9407). As well as removing a significant part of the nef gene these deletions also bring into phase a further 6 termination codons. While the polypurine tract (plus strand primer binding site) and the first 38 nucleotides of the LTR U3 region arc perfectly conserved, downstream U3 region sequences are disrupted by the 93 and 91 nucleotide deletions and the low homology region. The resulting U3 region lack recognition sequences for the transcription favors c-myb, USF and TCF1α as well as one of the suggested NF-AT sites (Gaynor et al, 1992). Downstream from the 91 nucleotide deletion, a 59 nucleotide region of low homology contains two extra NFKB enhancer sites 19 nucleotides upstream of the usual site of a pair of NFKB sites, the up one of which is altered in its 5′-half in D36. Sequence further downstream arc highly conserved with respect to HIV-1_{NL43}, including the position and number of Sp1 basal promoter sites, TATA box, TAR and polyadenylation signal sequences.

Similar to D36 PBMC, the C18 HIV_{StV} and HIV_{MBC} sequences show the tat third exon to be extended by 15 codons. All but two codons (altered by point mutations) are identical to those of D36 PBMC (FIG. 2a). The rev third exon of both C18 isolates is also extended (FIG. 2b) but by only three codons, identical to the first three codons of the D36 PBMC rev extension. The same 237 amino acid coding region of Env gp41 is found in both the C18 HIV DNA sequences (FIG. 3) and shows 85% identify increasing to 88% if similarities are included, with the same region of the HIV-1_{NL43} Env gp41.

It is in the nef gene and LTR regions that the major differences from wild-type HIV-1 arise, just as in D36 PBMC. The nef gene of C18 HIV_{StV} encodes 24 amino acids with 9 of the 10 N-terminal being identical to the HIV-1_{NL43} Nef protein (FIG. 4). Thereafter, deletions of 177 and 11 nucleotides cause a frameshift and termination at the 25th codon (FIG. 1). Downstream deletions of 120, 82 and 7 nucleotides cause Per loss of wild type nef gene sequence and bring into phase a further three termination codons.

The nef gene of C18 HIV_{MBC} encodes only 7 amino acids with only the initiator methionine identical to the HIV-1_{NL43} Nef protein. This loss of identity and early termination is brought about by a 250 nucleotide deletion after the fifth nucleotide of the nef gene. Downstream deletions of 120 and 86 nucleotides cause further loss of wild-type nef gene sequences. In both C18 isolates there is perfect conservation of the polypurine tract and 29/31 nucleotides at the 5′ end of the U3 region immediately before the 120 nucleotide deletion (FIG. 1). This deletion together with the downstream 82 and 7 nucleotide deletions in HIV_{StV} and 86 nucleotide deletion in HIV_{MBC} and the low homology region cause the loss of the 5′ half of the NRT-1 site (Yamamoto et al 1992) and the downstream NFAT site. A third NFKB site is present 31 (HIV_{stV}) and 33 (HIV_{MBC}) nucleotides upstream of the expected pair of NFKB sites which are themselves separated by 13 nucleotides instead of the 4 nucleotides in HIV-1_{NL43}. The 5′-most Sp1 site sequence is slightly altered but sequences downstream including the other 2 Sp1 sites, the TATA box, TAR and polyadenylation signal sequences are identical to HIV-1_{NL43} sequence.

The three sequence, D36 PBMC, C18 C18 HIV_{StV} and C18 HIV_{MBC} show a number of similarities consistent with the transmission of virus from person D36 to person C18 as well as a number of differences indicating post-transmission divergence of sequence. All three have tat open reading frames (ORFs) extended by 15 codons. All three have extended rev ORFs. Th new rev termination codon in both C18 HIV-1 isolates, thee codons downstream of the HIV-1_{NL43} rev termination codon, has a point mutation in D36 PBMC to make a Glu codon so that it continues for a further 11 codons (FIG. 2b) to terminate at a conserved TGA. The partial Env gp41 amino acid sequences are more closely related to each other (86% identity or 90% including similarities) than to HIV-1 (85% and 89%, respectively).

The nucleotide sequence of the nef and LTR region of the HIV-1 isolate from recipient C98 (C98 HIV) is 90.3% identical (1264/1399) to the HIV-1 sequence, ignoring deletions. Similar to the D36 PBMC and C18 HIV_{StV} and HIV_{MBC} isolates the C98 HIV sequence shows the third exon of tat to be extended by 15 codons with all but one being identical to the D36 PBMC tat extension. Also, the rev gene is extended by codons, 2 of which arm identical to the first 2 codons of the D36 PBMC rev extension. The sequence also encodes the C-terminal 223 amino acids of Env gp41 terminating at the normal termination codon. The C98 HIV Env amino acid sequences has 89% identity with HIV-1 Env sequence, increasing to 92% of similarities are included.

As with the D36 PBMC and the C98 HIV isolate sequences it is the nef gene and LTR regions that major differences from the HIV-1 sequence arise. The nef gene open reading frame of C98 HIV is much longer than in D36 PBMC, C18 HIV_{StV} and HIV_{MBC}, encoding 85 amino acids compared with 206 m acids for HIV-1_{NL43}. Sixty eight of those 85 amino acids are identical to the N-terminal sequence of HIV-1_{NL43} Nef. The single, small deletion (16 nucleotides) in the C98 HIV nef-alone regions (Table 3) occurs after nef codon 82 causing a frameshift and termination after a further 3 codons at the start of the highly conserved polypurine tract sequence immediately before the 3′-LTR. The nef/LTR region has two deletions totalling 142 nucleotides The 5′-most deletion of 42 nucleotides includes the splice acceptor 12 sequence. The NRT-1, dyad symmetry and myb response element sequences are all intact. However, the downstream 100 nucleotide deletion includes sequences from the 3′ end of the 5′-NF-AT and all of the 3′ NF-AT sequences as well as the USF transcription factor recognition sequence. The downstream low homology region of 77 nucleotides lacks the TCF-1α sequence but has two additional NFKB sites 13 nucleotides apart and 26 nucleotides of the 3′-half-remmant of the normal 5′-NFKB site. Sequence downstream, including the 3′-NFKB site, the 3 Sp1 sites, TATA box TAR and polyadenylation signal sequences are all highly conserved.

The main feature of the sequences is the series of deletions, with respect to HIV-1, in the nef gene-3′-LTR region. These can be grouped into two regions namely the nef-alone region, that part of the nef gene upstream of the LTR, and the nef/LTR region, where the nef gene and LTR U3 regions overlap. The deletions in these regions of each of the sequences start and end at the same or similar positions (Table 3). The deletions are larger in C18 HIV_{StV} and C18 HIV_{MBC} sequences where totals of 397 and 456 nucleotides have been deleted (relative to HIV-1_{NL43}) compared to 291 nucleotides, from D36 and 158 nucleotides from C98 HIV. In the nef-alone region the two deletions in C18 HIV_{StV} and the single deletion in C18 HIV_{MBC} occupy the same region as the three deletions in D36 PBMC. Similarly, the nef/LTR region in the three deletions in the C18 HIV_{StV}, the two deletions in the C18 HIV_{MBC} and the D36 PBMC sequences occupy the same region. These findings indicate that mutant virus was transmitted from D36 to C18 after which further deletions and rearrangements occurred. Similarly, the sequence of C98 HIV in the nef/LTR region indicates two deletions occupying the same region as the nef/LTR deletions in D36 and the C18 sequences. However, the size (only 16 nucleotides) and the position of the deletion in the nef-alone region of C98 HIV are distinct from those of the D36 PBMC and C18 sequences.

The timing of transmission of virus by transfusion was that recipient C18 was transfused approximately 19 months after C98. Consistent with the relative timing of transmission and the sequence similarities and differences is the suggestion that at the time of transmission to C98, the D36 sequence had deletions in the nef/LTR region but not in nef-alone region. After transmission to C98, the C98 virus developed further deletions and rearrangements, including the deletion in the nef-alone region. The D36 virus evolved so that at the time of transmission to C18, further deletions and rearrangements had occurred including deletion of sequence from the nef-alone region distinct from the C98 HIV nef-alone region deletion. After transmission to C18, further deletions and rearrangements occurred in the C18 virus giving rise to at least two sequences (HIV_{StV} and HIV_{MBC}).

The nef-alone deletion region may be mutation or recombination “hotspot” as it includes sequences that were found to be variably duplicated in 28 out of 54 Nef protein sequences derived from 8 of 12 patients analysed in a study (Shugars et al 1993). The sequence between the nef-alone and the nef/LTR region deletions is highly conserved and is important in provirus integration into the infected cell gnome and interacts with a number of cellular proteins. It is interesting that the sequence equivalent to HIV-1_{NL43} nucleotides 9209 to 9225 is retained in D36 and C98 HIV but lost in the C18 HIV sequences. This includes part of a sequence of dyad symmetry (9210 to 9231) and is a significant part of the binding site for NRT-1 (Yamamoto et al 1991) which has been shown to have a negative regulatory effect on HIV-1 expression. The presence of this sequence in D36 and C98 HIV and its absence from the C18 isolates may correlate with the inability to isolate virus from D36 PBMC and the poor replication of C98 HIV but the ability to isolate HIV-1 from C18 PBMC. The deletion of sequence equivalent to nucleotides 9281 to 9395 of HIV-1_{NL43} causes the loss of some transcription factor binding sites including NAT and USF from the D36, C18 HIV and C98 HIV sequences. A further similarity between the D36, C18 HIV_{StV}. C18 HIV_{MBC} and C98 HIV sequences is a region of low homology to HIV-1_{NL43} extending downstream of the nef/LTR deleted region to the NFKB enhancer/Sp1 promoter site region. This low homology region in fact consists of incomplete duplications of part of the NFKB/Sp1 region (FIG. 5) resulting In D36 and C98 HIV having 2 extra NFKB sites upstream of an altered 5′ NFKB site while the C18 sequences have one extra NFKB site and altered spacing the 5′ and 3′ wild type NFKB sites due to an insertion of 9 nucleotides.

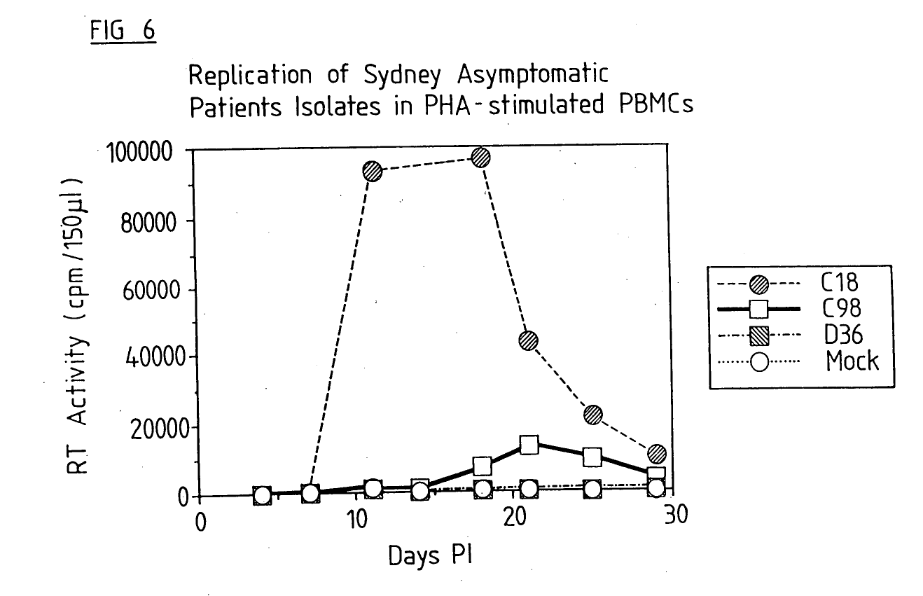

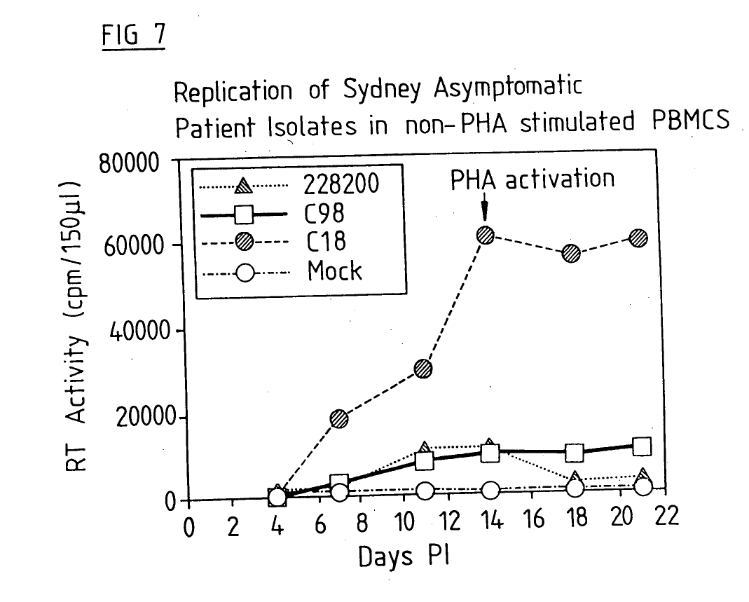

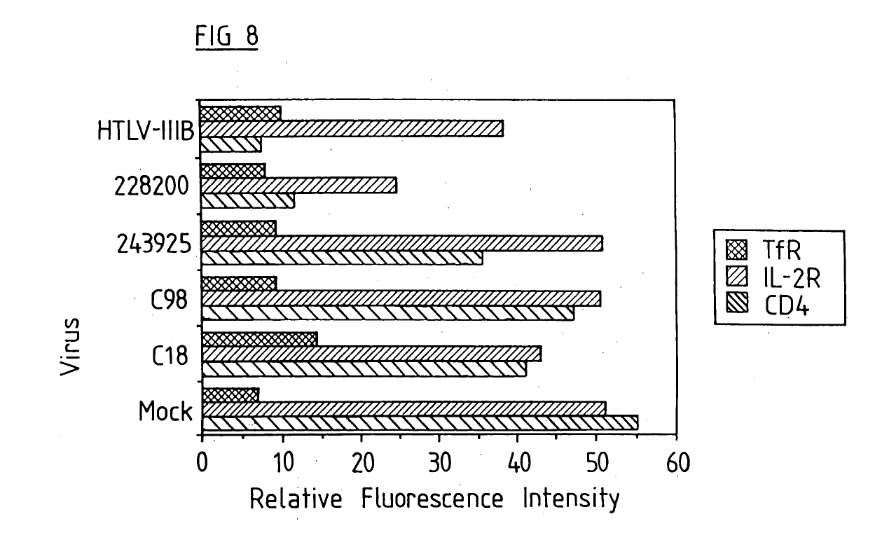

For the C18 and C98 HIV-1 isolates virus replication was assessed in PHA-stimulated and non-stimulated PBMCs (FIGS. 6 and 7). In PHA-stimulated PBMCs we also studied cell surface CD4 and IL-2R expression (FIG. 8). In comparison with HIV-1 wild-type SI and NSI isolates clearly both C18 HIV_{MBC} and C98 viruses are replication competent, though C98 HIV replicates more poorly than C18 HIV_{MBC} and are of the NSI phenotype when syncytium formation and CD4 and IL-2R surface expression are taken into account. Additionally, and more surprisingly, these two viruses replicated almost as efficiently in non-PHA stimulated PBMCs when compared to a typical local wild type SI isolate (HIV-1 228200, FIG. 7).

When protein expression was assessed for C18 HIV_{MBC} and C98 HIV_{MBC} although structural proteins were identified, no typical Nef protein was seen in infected cells. However, analysis of cell lysates prepared from PBMC infected with C18 HIV_{MBC} or PBMC infected with C98 HIV_{MBC} (which were subsequently stimulated by UV irradiation, see Valerie et al, 1988) by Western immunoblotting using two antibodies specific for the N-terminal region of Nef showed the presence of smaller proteins of 19 kDa and 21 kDa, respectively. These proteins were not observed in mock-injected control PBMC lysates and were not observed when the infected-cell lysates were probed were probed with antibodies reactive with the C-terminus of Nef.

Thus, although the C18 and C98 HIV isolates are replication competent in vitro they clearly replicate differently using different conditions for cell activation and from the known functions of HIV-1 Nef protein and the LTR show that the major deletion in the nef gene and/or the LTR is at least in part responsible for the outcome of infection, implicating the importance of Nef and/or the LTR in the clinical outcome of infection in vivo.

### EXAMPLE 9

### Determination of Degree of Relatedness Between Viruses

To determine the degree of relatedness between viruses such as between mutants or between mutants and a wild-type virus and to ascertain putative infected patients, the method of Delwart et al was employed.

### EXAMPLE 10

Immune responsiveness of subjects infected by non-pathogenic HIV-1 isolate In this example, the donor and recipients of the cohort were time typed and assessed for basic cellular immune responses Proliferative responses and IL-2 action to the mitogens ConA and PHA, to allogeneic mononuclear cells (irradiated pooled mononuclear cells from 20 random donors) and to recall antigens (e.g. influenza and tetanus toxoid) were within normal ranges. While at the immunogenetic level, HLA typing failed to identify a consistently common allele or haplotype within the group.

The conservation of CD4+ counts observed in the cohort, the relative integrity of their immune system and the varied HLA types of the donor and recipients further supports the fact that the symptomless condition of the cohort members is due to a non-pathogenic strain of HIV-1 or a strain of low virulence.

Accordingly, this provides a screening procedure for subjects putatively infected by a non-pathogenic HIV-1 isolate where such subjects are seropositive for HIV-1 (e.g. have antibodies to an HIV-1 glycoprotein) yet have normal proliferative responses and cytokine production to mitogens, allogeneic mononuclear cells and to recall antigens.

### EXAMPLE 11

### Clinical Immunology of Cohort

To establish that the donor and the recipients belonging to the cohort exhibit normal immunological profiles, members of the cohort were assayed for CD3, CD4, CD8, lymphocyte count, CD4/CD8 ratio and β-2-microglobulin over time since seroconversion.

Parameters considered normal in non-infected individuals range as follows:

| Parameter | | |
|---|---|---|
| CD3 | 55-82% | 620-2200 (×10⁶/L) |
| CD4 | 29-58% | 420-1410 (×10⁶/L) |
| CD8 | 12-43% | 200-980 (×10⁶/L) |
| Lymphocyte count | 1000-3500 | |
| | (×10⁶/L) | |
| CD4/CD8 | 0.7-3.7 | |
| β-2-microglobulin | 0.00-2.20 | |
| | mg/L | |

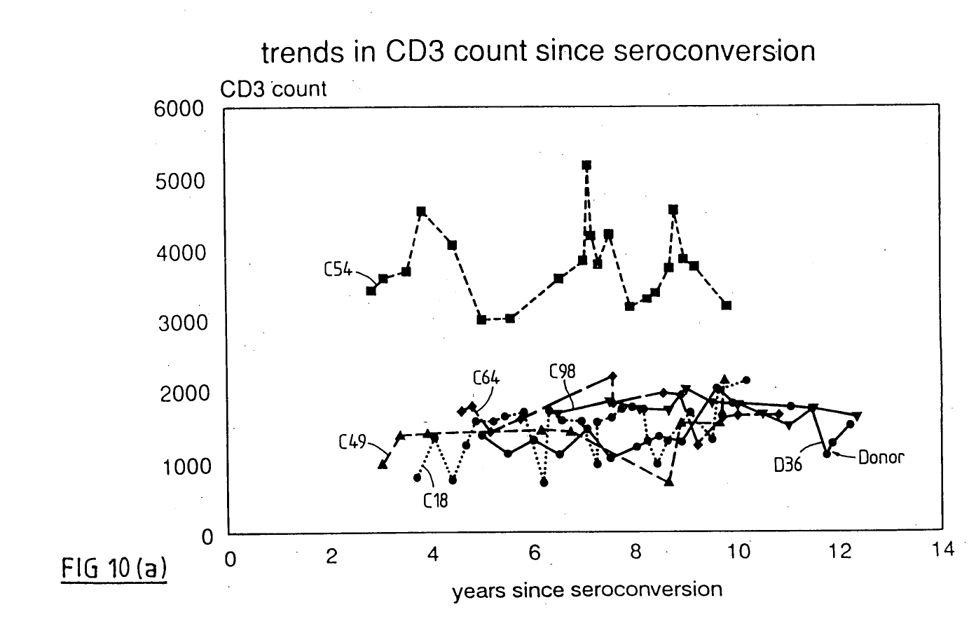

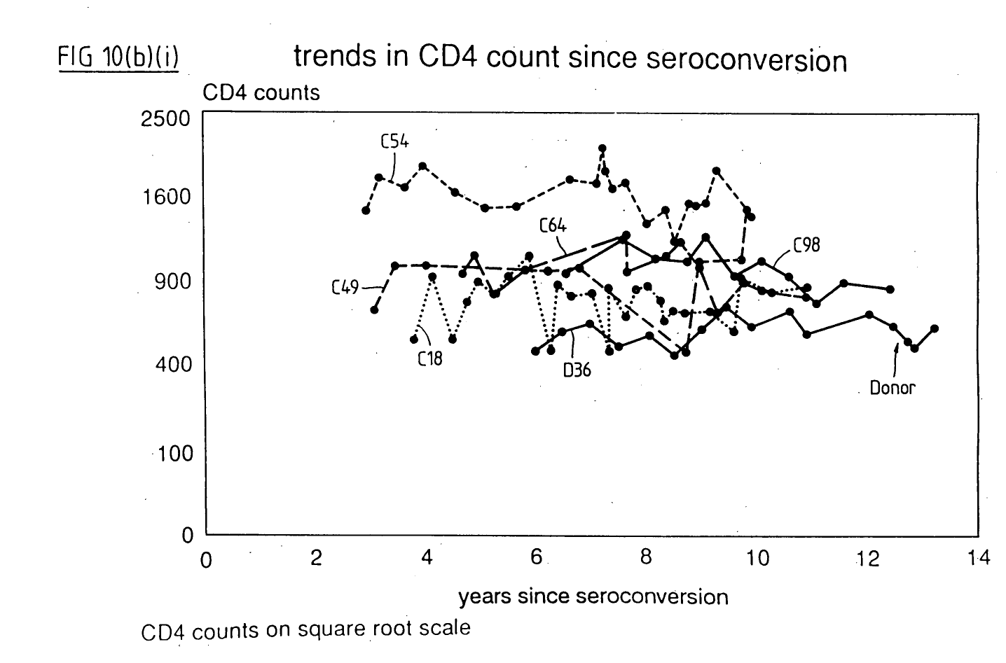

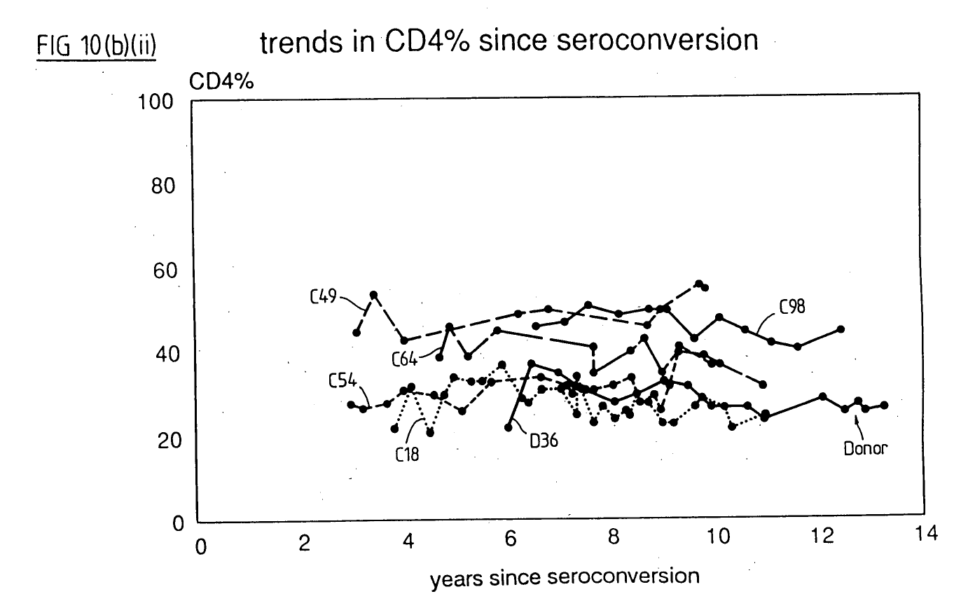

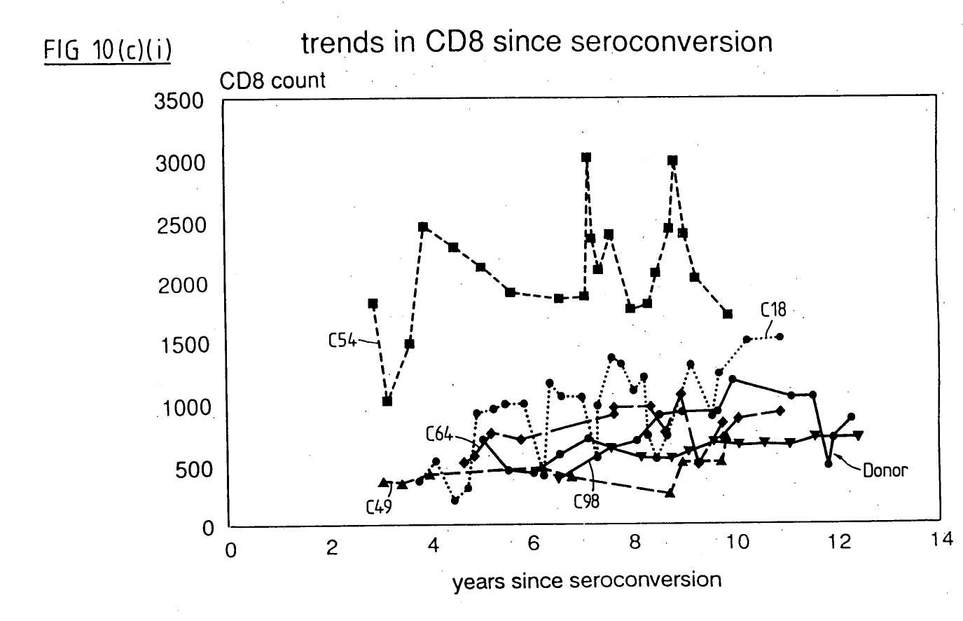

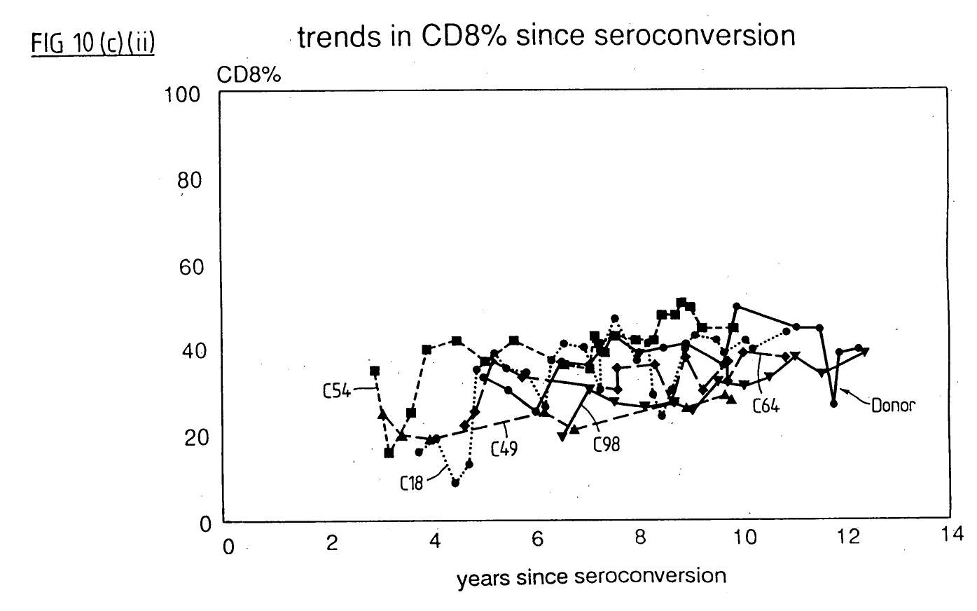

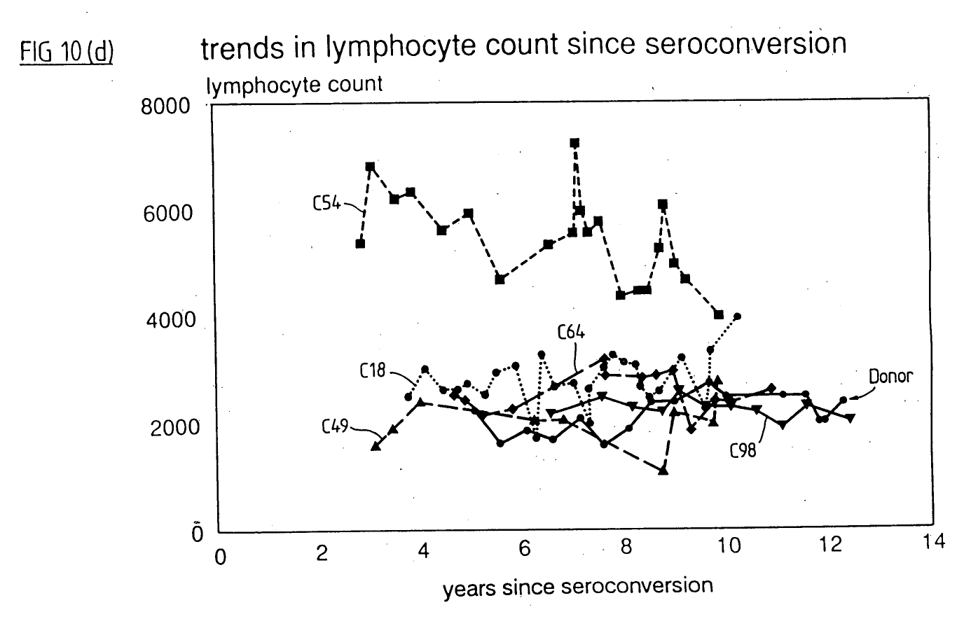

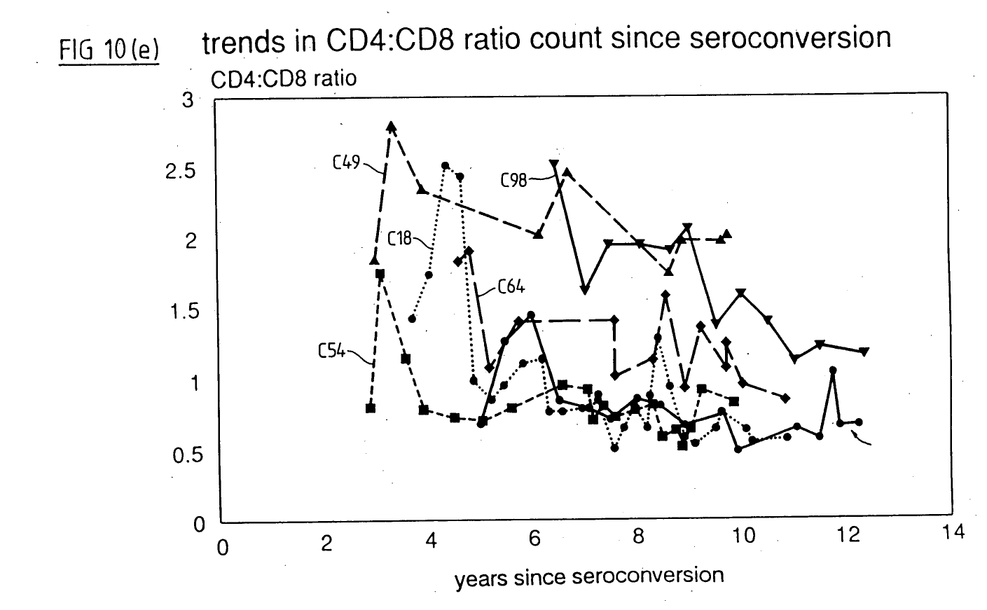

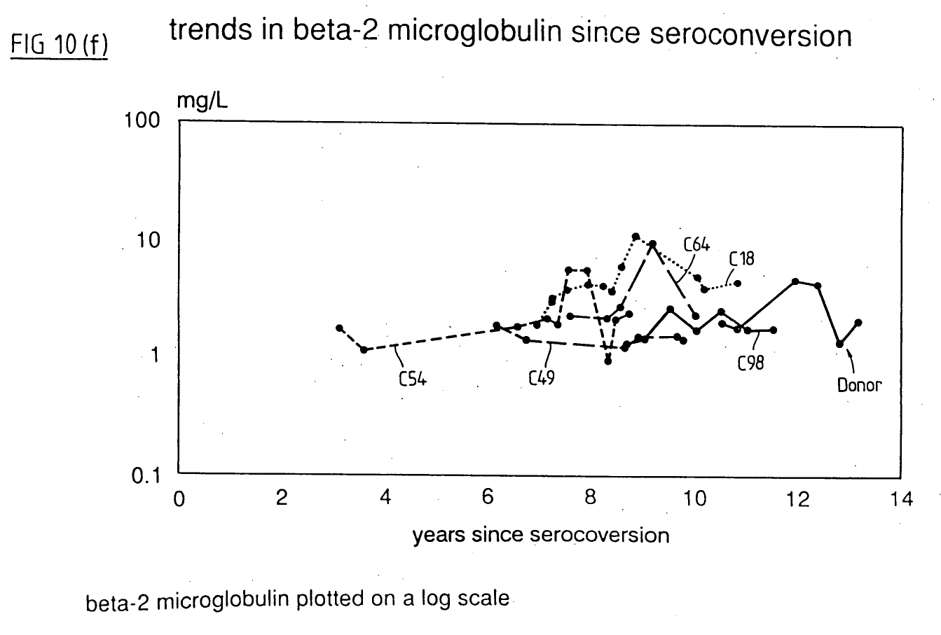

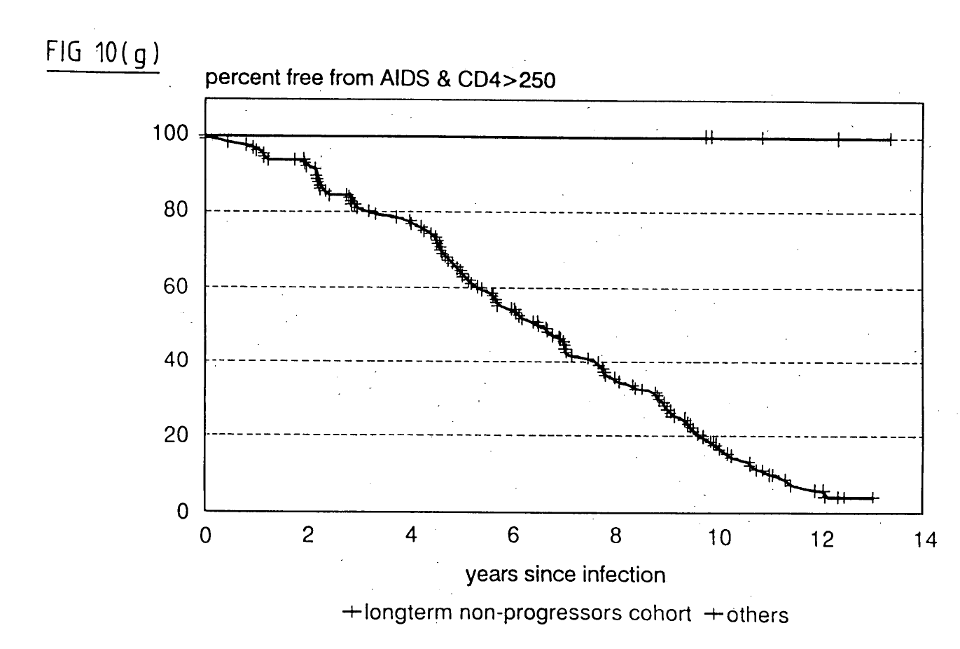

The results arc shown in FIGS. 10(a)-(g) and clearly show that the immunological profiles of cohort members arm substantially normal further highlighting the non-pathogenicity of the HIV-1 isolates of the present invention FIG. 10(g) shows a graph of the Kaplan-Meier (Ox and Oates, 1989) estimates of time to disease progression (AIDS or CD4>250). The results demonstrate that the difference is large in spite of the conservative bias, with a median time to progression of 6.2 yews in the main database. An exact logrank test (Cytel Software Corporation, 1989, StatXact: Statistical Software for Exact Nonparametric Inference. Cambridge, Mass.) was performed, demonstrating that the difference between the groups was highly statistically significant (logrank static 11.8, p<0.0001).

TABLE 3
Deletions and their sizes in the nef-alone and the nef/LTR regions of the
Long-Term Asymptomatic HIV-1 Sequences

| | | Region | | Region | Total |
|---|---|---|---|---|---|
| | | Deletion | | Deletion | Deletion |
| Sequence | nef-alone Region | (nt) | nef/LTR Region | (nt) | (nt) |
| D36 PBMC | 8830-8862 (33) | 107 | 9112-9204 (93) | 184 | 291 |
| | 8882-8928 (47) | | 9281-9371 (91) | | |
| | 9009-9035 (27) | | | | |
| C18 HIV_{StV} | 8830-9006 (177) | 188 | 9105-9224 (120) | 202 | 390 |
| | 9019-9029 (11) | | 9281-9362 (82) | | |
| C18 HIV_{MBC} | 8792-9041 (250) | 250 | 9105-9224 (120) | 206 | 456 |
| | | | 9281-9366 (86) | | |
| C98 HIV | 9033-9048 (16) | 16 | 9148-9189 (42) | 142 | 158 |
| | | | 9271-9370 (100) | | |
| C54 PBMC | incomplete | ? | 9281-9375 (95) | 95 | 95+ |

Sequence numbering relates to the equivalent position in HIV-1 NL4-3. Numbers in brackets are the deletion sizes in nucleotides (nt).
The nef ORF starts at nt 8787and the 3′-LTR starts at nt 9074 in HIV-1 NL4-3.

### EXAMPLE 12

### Sequencing of Isolate HIV-1 C18_{MBC}

The genome of variant HIV-1 designated C18 HIV-1_{MBC} was amplified by the polymerase chain reaction (PCR) as 7 overlapping agents using the sets of inner and outer oligonucleotide primers, designed using the programme PCRPLAN (IntelliGenetics), listed in Table 5 and either UITma (Applied Biosystems) or a mixture of KlenTaq and Pfu (KlenTaq LA, Ab Peptides Inc) polymerase (for faithful amplification of long fragments). The resulting fragments were cloned into the SmaI site of the plasmid vector pGEM7Zf+. Insert-containing clones representing each region of the full length variant HIV-1 were sequenced by a nested deletion strategy (Gou & Wu, 1982) and cycle sequencing with Taq polymerase and dye labelled primers complimentary to the T7 or SP6 sites within the cloning vector. Nucleotide sequences were entered and collated by ASSEMGEL and SEQIN (IntelliGenetics) and SEQED (Applied Biosystems) and translated to the encoded amino acid sequences using TRANSL (IntelliGenetics) programmes. Sequence alignments used NALIGN, CLUSTAL (IntelliGenetics) and SEQED programmes.

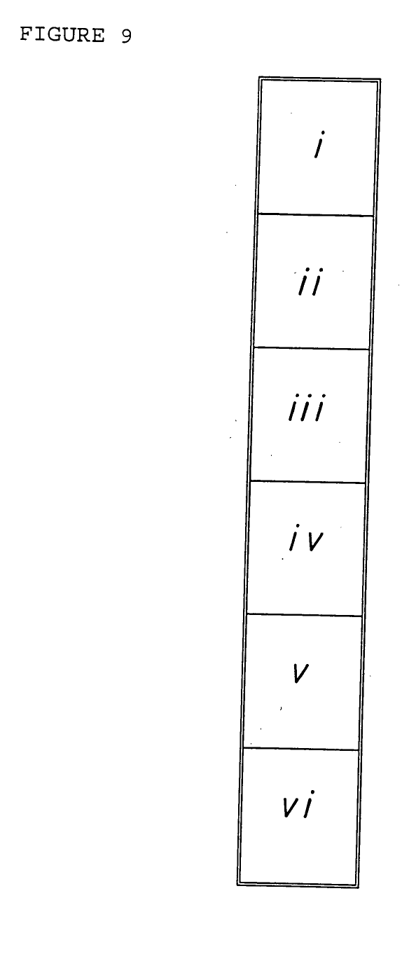

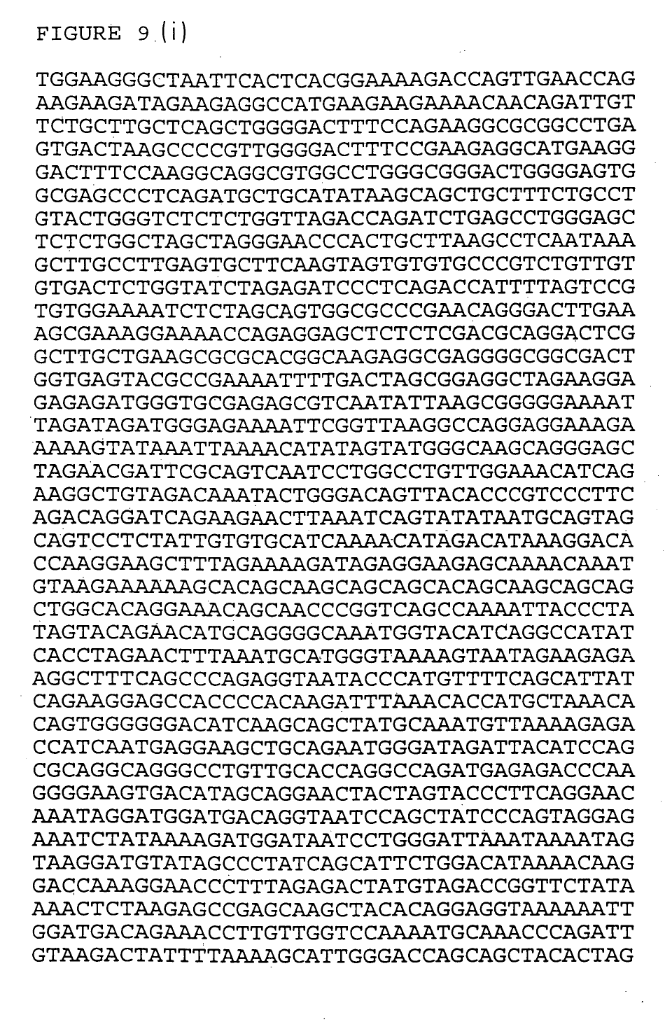

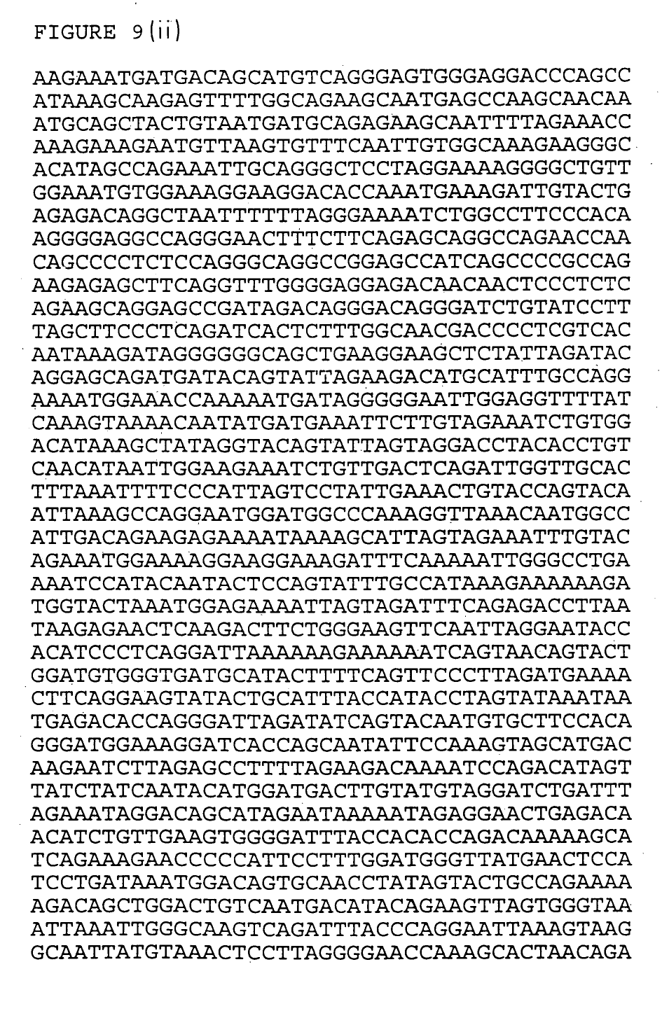

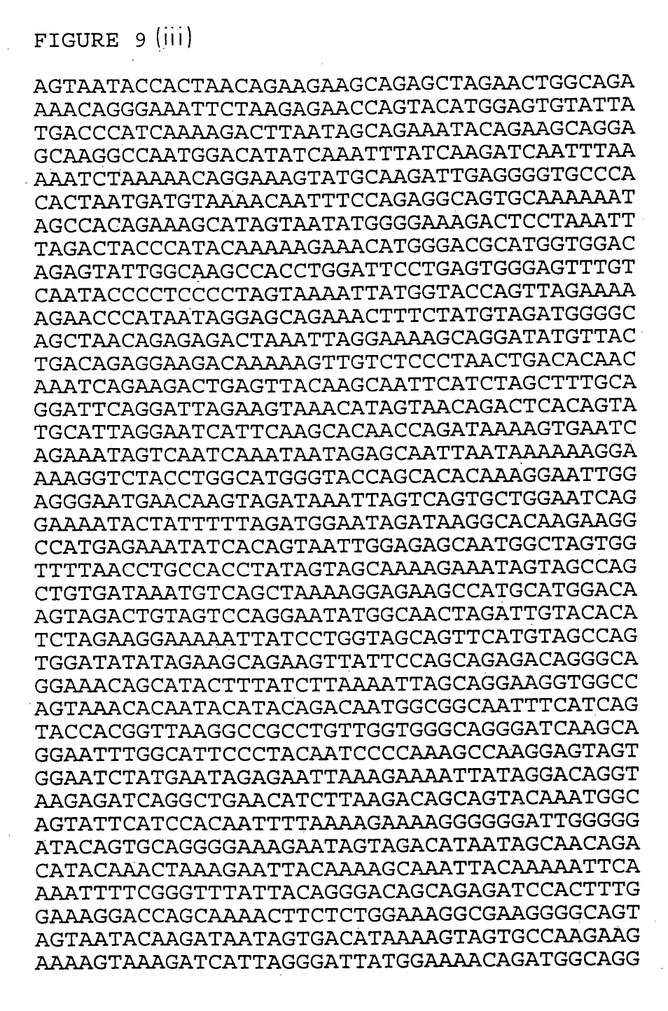

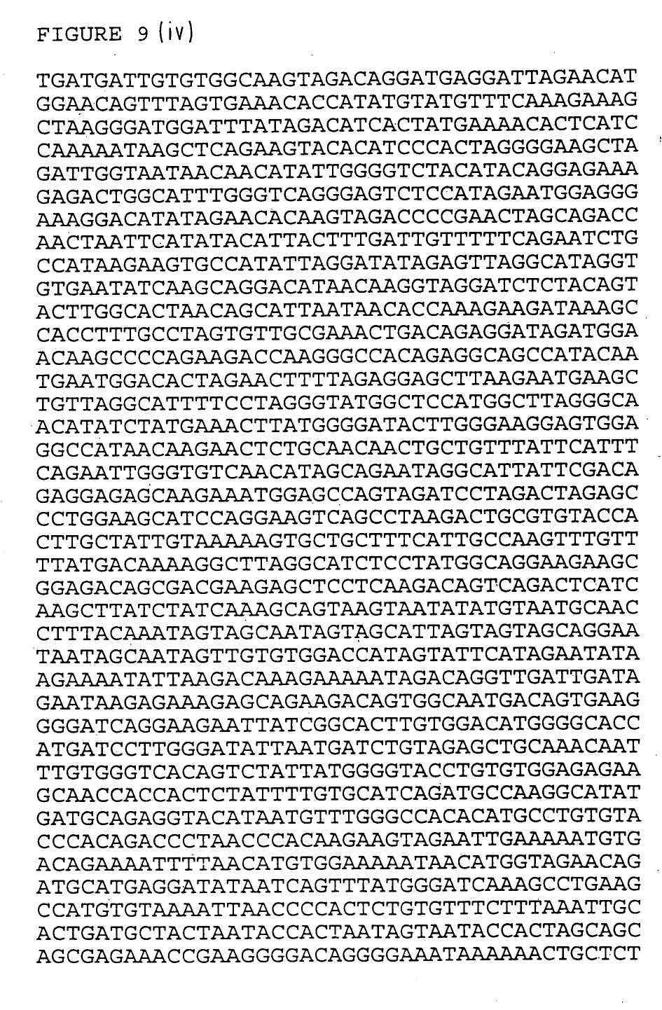

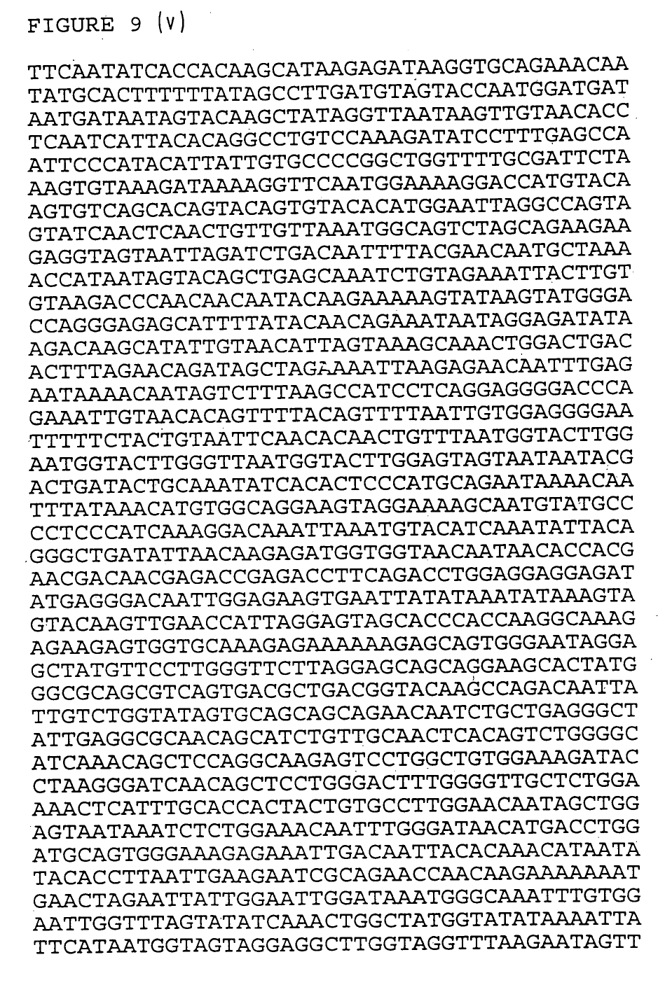

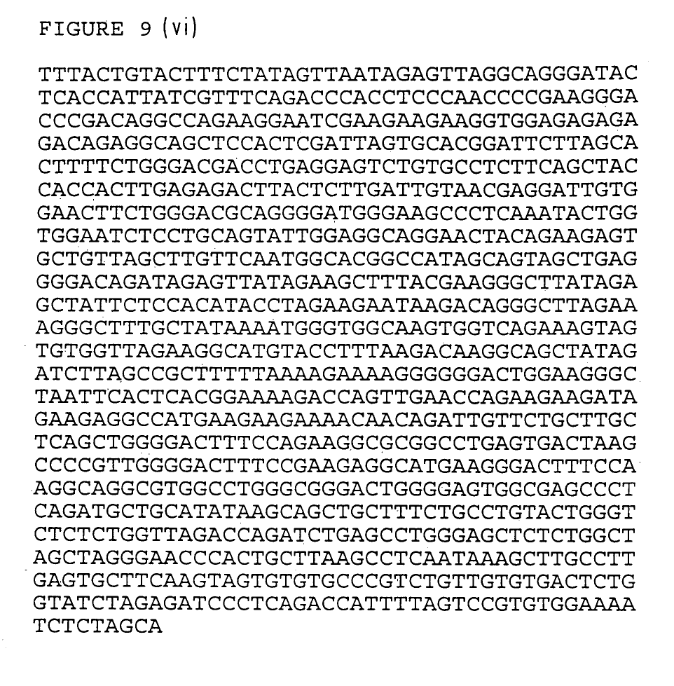

The full length sequence (FIG. 9, SEQ ID NO:800) of isolate HIV-1 C18_{MBC is} 9207 nucleotides long which is 506 nucleotides shorter than the HIV-1 sequence. This size difference is comprised of 126 nucleotides of insertions and 632 nucleotides of deletions, see Table 6. The most extensive differences between the HIV-1 C18_{MBC} sequence and HIV-1_{NL43} arm in the U3 region of the LTR and in the nef gene, as hereinafter described.

The 5′ LTR has deletions of 120 and 87 nucleotides and a region of low sequence homology, which is the result of an imperfect duplication of the downstream NFκB and Sp1 response sequences. These result in the loss of sequence from a number sites important in thc regulation of transcription of HIV-1 genes, including the negative response element (NRE) and the response elements for a number of transcription factors including NF-AT, NRT-1, USF and TCF-1α. Furthermore, the low homology region contains an extra NFκB and Sp1 sites as well as an insertion of 9 nucleotides between the usual NFκB sites. Downstream of the NFκB sites the sequence of the LTR has a high level of homology (96.2%) with the same region of HIV-1.

The gag gene contains 3 insertions, which represent direct repetitions of adjacent sequences. The first is a perfect repeat of 15 nucleotides after the equivalent of nucleotide 1134 of HIV-1_{NL43} and adds 5 amino acids to the C-terminus region of p17^{gag}. The remaining 2 insertions are imperfect and perfect repeats of 30 and 6 nucleotides, respectively, after the equivalent of HIV-1 nucleotides 2163 and 2232, respectively. These encode an extra 12 amino acids in the C-terminus region of p15^{gag} just downstream of the gag to pol frameshift sequences. The variation in sequence length of the gag gene at these two positions is unusual. The homology of the encoded amino acid sequence of HIV-1 C18_{MBC} and HIV-1 for the gag p17, p24, and p15 proteins is 87.1%, 93.5% and 94.3%, respectively.

In the pol ORF, the encoded proteins have high homology with the HIV-1_{NL43} sequences being 95.5% overall comprising p10 protease 92.9, p66 reverse transcriptase 95.4% and p34 integrase 95.8%. The amino acid sequence of the p61 RT lacks the mutations associated with resistance to the nucleoside (AZT, ddI, ddC) and non nucleoside (Nevirapine) analogue drugs used in the treatment of HIV-1-infected persons.

The vif gene encodes a 192 amino acid protein with 88.0% homology with that of HIV-1. The vpr gene encodes a 96 amino acid protein with 89.6% Y homology with that of HIV-1_{NL43}.

There are 2 insertions and 1 deletion of sequences in the vpu gene. The insertions of 3 and 9 nucleotides are after the equivalent of nucleotide 6071 and 6234, respectively of HIV-1_{NL43}. These add 1 amino acid after amino acid 3, and 3 amino acids after amino acid 59 of the encoded Vpu protein. The deletion of 12 nucleotides after the equivalent of HIV-1_{NL43} nucleotide 6261 deletes 4 amino acids from the C-terminal region of Vpu as well as from the signal peptide of the env polyprotein, which is encoded by an overlapping ring frame. Amino acid sequence homology of HIV-1 C18_{MBC} Vpu wt HIV-1_{NL43} is 85.2%.

The sequence encoding the env gp120 has 9 insertions totalling 45 nucleotides (encoding 15 amino acids) and the deletion of a total of 18 nucleotides (encoding 6 amino acids). These are listed in Table 6. AU of these events (insertions and deletions) are at positions in the env gene. This is within the env V3 coding region, immediately ups of the sequence encoding the so called V3 tip (or loop) amino acid sequence, Gly Pro Gly Arg. The V3 region sequence is that of a typical clade B subtype (North America, Europe and Australia) being identical to the clade B consensus sequence (based on 186 env sequences) at 29/35 positions. The type of amino acid at positions 11 and 28 of the V3 loop region (where position 1 is the Cys at amino acid 266 of the env gp120) is predictive of the viral non-syncytium/syncytium forming phenotype (Fouchier et al, 1992). The HIV-1 C18_{MBC} env gene encoded amino acid sequence has Ser at position 11 and Ile at position 28 of the V3 loop region. The lack of a positively charged amino acid at both positions is strongly indicative of a non-syncytium viral phenotype. The overall amino acid sequence homology with HIV-1_{NL43} (ignoring deletions and insertions) is 86.1%, comprising 85.5% for the gp120 region and 87.6% for the gp41 region.

Both the tat and rev second exon open reading frames (ORF) are longer than in HIV-1_{NL43}. A change of the tat termination codon from TAG to TCG extends the tat ORF to a downstream in phase termination codon extending the en tat amino acid sequence by 15 residues compared wit the 86 amino acid long HIV-1_{NL43} tat protein, to a total length of 101 amino acids However, this is the usual length of the HIV-1 tot protein.

Similarly, the normal rev termination codon is changed from TAO to GAO. This extends the rev ORF to an in-phase termination codon 3 codons downstream so that the encoded Rev protein is 119 amino acids long instead of the usual 116.

As mentioned above the most extensive differences between the sequences of the isolate HIV-1 C18_{MBC} and HIV-1_{NL43} are in the nef gene and the LTR region. While the nef gene overlaps the 3′ LTR, these differences are found in both the nef alone and the nef/LTR overlap regions. The HIV C18_{MBC}-encoded nef protein is only 24 amino acids long compared with the normal let of 206 amino acids. This severe shortening of the nef protein is due to the deletion of 188 nucleotides (the 177 and 11 nucleotide deletions) from the nef-alone region which also brings into phase a termination codon, TAG, at the resulting 25th codon. Downstream there is further loss of potential nef gene sequences by the 120 and 87 nucleotide deletions situated in the nef/LTR overlap region. The resulting 24 amino acid nef protein is identical to the N-terminus of the HIV-1_{NL43} nef at 9 of the first 10 positions. Thereafter, homology is lost completely.

Some sequences used in the generation of mature mRNAs are altered or lost in C18_{MBC}. The dinucleotide immediately after the splice donor site 2 (SD2) at nts 4818-4819 (HIV-1_{NL43} equivalent nts 4963-4964) is changed from the conserved GT to GC. It is expected that this change would lead to loss of function of this site as a splice donor. Splice donor 2 is used in the processing of HIV-1 transcripts to some of the mRNAs that encode Tat, Rev and nef proteins. Similarly the splice acceptor site 7 (SA7) sequence at nts 6477-6478 (HIV-1_{NL43} equivalent nts 6602-6603) is changed from the conserved AG dinucleotide to TC. This change is expected to lead to loss of function of this site as a splice acceptor. While this SA site is used in HIV-1 mRNA processing it is not a major site and is not used in the production of the regulatory proteins (Tat, Rev or nef) mRNAs. The splice donor 12 site is absent from the C18_{MBC} sequence (NL43 equivalent nts 9161-9162) as it is within the first deletion region in the nef/LTR overlap region which occurs at nt 8797 and results in the loss of NL43 nucleotides 9105 to 9224. It is significant that the SA12 site is absent from the sequence of all of the cohort virus isolates so far obtained as well as from the sequence of D36 PBMC, however, the C54 PBMC sequence does contain the SA12 site. SA12 is not used in the processing of mRNAs that encode the viral regulatory proteins. Normally SA12 is used in splicing in conjunction with SD1, 2, 3 and 4 and the resulting spliced RNA is probably not a mRNA but may have a regulatory role involving binding to cellular proteins (Smith et al, 1992).

An interesting feature of the sequence of the HIV-1 C18_{MBC} isolate is the deletion and rearrangement of sequence from the 5′-LTR, U3 region and the deletion of sequence from the nef gene (both nef alone and nef/3′ LTR regions). These being the only features of the sequence distinct from disease-causing HIV-1. The lack of AIDS or AIDS-like symptoms in the patient C18 is attributed to the effects of the loss of LTR sequence and/or the loss of nef coding sequences and their role in the pathogenesis of AIDS.

TABLE 4
Primers used to Amplify Overlapping regions of HIV-1 C18_{MBC}

| | 5′- | Direction | Primer | Sequence |
|---|---|---|---|---|
| Primer | Coordinate | (+/−) | Length | (nt) |
| CL 1A | 1 | + | 30 | TGGAAGGGCTAATTTACTCCCAAAAAAGAC |
| CL 14 | 896 | − | 25 | AATCGTTCTAGCTCCCT [symbol] CTTGCCC |
| CL 1B | 1 | + | 30 | AATCCCGGGTGGAAGGGCTAATTTACTCCC |
| CL 13 | 796 | − | 31 | CCTCTAGACCGCTTAATACTGACGCTCTCGC |
| CL 11 | 682 | + | 23 | TCTCTCGACGCA [symbol] ACTGCTT |
| CL 1 [symbol] | 3440 | − | 30 | CTGTTTTCT [symbol] CCAGTTCTAGCTCTGCTTCT |
| CL 12A | 732 | + | 26 | TTTCCCGGG [symbol] T [symbol] TAC |
| CL 17 | 3330 | − | 32 | CCCTCTAGACTT [symbol] CCCAATTCAATTTTCCCAC |
| CL 26 | 3193 | + | 39 | CCACACCA [symbol] CAAAA [symbol] CCCCCSTTCC |
| CL 6 [symbol] | 9671 | − | 39 | TGCTA [symbol] GATTTTCCACA [symbol] CTAAAATGGTCT [symbol] |
| CL 27 | 3251 | + | 39 | CCATCCTGATAAATGGACA [symbol] TGCC |
| CL 28 | 639 | − | 37 | TGGCCCAAACATTATGTACCTCT [symbol] CATCATATGC |
| CL 19 | 5448 | + | 30 | AGCAGGACATAACAAGGTA [symbol] ATCTCTACA |
| CL 24 | 8422 | − | 28 | GGATCTGTCTCTGTCTCTCTCTCCACCT |

Underlined sequences depict added restriction enzyme site
+ and − orientations refer to sense and antisense strands of the double stranded DNA sequence

Underlined sequences depict added restriction enzyme site

+ and − orientations refer to sense and antisense strands of the double stranded DNA sequence

TABLE 5
Sequence Deletions and Insertions in HIV-1 C18_{MBC}
Compared with HIV-1_{NL43}

| | | |
|---|---|---|
| Position (nt) | Deletions | Insertions |

| Gene or Region | C18_{MBC} | NL43 | (nt) | (nt) |
|---|---|---|---|---|
| 5′-LTR U3 | 29 | 29 | 120 | — |
| 5′-LTR U3 | 85 | 205 | 87 | — |
| 5′-LTR U3 | 154 | 360 | — | 9 |
| gag p17 | 939 | 1134 | — | 15 |
| gag p15 | 1982 | 2163 | — | 30 |
| gag p15 | 2081 | 2232 | — | 6 |
| vpu | 5927 | 6062 | — | 3 |
| vpu/env | 6092 | 6234 | — | 9 |
| vpu/env | 6128 | 6261 | 12 | — |
| env | 6483 | 6628 | — | 6 |
| env | 6514 | 6653 | 2 | — |
| env | 6524 | 6665 | 1 | — |
| env | 6630 | 6772 | — | 9 |
| env | 6646 | 6778 | — | 3 |
| env | 7011 | 7141 | 6 | — |
| env | 7140 | 7276 | 3 | — |
| env | 7195 | 7334 | — | 6 |
| env | 7266 | 7399 | 3 | — |
| env | 7278 | 7414 | — | 6 |
| env | 7290 | 7420 | — | 2 |
| env | 7300 | 7429 | — | 1 |
| env | 7314 | 7441 | 3 | — |
| env | 7463 | 7593 | — | 3 |
| env | 7471 | 7598 | — | 9 |
| nef | 8711 | 8829 | 177 | — |
| nef | 8723 | 9018 | 11 | — |
| nef/LTR | 8798 | 9104 | 120 | — |
| nef/LTR | 8854 | 9280 | 87 | — |
| LTR U3 | 8923 | 9435 | — | 9 |
| | | | 632 | 126 |

### EXAMPLE 13

### Macrophage Isolates of HIV-1 C18 and HIV-1 C98

HIV-1 has been isolated from the macrophages of patients C18 and C98.

Patient monocytes were prepared as follows. Whole blood was spun at 2000 rpm for 10 minutes. Plasma was removed into a separate tube and the remaining cells were diluted 1:2 in PBS (magnesium and calcium free phosphate buffered saline). This was underlaid with 10 ml of Ficoll Isopaque and spun at 2000 rpm for 20 minutes. Cells were collected from the interphase and washed three times with PBS. These cells were then seeded into a 6 well Costar tray at a concentration of 1.0×10⁷/ml and allowed to adhere for 1 hour. Any non-adherent cells were removed by aspiration.

Donor HIV-1 negative macrophages for use in co-cultivation Arc prepared as follows Peripheral blood mononuclear cells were purified from whole blood using Ficoll/Isopaque density gradient. These cells were seeded at a concentration of 2.0×10⁶/ml in teflon. PBMC were cultured in the presence of 3 μg/ml of PHA and 1000 U/ml of M-CSF 3 days prior to co-culture.

On day of co-culture, donor PBMC were CD8 depleted. Dyna beads coated with anti-CD8 were used for this purpose. Dyna beads were washed once in PBS and then applied to a magnet for 3 minutes. Supernatant was removed and the beads were then resuspended in 250 μl of RF-10. Aliquots of 2.0×10⁸ patient cells were then added to 250 μl (3 beads:1 CD8 T-cell) of Dyna beads and allowed to incubate for 30 minutes on ice with occasional mixing. After 30 minutes the cell suspension was placed onto a magnet for 3 minutes. The supernatant was then removed placed into a second tube containing 142 μl (1 bead:1 CD8 T-cell) of Dyna beads. This suspension was placed on ice for an additional 30 minutes with occasional mixing. After 30 minutes cell suspension was placed onto a magnet for 3 minutes. Supernatant was removed and washed once in RF-10.

For co-culture, CD8 depleted PBMC were then added to patient monocytes. Half media changes were done every 7 days for a period of 21 days. Aliquots of 2.5 ml of medium was removed from these cultures and replaced with CD8 depleted donor PBMC in Iscoves containing 10% HuS (Human serum), 5% v/v FCS and 5% w/v IL-2 and 1000 U/ml of M-CSF. Harvested supernatants were spun at 1400 rpm for 10 minutes and stored as 1 ml aliquots. Cell pellets were lysed in 200 μl of lysis buffer for PCR analysis. Infection was quantitated using a p24 EIA Kit.

Cells were harvested from the co-cultures and used to prepare DNA as described above. The nef/3′-LTR region of both virus isolates was amplified by PCR using the above described primer sets and conditions (Example 12). The resulting amplimers were cloned into the plasmid vector pT7T3U19 and the nucleotide sequence determined by the Taq cycle sequencing method with dye-labelled primers.

The C18 macrophage sequence has 3 deletions staring and finishing at positions within 3 nucleotides of the same deletions in C18_{MBC}. The encoded nef protein is 3 amino acids long compared with 7 amino acids for C18_{MBC}. The low homology region of the LTR U 3 region of C18 macrophage is very similar in sequence to C18_{MBC} and similarly it has one extra upstream NFκB site.

On the other hand, the sequence of C98 macrophage has a number of differences from the C98 isolate. While it has exactly the same first deletion of 16 nucleotides just upstream of the polypurine tract (PPT), in the nef-alone region, and exactly the same second deletion (position and size) it has an extra deletion of 18 nucleotides at HIV-1_{NL43} equivalent nucleotides 9206 to 9223. The final deletion is in approximately the same position as in the C98 isolate but is 5 nucleotides longer. The encoded nef protein is 34 amino acids long compared with 86 amino acids for the C98 isolate. The low homology region is very similar to the C98 isolate, having the same 2 extra upstream NFκB sites and completely lacking the normal 5′-NFκB site.

### EXAMPLE 14

### Construction and Use of an Infectious Molecular Clone

Molecular biological techniques can be used to construct a molecular clone of, for example, HIV-1 C18_{MBC}. Two schemes may be used. In the first scheme genomic DNA, extracted from either the CD4 positive PBMC of the patient C18 or donor PBMC that have been infected with the isolate HIV-1 C18_{MBC}, is used as the template for polymerase chain reaction amplification, using thermostable polymerase of high transcriptional fidelity (eg UITma polymerase or KlenTaq/Pfu polymerase mixture), of long (6 to 7 kb) overlapping fragments representing the 5′- and 3′-parts of the HIV-1 C18_{MBC} proviral genome of total length 9207 nts. The amplified fragments may then be ligated together after digestion with a restriction enzyme that cleaves at a unique site common to the overlapping region of the amplified fragments, for ample the unique Bgl I or Nco I sites Ligation of this fill length proviral DNA into a plasmid vector win allow its propagation in E coli and the subsequent preparation of large (mg) quantities of this molecularly cloned proviral DNA.

In the second scheme donor PBMC that have been infected with the isolate HIV-1 C18_{MBC} are used as a source of non-integrated proviral DNA which can be extracted from the infected cells by the Hirt extraction method (Hirt, 1967). Circular proviral DNA molecules may be linearised by digestion with a restriction enzyme that cleaves at a unique position in the genome (eg the Bgl I or Nco I sites). The resulting linearised molecules can be ligated into a plasmid or, more usually, a bacteriophage lambda (λ) based vector (eg Charon 4a, λWES) after modification of the end to provide blunt or cohesive ends compatible with the vector. Transformation or transduction of E coli with the recombinant plasmid or bacteriophage material, respectively allows the propagation of the proviral DNA. Clones of E. coli containing proviral DNA may be selected and DNA prepared. Molecular clones of retroviral genomes prepared in this way are often permuted. Rearrangement to the functional arrangement of sequence is achieved by restriction enzyme cleavage and religation of fragments to reconstruct the correctly permuted proviral genome.

The molecularly cloned DNA products of both schemes can be used to prepare variant proviral genomes that may be used as the basis of a biologically attenuated HIV-1 vaccine strain. Similarly, they may be modified to contain extra DNA sequences in the nef-alone deletion region that may deliver sequences that may be of therapeutic advantage (eg antisense or ribozyme sequences).

Infectious virus particles of HIV-1 C18_{MBC}, or modified virus, can be produced by transfection of human cells (eg HeLa cells) which will produce, and release to the culture medium, virus particles of HIV-1 C18_{MBC}, or modified virus. These virus particles can be used to infect a variety of CD4 positive cells for further propagation or experimentation.

### EXAMPLE 15

### In Vivo Primate Model

Following construction of infectious molecular clones of the mutant HIV-1 strains, studies are then undertaken in primates to establish attenuation, immunogenicity and vaccine prophylactic efficacy. All studies compare mutant clones of HIV-1 with isogenic wild-type (WT) virus. Initial studies ar performed using the macaque (M. nemistrina) model of HIV-1 infection. Macaque-infectious WT HIV-1 and mutant clones are compared with respect to duration of viremia, anatomic sites of replication, and cellular and humoral immune responses. Where the mutant HIV-1 clones induce an immune response in the macques infected, challenge studies with WT virus are also performed. Studies are performed in a limited number of chimpanzees, generally in parallel with the macaque studies. Relevant mutations are into WT HIV-1 clones previously shown to produce chronic infection in chimps, and the course of chimp infection with mutant clones compared with historical controls. If infection is established, WT challenge studies is also performed.

Examples 16 to 21 relate to the screening of antibodies in HIV-1 infected individuals to peptides covering regions of Nef.

### EXAMPLE 16

### Study Subjects

Serum samples were obtained from seven HIV+ve individual D-36, C-124, C-98, C464, C-18, C-49 and C-54. Individuals C124, C-98, C-64, C18, C49 and C-54 (recipients) were infected through units of blood or blood products from donor D36, over a 2-year period. Long-term follow-up of the six recipients and the donor, shows persistent long term asymptomatic infection. This group is referred to herein as the long term non-progressor 1 (LTNP1) cohort. Members of this cohort have been infected for an average of 11 years (10.75 to 14 years) and it was established that the donor had been infected since April, 1981 (Learmont et al, 1992). Regular follow-up includes history, physical examination, full blood count, T-cell subset counting and measurement of serum p24 antigen and β2-microglobulin concentrations (Learmont et al, 1992; Learmont et al, 1995). As controls, sera were obtained from 14 HIV-1 negative individuals (HIV-1-ve). 4 patients who were infected with HIV-1 through sexual activity or through blood transfusion and are also considered long-term non-progressors and 12 HIV-1 positive (HIV-1+ve) individuals that have developed clinical infections (long term progressors LTP). Sera from patients with autoimmune disease and who were HIV-1-ve were also employed.

### EXAMPLE 17

### Peptide Synthesis

Peptides corresponding to amino acid residues 1 to 19, 20 to 36, 44 to 65, 72 to 83, 89 to 97, 109 to 114, 121 to 136, 162 to 177, 164 to 186 and 187 to 206 of HIV-1 nef (HIV-1_{NL43}) were synthesized using standard t-Boc chemistry and purified by high pressure chromatography as described elsewhere (Fecondo et al. 1993).

### EXAMPLE 18

### Expression of Recombinant HIV-1 Nef Protein in E. coli

Thc large scale expression of the 27 kDa form of HIV-1_{NL43} in E. coli and subsequent purification were as described by Azad et al (1994).

### EXAMPLE 19

### Screening of Sera from HIV-1+ve Individuals and Control HIV-1−ve Groups for Reactivity Against Nef Protein and Derived Peptides by Direct EIA

For detecting antibodies that recognise the Nef protein or its peptide derivatives, highly purified full length Nef protein or peptides corresponding to the HIV-1_{NL43} Nef amino acid sequence were coated onto the wells of 96-well polysyrene microtitre plates at 100 ng/well or 500 ng/well in PBS, respectively. Peptides and proteins were allowed to coat for 2 h at 37° C. After this incubation period the wells were washed three times with PBS containing 0.05% v/v Tween 20 (PBS-Tween) and any available sites on the wells blocked by incubation of 150 μl of gelatine (1% w/v) in PBS for 1 h at 37° C. Following washing with PBS-Tween as described above 50 μl of serum diluted in PBS/BSA (1% w/v) was added to the wells and incubated for 1.5 h at 37° C. Sera added to the wells included that from the seven cohort member (D36, C98, C18, C54, C49, C64 and C124 [see Learmont et al, 1992]) and the control groups described above. The wells were again wed with PBS/Tween and subsequently incubated with 50 μl of biotinylated sheep anti-human Ig (diluted 1:1000 in 1% w/v BSA/PBS; Amersham) for 1 h at 37° C. Following further washing, 50 μl of Streptavidin-HRP (diluted 1:1000 in 1% w/v BSA/PBS; Dakopatts) was added to the wells and the plate incubated at 37° C. for 30 min. An aliquot of 100 μl of substrate (O-phenylenediamine, Sigma) was finally added after washing and the plate allowed to incubate at room temperature for 15 min. The reaction was stopped by the addition of 1 N H₂SO₄ and the plate read at 450/630 nm using a Titertek plate reader.

### EXAMPLE 20

### Recognition of Full Length Recombinant Nef Protein by Patient Sera

The prevalence of a Nef specific antibody response the cohort members (referred to herein as (LTNP1), long term progressors (LTP) HIV-1+ve individuals and another group of long term non-progressors (LTNP2) patients who were infected by different donors was assessed by EIA. Sera obtained from 14 normal individuals (HIV-1-ve) and 14 individuals with autoimmune disease (A/HIV-1-ve) were used as controls.

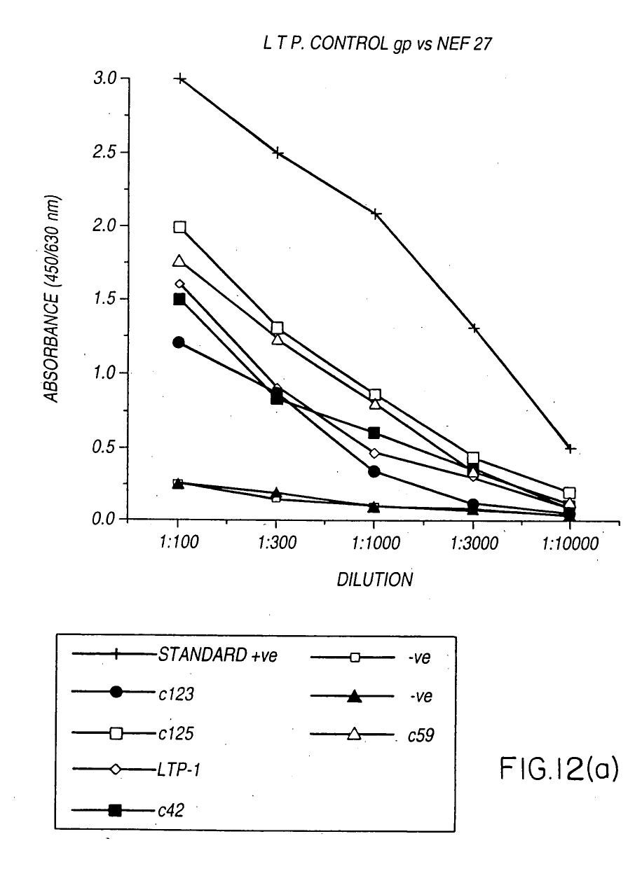

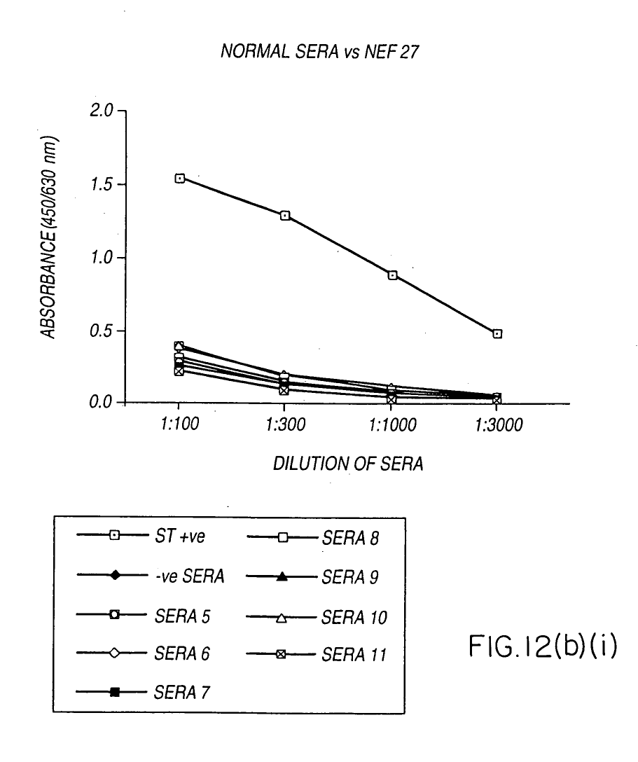

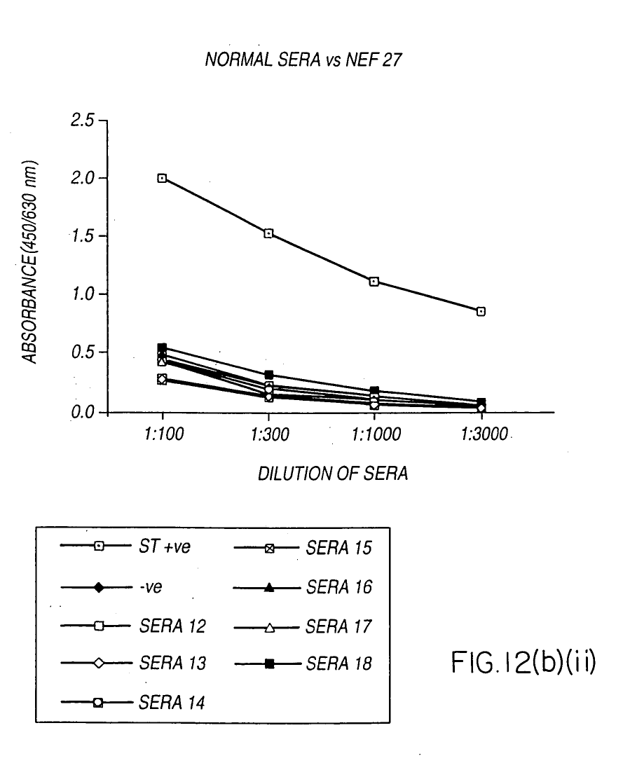

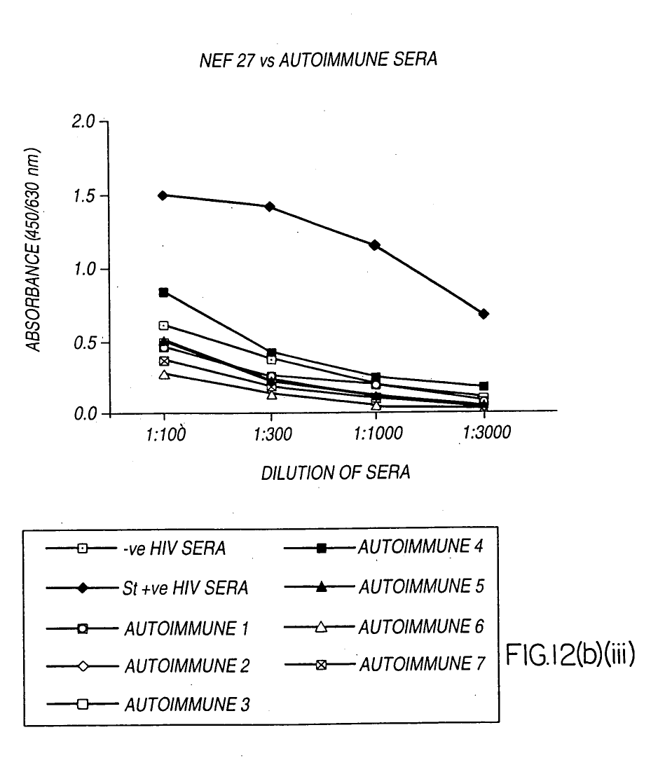

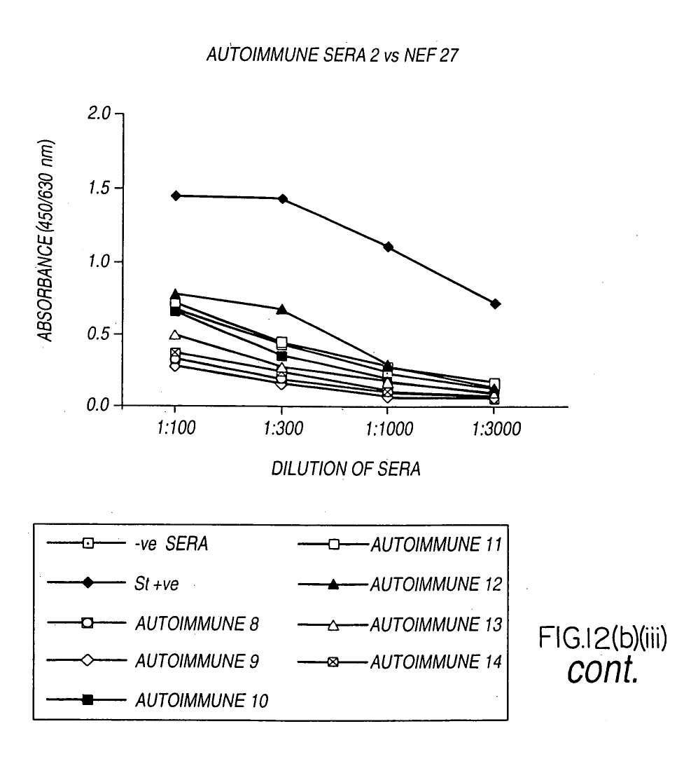

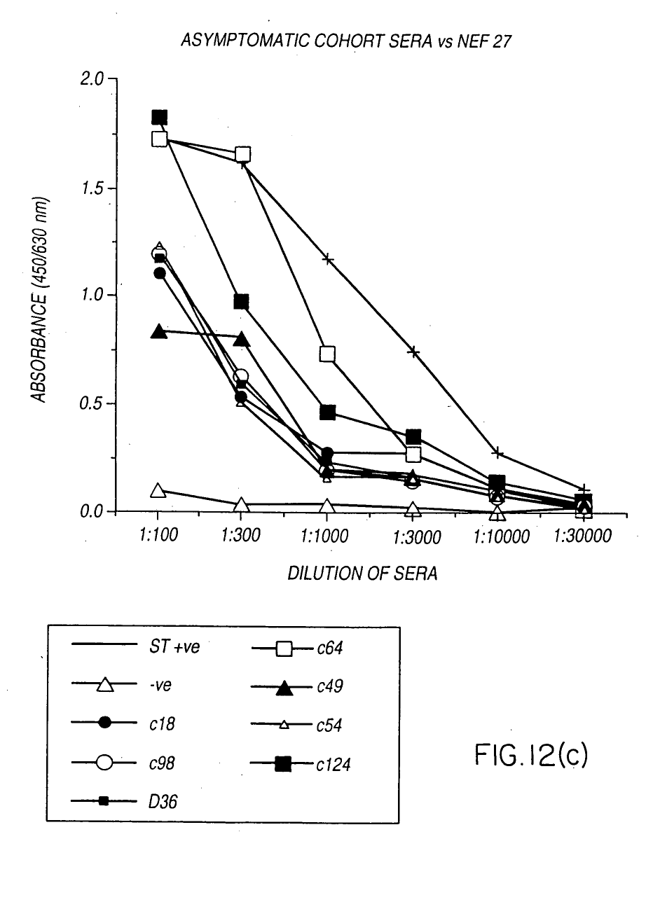

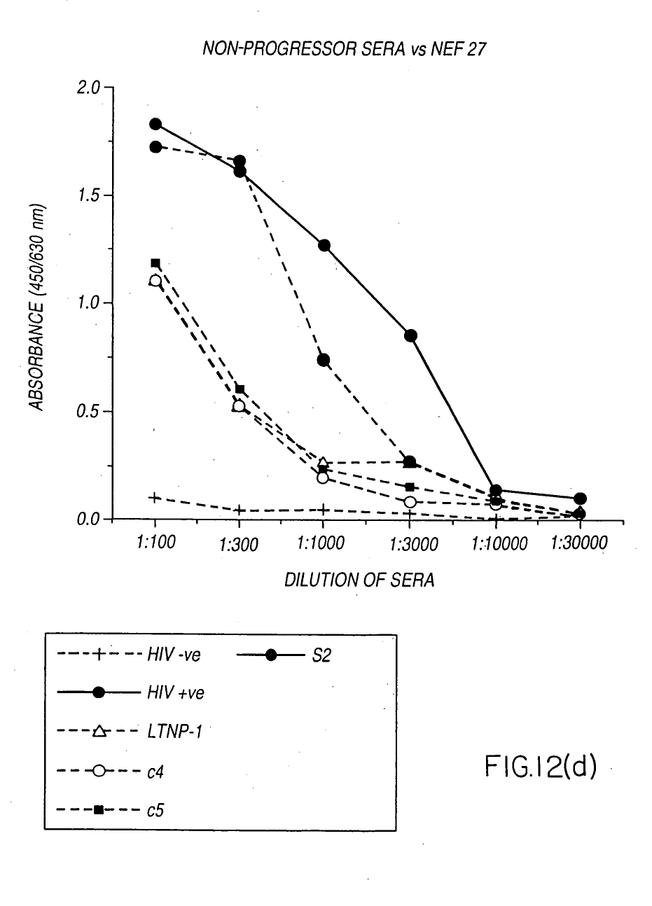

All individuals who were classified as LTP patients demonstrated high levels of antibodies that recognised fill length Nef protein (FIG. 12a). Sera obtained from HIV-1-ve or the A/MV-1-ve groups showed only low level recognition, which was considered at background levels, towards Nef protein (FIG. 12b(i)(ii)). In contrast to normal individuals, sea obtained from the LTNP1 cohort showed high recognition of Nef (FIG. 12c), indicating the presence of significant levels of Nef antibodies in the sera of these individuals. The sera titered out to approximately 1:3000. Similar levels of Nef antibodies were observed in the LTP group (FIG. 128). Nef-positive antibodies were also detected in the LTNP2 group and again titered at 1:3000 dilution (FIG. 12d).

### EXAMPLE 21

### Recognition of Nef-Derived Peptides by Sera

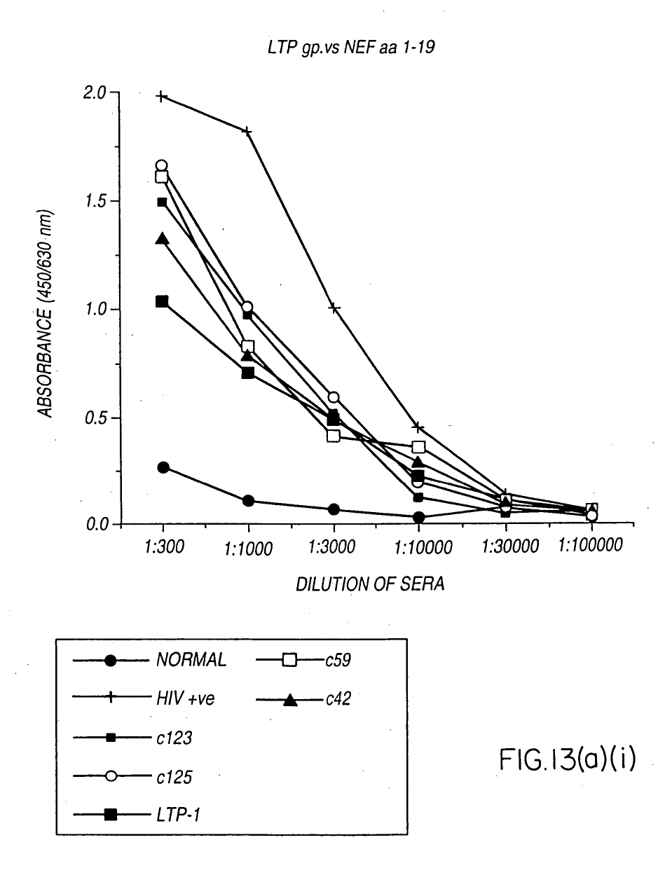

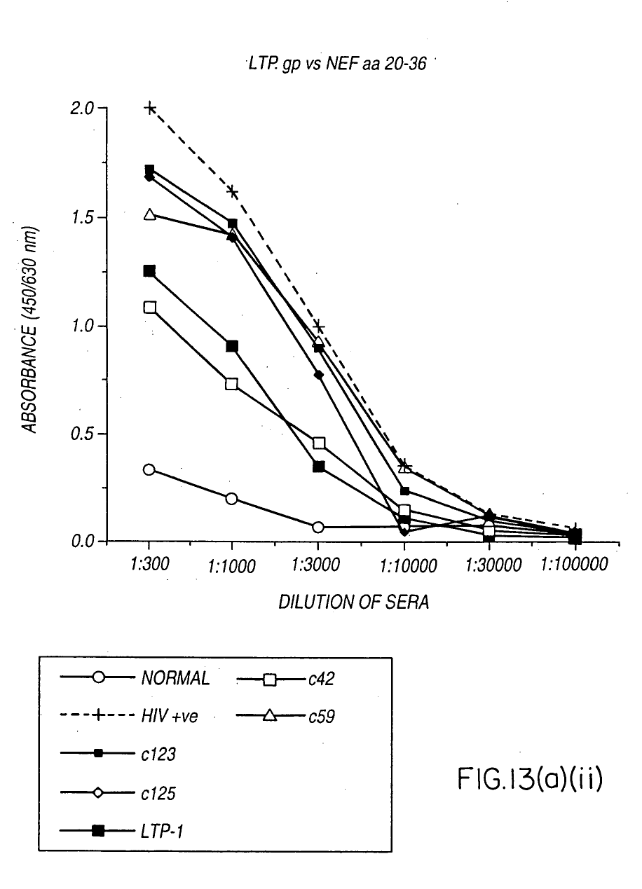

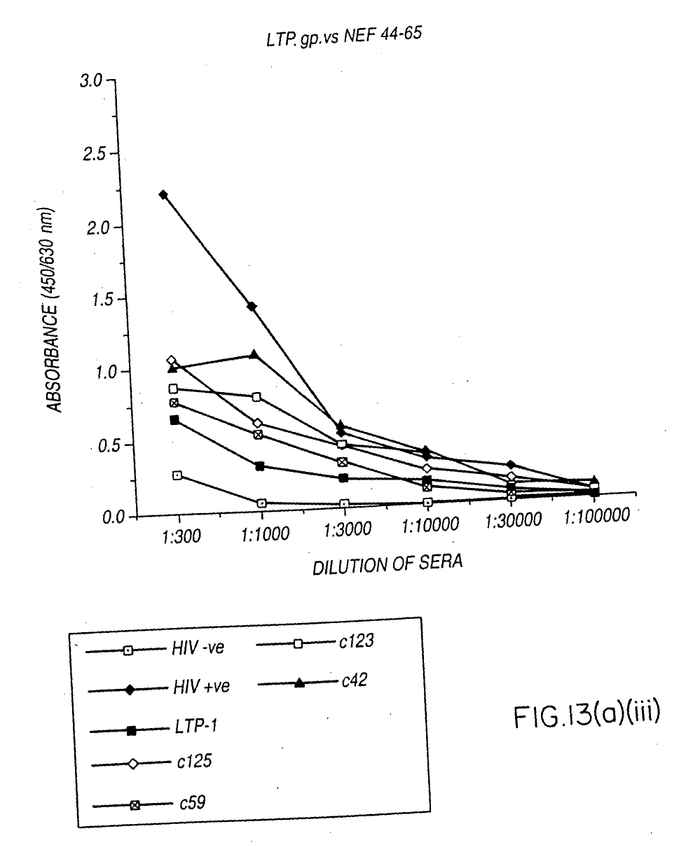

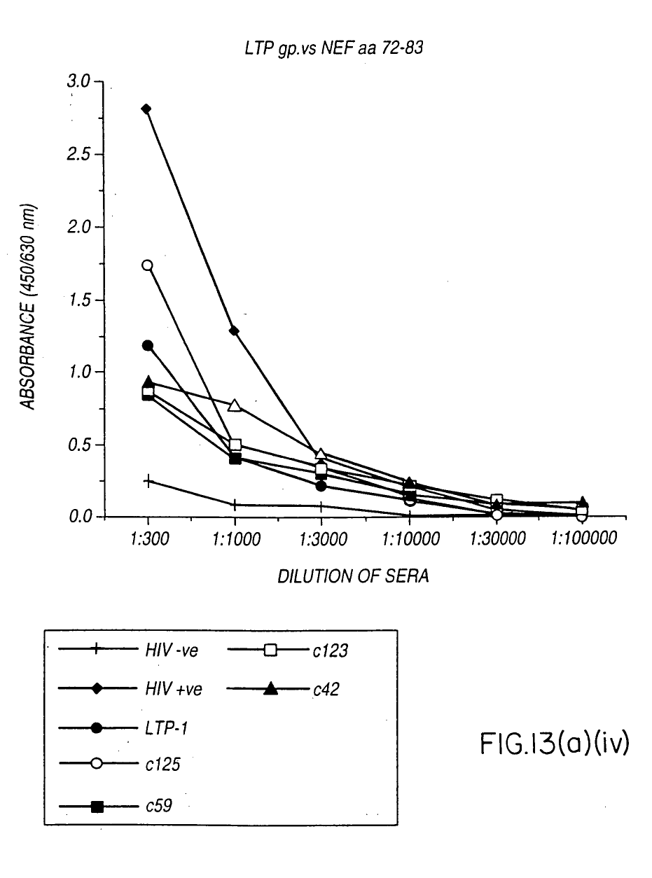

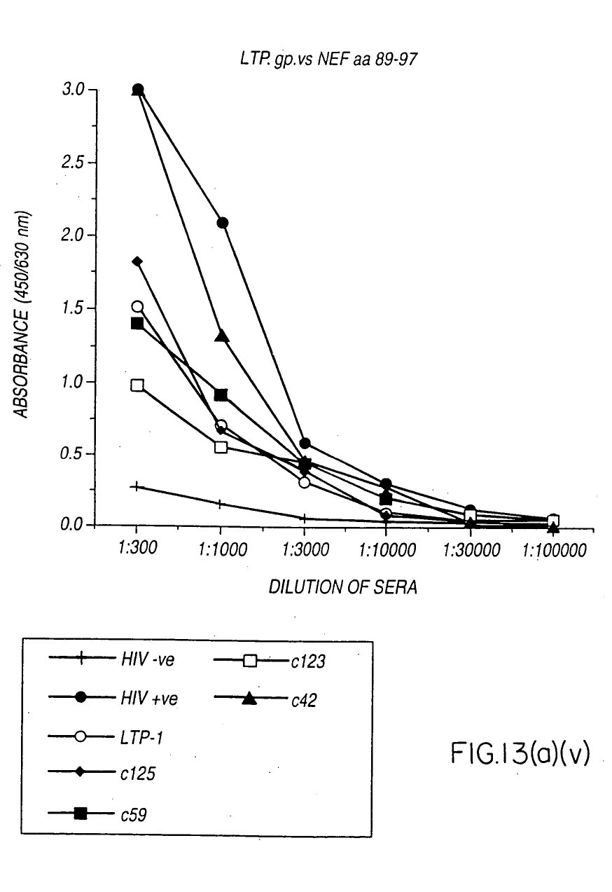

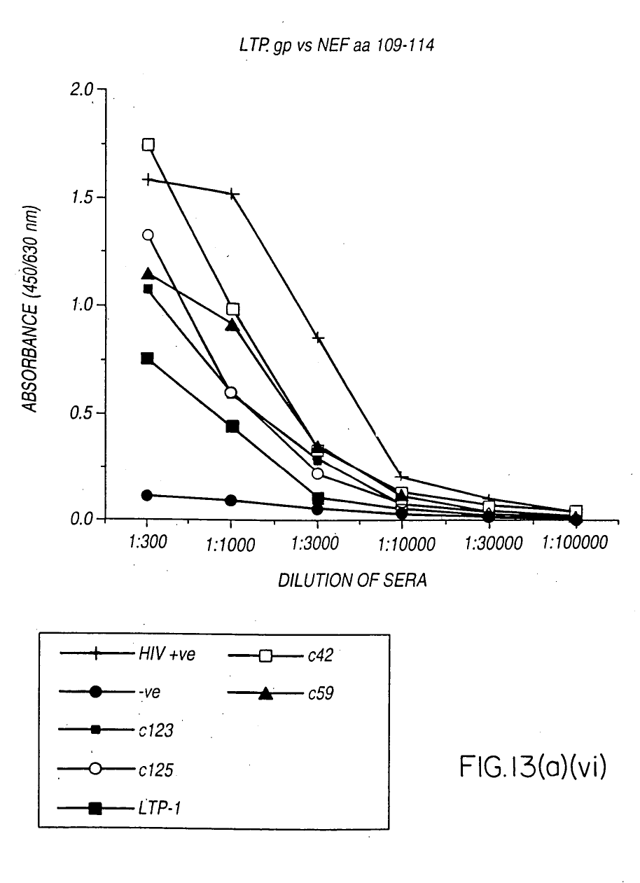

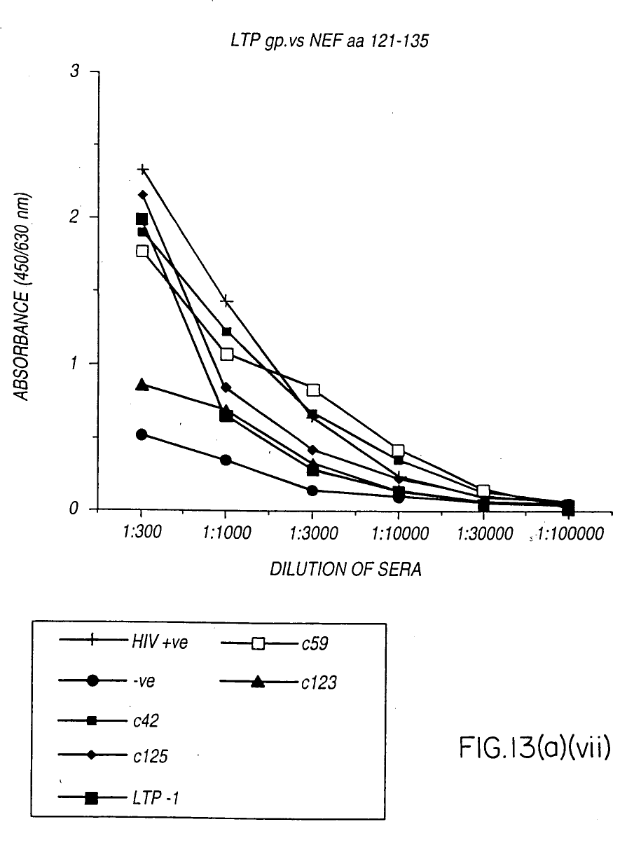

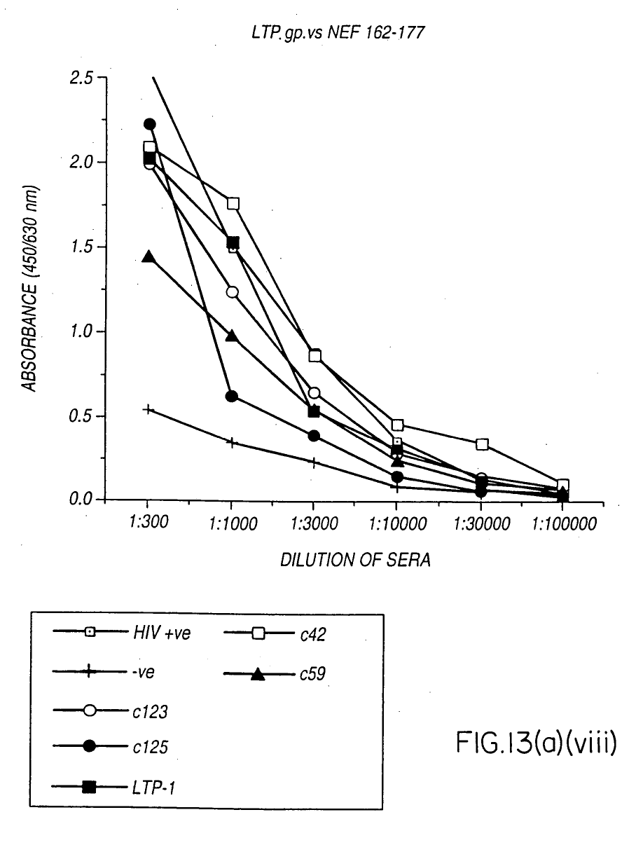

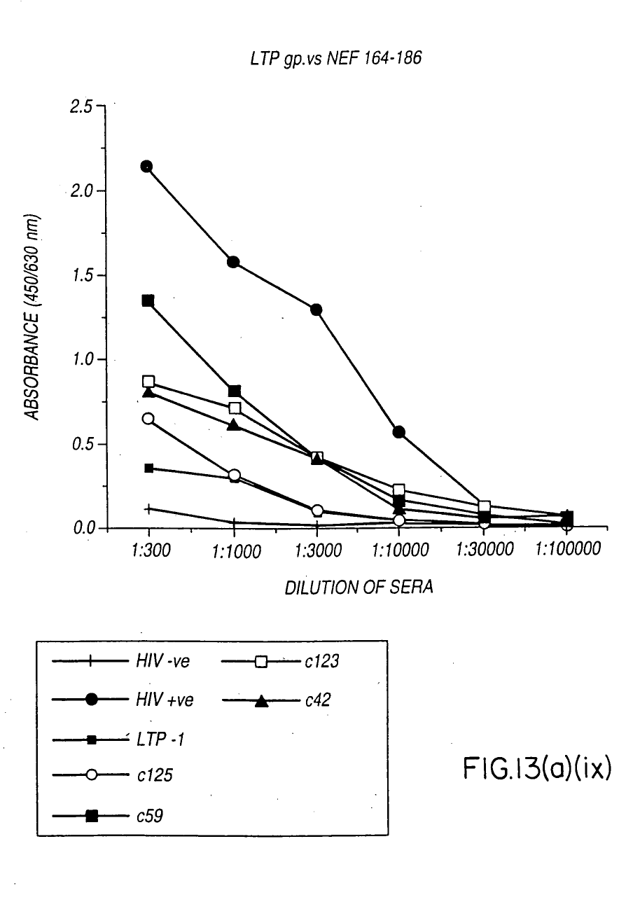

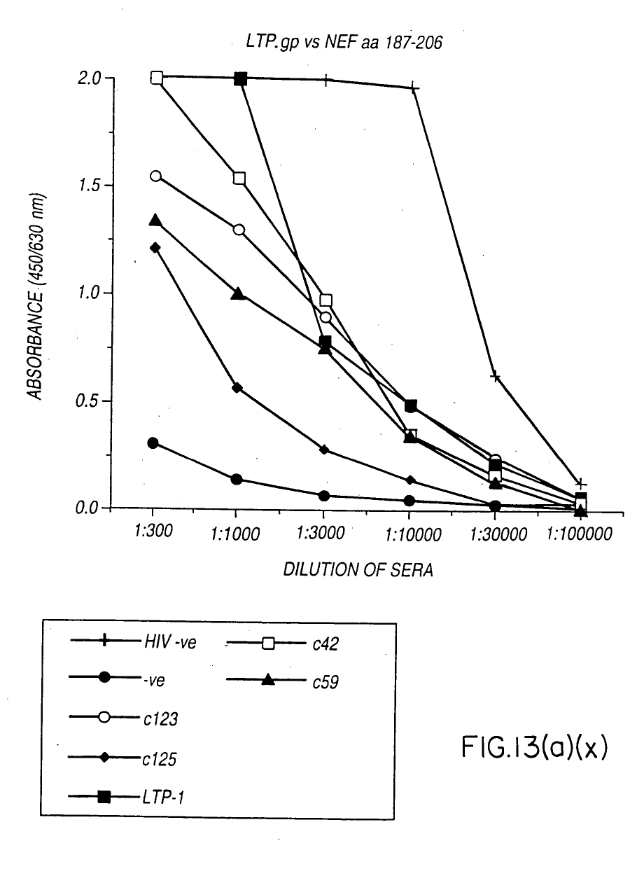

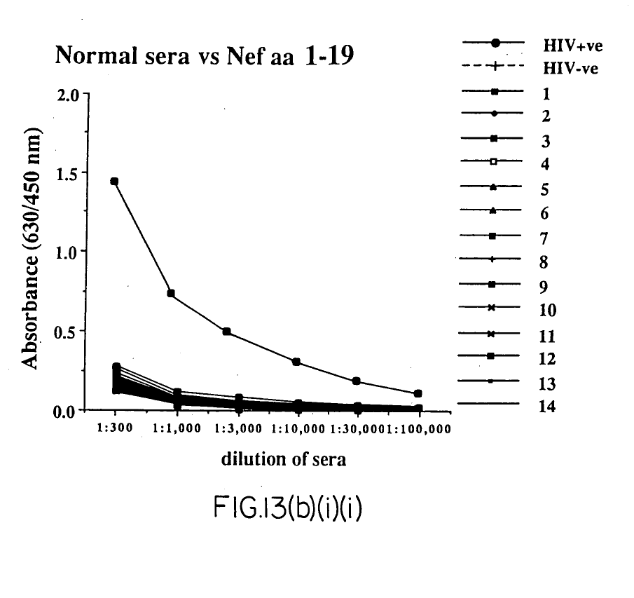

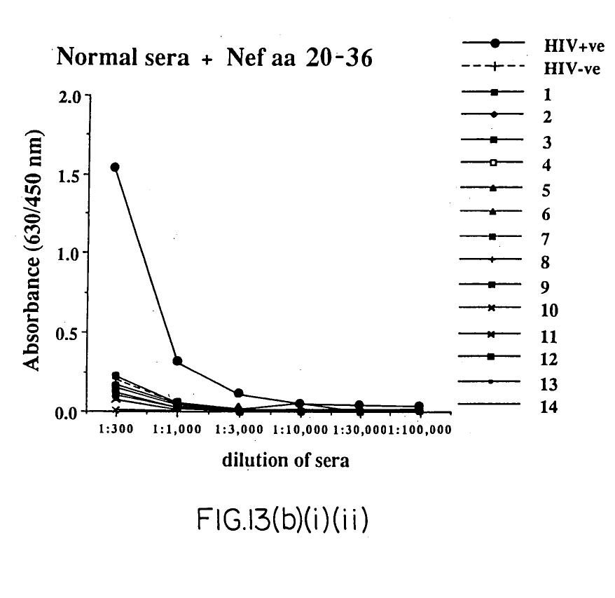

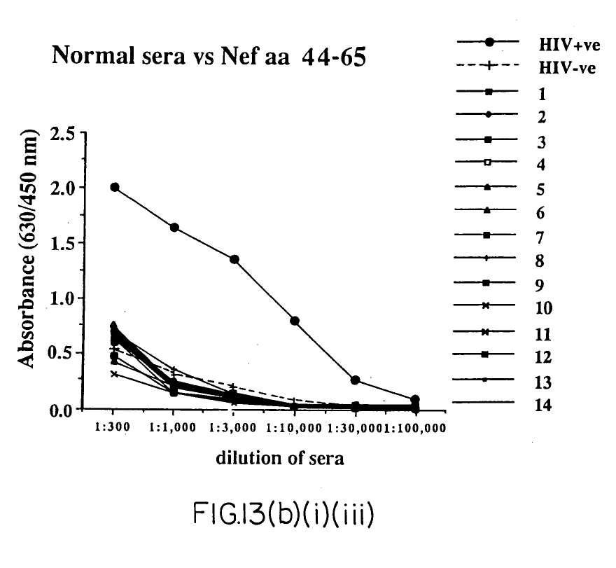

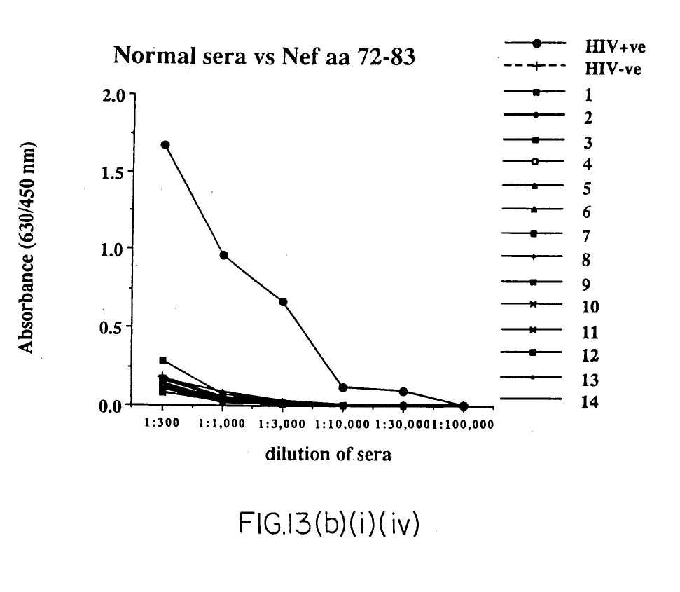

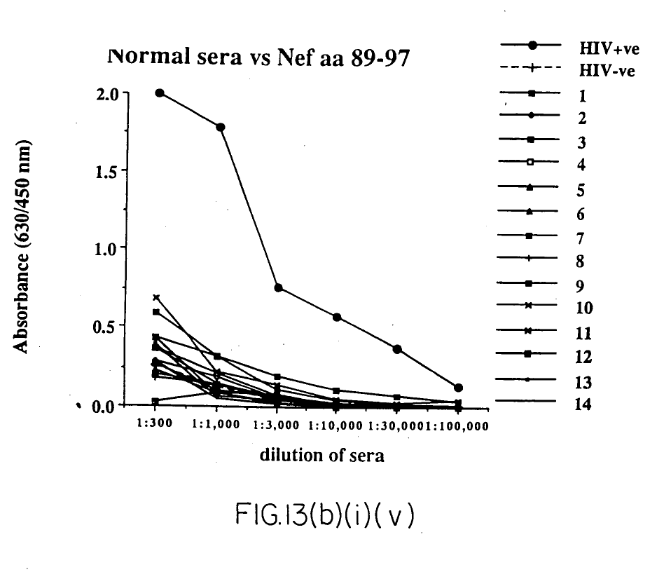

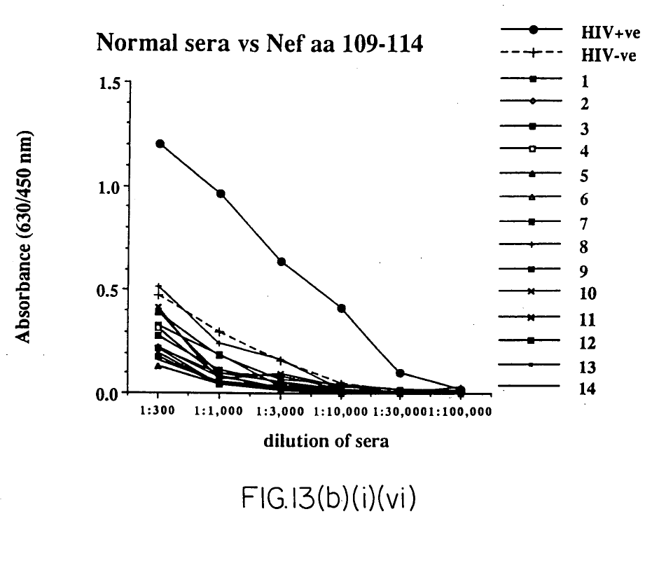

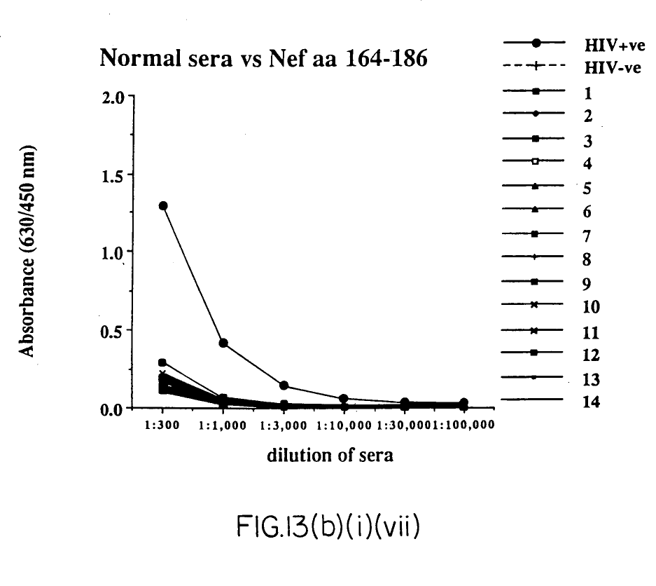

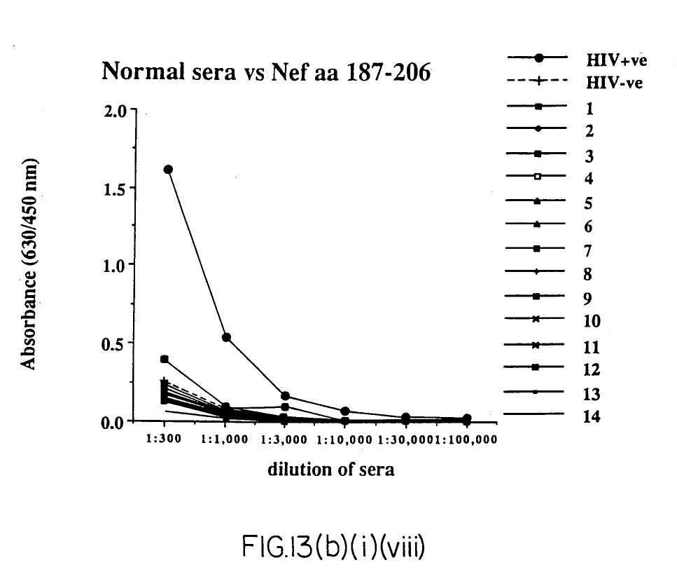

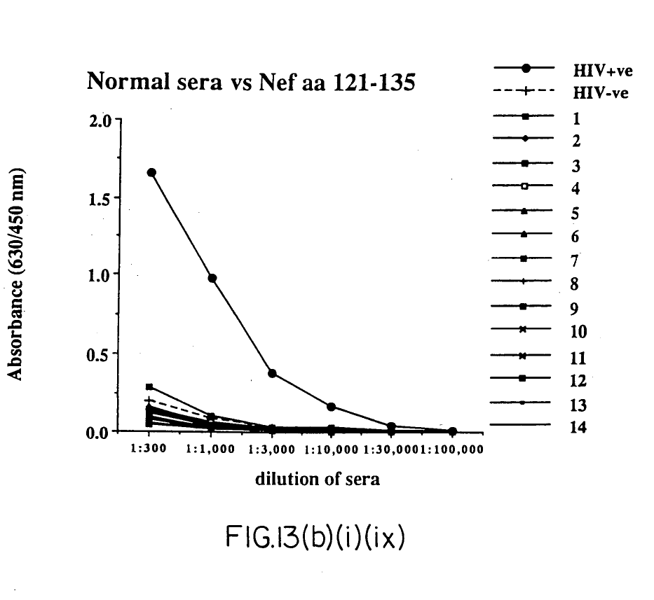

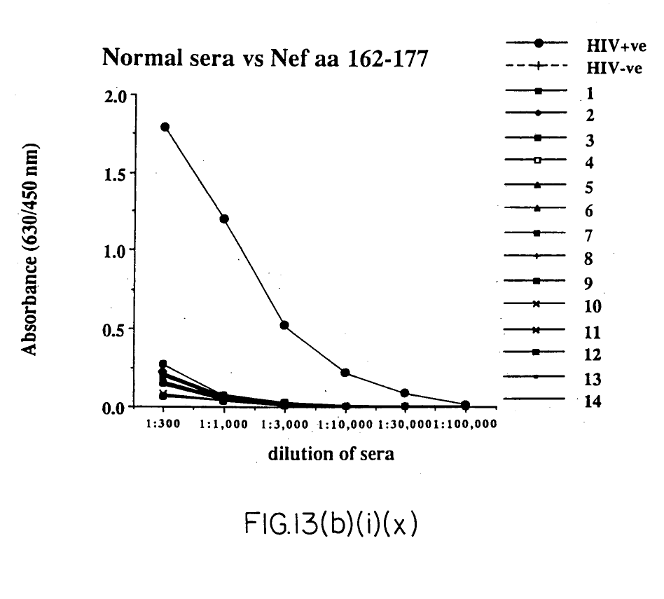

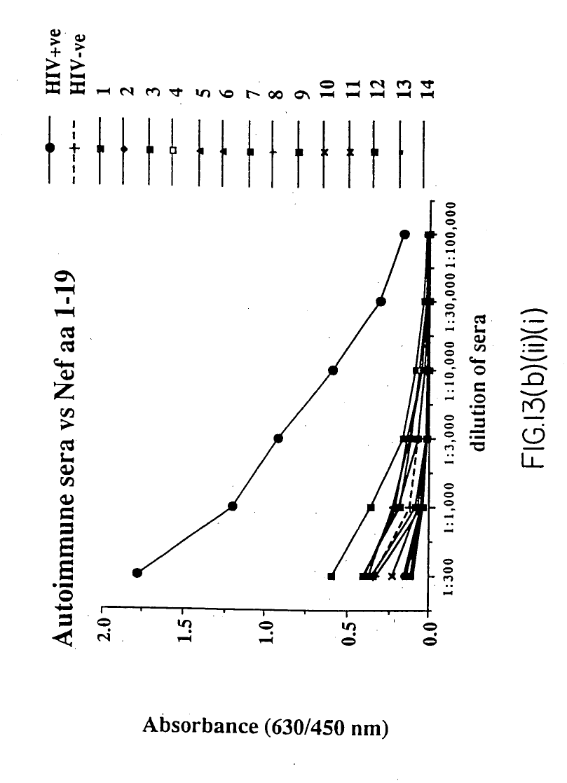

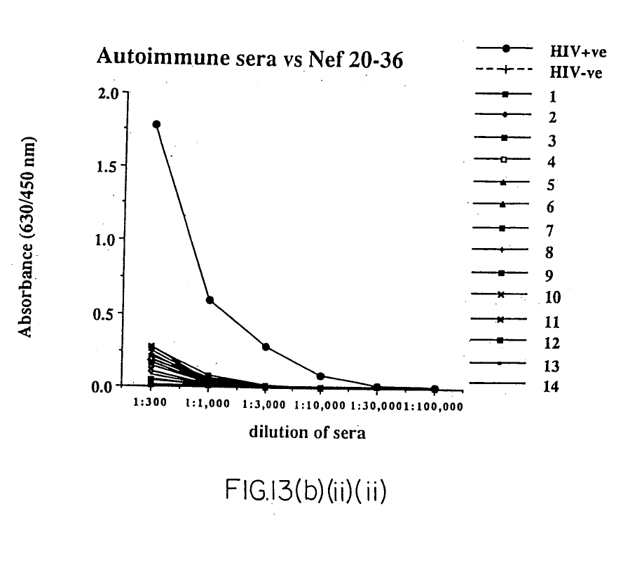

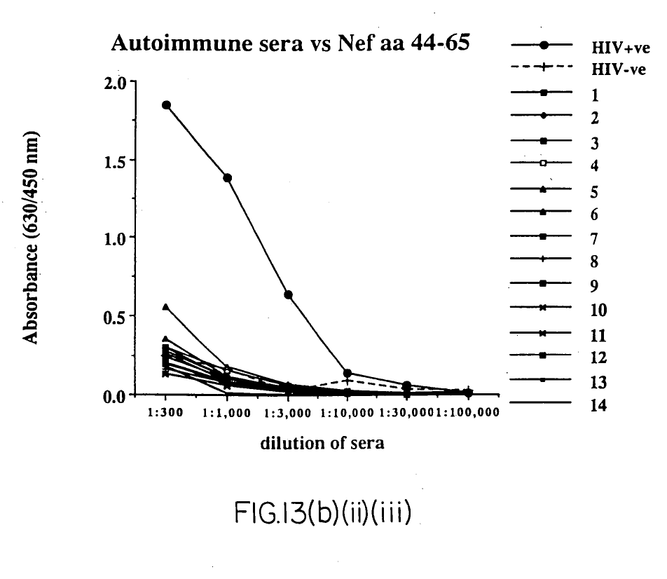

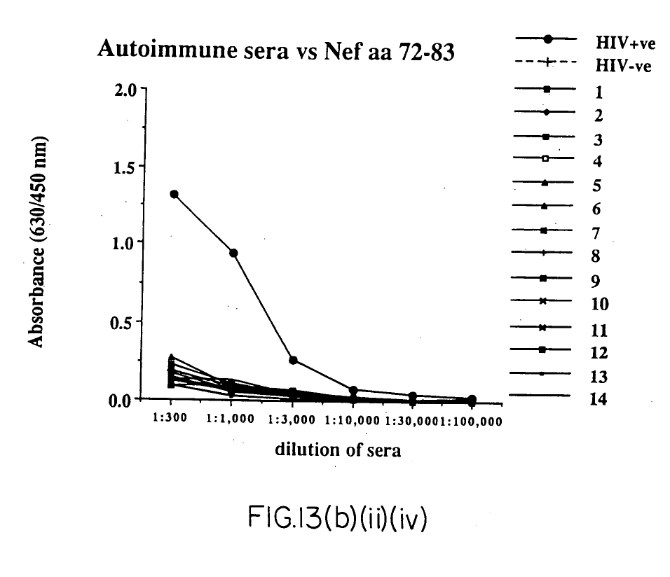

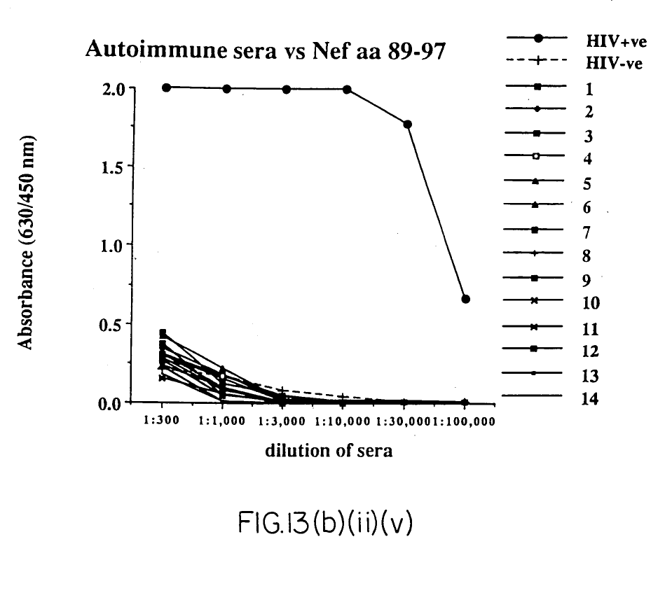

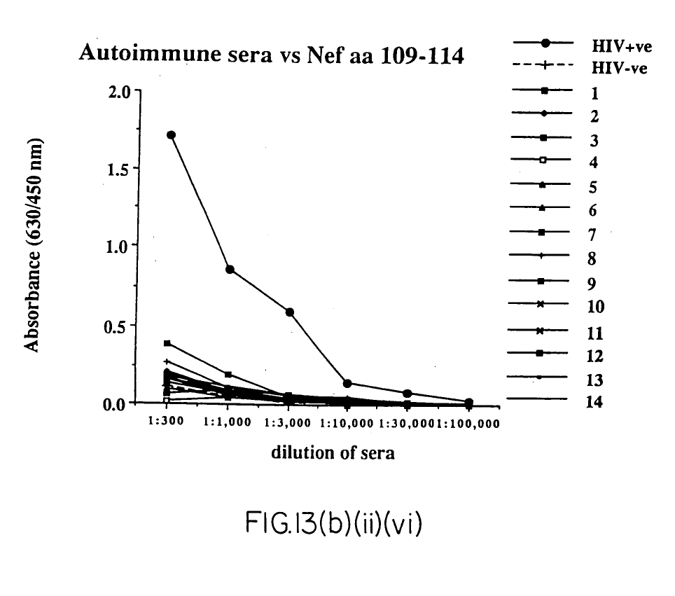

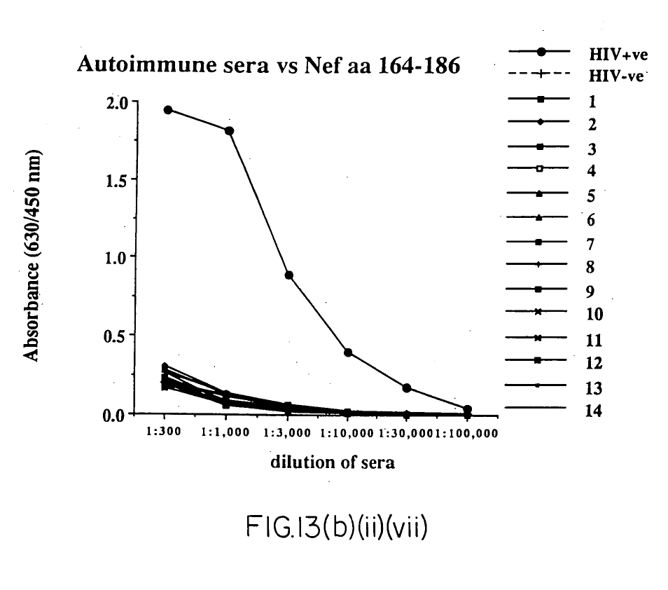

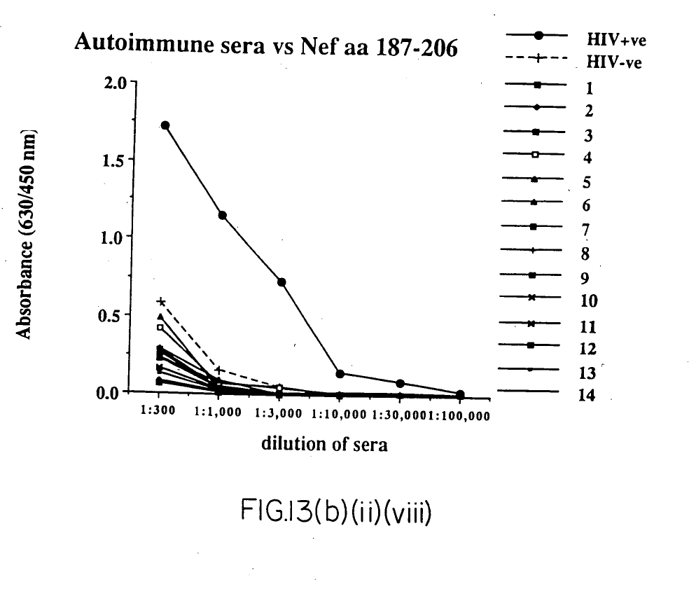

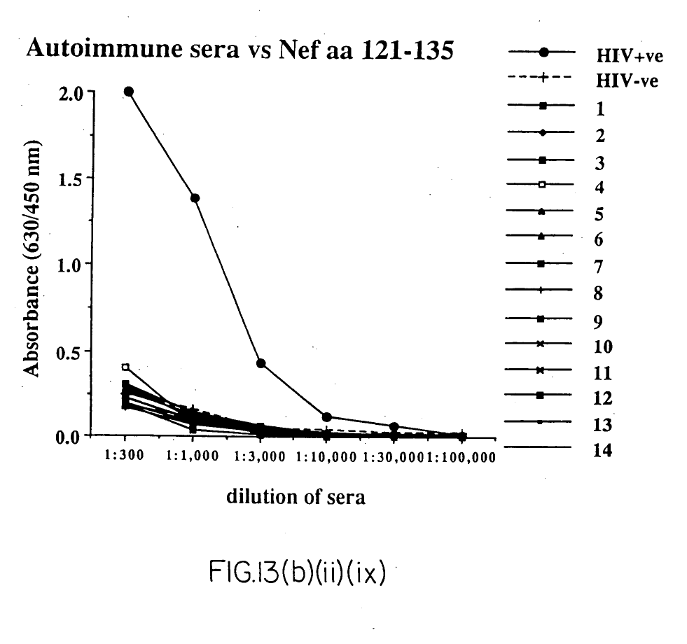

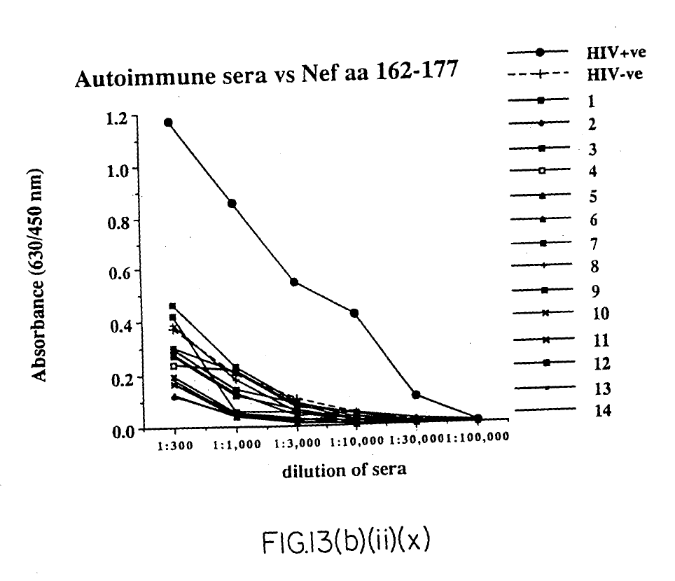

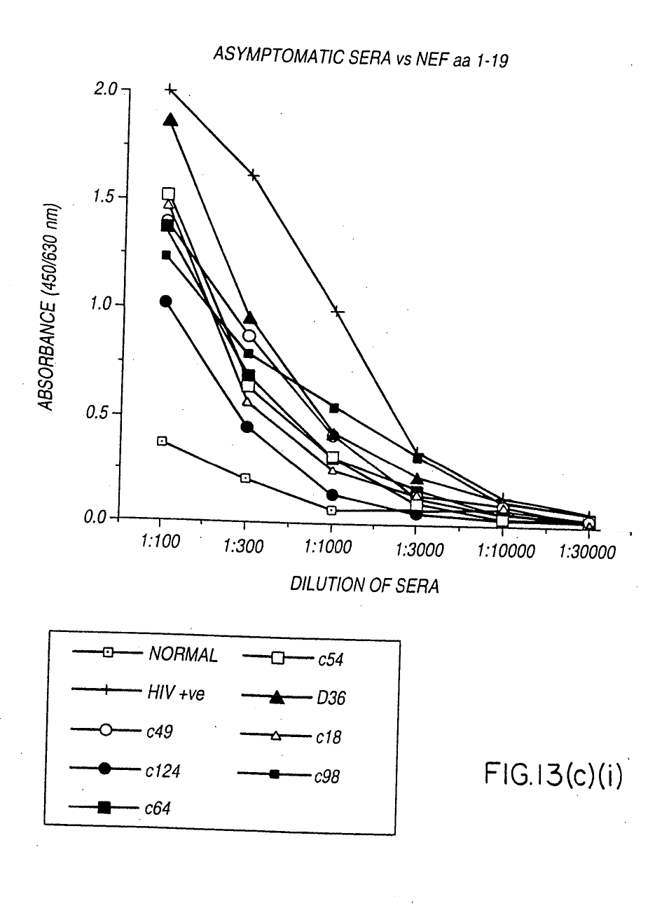

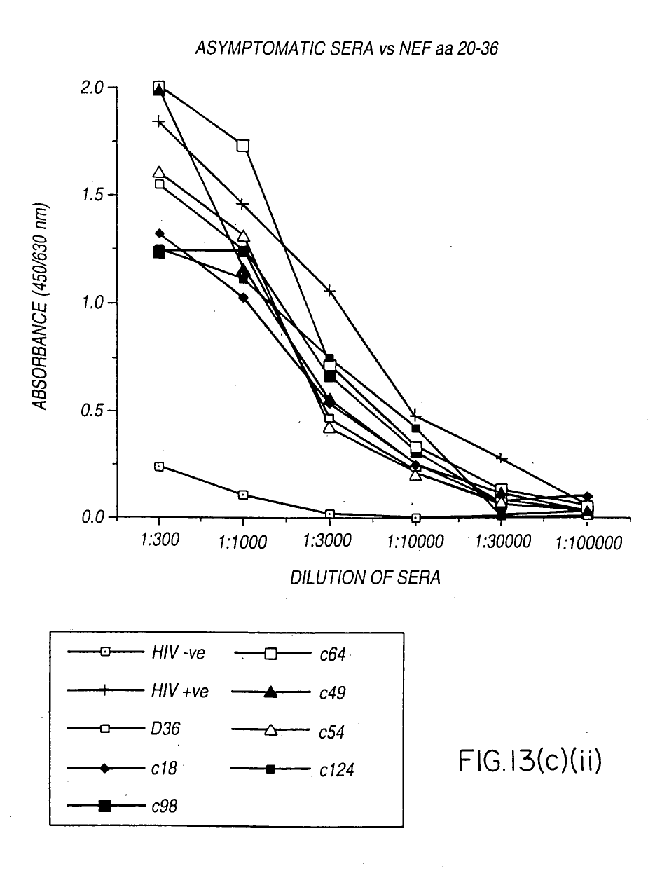

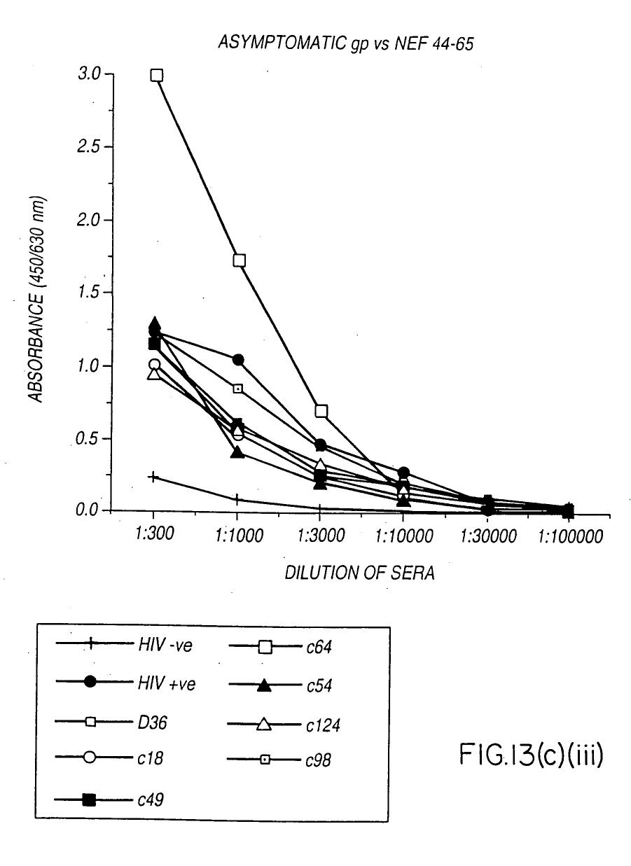

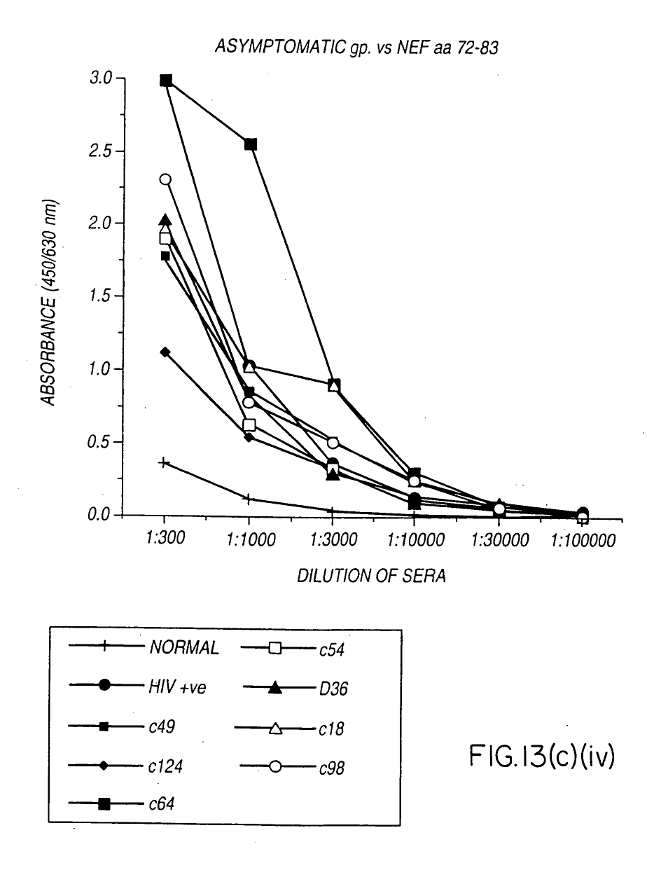

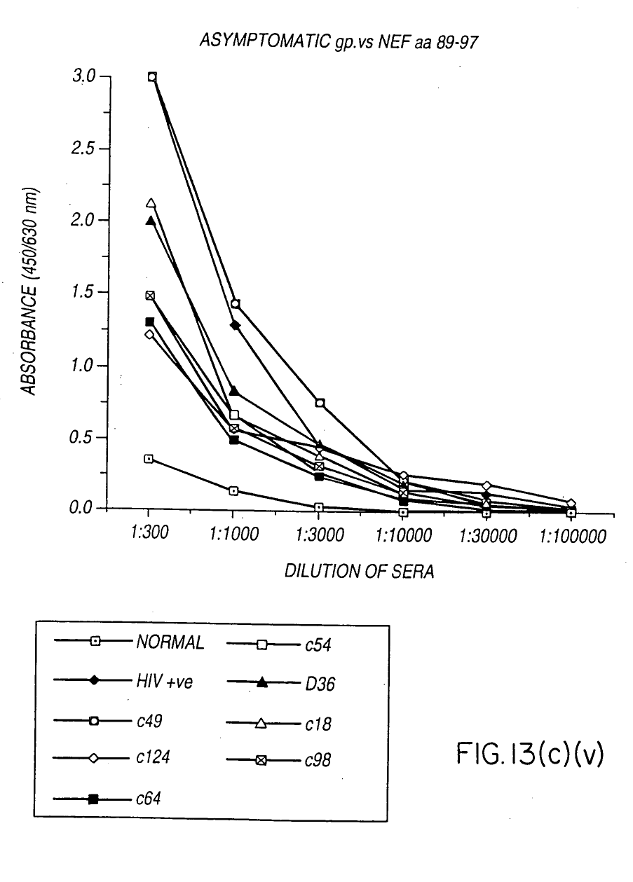

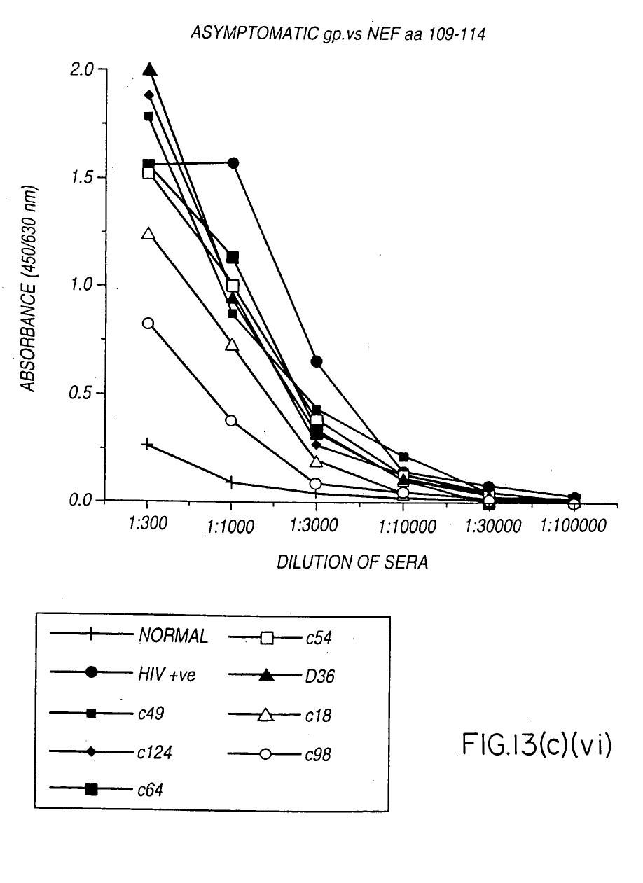

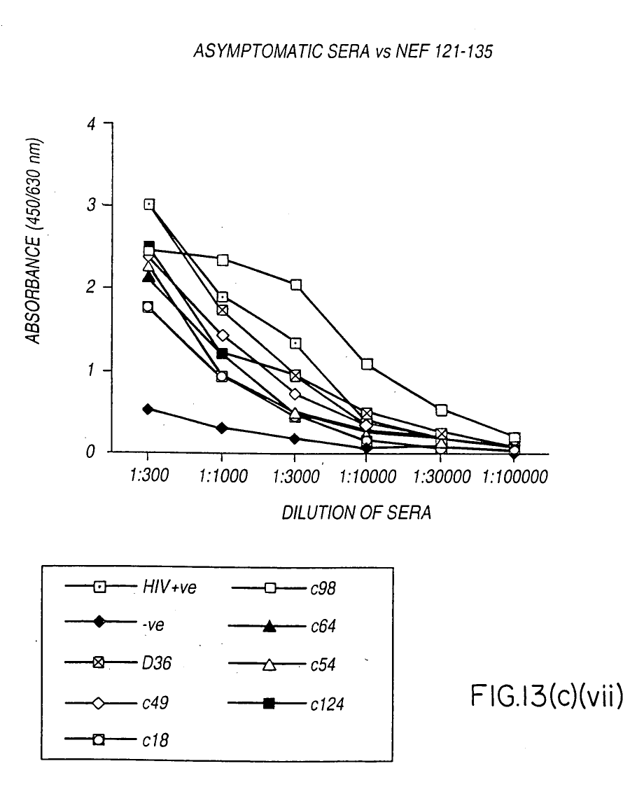

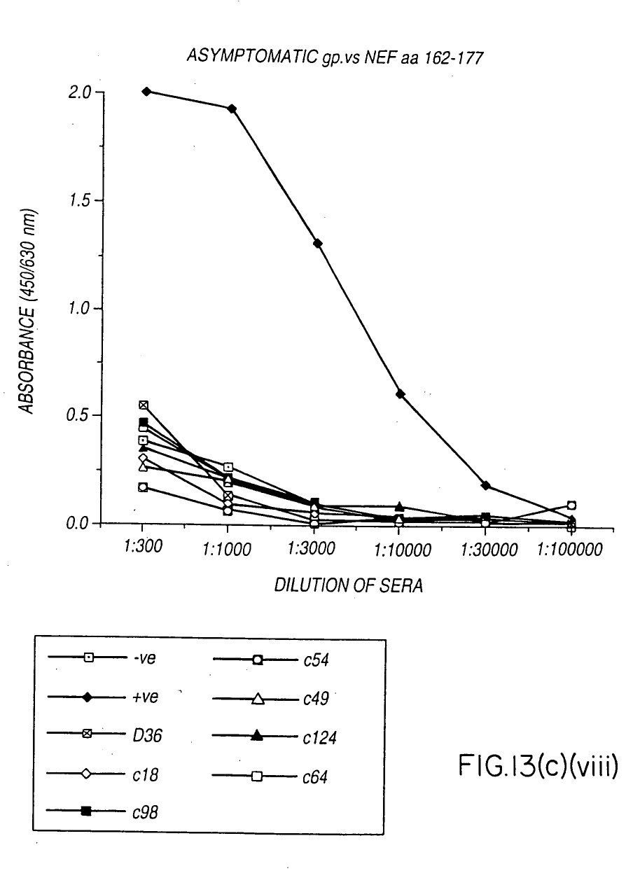

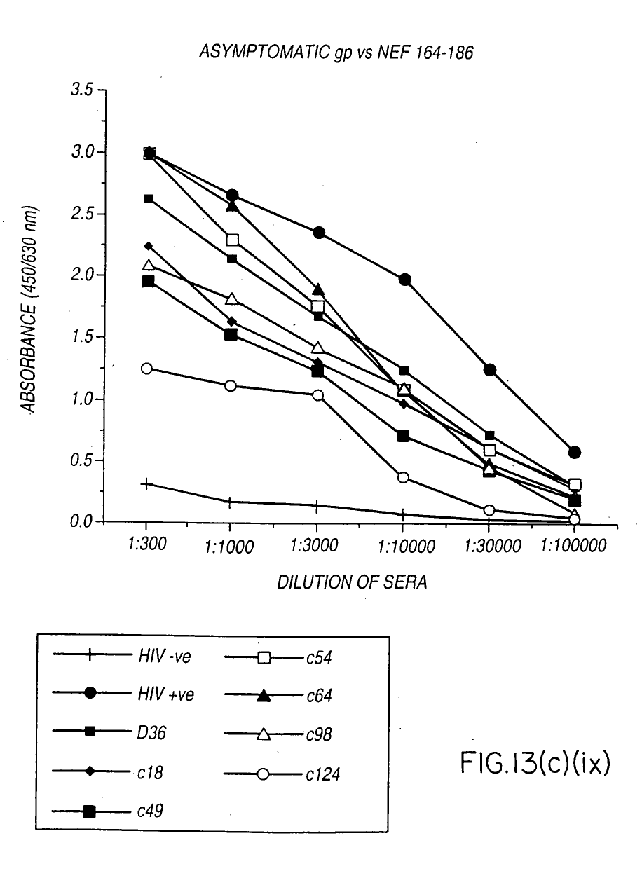

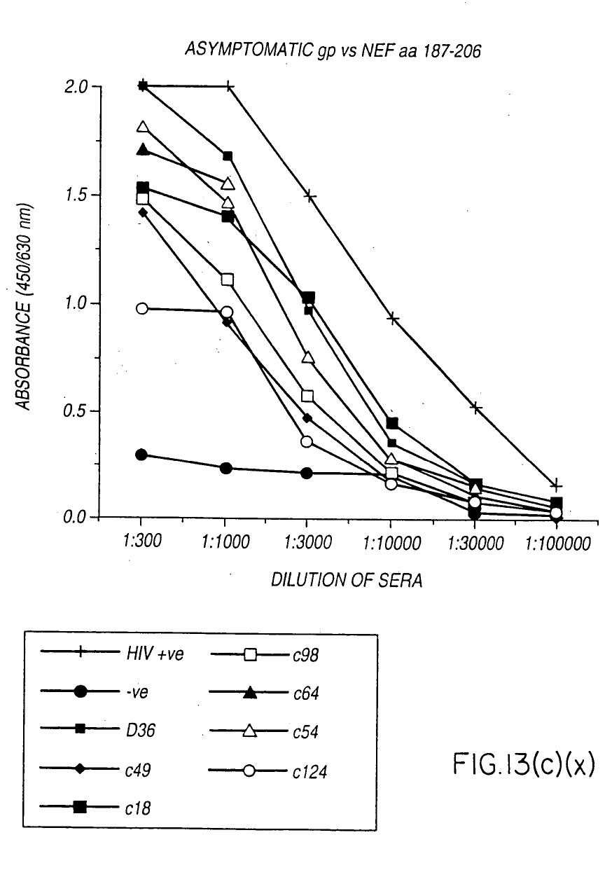

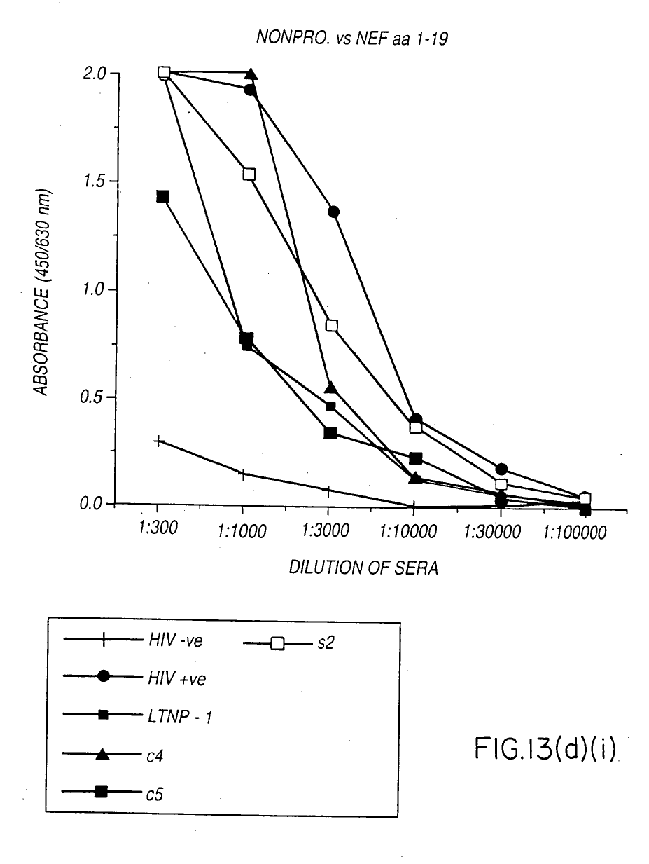

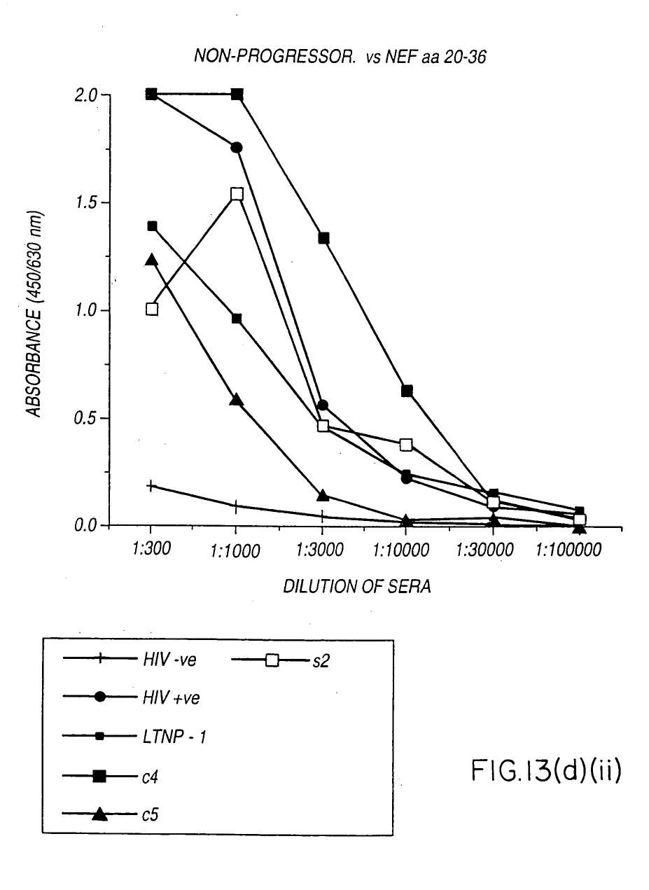

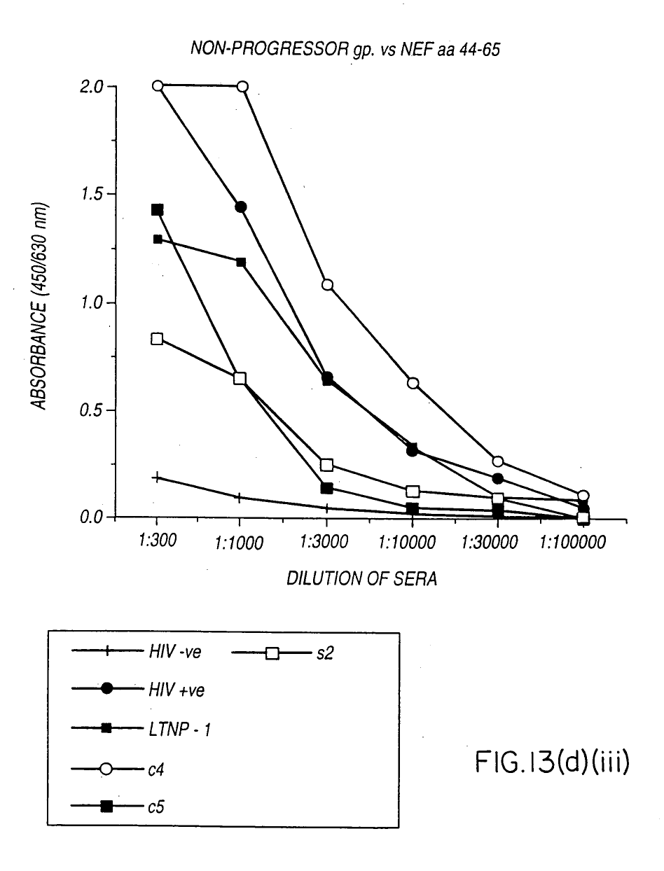

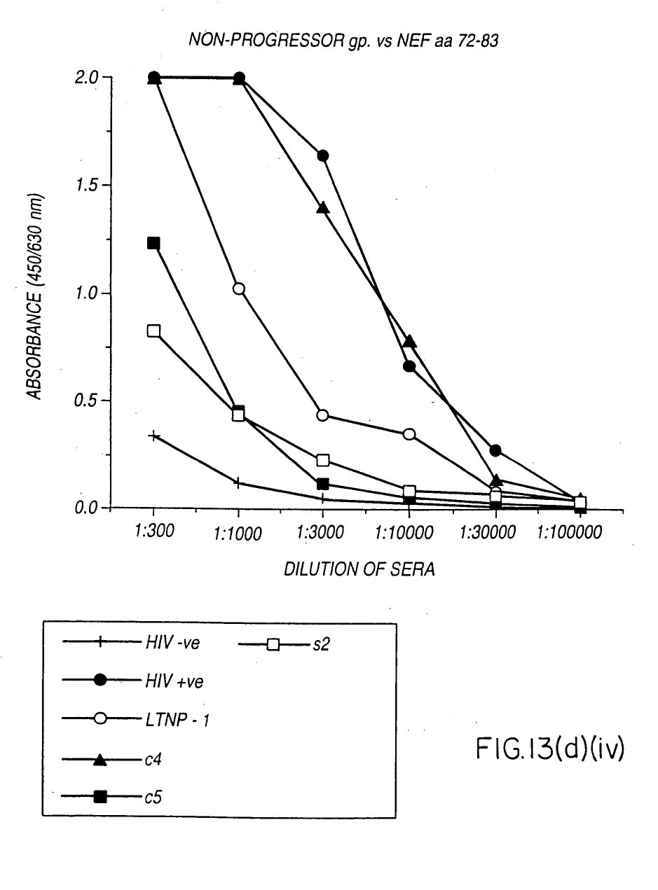

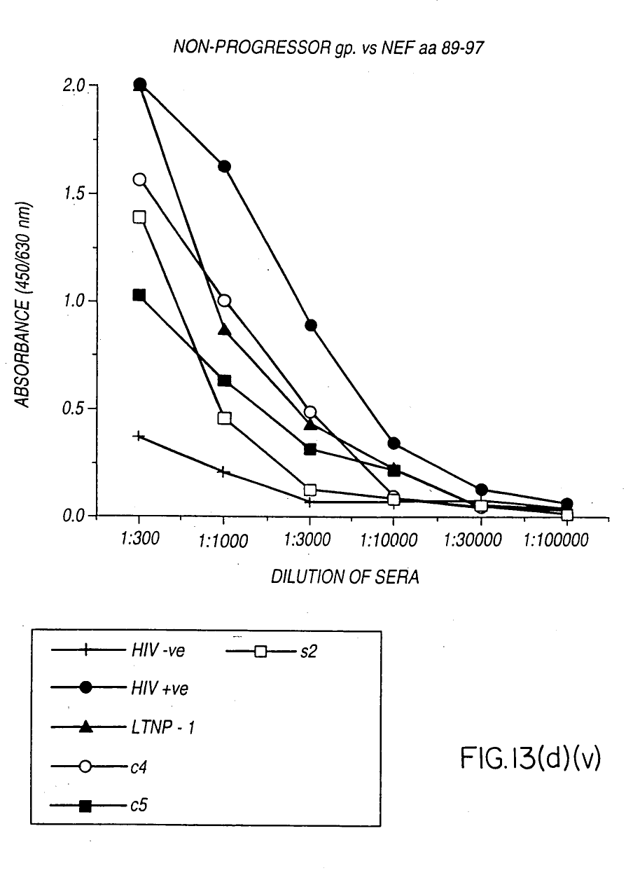

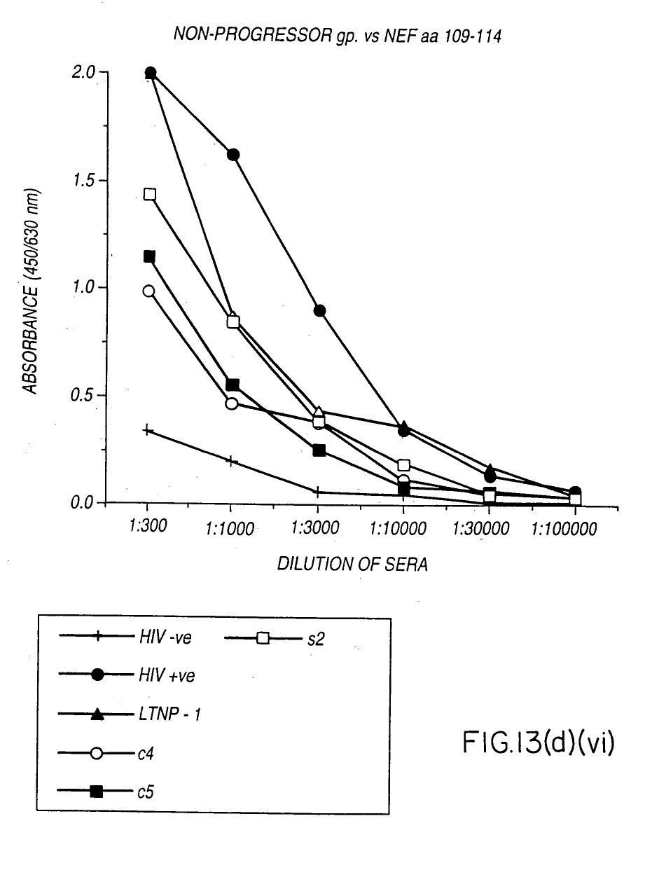

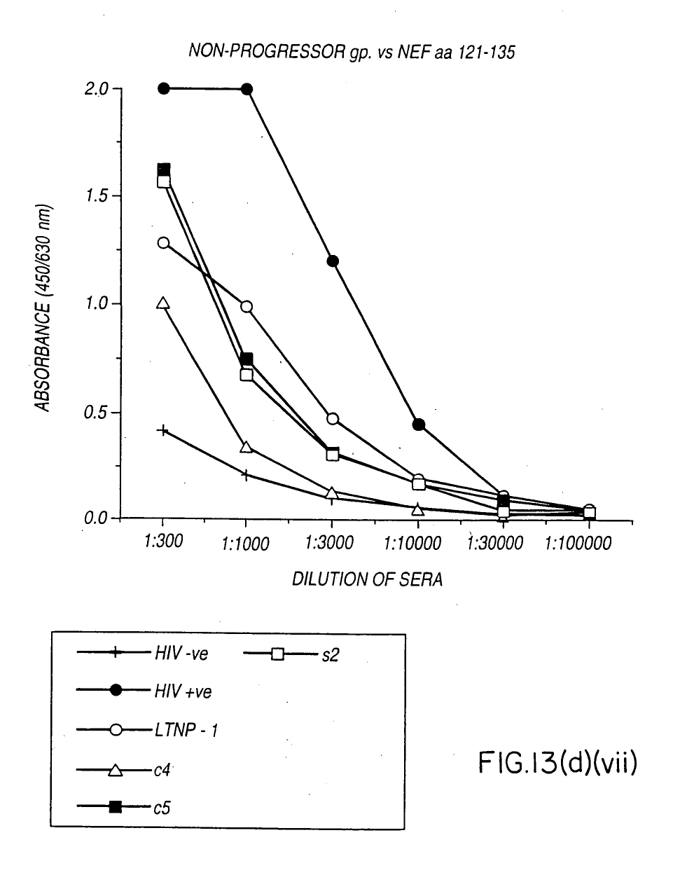

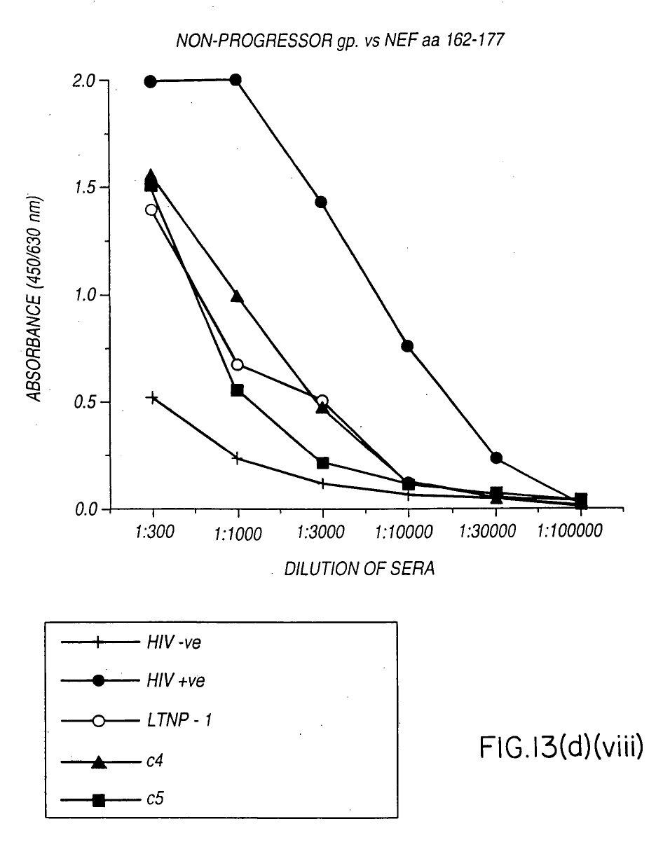

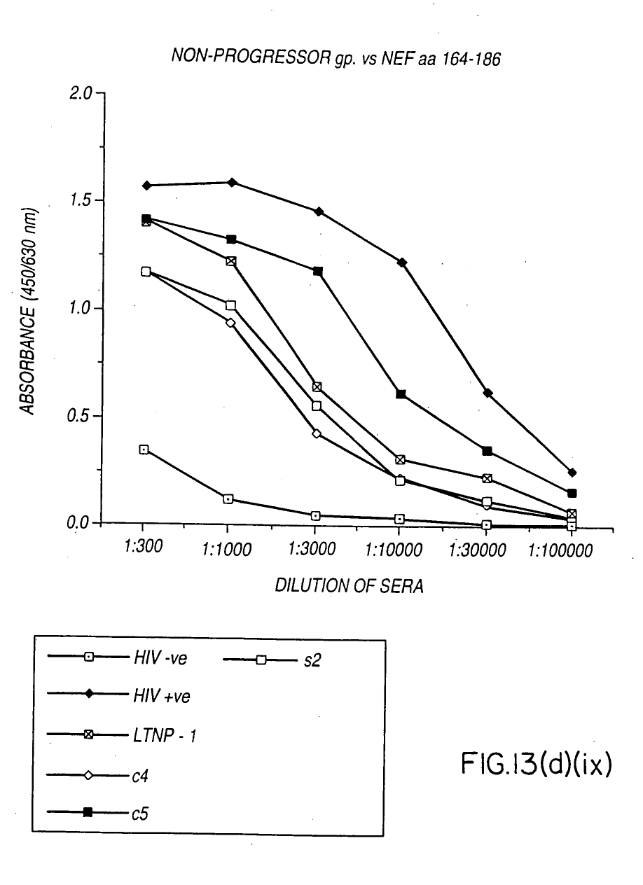

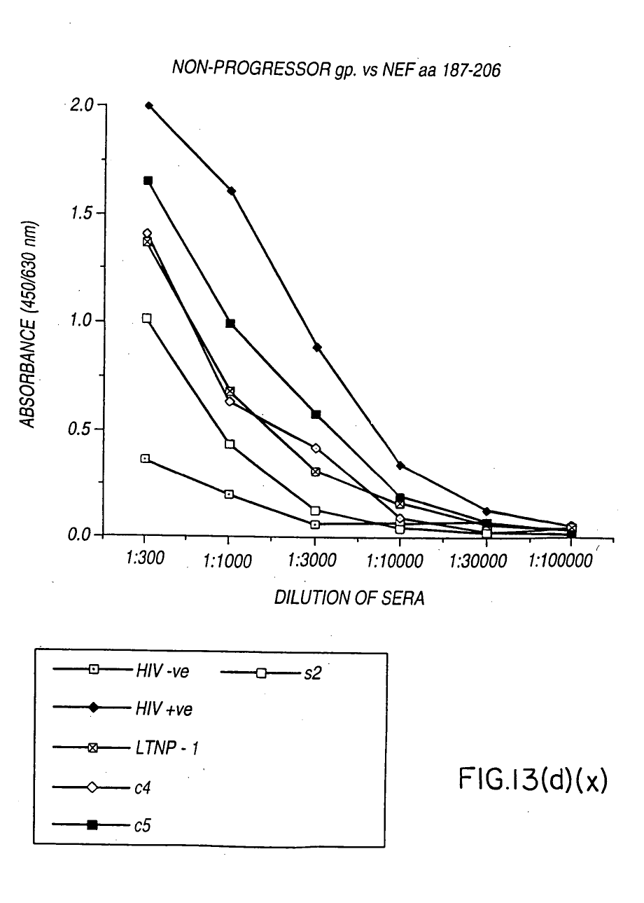

Recognition of synthetic peptides, which correspond to amino acid sequences of Nef, by the LTNP1 cohort was assessed. Various peptides were asses to detect those antigenic epitopes of Nef protein recognised by these individuals. Peptides corresponding to amino acid sequences 1 to 19; 20 to 36; 44 to 65; 72 to 83; 89 to 97; 109 to 114; 164 to 186; 187 to 206; 121 to 135 and 162 to 177 of HIV-1_{NL43} Nef protein were used to screen sera for the presence of specific antibodies. All sera from the LTP group recognised all Nef-derived peptides tested (FIG. 13a(i)-(x)). Sera titered between 1:1000 and 1:10,000. Sera from patients with autoimmune disease displayed only low background non-specific recognition. Normal sera from HIV-1-ve individuals tested to date also displayed only background activity (FIG. 13b). Sera from the LTNP1 and LTNP2 groups also showed significant reactive against Nef peptides corresponding to Nef amino acid sequences 1-19, 2036, 44-65, 72-83, 89-97, 109-114, 121-135, 164186 and 187-206 (FIG. 13c(i)-(x) and d(i)(x)). Sera from the LTNP2 group also showed significant reactivity against Nef peptide 162-177 (FIG. 13d(i)-(x)), similar to that showed by the LTP group, indicating that this region of Nef was immunogenic. However, sera from the LTNP1 cohort showed no significant reactivity towards peptide 162-177 above background levels obtained with normal HIV-1-ve sera (FIG. 13c(i)-(x)), indicating that this group of individuals were exposed to cells expressing a Nef protein which did not contain this region. While sera from the LTNP1 cohort did not react with the peptide corresponding to amino acid residues 162-177 of Ne the sera from all patients did recognise a longer peptide, 164 to 186, which encompassed most of Nef 162-177. This clearly indicates that the sera recognised antigenic epitopes between 177-186.

These results clearly indicate that all individuals from the LNTP1 cohort were exposed at some time to HIV-1 infected cells expressing a Nef protein that only had a small deletion encompassing amino acids 162 to 177.

The antibody testing has identified an antigenic region in the Nef protein which if deleted gives rise to attenuated HIV-1 viral strains. Hence, testing of the HIV-1 positive population may identify further examples of individuals infected with attenuated viral quasispecies. Additionally, lack of recognition of this antigenic epitope offers an antibody assay for testing animals experimentally infected with an HIV-1 attenuated viral strain, in particular deleted in the region covering Nef amino acids 162 to 177 (relative to HIV-1_{NL43} Nef).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

AZAD et al (1994) J. Gen. Virol. 75: 651-655.
BACHMAN B, LÜKE W AND HUNSMANN G (1990). Nucl Acids Res 18: 1309.

BARRE-SINOUSSI F. CHERMANN J C, REY F. et al (1983) Science 220: 868-871.
BUSCH M P. EL AMAD Z, SHEPPARD H W, ASCHER M S. LANG W (1991) N Engl J Med 325: 733-735.
CHENG-MAYER C, SHIODA T, LEVY J A (1991) J Virol 65: 6931-6941.
CLERICI M, STOCKS N I, ZAJAC R A. et al (1989) J Clin Invest 84: 1892-1899.
COX and OAKES (1989) Survival Analysis, Chapman & Hall.
DANIEL M D, KIRCHHOFF F, CZAJAK S C, SEHGAL P AND DESROSIERS R C (1992). Science 258: 1938-1941.
DELWART E L, et al (1993) Science 262: 1257-1261.
FECONDO et at (1993) AIDS Res. Hum Retro. 9: 733-740.
FOUCHER RAMS et at (1992) J. Virol. 66: 3183-3187.
GAYNOR R (1992). AIDS 6m 347-363.
GOU L-H and WU R. (1982). Nucleic Acids Research 10: 2065-2084.
GREENWAY et al (1994) Virology 198: 245-256.
GROENINK M, FOUCHIER RAM, BROERSEN S, et at (1993) Science 260: 1513-1515.
HAMMES S R, DIXON E P, MALIM M H, CULLEN B R and GREENE W C, (1989) Proc Natl Acad Sci USA 86: 9549-9553.
HIRT B. (1967) J Mol Biol 26: 365-369.
HWANG S S, BOYLE T J, LYERLY H K, CULLEN B R (1991) Science 253: 71-74.
KASLOW R A, DUQUESNOY R, VAN RADEN K, KINGSLEY L. MARRARI M. (1990) Lancet 335: 927-930.
KEMP B E, RYLATT D B, BUNDESEN P G, DOHERTY R R, MCPHEE D A, STAPLETON D, COTTIS L E, WILSON K, JOHN M A, KAN J, DINH D P, MILES S & HILLYARD C J (1988). Science 241: 1352-1354.
KESTLER H W, RINGLER D J, MORI K, PANICALI D L, SEHGAL P K, DANIEL M D & DESROSIERS R C (1991). Cell 65: 651-662.
KIERNAN R, et al (1990) AIDS Res. Hum. Retroviruses 6: 743-752.
KIM S, IKEUCHI K, BYRN R, GROOPMAN J and BALTIMORE D (1989) Proc Natl Acad Sci USA 86: 9544-9548.
LANG W, PERKINS H, ANDERSON R E, ROYCE R, JEWELL N. WILKELSTEIN W. (1989) J Acquir Immune Defic Syndr 2: 63-69.
LEARMONT J, TINDALL B, EVANS L, CUNNINGHAM A, CUNNINGHAM P, WELLS J, PENNY R, KALDOR J AND COOPER D A. (1992). Lancet 340: 863-867.
LEARMONT J et al (1995) AIDS Res. Hum. Retro. 11: 1.
LEGUERN M, SHIODA T, LEVY J A, CHENG-MAYER C. (1993) Virology 195; 441-447.
LEVY J A, (1993) AIDS 7; 1401-1410.
LIFSON A R, BUCHBINDER S P, SHEPPARD H W, et al (1991) J infect Dis 163: 959-965.
LUCIW P A, CHENG-MAYER C and LEVY J A (1987). Proc Natl Acad Sci USA 84: 1434-1438.
MANIATIS T et al (1982). Molecular cloning. A Laboratory Manual, 1st edition Pub. Cold Spring Harbor Laboratory Press.
MOSIER D E, GULIZIA R J, MACISAAC P D, TORBETT B E, LEVY J A (1993) Science 260: 689-692.
MYERS A, KORBAR B, BERZOFSKY J A, SMITH R F & PAVLAKIS S A eds (1992; 1993; 1994). Human retroviruses and AIDS. A compilation and analysis of nucleic acid and amino acid sequences. Pub Theoretical Biology and Biophysics Group. Los Alamos National Laboratory, Los Alamos, N. Mex. USA.
NEATE E V, HEALY D S, PRINGLE R C, GUST I D, AND JOWLETT J M B (1987). Aust NZ J Med 17: 461-466.
NIEDERMAN T M J, THIELAN B J, and RATNER L. (1989) Proc Natl Acad Sci USA 86: 1128-1132.
PEPER R J, TINA, W Z, & MICHELSON M M (1968). J Lab Cin Med 72: 842-846.
SAMBROOK et al (1989) Molecular Cloning. A Laboratory Mammal. Cold Spring Harbor Laboratory, Cold Spring Harbor, N.Y.
SANGER F, NICKLEN S AND COULSON A R (1977). Proc Natl Acad Sci USA 4: 5463-67.
SHEPPARD H W, LANG W, ASCHER M S, VITTINGHOF E and WINKELSTEIN W. (1993) AIDS 7: 1159-1166.
SHIODA T, LEVY J A. CHENG-MAYER C (1992) Proc Natl Acad Sci USA 89: 9434-9438.
SHUGARS D C, SMITH M S, GLUECK D H, NANTERMET P V, SEILLIER-MOISEIWITSCH F AND SWANSTROM R (1993). J Virol 67: 4639-4650.
SMITH J, AZAD A A, and DEACON N J (1992) J Gen Viol 73: 1825-1828.
SULLIVAN N, THALI M, FURMAN C, HO D D, SODROSKI I (1993) J Virol 67: 3674-3679.
TEEUWSEN V J P, SIEBELINK K H J, DE WOLF F, GOUDSMIT J, UYTDEHAAG F G C M, OSTERHAUS A D M E (1990) AIDS 4: 77-81.
TERSMETTE M, DE GOEDE REY, BERT J M, et al (1988) J Virol 62: 20262032. TERWILLIGER E, SODROSKI J G, ROSEN C A, and HASELTINE W A, (1986) J Virol 60: 754-760.
YAMANOTO K MORI S, OKAMOTO T, SHINOTOHNO K AND KYOGOKU Y (1991). Nucl Acids Res 22: 6107-6112.
YANISCH-PERRON C, VIEIRA J, and MESSING J Gene 13: 103-119.

1. A method for vaccinating an individual against the development of AIDS or AIDS related diseases, said method comprising administering to said individual a non-pathogenic isolate of HIV-1 in an amount effective to infect target cells and to generate target cells carrying DNA derived from said non-pathogenic isolate of HIV-1, wherein said isolate comprises a genomic deletion in the region corresponding to nucleotides 9281-3438 of the nef gene and U3 long terminal repeat, wherein said nucleotide numbering is based upon HIV-1 strain NL4-3 and said region comprises the nucleotide numbering is based upon HIV-1 strain NL4-3 and said region comprises the nucleotide sequence coding for amino acids 166-206 of the nef protein.

2. The method according to claim 1 wherein said isolate is capable of stimulating an immune response in primates against at least one glycoprotein on HIV-1 while not substantially reducing proliferative responses and cytokine production to a mitogen in said primates.

3. The method according to claim 1 wherein the HIV-1 isolate is recognized by an antibody to a glycoprotein of HIV-1, and is capable of inducing an immune response to at least one of the gag, pol or env proteins.

4. The method according to claim 3 wherein said glycoprotein is at least one of gp41-45, gp120 or gp160.

5. A vaccine composition comprising a non-pathogenic isolate of HIV-1 and at least one of a pharmaceutical acceptable carrier or a diluent, wherein said isolate comprises a genomic deletion in the region corresponding to nucleotides 9281-3438 of the nef gene and U3 long terminal repeat, wherein said nucleotide numbering is based upon HIV-1 strain NL4-3 and said region comprises the nucleotide sequence coding for amino acids 166-206 of the nef protein.

6. The method of claim 1 wherein said deletion results in reduced expression of the nef gene product.

7. The method of claim 1 wherein said deletion results in the expression of a truncated nef gene product.

8. The method of claim 1, wherein said deletion comprises at least 10 nucleotides.

9. The method of claim 1, wherein said isolate of HIV-1 is selected from the group of viruses consisting of V94101706, V941031169, and V95031022.

10. A method for vaccinating an individual against the development of AIDS or AIDS related diseases, said method comprising administering to said individual a non-pathogenic isolate of HIV-1 in an amount effective to infect target cells and to generate target cells carrying DNA derived from said non-pathogenic isolate of HIV-1, wherein said isolate comprises a genomic deletion of at least 10 nucleotides in the region corresponding to nucleotides 9281-9438 of the nef gene and U3 long terminal repeat, wherein said nucleotide numbering is based upon HIV-1 strain NL4-3.

11. The method of claim 10, wherein said isolate is recognized by an antibody to one of gp41-45, gp-120, or gp160 of HIV-1, said HIV strain is capable of stimulating an immune response in primates to at least one of the gag, pol, or env gene products without reducing proliferative responses and cytokine production to a mitogen in said primates.

12. A method for vaccinating an individual against the development of AIDS or AIDS related diseases, said method comprising administering to said individual a non-pathogenic isolate of HIV-1 in an amount effective to infect target cells and to generate target cells carrying DNA derived from said non-pathogenic isolate of HIV-1, wherein said isolate is selected from the group of viruses consisting of V94101706, V941031169, and V95031022.

13. The vaccine composition of claim 5, wherein said deletion results in reduced expression of the nef gene product.

14. The vaccine composition of claim 5, wherein said deletion results in the expression of a truncated nef gene product.

15. The vaccine composition of claim 5, wherein said deletion comprises at least 10 nucleotides.

16. The vaccine composition of claim 5, wherein the HIV-1 isolate is recognized by an antibody to a glycoprotein of HIV-1, and is capable of inducing an immune response in primates against at least one glycoprotein on HIV-1 while not substantially reducing proliferative responses and cytokine production to a mitogen in said primates.

17. The vaccine composition of claim 16, wherein said isolate is recognized by an antibody to one of gp41-45, gp120, or gp160 of HIV-1.

18. The vaccine composition of claim 16, wherein said immune response is against one of the gag, pol or env gene products.

19. The vaccine composition of claim 5, wherein said HIV-1 strain is selected from the group of viruses consisting of V94101706, V941031169, and V95031022.

20. A vaccine composition comprising a non-pathogenic isolate of HIV-1 and at least one of a pharmaceutical acceptable carrier or a diluent, wherein said HIV-1 strain comprises a genomic deletion of a least 10 nucleotides in the region corresponding to nucleotides 9281-9438 of the nef gene and U3 long terminal repeat, wherein said nucleotide numbering is based upon HIV-1 strain NIA-3.

21. The vaccine composition of claim 20, wherein said HIV strain is recognized by an antibody specific for one of HIV-1 gp41-45, HIV-1 gp120, or HIV-1 gp160, and said HIV strain is capable of stimulating an immune response in primates to at least one of the gag, pol, or env gene product without reducing proliferative responses and cytokine production of a mitogen.

22. A vaccine composition comprising a non-pathogenic isolate of HIV-1 and at least one of a pharmaceutical acceptable carrier or a diluent, wherein said HIV-1 strain is selected from the group of viruses consisting of V94101706, V941031169, and V95030122.

23. A method for inducing in an individual an immune response against HIV-1, comprising administering to said individual a non-pathogenic isolate of HIV-1 in an amount effective to infect target cells and to generate target cells carrying DNA derived from said non-pathogenic isolate of HIV-1, wherein said isolate comprises a genomic deletion in the region corresponding to nucleotides 9281-9438 of the nef gene and U3 long terminal repeat, wherein said nucleotide numbering is based is based upon HIV-1 strain NL4-3 and said region comprises the nucleotide sequence coding for amino acids 166-206 of the nef protein.

24. The method according to claim 23, wherein said isolate is capable of stimulating an immune response in primates against at least one glycoprotein on HIV-1 while not substantially reducing proliferative responses and cytokine production to a mitogen in said primates.

25. The method according to claim 23, wherein the HIV-1 isolate is recognized by an antibody to a glycoprotein of HIV-1, and is capable of inducing an immune response to at least one of the gag, pol or env proteins.

26. The method according to claim 25, wherein said glycoprotein is at least one of gp41-45, gp120 or gp160.

27. The method of claim 23, wherein said deletion results in reduced expression of the nef gene product.

28. The method of claim 23, wherein said deletion results in the expression of a truncated nef gene product.

29. The method of claim 23, wherein said deletion comprises at least 10 nucleotides.

30. The method of claim 23, wherein said isolate of HIV-1 is selected from the group of viruses consisting of V94101706, V941031169, and V95031022.

31. An immunogenic composition capable of inducing in an individual an immune response against HIV-1, comprising a non-pathogenic isolate of HIV-1, and at least one of a pharmaceutical acceptable carrier or a diluent, wherein said isolate comprises a genomic deletion in the region corresponding to nucleotides 9281-9438 of the nef gene and U3 long terminal repeat, wherein said nucleotide numbering is based upon HIV-1 strain NL4-3 and said region comprises the nucleotide sequence coding for amino acids 166-206 of the nef protein.

32. The immunogenic composition according to claim 31, wherein said isolate is capable of stimulating an immune response in primates against at least one glycoprotein on HIV-1 while not substantially reducing proliferative responses and cytokine production to a mitogen in said primates.

33. The immunogenic composition according to claim 31, wherein the HIV-1 isolate is recognized by an antibody to a glycoprotein of HIV-1, and is capable of inducing an immune response to at least one of the gag, pol or env proteins.

34. The immunogenic composition according to claim 33, wherein said glycoprotein is at least one of gp41-45, gp120 or gp160.

35. The immunogenic composition according to claim 31, wherein said deletion results in reduced expression of the nef gene product.

36. The immunogenic composition according to claim 31, wherein said deletion results in the expression of a truncated nef gene product.

37. The immunogenic composition according to claim 31, wherein said deletion comprises at least 10 nucleotides.

38. The immunogenic composition according to claim 31, wherein said isolate of HIV-1 is selected from the group of viruses consisting of V94101706, V941031169, and V95031022.

39. The method according to claim 2, wherein said primates are humans.

40. The method according to claim 11, wherein said primates are humans.

41. The vaccine composition according to claim 16, wherein said primates are humans.

42. The vaccine composition according to claim 21, wherein said primates are humans.

43. The method according to claim 24, wherein said primates are humans.

44. The immunogenic composition according to claim 32, wherein said primates are humans.


SEQUENCE LISTING (841 sequences)

SEQ ID NO: 1 - DNA, 9709 bp; single-stranded, linear
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaaataaggagagaagaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatatgtatatttcaaggaaagctaaggactggttttatagacatcactatgaaagtactaatccaaaaataagttcagaagtacacatcccactaggggatgctaaattagtaataacaacatattggggtctgcatacaggagaaagagactggcatttgggtcagggagtctccatagaatggaggaaaaagagatatagcacacaagtagaccctgacctagcagaccaactaattcatctgcactattttgattgtttttcagaatctgctataagaaataccatattaggacgtatagttagtcctaggtgtgaatatcaagcaggacataacaaggtaggatctctacagtacttggcactagcagcattaataaaaccaaaacagataaagccacctttgcctagtgttaggaaactgacagaggacagatggaacaagccccagaagaccaagggccacagagggagccatacaatgaatggacactagagcttttagaggaacttaagagtgaagctgttagacattttcctaggatatggctccataacttaggacaacatatctatgaaacttacggggatacttgggcaggagtggaagccataataagaattctgcaacaactgctgtttatccatttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcatgacaaaagccttaggcatctcctatggcaggaagaagcggagacagcgacgaagagctcatcagaacagtcagactcatcaagcttctctatcaaagcagtaagtagtacatgtaatgcaacctataatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagagataaggtgcagaaagaatatgcattcttttataaacttgatatagtaccaatagataataccagctataggttgataagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaatcaggccagtagtatcaactcaactgctgttaaatggcagtctagcagaagaagatgtagtaattagatctgccaatttcacagacaatgctaaaaccataatagtacagctgaacacatctgtagaaattaattgtacaagacccaacaacaatacaagaaaaagtatccgtatccagaggggaccagggagagcatttgttacaataggaaaaataggaaatatgagacaagcacattgtaacattagtagagcaaaatggaatgccactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcacactcccatgcagaataaaacaatttataaacatgtggcaggaagtaggaaaagcaatgtatgcccctcccatcagtggacaaattagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggctgcacgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - DNA, 10 bp; single-stranded, linear
atgggtggca

SEQ ID NO: 3 - DNA, 10 bp; single-stranded, linear
tgggtggcaa

SEQ ID NO: 4 - DNA, 10 bp; single-stranded, linear
gggtggcaag

SEQ ID NO: 5 - DNA, 10 bp; single-stranded, linear
ggtggcaagt

SEQ ID NO: 6 - DNA, 10 bp; single-stranded, linear
gtggcaagtg

SEQ ID NO: 7 - DNA, 10 bp; single-stranded, linear
tggcaagtgg

SEQ ID NO: 8 - DNA, 10 bp; single-stranded, linear
ggcaagtggt

SEQ ID NO: 9 - DNA, 10 bp; single-stranded, linear
gcaagtggtc

SEQ ID NO: 10 - DNA, 10 bp; single-stranded, linear
caagtggtca

SEQ ID NO: 11 - DNA, 10 bp; single-stranded, linear
aagtggtcaa

SEQ ID NO: 12 - DNA, 10 bp; single-stranded, linear
agtggtcaaa

SEQ ID NO: 13 - DNA, 10 bp; single-stranded, linear
gtggtcaaaa

SEQ ID NO: 14 - DNA, 10 bp; single-stranded, linear
tggtcaaaaa

SEQ ID NO: 15 - DNA, 10 bp; single-stranded, linear
ggtcaaaaag

SEQ ID NO: 16 - DNA, 10 bp; single-stranded, linear
gtcaaaaagt

SEQ ID NO: 17 - DNA, 10 bp; single-stranded, linear
tcaaaaagta

SEQ ID NO: 18 - DNA, 10 bp; single-stranded, linear
caaaaagtag

SEQ ID NO: 19 - DNA, 10 bp; single-stranded, linear
aaaaagtagt

SEQ ID NO: 20 - DNA, 10 bp; single-stranded, linear
aaaagtagtg

SEQ ID NO: 21 - DNA, 10 bp; single-stranded, linear
aaagtagtgt

SEQ ID NO: 22 - DNA, 10 bp; single-stranded, linear
aagtagtgtg

SEQ ID NO: 23 - DNA, 10 bp; single-stranded, linear
agtagtgtga

SEQ ID NO: 24 - DNA, 10 bp; single-stranded, linear
gtagtgtgat

SEQ ID NO: 25 - DNA, 10 bp; single-stranded, linear
tagtgtgatt

SEQ ID NO: 26 - DNA, 10 bp; single-stranded, linear
agtgtgattg

SEQ ID NO: 27 - DNA, 10 bp; single-stranded, linear
gtgtgattgg

SEQ ID NO: 28 - DNA, 10 bp; single-stranded, linear
tgtgattgga

SEQ ID NO: 29 - DNA, 10 bp; single-stranded, linear
gtgattggat

SEQ ID NO: 30 - DNA, 10 bp; single-stranded, linear
tgattggatg

SEQ ID NO: 31 - DNA, 10 bp; single-stranded, linear
gattggatgg

SEQ ID NO: 32 - DNA, 10 bp; single-stranded, linear
attggatggc

SEQ ID NO: 33 - DNA, 10 bp; single-stranded, linear
ttggatggcc

SEQ ID NO: 34 - DNA, 10 bp; single-stranded, linear
tggatggcct

SEQ ID NO: 35 - DNA, 10 bp; single-stranded, linear
ggatggcctg

SEQ ID NO: 36 - DNA, 10 bp; single-stranded, linear
gatggcctgc

SEQ ID NO: 37 - DNA, 10 bp; single-stranded, linear
atggcctgct

SEQ ID NO: 38 - DNA, 10 bp; single-stranded, linear
tggcctgctg

SEQ ID NO: 39 - DNA, 10 bp; single-stranded, linear
ggcctgctgt

SEQ ID NO: 40 - DNA, 10 bp; single-stranded, linear
gcctgctgta

SEQ ID NO: 41 - DNA, 10 bp; single-stranded, linear
cctgctgtaa

SEQ ID NO: 42 - DNA, 10 bp; single-stranded, linear
ctgctgtaag

SEQ ID NO: 43 - DNA, 10 bp; single-stranded, linear
tgctgtaagg

SEQ ID NO: 44 - DNA, 10 bp; single-stranded, linear
gctgtaaggg

SEQ ID NO: 45 - DNA, 10 bp; single-stranded, linear
ctgtaaggga

SEQ ID NO: 46 - DNA, 10 bp; single-stranded, linear
tgtaagggaa

SEQ ID NO: 47 - DNA, 10 bp; single-stranded, linear
gtaagggaaa

SEQ ID NO: 48 - DNA, 10 bp; single-stranded, linear
taagggaaag

SEQ ID NO: 49 - DNA, 10 bp; single-stranded, linear
aagggaaaga

SEQ ID NO: 50 - DNA, 10 bp; single-stranded, linear
agggaaagaa

SEQ ID NO: 51 - DNA, 10 bp; single-stranded, linear
gggaaagaat

SEQ ID NO: 52 - DNA, 10 bp; single-stranded, linear
ggaaagaatg

SEQ ID NO: 53 - DNA, 10 bp; single-stranded, linear
gaaagaatga

SEQ ID NO: 54 - DNA, 10 bp; single-stranded, linear
aaagaatgag

SEQ ID NO: 55 - DNA, 10 bp; single-stranded, linear
aagaatgaga

SEQ ID NO: 56 - DNA, 10 bp; single-stranded, linear
agaatgagac

SEQ ID NO: 57 - DNA, 10 bp; single-stranded, linear
gaatgagacg

SEQ ID NO: 58 - DNA, 10 bp; single-stranded, linear
aatgagacga

SEQ ID NO: 59 - DNA, 10 bp; single-stranded, linear
atgagacgag

SEQ ID NO: 60 - DNA, 10 bp; single-stranded, linear
tgagacgagc

SEQ ID NO: 61 - DNA, 10 bp; single-stranded, linear
gagacgagct

SEQ ID NO: 62 - DNA, 10 bp; single-stranded, linear
agacgagctg

SEQ ID NO: 63 - DNA, 10 bp; single-stranded, linear
gacgagctga

SEQ ID NO: 64 - DNA, 10 bp; single-stranded, linear
acgagctgag

SEQ ID NO: 65 - DNA, 10 bp; single-stranded, linear
cgagctgagc

SEQ ID NO: 66 - DNA, 10 bp; single-stranded, linear
gagctgagcc

SEQ ID NO: 67 - DNA, 10 bp; single-stranded, linear
agctgagcca

SEQ ID NO: 68 - DNA, 10 bp; single-stranded, linear
gctgagccag

SEQ ID NO: 69 - DNA, 10 bp; single-stranded, linear
ctgagccagc

SEQ ID NO: 70 - DNA, 10 bp; single-stranded, linear
tgagccagca

SEQ ID NO: 71 - DNA, 10 bp; single-stranded, linear
gagccagcag

SEQ ID NO: 72 - DNA, 10 bp; single-stranded, linear
agccagcagc

SEQ ID NO: 73 - DNA, 10 bp; single-stranded, linear
gccagcagca

SEQ ID NO: 74 - DNA, 10 bp; single-stranded, linear
ccagcagcag

SEQ ID NO: 75 - DNA, 10 bp; single-stranded, linear
cagcagcaga

SEQ ID NO: 76 - DNA, 10 bp; single-stranded, linear
agcagcagat

SEQ ID NO: 77 - DNA, 10 bp; single-stranded, linear
gcagcagatg

SEQ ID NO: 78 - DNA, 10 bp; single-stranded, linear
cagcagatgg

SEQ ID NO: 79 - DNA, 10 bp; single-stranded, linear
agcagatggg

SEQ ID NO: 80 - DNA, 10 bp; single-stranded, linear
gcagatgggg

SEQ ID NO: 81 - DNA, 10 bp; single-stranded, linear
cagatggggt

SEQ ID NO: 82 - DNA, 10 bp; single-stranded, linear
agatggggtg

SEQ ID NO: 83 - DNA, 10 bp; single-stranded, linear
gatggggtgg

SEQ ID NO: 84 - DNA, 10 bp; single-stranded, linear
atggggtggg

SEQ ID NO: 85 - DNA, 10 bp; single-stranded, linear
tggggtggga

SEQ ID NO: 86 - DNA, 10 bp; single-stranded, linear
ggggtgggag

SEQ ID NO: 87 - DNA, 10 bp; single-stranded, linear
gggtgggagc

SEQ ID NO: 88 - DNA, 10 bp; single-stranded, linear
ggtgggagca

SEQ ID NO: 89 - DNA, 10 bp; single-stranded, linear
gtgggagcag

SEQ ID NO: 90 - DNA, 10 bp; single-stranded, linear
tgggagcagt

SEQ ID NO: 91 - DNA, 10 bp; single-stranded, linear
gggagcagta

SEQ ID NO: 92 - DNA, 10 bp; single-stranded, linear
ggagcagtat

SEQ ID NO: 93 - DNA, 10 bp; single-stranded, linear
gagcagtatc

SEQ ID NO: 94 - DNA, 10 bp; single-stranded, linear
agcagtatct

SEQ ID NO: 95 - DNA, 10 bp; single-stranded, linear
gcagtatctc

SEQ ID NO: 96 - DNA, 10 bp; single-stranded, linear
cagtatctcg

SEQ ID NO: 97 - DNA, 10 bp; single-stranded, linear
agtatctcga

SEQ ID NO: 98 - DNA, 10 bp; single-stranded, linear
gtatctcgag

SEQ ID NO: 99 - DNA, 10 bp; single-stranded, linear
tatctcgaga

SEQ ID NO: 100 - DNA, 10 bp; single-stranded, linear
atctcgagac

SEQ ID NO: 101 - DNA, 10 bp; single-stranded, linear
tctcgagacc

SEQ ID NO: 102 - DNA, 10 bp; single-stranded, linear
ctcgagacct

SEQ ID NO: 103 - DNA, 10 bp; single-stranded, linear
tcgagaccta

SEQ ID NO: 104 - DNA, 10 bp; single-stranded, linear
cgagacctag

SEQ ID NO: 105 - DNA, 10 bp; single-stranded, linear
gagacctaga

SEQ ID NO: 106 - DNA, 10 bp; single-stranded, linear
agacctagaa

SEQ ID NO: 107 - DNA, 10 bp; single-stranded, linear
gacctagaaa

SEQ ID NO: 108 - DNA, 10 bp; single-stranded, linear
acctagaaaa

SEQ ID NO: 109 - DNA, 10 bp; single-stranded, linear
cctagaaaaa

SEQ ID NO: 110 - DNA, 10 bp; single-stranded, linear
ctagaaaaac

SEQ ID NO: 111 - DNA, 10 bp; single-stranded, linear
tagaaaaaca

SEQ ID NO: 112 - DNA, 10 bp; single-stranded, linear
agaaaaacat

SEQ ID NO: 113 - DNA, 10 bp; single-stranded, linear
gaaaaacatg

SEQ ID NO: 114 - DNA, 10 bp; single-stranded, linear
aaaaacatgg

SEQ ID NO: 115 - DNA, 10 bp; single-stranded, linear
aaaacatgga

SEQ ID NO: 116 - DNA, 10 bp; single-stranded, linear
aaacatggag

SEQ ID NO: 117 - DNA, 10 bp; single-stranded, linear
aacatggagc

SEQ ID NO: 118 - DNA, 10 bp; single-stranded, linear
acatggagca

SEQ ID NO: 119 - DNA, 10 bp; single-stranded, linear
catggagcaa

SEQ ID NO: 120 - DNA, 10 bp; single-stranded, linear
atggagcaat

SEQ ID NO: 121 - DNA, 10 bp; single-stranded, linear
tggagcaatc

SEQ ID NO: 122 - DNA, 10 bp; single-stranded, linear
ggagcaatca

SEQ ID NO: 123 - DNA, 10 bp; single-stranded, linear
gagcaatcac

SEQ ID NO: 124 - DNA, 10 bp; single-stranded, linear
agcaatcaca

SEQ ID NO: 125 - DNA, 10 bp; single-stranded, linear
gcaatcacaa

SEQ ID NO: 126 - DNA, 10 bp; single-stranded, linear
caatcacaag

SEQ ID NO: 127 - DNA, 10 bp; single-stranded, linear
aatcacaagt

SEQ ID NO: 128 - DNA, 10 bp; single-stranded, linear
atcacaagta

SEQ ID NO: 129 - DNA, 10 bp; single-stranded, linear
tcacaagtag

SEQ ID NO: 130 - DNA, 10 bp; single-stranded, linear
cacaagtagc

SEQ ID NO: 131 - DNA, 10 bp; single-stranded, linear
acaagtagca

SEQ ID NO: 132 - DNA, 10 bp; single-stranded, linear
caagtagcaa

SEQ ID NO: 133 - DNA, 10 bp; single-stranded, linear
aagtagcaat

SEQ ID NO: 134 - DNA, 10 bp; single-stranded, linear
agtagcaata

SEQ ID NO: 135 - DNA, 10 bp; single-stranded, linear
gtagcaatac

SEQ ID NO: 136 - DNA, 10 bp; single-stranded, linear
tagcaataca

SEQ ID NO: 137 - DNA, 10 bp; single-stranded, linear
agcaatacag

SEQ ID NO: 138 - DNA, 10 bp; single-stranded, linear
gcaatacagc

SEQ ID NO: 139 - DNA, 10 bp; single-stranded, linear
caatacagca

SEQ ID NO: 140 - DNA, 10 bp; single-stranded, linear
aatacagcag

SEQ ID NO: 141 - DNA, 10 bp; single-stranded, linear
atacagcagc

SEQ ID NO: 142 - DNA, 10 bp; single-stranded, linear
tacagcagct

SEQ ID NO: 143 - DNA, 10 bp; single-stranded, linear
acagcagcta

SEQ ID NO: 144 - DNA, 10 bp; single-stranded, linear
cagcagctaa

SEQ ID NO: 145 - DNA, 10 bp; single-stranded, linear
agcagctaac

SEQ ID NO: 146 - DNA, 10 bp; single-stranded, linear
gcagctaaca

SEQ ID NO: 147 - DNA, 10 bp; single-stranded, linear
cagctaacaa

SEQ ID NO: 148 - DNA, 10 bp; single-stranded, linear
agctaacaat

SEQ ID NO: 149 - DNA, 10 bp; single-stranded, linear
gctaacaatg

SEQ ID NO: 150 - DNA, 10 bp; single-stranded, linear
ctaacaatgc

SEQ ID NO: 151 - DNA, 10 bp; single-stranded, linear
taacaatgct

SEQ ID NO: 152 - DNA, 10 bp; single-stranded, linear
aacaatgctg

SEQ ID NO: 153 - DNA, 10 bp; single-stranded, linear
acaatgctgc

SEQ ID NO: 154 - DNA, 10 bp; single-stranded, linear
caatgctgct

SEQ ID NO: 155 - DNA, 10 bp; single-stranded, linear
aatgctgctt

SEQ ID NO: 156 - DNA, 10 bp; single-stranded, linear
atgctgcttg

SEQ ID NO: 157 - DNA, 10 bp; single-stranded, linear
tgctgcttgt

SEQ ID NO: 158 - DNA, 10 bp; single-stranded, linear
gctgcttgtg

SEQ ID NO: 159 - DNA, 10 bp; single-stranded, linear
ctgcttgtgc

SEQ ID NO: 160 - DNA, 10 bp; single-stranded, linear
tgcttgtgcc

SEQ ID NO: 161 - DNA, 10 bp; single-stranded, linear
gcttgtgcct

SEQ ID NO: 162 - DNA, 10 bp; single-stranded, linear
cttgtgcctg

SEQ ID NO: 163 - DNA, 10 bp; single-stranded, linear
ttgtgcctgg

SEQ ID NO: 164 - DNA, 10 bp; single-stranded, linear
tgtgcctggc

SEQ ID NO: 165 - DNA, 10 bp; single-stranded, linear
gtgcctggct

SEQ ID NO: 166 - DNA, 10 bp; single-stranded, linear
tgcctggcta

SEQ ID NO: 167 - DNA, 10 bp; single-stranded, linear
gcctggctag

SEQ ID NO: 168 - DNA, 10 bp; single-stranded, linear
cctggctaga

SEQ ID NO: 169 - DNA, 10 bp; single-stranded, linear
ctggctagaa

SEQ ID NO: 170 - DNA, 10 bp; single-stranded, linear
tggctagaag

SEQ ID NO: 171 - DNA, 10 bp; single-stranded, linear
ggctagaagc

SEQ ID NO: 172 - DNA, 10 bp; single-stranded, linear
gctagaagca

SEQ ID NO: 173 - DNA, 10 bp; single-stranded, linear
ctagaagcac

SEQ ID NO: 174 - DNA, 10 bp; single-stranded, linear
tagaagcaca

SEQ ID NO: 175 - DNA, 10 bp; single-stranded, linear
agaagcacaa

SEQ ID NO: 176 - DNA, 10 bp; single-stranded, linear
gaagcacaag

SEQ ID NO: 177 - DNA, 10 bp; single-stranded, linear
aagcacaaga

SEQ ID NO: 178 - DNA, 10 bp; single-stranded, linear
agcacaagag

SEQ ID NO: 179 - DNA, 10 bp; single-stranded, linear
gcacaagagg

SEQ ID NO: 180 - DNA, 10 bp; single-stranded, linear
cacaagagga

SEQ ID NO: 181 - DNA, 10 bp; single-stranded, linear
acaagaggag

SEQ ID NO: 182 - DNA, 10 bp; single-stranded, linear
caagaggagg

SEQ ID NO: 183 - DNA, 10 bp; single-stranded, linear
aagaggagga

SEQ ID NO: 184 - DNA, 10 bp; single-stranded, linear
agaggaggaa

SEQ ID NO: 185 - DNA, 10 bp; single-stranded, linear
gaggaggaag

SEQ ID NO: 186 - DNA, 10 bp; single-stranded, linear
aggaggaaga

SEQ ID NO: 187 - DNA, 10 bp; single-stranded, linear
ggaggaagag

SEQ ID NO: 188 - DNA, 10 bp; single-stranded, linear
gaggaagagg

SEQ ID NO: 189 - DNA, 10 bp; single-stranded, linear
aggaagaggt

SEQ ID NO: 190 - DNA, 10 bp; single-stranded, linear
ggaagaggtg

SEQ ID NO: 191 - DNA, 10 bp; single-stranded, linear
gaagaggtgg

SEQ ID NO: 192 - DNA, 10 bp; single-stranded, linear
aagaggtggg

SEQ ID NO: 193 - DNA, 10 bp; single-stranded, linear
agaggtgggt

SEQ ID NO: 194 - DNA, 10 bp; single-stranded, linear
gaggtgggtt

SEQ ID NO: 195 - DNA, 10 bp; single-stranded, linear
aggtgggttt

SEQ ID NO: 196 - DNA, 10 bp; single-stranded, linear
ggtgggtttt

SEQ ID NO: 197 - DNA, 10 bp; single-stranded, linear
gtgggttttc

SEQ ID NO: 198 - DNA, 10 bp; single-stranded, linear
tgggttttcc

SEQ ID NO: 199 - DNA, 10 bp; single-stranded, linear
gggttttcca

SEQ ID NO: 200 - DNA, 10 bp; single-stranded, linear
ggttttccag

SEQ ID NO: 201 - DNA, 10 bp; single-stranded, linear
gttttccagt

SEQ ID NO: 202 - DNA, 10 bp; single-stranded, linear
ttttccagtc

SEQ ID NO: 203 - DNA, 10 bp; single-stranded, linear
tttccagtca

SEQ ID NO: 204 - DNA, 10 bp; single-stranded, linear
ttccagtcac

SEQ ID NO: 205 - DNA, 10 bp; single-stranded, linear
tccagtcaca

SEQ ID NO: 206 - DNA, 10 bp; single-stranded, linear
ccagtcacac

SEQ ID NO: 207 - DNA, 10 bp; single-stranded, linear
cagtcacacc

SEQ ID NO: 208 - DNA, 10 bp; single-stranded, linear
agtcacacct

SEQ ID NO: 209 - DNA, 10 bp; single-stranded, linear
gtcacacctc

SEQ ID NO: 210 - DNA, 10 bp; single-stranded, linear
tcacacctca

SEQ ID NO: 211 - DNA, 10 bp; single-stranded, linear
cacacctcag

SEQ ID NO: 212 - DNA, 10 bp; single-stranded, linear
acacctcagg

SEQ ID NO: 213 - DNA, 10 bp; single-stranded, linear
cacctcaggt

SEQ ID NO: 214 - DNA, 10 bp; single-stranded, linear
acctcaggta

SEQ ID NO: 215 - DNA, 10 bp; single-stranded, linear
cctcaggtac

SEQ ID NO: 216 - DNA, 10 bp; single-stranded, linear
ctcaggtacc

SEQ ID NO: 217 - DNA, 10 bp; single-stranded, linear
tcaggtacct

SEQ ID NO: 218 - DNA, 10 bp; single-stranded, linear
caggtacctt

SEQ ID NO: 219 - DNA, 10 bp; single-stranded, linear
aggtaccttt

SEQ ID NO: 220 - DNA, 10 bp; single-stranded, linear
ggtaccttta

SEQ ID NO: 221 - DNA, 10 bp; single-stranded, linear
gtacctttaa

SEQ ID NO: 222 - DNA, 10 bp; single-stranded, linear
tacctttaag

SEQ ID NO: 223 - DNA, 10 bp; single-stranded, linear
acctttaaga

SEQ ID NO: 224 - DNA, 10 bp; single-stranded, linear
cctttaagac

SEQ ID NO: 225 - DNA, 10 bp; single-stranded, linear
ctttaagacc

SEQ ID NO: 226 - DNA, 10 bp; single-stranded, linear
tttaagacca

SEQ ID NO: 227 - DNA, 10 bp; single-stranded, linear
ttaagaccaa

SEQ ID NO: 228 - DNA, 10 bp; single-stranded, linear
taagaccaat

SEQ ID NO: 229 - DNA, 10 bp; single-stranded, linear
aagaccaatg

SEQ ID NO: 230 - DNA, 10 bp; single-stranded, linear
agaccaatga

SEQ ID NO: 231 - DNA, 10 bp; single-stranded, linear
gaccaatgac

SEQ ID NO: 232 - DNA, 10 bp; single-stranded, linear
accaatgact

SEQ ID NO: 233 - DNA, 10 bp; single-stranded, linear
ccaatgactt

SEQ ID NO: 234 - DNA, 10 bp; single-stranded, linear
caatgactta

SEQ ID NO: 235 - DNA, 10 bp; single-stranded, linear
aatgacttac

SEQ ID NO: 236 - DNA, 10 bp; single-stranded, linear
atgacttaca

SEQ ID NO: 237 - DNA, 10 bp; single-stranded, linear
tgacttacaa

SEQ ID NO: 238 - DNA, 10 bp; single-stranded, linear
gacttacaag

SEQ ID NO: 239 - DNA, 10 bp; single-stranded, linear
acttacaagg

SEQ ID NO: 240 - DNA, 10 bp; single-stranded, linear
cttacaaggc

SEQ ID NO: 241 - DNA, 10 bp; single-stranded, linear
ttacaaggca

SEQ ID NO: 242 - DNA, 10 bp; single-stranded, linear
tacaaggcag

SEQ ID NO: 243 - DNA, 10 bp; single-stranded, linear
acaaggcagc

SEQ ID NO: 244 - DNA, 10 bp; single-stranded, linear
caaggcagct

SEQ ID NO: 245 - DNA, 10 bp; single-stranded, linear
aaggcagctg

SEQ ID NO: 246 - DNA, 10 bp; single-stranded, linear
aggcagctgt

SEQ ID NO: 247 - DNA, 10 bp; single-stranded, linear
ggcagctgta

SEQ ID NO: 248 - DNA, 10 bp; single-stranded, linear
gcagctgtag

SEQ ID NO: 249 - DNA, 10 bp; single-stranded, linear
cagctgtaga

SEQ ID NO: 250 - DNA, 10 bp; single-stranded, linear
agctgtagat

SEQ ID NO: 251 - DNA, 10 bp; single-stranded, linear
gctgtagatc

SEQ ID NO: 252 - DNA, 10 bp; single-stranded, linear
ctgtagatct

SEQ ID NO: 253 - DNA, 10 bp; single-stranded, linear
tgtagatctt

SEQ ID NO: 254 - DNA, 10 bp; single-stranded, linear
gtagatctta

SEQ ID NO: 255 - DNA, 10 bp; single-stranded, linear
tagatcttag

SEQ ID NO: 256 - DNA, 10 bp; single-stranded, linear
agatcttagc

SEQ ID NO: 257 - DNA, 10 bp; single-stranded, linear
gatcttagcc

SEQ ID NO: 258 - DNA, 10 bp; single-stranded, linear
atcttagcca

SEQ ID NO: 259 - DNA, 10 bp; single-stranded, linear
tcttagccac

SEQ ID NO: 260 - DNA, 10 bp; single-stranded, linear
cttagccact

SEQ ID NO: 261 - DNA, 10 bp; single-stranded, linear
ttagccactt

SEQ ID NO: 262 - DNA, 10 bp; single-stranded, linear
tagccacttt

SEQ ID NO: 263 - DNA, 10 bp; single-stranded, linear
agccactttt

SEQ ID NO: 264 - DNA, 10 bp; single-stranded, linear
gccacttttt

SEQ ID NO: 265 - DNA, 10 bp; single-stranded, linear
ccacttttta

SEQ ID NO: 266 - DNA, 10 bp; single-stranded, linear
cactttttaa

SEQ ID NO: 267 - DNA, 10 bp; single-stranded, linear
actttttaaa

SEQ ID NO: 268 - DNA, 10 bp; single-stranded, linear
ctttttaaaa

SEQ ID NO: 269 - DNA, 10 bp; single-stranded, linear
tttttaaaag

SEQ ID NO: 270 - DNA, 10 bp; single-stranded, linear
ttttaaaaga

SEQ ID NO: 271 - DNA, 10 bp; single-stranded, linear
tttaaaagaa

SEQ ID NO: 272 - DNA, 10 bp; single-stranded, linear
ttaaaagaaa

SEQ ID NO: 273 - DNA, 10 bp; single-stranded, linear
taaaagaaaa

SEQ ID NO: 274 - DNA, 10 bp; single-stranded, linear
aaaagaaaag

SEQ ID NO: 275 - DNA, 10 bp; single-stranded, linear
aaagaaaagg

SEQ ID NO: 276 - DNA, 10 bp; single-stranded, linear
aagaaaaggg

SEQ ID NO: 277 - DNA, 10 bp; single-stranded, linear
agaaaagggg

SEQ ID NO: 278 - DNA, 10 bp; single-stranded, linear
gaaaaggggg

SEQ ID NO: 279 - DNA, 10 bp; single-stranded, linear
aaaagggggg

SEQ ID NO: 280 - DNA, 10 bp; single-stranded, linear
aaagggggga

SEQ ID NO: 281 - DNA, 10 bp; single-stranded, linear
aaggggggac

SEQ ID NO: 282 - DNA, 10 bp; single-stranded, linear
aggggggact

SEQ ID NO: 283 - DNA, 10 bp; single-stranded, linear
ggggggactg

SEQ ID NO: 284 - DNA, 10 bp; single-stranded, linear
gggggactgg

SEQ ID NO: 285 - DNA, 10 bp; single-stranded, linear
ggggactgga

SEQ ID NO: 286 - DNA, 10 bp; single-stranded, linear
gggactggaa

SEQ ID NO: 287 - DNA, 10 bp; single-stranded, linear
ggactggaag

SEQ ID NO: 288 - DNA, 10 bp; single-stranded, linear
gactggaagg

SEQ ID NO: 289 - DNA, 10 bp; single-stranded, linear
actggaaggg

SEQ ID NO: 290 - DNA, 10 bp; single-stranded, linear
ctggaagggc

SEQ ID NO: 291 - DNA, 10 bp; single-stranded, linear
tggaagggct

SEQ ID NO: 292 - DNA, 10 bp; single-stranded, linear
ggaagggcta

SEQ ID NO: 293 - DNA, 10 bp; single-stranded, linear
gaagggctaa

SEQ ID NO: 294 - DNA, 10 bp; single-stranded, linear
aagggctaat

SEQ ID NO: 295 - DNA, 10 bp; single-stranded, linear
agggctaatt

SEQ ID NO: 296 - DNA, 10 bp; single-stranded, linear
gggctaattc

SEQ ID NO: 297 - DNA, 10 bp; single-stranded, linear
ggctaattca

SEQ ID NO: 298 - DNA, 10 bp; single-stranded, linear
gctaattcac

SEQ ID NO: 299 - DNA, 10 bp; single-stranded, linear
ctaattcact

SEQ ID NO: 300 - DNA, 10 bp; single-stranded, linear
taattcactc

SEQ ID NO: 301 - DNA, 10 bp; single-stranded, linear
aattcactcc

SEQ ID NO: 302 - DNA, 10 bp; single-stranded, linear
attcactccc

SEQ ID NO: 303 - DNA, 10 bp; single-stranded, linear
ttcactccca

SEQ ID NO: 304 - DNA, 10 bp; single-stranded, linear
tcactcccaa

SEQ ID NO: 305 - DNA, 10 bp; single-stranded, linear
cactcccaaa

SEQ ID NO: 306 - DNA, 10 bp; single-stranded, linear
actcccaaag

SEQ ID NO: 307 - DNA, 10 bp; single-stranded, linear
ctcccaaaga

SEQ ID NO: 308 - DNA, 10 bp; single-stranded, linear
tcccaaagaa

SEQ ID NO: 309 - DNA, 10 bp; single-stranded, linear
cccaaagaag

SEQ ID NO: 310 - DNA, 10 bp; single-stranded, linear
ccaaagaaga

SEQ ID NO: 311 - DNA, 10 bp; single-stranded, linear
caaagaagac

SEQ ID NO: 312 - DNA, 10 bp; single-stranded, linear
aaagaagaca

SEQ ID NO: 313 - DNA, 10 bp; single-stranded, linear
aagaagacaa

SEQ ID NO: 314 - DNA, 10 bp; single-stranded, linear
agaagacaag

SEQ ID NO: 315 - DNA, 10 bp; single-stranded, linear
gaagacaaga

SEQ ID NO: 316 - DNA, 10 bp; single-stranded, linear
aagacaagat

SEQ ID NO: 317 - DNA, 10 bp; single-stranded, linear
agacaagata

SEQ ID NO: 318 - DNA, 10 bp; single-stranded, linear
gacaagatat

SEQ ID NO: 319 - DNA, 10 bp; single-stranded, linear
acaagatatc

SEQ ID NO: 320 - DNA, 10 bp; single-stranded, linear
caagatatcc

SEQ ID NO: 321 - DNA, 10 bp; single-stranded, linear
aagatatcct

SEQ ID NO: 322 - DNA, 10 bp; single-stranded, linear
agatatcctt

SEQ ID NO: 323 - DNA, 10 bp; single-stranded, linear
gatatccttg

SEQ ID NO: 324 - DNA, 10 bp; single-stranded, linear
atatccttga

SEQ ID NO: 325 - DNA, 10 bp; single-stranded, linear
tatccttgat

SEQ ID NO: 326 - DNA, 10 bp; single-stranded, linear
atccttgatc

SEQ ID NO: 327 - DNA, 10 bp; single-stranded, linear
tccttgatct

SEQ ID NO: 328 - DNA, 10 bp; single-stranded, linear
ccttgatctg

SEQ ID NO: 329 - DNA, 10 bp; single-stranded, linear
cttgatctgt

SEQ ID NO: 330 - DNA, 10 bp; single-stranded, linear
ttgatctgtg

SEQ ID NO: 331 - DNA, 10 bp; single-stranded, linear
tgatctgtgg

SEQ ID NO: 332 - DNA, 10 bp; single-stranded, linear
gatctgtgga

SEQ ID NO: 333 - DNA, 10 bp; single-stranded, linear
atctgtggat

SEQ ID NO: 334 - DNA, 10 bp; single-stranded, linear
tctgtggatc

SEQ ID NO: 335 - DNA, 10 bp; single-stranded, linear
ctgtggatct

SEQ ID NO: 336 - DNA, 10 bp; single-stranded, linear
tgtggatcta

SEQ ID NO: 337 - DNA, 10 bp; single-stranded, linear
gtggatctac

SEQ ID NO: 338 - DNA, 10 bp; single-stranded, linear
tggatctacc

SEQ ID NO: 339 - DNA, 10 bp; single-stranded, linear
ggatctacca

SEQ ID NO: 340 - DNA, 10 bp; single-stranded, linear
gatctaccac

SEQ ID NO: 341 - DNA, 10 bp; single-stranded, linear
atctaccaca

SEQ ID NO: 342 - DNA, 10 bp; single-stranded, linear
tctaccacac

SEQ ID NO: 343 - DNA, 10 bp; single-stranded, linear
ctaccacaca

SEQ ID NO: 344 - DNA, 10 bp; single-stranded, linear
taccacacac

SEQ ID NO: 345 - DNA, 10 bp; single-stranded, linear
accacacaca

SEQ ID NO: 346 - DNA, 10 bp; single-stranded, linear
ccacacacaa

SEQ ID NO: 347 - DNA, 10 bp; single-stranded, linear
cacacacaag

SEQ ID NO: 348 - DNA, 10 bp; single-stranded, linear
acacacaagg

SEQ ID NO: 349 - DNA, 10 bp; single-stranded, linear
cacacaaggc

SEQ ID NO: 350 - DNA, 10 bp; single-stranded, linear
acacaaggct

SEQ ID NO: 351 - DNA, 10 bp; single-stranded, linear
cacaaggcta

SEQ ID NO: 352 - DNA, 10 bp; single-stranded, linear
acaaggctac

SEQ ID NO: 353 - DNA, 10 bp; single-stranded, linear
caaggctact

SEQ ID NO: 354 - DNA, 10 bp; single-stranded, linear
aaggctactt

SEQ ID NO: 355 - DNA, 10 bp; single-stranded, linear
aggctacttc

SEQ ID NO: 356 - DNA, 10 bp; single-stranded, linear
ggctacttcc

SEQ ID NO: 357 - DNA, 10 bp; single-stranded, linear
gctacttccc

SEQ ID NO: 358 - DNA, 10 bp; single-stranded, linear
ctacttccct

SEQ ID NO: 359 - DNA, 10 bp; single-stranded, linear
tacttccctg

SEQ ID NO: 360 - DNA, 10 bp; single-stranded, linear
acttccctga

SEQ ID NO: 361 - DNA, 10 bp; single-stranded, linear
cttccctgat

SEQ ID NO: 362 - DNA, 10 bp; single-stranded, linear
ttccctgatt

SEQ ID NO: 363 - DNA, 10 bp; single-stranded, linear
tccctgattg

SEQ ID NO: 364 - DNA, 10 bp; single-stranded, linear
ccctgattgg

SEQ ID NO: 365 - DNA, 10 bp; single-stranded, linear
cctgattggc

SEQ ID NO: 366 - DNA, 10 bp; single-stranded, linear
ctgattggca

SEQ ID NO: 367 - DNA, 10 bp; single-stranded, linear
tgattggcag

SEQ ID NO: 368 - DNA, 10 bp; single-stranded, linear
gattggcaga

SEQ ID NO: 369 - DNA, 10 bp; single-stranded, linear
attggcagaa

SEQ ID NO: 370 - DNA, 10 bp; single-stranded, linear
ttggcagaac

SEQ ID NO: 371 - DNA, 10 bp; single-stranded, linear
tggcagaact

SEQ ID NO: 372 - DNA, 10 bp; single-stranded, linear
ggcagaacta

SEQ ID NO: 373 - DNA, 10 bp; single-stranded, linear
gcagaactac

SEQ ID NO: 374 - DNA, 10 bp; single-stranded, linear
cagaactaca

SEQ ID NO: 375 - DNA, 10 bp; single-stranded, linear
agaactacac

SEQ ID NO: 376 - DNA, 10 bp; single-stranded, linear
gaactacaca

SEQ ID NO: 377 - DNA, 10 bp; single-stranded, linear
aactacacac

SEQ ID NO: 378 - DNA, 10 bp; single-stranded, linear
actacacacc

SEQ ID NO: 379 - DNA, 10 bp; single-stranded, linear
ctacacacca

SEQ ID NO: 380 - DNA, 10 bp; single-stranded, linear
tacacaccag

SEQ ID NO: 381 - DNA, 10 bp; single-stranded, linear
acacaccagg

SEQ ID NO: 382 - DNA, 10 bp; single-stranded, linear
cacaccaggg

SEQ ID NO: 383 - DNA, 10 bp; single-stranded, linear
acaccagggc

SEQ ID NO: 384 - DNA, 10 bp; single-stranded, linear
caccagggcc

SEQ ID NO: 385 - DNA, 10 bp; single-stranded, linear
accagggcca

SEQ ID NO: 386 - DNA, 10 bp; single-stranded, linear
ccagggccag

SEQ ID NO: 387 - DNA, 10 bp; single-stranded, linear
cagggccagg

SEQ ID NO: 388 - DNA, 10 bp; single-stranded, linear
agggccaggg

SEQ ID NO: 389 - DNA, 10 bp; single-stranded, linear
gggccagggg

SEQ ID NO: 390 - DNA, 10 bp; single-stranded, linear
ggccaggggt

SEQ ID NO: 391 - DNA, 10 bp; single-stranded, linear
gccaggggtc

SEQ ID NO: 392 - DNA, 10 bp; single-stranded, linear
ccaggggtca

SEQ ID NO: 393 - DNA, 10 bp; single-stranded, linear
caggggtcag

SEQ ID NO: 394 - DNA, 10 bp; single-stranded, linear
aggggtcaga

SEQ ID NO: 395 - DNA, 10 bp; single-stranded, linear
ggggtcagat

SEQ ID NO: 396 - DNA, 10 bp; single-stranded, linear
gggtcagata

SEQ ID NO: 397 - DNA, 10 bp; single-stranded, linear
ggtcagatat

SEQ ID NO: 398 - DNA, 10 bp; single-stranded, linear
gtcagatatc

SEQ ID NO: 399 - DNA, 10 bp; single-stranded, linear
tcagatatcc

SEQ ID NO: 400 - DNA, 10 bp; single-stranded, linear
cagatatcca

SEQ ID NO: 401 - DNA, 10 bp; single-stranded, linear
agatatccac

SEQ ID NO: 402 - DNA, 10 bp; single-stranded, linear
gatatccact

SEQ ID NO: 403 - DNA, 10 bp; single-stranded, linear
atatccactg

SEQ ID NO: 404 - DNA, 10 bp; single-stranded, linear
tatccactga

SEQ ID NO: 405 - DNA, 10 bp; single-stranded, linear
atccactgac

SEQ ID NO: 406 - DNA, 10 bp; single-stranded, linear
tccactgacc

SEQ ID NO: 407 - DNA, 10 bp; single-stranded, linear
ccactgacct

SEQ ID NO: 408 - DNA, 10 bp; single-stranded, linear
cactgacctt

SEQ ID NO: 409 - DNA, 10 bp; single-stranded, linear
actgaccttt

SEQ ID NO: 410 - DNA, 10 bp; single-stranded, linear
ctgacctttg

SEQ ID NO: 411 - DNA, 10 bp; single-stranded, linear
tgacctttgg

SEQ ID NO: 412 - DNA, 10 bp; single-stranded, linear
gacctttgga

SEQ ID NO: 413 - DNA, 10 bp; single-stranded, linear
acctttggat

SEQ ID NO: 414 - DNA, 10 bp; single-stranded, linear
cctttggatg

SEQ ID NO: 415 - DNA, 10 bp; single-stranded, linear
ctttggatgg

SEQ ID NO: 416 - DNA, 10 bp; single-stranded, linear
tttggatggt

SEQ ID NO: 417 - DNA, 10 bp; single-stranded, linear
ttggatggtg

SEQ ID NO: 418 - DNA, 10 bp; single-stranded, linear
tggatggtgc

SEQ ID NO: 419 - DNA, 10 bp; single-stranded, linear
ggatggtgct

SEQ ID NO: 420 - DNA, 10 bp; single-stranded, linear
gatggtgcta

SEQ ID NO: 421 - DNA, 10 bp; single-stranded, linear
atggtgctac

SEQ ID NO: 422 - DNA, 10 bp; single-stranded, linear
tggtgctaca

SEQ ID NO: 423 - DNA, 10 bp; single-stranded, linear
ggtgctacaa

SEQ ID NO: 424 - DNA, 10 bp; single-stranded, linear
gtgctacaag

SEQ ID NO: 425 - DNA, 10 bp; single-stranded, linear
tgctacaagc

SEQ ID NO: 426 - DNA, 10 bp; single-stranded, linear
gctacaagct

SEQ ID NO: 427 - DNA, 10 bp; single-stranded, linear
ctacaagcta

SEQ ID NO: 428 - DNA, 10 bp; single-stranded, linear
tacaagctag

SEQ ID NO: 429 - DNA, 10 bp; single-stranded, linear
acaagctagt

SEQ ID NO: 430 - DNA, 10 bp; single-stranded, linear
caagctagta

SEQ ID NO: 431 - DNA, 10 bp; single-stranded, linear
aagctagtac

SEQ ID NO: 432 - DNA, 10 bp; single-stranded, linear
agctagtacc

SEQ ID NO: 433 - DNA, 10 bp; single-stranded, linear
gctagtacca

SEQ ID NO: 434 - DNA, 10 bp; single-stranded, linear
ctagtaccag

SEQ ID NO: 435 - DNA, 10 bp; single-stranded, linear
tagtaccagt

SEQ ID NO: 436 - DNA, 10 bp; single-stranded, linear
agtaccagtt

SEQ ID NO: 437 - DNA, 10 bp; single-stranded, linear
gtaccagttg

SEQ ID NO: 438 - DNA, 10 bp; single-stranded, linear
taccagttga

SEQ ID NO: 439 - DNA, 10 bp; single-stranded, linear
accagttgag

SEQ ID NO: 440 - DNA, 10 bp; single-stranded, linear
ccagttgagc

SEQ ID NO: 441 - DNA, 10 bp; single-stranded, linear
cagttgagcc

SEQ ID NO: 442 - DNA, 10 bp; single-stranded, linear
agttgagcca

SEQ ID NO: 443 - DNA, 10 bp; single-stranded, linear
gttgagccag

SEQ ID NO: 444 - DNA, 10 bp; single-stranded, linear
ttgagccaga

SEQ ID NO: 445 - DNA, 10 bp; single-stranded, linear
tgagccagat

SEQ ID NO: 446 - DNA, 10 bp; single-stranded, linear
gagccagata

SEQ ID NO: 447 - DNA, 10 bp; single-stranded, linear
agccagataa

SEQ ID NO: 448 - DNA, 10 bp; single-stranded, linear
gccagataag

SEQ ID NO: 449 - DNA, 10 bp; single-stranded, linear
ccagataagg

SEQ ID NO: 450 - DNA, 10 bp; single-stranded, linear
cagataaggt

SEQ ID NO: 451 - DNA, 10 bp; single-stranded, linear
agataaggta

SEQ ID NO: 452 - DNA, 10 bp; single-stranded, linear
gataaggtag

SEQ ID NO: 453 - DNA, 10 bp; single-stranded, linear
ataaggtaga

SEQ ID NO: 454 - DNA, 10 bp; single-stranded, linear
taaggtagaa

SEQ ID NO: 455 - DNA, 10 bp; single-stranded, linear
aaggtagaag

SEQ ID NO: 456 - DNA, 10 bp; single-stranded, linear
aggtagaaga

SEQ ID NO: 457 - DNA, 10 bp; single-stranded, linear
ggtagaagag

SEQ ID NO: 458 - DNA, 10 bp; single-stranded, linear
gtagaagagg

SEQ ID NO: 459 - DNA, 10 bp; single-stranded, linear
tagaagaggc

SEQ ID NO: 460 - DNA, 10 bp; single-stranded, linear
agaagaggcc

SEQ ID NO: 461 - DNA, 10 bp; single-stranded, linear
gaagaggcca

SEQ ID NO: 462 - DNA, 10 bp; single-stranded, linear
aagaggccaa

SEQ ID NO: 463 - DNA, 10 bp; single-stranded, linear
agaggccaat

SEQ ID NO: 464 - DNA, 10 bp; single-stranded, linear
gaggccaata

SEQ ID NO: 465 - DNA, 10 bp; single-stranded, linear
aggccaataa

SEQ ID NO: 466 - DNA, 10 bp; single-stranded, linear
ggccaataaa

SEQ ID NO: 467 - DNA, 10 bp; single-stranded, linear
gccaataaag

SEQ ID NO: 468 - DNA, 10 bp; single-stranded, linear
ccaataaagg

SEQ ID NO: 469 - DNA, 10 bp; single-stranded, linear
caataaagga

SEQ ID NO: 470 - DNA, 10 bp; single-stranded, linear
aataaaggag

SEQ ID NO: 471 - DNA, 10 bp; single-stranded, linear
ataaaggaga

SEQ ID NO: 472 - DNA, 10 bp; single-stranded, linear
taaaggagag

SEQ ID NO: 473 - DNA, 10 bp; single-stranded, linear
aaaggagaga

SEQ ID NO: 474 - DNA, 10 bp; single-stranded, linear
aaggagagaa

SEQ ID NO: 475 - DNA, 10 bp; single-stranded, linear
aggagagaac

SEQ ID NO: 476 - DNA, 10 bp; single-stranded, linear
ggagagaaca

SEQ ID NO: 477 - DNA, 10 bp; single-stranded, linear
gagagaacac

SEQ ID NO: 478 - DNA, 10 bp; single-stranded, linear
agagaacacc

SEQ ID NO: 479 - DNA, 10 bp; single-stranded, linear
gagaacacca

SEQ ID NO: 480 - DNA, 10 bp; single-stranded, linear
agaacaccag

SEQ ID NO: 481 - DNA, 10 bp; single-stranded, linear
gaacaccagc

SEQ ID NO: 482 - DNA, 10 bp; single-stranded, linear
aacaccagct

SEQ ID NO: 483 - DNA, 10 bp; single-stranded, linear
acaccagctt

SEQ ID NO: 484 - DNA, 10 bp; single-stranded, linear
caccagcttg

SEQ ID NO: 485 - DNA, 10 bp; single-stranded, linear
accagcttgt

SEQ ID NO: 486 - DNA, 10 bp; single-stranded, linear
ccagcttgtt

SEQ ID NO: 487 - DNA, 10 bp; single-stranded, linear
cagcttgtta

SEQ ID NO: 488 - DNA, 10 bp; single-stranded, linear
agcttgttac

SEQ ID NO: 489 - DNA, 10 bp; single-stranded, linear
gcttgttaca

SEQ ID NO: 490 - DNA, 10 bp; single-stranded, linear
cttgttacac

SEQ ID NO: 491 - DNA, 10 bp; single-stranded, linear
ttgttacacc

SEQ ID NO: 492 - DNA, 10 bp; single-stranded, linear
tgttacaccc

SEQ ID NO: 493 - DNA, 10 bp; single-stranded, linear
gttacaccct

SEQ ID NO: 494 - DNA, 10 bp; single-stranded, linear
ttacaccctg

SEQ ID NO: 495 - DNA, 10 bp; single-stranded, linear
tacaccctgt

SEQ ID NO: 496 - DNA, 10 bp; single-stranded, linear
acaccctgtg

SEQ ID NO: 497 - DNA, 10 bp; single-stranded, linear
caccctgtga

SEQ ID NO: 498 - DNA, 10 bp; single-stranded, linear
accctgtgag

SEQ ID NO: 499 - DNA, 10 bp; single-stranded, linear
ccctgtgagc

SEQ ID NO: 500 - DNA, 10 bp; single-stranded, linear
cctgtgagcc

SEQ ID NO: 501 - DNA, 10 bp; single-stranded, linear
ctgtgagcct

SEQ ID NO: 502 - DNA, 10 bp; single-stranded, linear
tgtgagcctg

SEQ ID NO: 503 - DNA, 10 bp; single-stranded, linear
gtgagcctgc

SEQ ID NO: 504 - DNA, 10 bp; single-stranded, linear
tgagcctgca

SEQ ID NO: 505 - DNA, 10 bp; single-stranded, linear
gagcctgcat

SEQ ID NO: 506 - DNA, 10 bp; single-stranded, linear
agcctgcatg

SEQ ID NO: 507 - DNA, 10 bp; single-stranded, linear
gcctgcatgg

SEQ ID NO: 508 - DNA, 10 bp; single-stranded, linear
cctgcatgga

SEQ ID NO: 509 - DNA, 10 bp; single-stranded, linear
ctgcatggaa

SEQ ID NO: 510 - DNA, 10 bp; single-stranded, linear
tgcatggaat

SEQ ID NO: 511 - DNA, 10 bp; single-stranded, linear
gcatggaatg

SEQ ID NO: 512 - DNA, 10 bp; single-stranded, linear
catggaatgg

SEQ ID NO: 513 - DNA, 10 bp; single-stranded, linear
atggaatgga

SEQ ID NO: 514 - DNA, 10 bp; single-stranded, linear
tggaatggat

SEQ ID NO: 515 - DNA, 10 bp; single-stranded, linear
ggaatggatg

SEQ ID NO: 516 - DNA, 10 bp; single-stranded, linear
gaatggatga

SEQ ID NO: 517 - DNA, 10 bp; single-stranded, linear
aatggatgac

SEQ ID NO: 518 - DNA, 10 bp; single-stranded, linear
atggatgacc

SEQ ID NO: 519 - DNA, 10 bp; single-stranded, linear
tggatgaccc

SEQ ID NO: 520 - DNA, 10 bp; single-stranded, linear
ggatgaccct

SEQ ID NO: 521 - DNA, 10 bp; single-stranded, linear
gatgaccctg

SEQ ID NO: 522 - DNA, 10 bp; single-stranded, linear
atgaccctga

SEQ ID NO: 523 - DNA, 10 bp; single-stranded, linear
tgaccctgag

SEQ ID NO: 524 - DNA, 10 bp; single-stranded, linear
gaccctgaga

SEQ ID NO: 525 - DNA, 10 bp; single-stranded, linear
accctgagag

SEQ ID NO: 526 - DNA, 10 bp; single-stranded, linear
ccctgagaga

SEQ ID NO: 527 - DNA, 10 bp; single-stranded, linear
cctgagagag

SEQ ID NO: 528 - DNA, 10 bp; single-stranded, linear
ctgagagaga

SEQ ID NO: 529 - DNA, 10 bp; single-stranded, linear
tgagagagaa

SEQ ID NO: 530 - DNA, 10 bp; single-stranded, linear
gagagagaag

SEQ ID NO: 531 - DNA, 10 bp; single-stranded, linear
agagagaagt

SEQ ID NO: 532 - DNA, 10 bp; single-stranded, linear
gagagaagtg

SEQ ID NO: 533 - DNA, 10 bp; single-stranded, linear
agagaagtgt

SEQ ID NO: 534 - DNA, 10 bp; single-stranded, linear
gagaagtgtt

SEQ ID NO: 535 - DNA, 10 bp; single-stranded, linear
agaagtgtta

SEQ ID NO: 536 - DNA, 10 bp; single-stranded, linear
gaagtgttag

SEQ ID NO: 537 - DNA, 10 bp; single-stranded, linear
aagtgttaga

SEQ ID NO: 538 - DNA, 10 bp; single-stranded, linear
agtgttagag

SEQ ID NO: 539 - DNA, 10 bp; single-stranded, linear
gtgttagagt

SEQ ID NO: 540 - DNA, 10 bp; single-stranded, linear
tgttagagtg

SEQ ID NO: 541 - DNA, 10 bp; single-stranded, linear
gttagagtgg

SEQ ID NO: 542 - DNA, 10 bp; single-stranded, linear
ttagagtgga

SEQ ID NO: 543 - DNA, 10 bp; single-stranded, linear
tagagtggag

SEQ ID NO: 544 - DNA, 10 bp; single-stranded, linear
agagtggagg

SEQ ID NO: 545 - DNA, 10 bp; single-stranded, linear
gagtggaggt

SEQ ID NO: 546 - DNA, 10 bp; single-stranded, linear
agtggaggtt

SEQ ID NO: 547 - DNA, 10 bp; single-stranded, linear
gtggaggttt

SEQ ID NO: 548 - DNA, 10 bp; single-stranded, linear
tggaggtttg

SEQ ID NO: 549 - DNA, 10 bp; single-stranded, linear
ggaggtttga

SEQ ID NO: 550 - DNA, 10 bp; single-stranded, linear
gaggtttgac

SEQ ID NO: 551 - DNA, 10 bp; single-stranded, linear
aggtttgaca

SEQ ID NO: 552 - DNA, 10 bp; single-stranded, linear
ggtttgacag

SEQ ID NO: 553 - DNA, 10 bp; single-stranded, linear
gtttgacagc

SEQ ID NO: 554 - DNA, 10 bp; single-stranded, linear
tttgacagcc

SEQ ID NO: 555 - DNA, 10 bp; single-stranded, linear
ttgacagccg

SEQ ID NO: 556 - DNA, 10 bp; single-stranded, linear
tgacagccgc

SEQ ID NO: 557 - DNA, 10 bp; single-stranded, linear
gacagccgcc

SEQ ID NO: 558 - DNA, 10 bp; single-stranded, linear
acagccgcct

SEQ ID NO: 559 - DNA, 10 bp; single-stranded, linear
cagccgccta

SEQ ID NO: 560 - DNA, 10 bp; single-stranded, linear
agccgcctag

SEQ ID NO: 561 - DNA, 10 bp; single-stranded, linear
gccgcctagc

SEQ ID NO: 562 - DNA, 10 bp; single-stranded, linear
ccgcctagca

SEQ ID NO: 563 - DNA, 10 bp; single-stranded, linear
cgcctagcat

SEQ ID NO: 564 - DNA, 10 bp; single-stranded, linear
gcctagcatt

SEQ ID NO: 565 - DNA, 10 bp; single-stranded, linear
cctagcattt

SEQ ID NO: 566 - DNA, 10 bp; single-stranded, linear
ctagcatttc

SEQ ID NO: 567 - DNA, 10 bp; single-stranded, linear
tagcatttca

SEQ ID NO: 568 - DNA, 10 bp; single-stranded, linear
agcatttcat

SEQ ID NO: 569 - DNA, 10 bp; single-stranded, linear
gcatttcatc

SEQ ID NO: 570 - DNA, 10 bp; single-stranded, linear
catttcatca

SEQ ID NO: 571 - DNA, 10 bp; single-stranded, linear
atttcatcac

SEQ ID NO: 572 - DNA, 10 bp; single-stranded, linear
tttcatcacg

SEQ ID NO: 573 - DNA, 10 bp; single-stranded, linear
ttcatcacgt

SEQ ID NO: 574 - DNA, 10 bp; single-stranded, linear
tcatcacgtg

SEQ ID NO: 575 - DNA, 10 bp; single-stranded, linear
catcacgtgg

SEQ ID NO: 576 - DNA, 10 bp; single-stranded, linear
atcacgtggc

SEQ ID NO: 577 - DNA, 10 bp; single-stranded, linear
tcacgtggcc

SEQ ID NO: 578 - DNA, 10 bp; single-stranded, linear
cacgtggccc

SEQ ID NO: 579 - DNA, 10 bp; single-stranded, linear
acgtggcccg

SEQ ID NO: 580 - DNA, 10 bp; single-stranded, linear
cgtggcccga

SEQ ID NO: 581 - DNA, 10 bp; single-stranded, linear
gtggcccgag

SEQ ID NO: 582 - DNA, 10 bp; single-stranded, linear
tggcccgaga

SEQ ID NO: 583 - DNA, 10 bp; single-stranded, linear
ggcccgagag

SEQ ID NO: 584 - DNA, 10 bp; single-stranded, linear
gcccgagagc

SEQ ID NO: 585 - DNA, 10 bp; single-stranded, linear
cccgagagct

SEQ ID NO: 586 - DNA, 10 bp; single-stranded, linear
ccgagagctg

SEQ ID NO: 587 - DNA, 10 bp; single-stranded, linear
cgagagctgc

SEQ ID NO: 588 - DNA, 10 bp; single-stranded, linear
gagagctgca

SEQ ID NO: 589 - DNA, 10 bp; single-stranded, linear
agagctgcat

SEQ ID NO: 590 - DNA, 10 bp; single-stranded, linear
gagctgcatc

SEQ ID NO: 591 - DNA, 10 bp; single-stranded, linear
agctgcatcc

SEQ ID NO: 592 - DNA, 10 bp; single-stranded, linear
gctgcatccg

SEQ ID NO: 593 - DNA, 10 bp; single-stranded, linear
ctgcatccgg

SEQ ID NO: 594 - DNA, 10 bp; single-stranded, linear
tgcatccgga

SEQ ID NO: 595 - DNA, 10 bp; single-stranded, linear
gcatccggag

SEQ ID NO: 596 - DNA, 10 bp; single-stranded, linear
catccggagt

SEQ ID NO: 597 - DNA, 10 bp; single-stranded, linear
atccggagta

SEQ ID NO: 598 - DNA, 10 bp; single-stranded, linear
tccggagtac

SEQ ID NO: 599 - DNA, 10 bp; single-stranded, linear
ccggagtact

SEQ ID NO: 600 - DNA, 10 bp; single-stranded, linear
cggagtactt

SEQ ID NO: 601 - DNA, 10 bp; single-stranded, linear
ggagtacttc

SEQ ID NO: 602 - DNA, 10 bp; single-stranded, linear
gagtacttca

SEQ ID NO: 603 - DNA, 10 bp; single-stranded, linear
agtacttcaa

SEQ ID NO: 604 - DNA, 10 bp; single-stranded, linear
gtacttcaag

SEQ ID NO: 605 - DNA, 10 bp; single-stranded, linear
tacttcaaga

SEQ ID NO: 606 - DNA, 10 bp; single-stranded, linear
acttcaagaa

SEQ ID NO: 607 - DNA, 10 bp; single-stranded, linear
cttcaagaac

SEQ ID NO: 608 - DNA, 10 bp; single-stranded, linear
ttcaagaact

SEQ ID NO: 609 - DNA, 10 bp; single-stranded, linear
tcaagaactg

SEQ ID NO: 610 - DNA, 10 bp; single-stranded, linear
caagaactgc

SEQ ID NO: 611 - DNA, 10 bp; single-stranded, linear
aagaactgct

SEQ ID NO: 612 - DNA, 10 bp; single-stranded, linear
agaactgctg

SEQ ID NO: 613 - DNA, 10 bp; single-stranded, linear
gaactgctga

SEQ ID NO: 614 - DNA, 1305 bp; single-stranded, linear
gaagagatttgggagaacatgacctggatgcagtgggaaaaagaaattcacaatcacacaaaatacatatactccttacttgaaaaatcgcagaaccaacaagaaaagaatgaacaagaactattggaattggatcaatgggcaagtttgtggaattggtttgacataacaaaatggctgtggtatataaaaatattcataatggtagtaggaggcttgataggtttaagaatagtttttgctgtactttctatagtgaatagagttaggcagggatactcaccattgtcgtttcagaccctcctcccaaccccgaggggacccgacaggcccgaaggaatcgaagaagaaggtggagagagagacagagacagatccactcgattagtacacggattcttagcacttttctgggacgacctgaggagcctgtgcctcttcctctaccaccacttgagagacttactcttgattgtaacaaggattgtggaacttctgggacgcaggggatgggaagccctcaaatattggtggaacctcctaaagtattggagccaggaactgcagaagagtgctgttatcttgctcaatgccaccgccatagcagtagctgaggggacagatagagttttagaagtattacaaagagcttatagagctatcctccacatacctagaagaataagacagggcctcgaaatggctttgctataaaatgggtggcaagtgagcaaaaagtagtgtagtcagatagcatgcatcataaggggtgggggccaacaactaacaatgctgatcgtgcctggctagaagcacaagagaaggaagaagcgggttttccagtcaaacctcaggtagctgtagatcttagccactttttaaaagaaaaggggggactggaagggctaattcactcccaaagaagacaagatacacagtgctgcaaactattaccagtggagtcagcgaagatagaagaggccaatggaggagaaaaccacagattgttctgttggggactttccatccgttggggactttccaaggcggcgtggcctgggtgactagttccggtggggactttccaagaaggcgcggcctgggcgggactggggagtggcgagccctcagatgctgcatataagcagctgctttctgctgttactgggtctctcgggttagaccagatctgagcctgggagctctctggctaactagggaacccactgcttaagcctcaataaagcttgccttgagtgcttcaagtagtgtgtgcccgtctgttgtgtgactctggtatctaga

SEQ ID NO: 615 - DNA, 1208 bp; single-stranded, linear
gaaacaatttgggataacatgacctggatgcagtgggaaagagaaattgacaattacacaaacataatatacaccttaattgaagaatcgcagaaccaacaagaaaaaaatgaactagaattattggaattggataaatgggcaaatttgtggaattggtttagtatatcaaactggctatggtatataaaattattcataatggtagtaggaggcttggtaggtttaagaatagtttttactgtactttctatagtgaatagagttaggcagggatactcaccattgtcgtttcagacccacctcccaaccccgaagggacccgacaggccagaaggaatcgaagaagaaggtggagagagagacagaggcagctccactcgattagtgcacggattcttagcacttttctgggacgacctgaggagtctgtgcctcttcagctaccaccacttgagagacttactcttgattgtaacgaggattgtggaacttctgggacgcaggggatgggaagccctcaaatactggtggaatctcctgcagtattggaggcaggaactacagaagagtgctgttagcttgttcaatggcacggccatagcagtagctgaggggacagatagagttatagaagctttacgaagggcttatagagctattctccacatacctagaagaataagacagggcttagaaagggctttgctataaaatgggtggcaagtggtcagaaagtagtgtggttagaaggcatgtacctttaagacaaggcagctatagatcttagccgctttttaaaagaaaaggggggactggaagggctaattcactcacagagaagatcagttgaaccagaagaagatagaagaggccatgaagaagaaaacaacagattgttccgtttgttccgttggggactttccaggagacgtggcctgagtgataagccgctggggactttccgaagaggcgtgacgggactttccaaggcgacgtggcctgggcgggactggggagtggcgagccctcagatgctgcatataagcagctgctttctgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtatctaga

SEQ ID NO: 616 - DNA, 18 bp; single-stranded, linear
tggaagggctaatttggt

SEQ ID NO: 617 - DNA, 25 bp; single-stranded, linear
atcttccctaaaaaattagcctgtc

SEQ ID NO: 618 - DNA, 20 bp; single-stranded, linear
aggctcagatctggtctaac

SEQ ID NO: 619 - DNA, 20 bp; single-stranded, linear
agcagcaggaagcactatgg

SEQ ID NO: 620 - DNA, 19 bp; single-stranded, linear
tgctagagattttccacac

SEQ ID NO: 621 - DNA, 20 bp; single-stranded, linear
agtgaatagagttaggcagg

SEQ ID NO: 622 - DNA, 21 bp; single-stranded, linear
gtaagacagtatgatcagata

SEQ ID NO: 623 - DNA, 21 bp; single-stranded, linear
ttgtagggaattccaaattcc

SEQ ID NO: 624 - DNA, 26 bp; single-stranded, linear
caggatcctacacctgtcaacataat

SEQ ID NO: 625 - DNA, 22 bp; single-stranded, linear
gggaattccttattcctgcttg

SEQ ID NO: 626 - DNA, 18 bp; single-stranded, linear
ccagaagttccacaatcc

SEQ ID NO: 627 - DNA, 19 bp; single-stranded, linear
ttcttctaggtatgtggag

SEQ ID NO: 628 - DNA, 19 bp; single-stranded, linear
agtgaattagcccttccag

SEQ ID NO: 629 - DNA, 19 bp; single-stranded, linear
tgctagagattttccacac

SEQ ID NO: 630 - DNA, 17 bp; single-stranded, linear
tgctctggaaaactcat

SEQ ID NO: 631 - DNA, 19 bp; single-stranded, linear
ctttctatagtgaatagag

SEQ ID NO: 632 - DNA, 17 bp; single-stranded, linear
tattggagtcaggaact

SEQ ID NO: 633 - DNA, 17 bp; single-stranded, linear
ggtctaaccagagagac

SEQ ID NO: 634 - protein, 13 aa
AVRERMRRAEPAA

SEQ ID NO: 635 - protein, 32 aa
PTSQSRGDPTGPKEKKKVERETETDPFDTDPH

SEQ ID NO: 636 - protein, 34 aa
PSSQPRGDPTGPKESKKKVERETETDPLDYTDSH

SEQ ID NO: 637 - protein, 34 aa
PTSQPRRDPTGQKESKKKVERETEAAPLDCTDSH

SEQ ID NO: 638 - protein, 106 aa
DPPPNPEGTRQARRNRRRRWRERQRQIHSISERILSTYLGRSAEPVPLQLPPLERLTLDCNEDCGTSGTQGVGSPQILVESPTVLESGTKECCLAQCHSHSSSGDR

SEQ ID NO: 639 - protein, 108 aa
DPPPNPEGTRQARRNRRRRWRERQRQIHSISTRILSTFLGRPEEPVPLPLPPLERLTLDCNKDCGTSGTQGMGSPQILVEPPKVLEPGTAEECCYLAQCHRHSSSGDR

SEQ ID NO: 640 - protein, 107 aa
DPPPNPEGTRQARRNRRRRWRERQRQLHSISARILSTFLGRPEESVPLQLPPLERLTLDCNEDCGTSGTQGMGSPQILVESPAVLEAGTTEECCLVQWHGHSSSGDR

SEQ ID NO: 641 - protein, 237 aa
EQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPIPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIEVLQAAYRAIRHIPRRIRQGLERILL

SEQ ID NO: 642 - protein, 237 aa
EEIWENMTWMQWEKEIHNHTKYIYSLLEKSQNQQEKNEQELLELDQWASLWNWFDITKWLWYIKIFIMVVGGLIGLRIVFAVLSIVNRVRQGYSPLSFQTLLPTPRGPDRPEGIEEMGGERDRDRSTRLVHGFLALFWDDLRSLCLFLYHHLRDLLLIVTRIVELLGRRGWEALKYWWNLLKYWSQELQKSAVILLNATAIAVAEGTDRVLEVLQRAYRAILHIPRRIRQGLEMALL

SEQ ID NO: 643 - protein, 237 aa
ETIWDNMTWMQWEREIDNYTNIIYTLIEESQNQQEKNELELLELDKWANLWNWFSISNWLWYIKLFIMVVGGLVGLRIVFTVLSIVNRVRQGYSPLSFQTHLPTPKGPDRPEGIEEEGGERDRGSSTRLVHGFLALFWDDLRSLCLFSYHHLRDLLLIVTRIVELLGRRGWEALKYWWNLLQYWRQELQKSAVSLFNGTAIAVAEGTDRVIEALRRAYRAILHIPRRIRQGLERALL

SEQ ID NO: 644 - protein, 206 aa
MGGKWSKSSVIGWPAVRERMRRAEPAADGVGAVSRDLEKHGAITSSNTAANNAACAWLEAQEEEEVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 645 - protein, 22 aa
MGGKAKSSVVRHASGVGANNGC

SEQ ID NO: 646 - protein, 26 aa
MGGKWSESSVVRRHVPLRQGSYRSPL

SEQ ID NO: 647 - DNA, 74 bp; single-stranded, linear
ctacaagggactttccgctggggactttccagggaggcgtggcctgggcgggactggggagtggcgagccctca

SEQ ID NO: 648 - DNA, 78 bp; single-stranded, linear
ctgttggggactttccatccgttggggactttccaaggcggcgtggcctgggtgactagttccggtggggactttcca

SEQ ID NO: 649 - DNA, 67 bp; single-stranded, linear
ccgtttgttccgttggggactttccaggagacgtggcctgagtgactaagccgctggggactttccg

SEQ ID NO: 650 - DNA, 621 bp; single-stranded, linear
atgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctga

SEQ ID NO: 651 - DNA, 1596 bp; single-stranded, linear
gaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttaacttgctcaatgccacagccatagcagtagctgaggggacagatagggttatagaagtattacaagcagcttatagagctattcgccacatacctagaagaataagacagggcttggaaaggattttgctataagatgggtggcaagtggtcaaaaagtagtgtgattggatggcctgctgtaagggaaagaatgagacgagctgagccagcagcagatggggtgggagcagtatctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactaga

SEQ ID NO: 652 - DNA, 10 bp; single-stranded, linear
gctttttgcc

SEQ ID NO: 653 - DNA, 10 bp; single-stranded, linear
ctttttgcct

SEQ ID NO: 654 - DNA, 10 bp; single-stranded, linear
tttttgcctg

SEQ ID NO: 655 - DNA, 10 bp; single-stranded, linear
ttttgcctgt

SEQ ID NO: 656 - DNA, 10 bp; single-stranded, linear
tttgcctgta

SEQ ID NO: 657 - DNA, 10 bp; single-stranded, linear
ttgcctgtac

SEQ ID NO: 658 - DNA, 10 bp; single-stranded, linear
tgcctgtact

SEQ ID NO: 659 - DNA, 10 bp; single-stranded, linear
gcctgtactg

SEQ ID NO: 660 - DNA, 10 bp; single-stranded, linear
cctgtactgg

SEQ ID NO: 661 - DNA, 10 bp; single-stranded, linear
ctgtactggg

SEQ ID NO: 662 - DNA, 10 bp; single-stranded, linear
tgtactgggt

SEQ ID NO: 663 - DNA, 10 bp; single-stranded, linear
gtactgggtc

SEQ ID NO: 664 - DNA, 10 bp; single-stranded, linear
tactgggtct

SEQ ID NO: 665 - DNA, 10 bp; single-stranded, linear
actgggtctc

SEQ ID NO: 666 - DNA, 10 bp; single-stranded, linear
ctgggtctct

SEQ ID NO: 667 - DNA, 10 bp; single-stranded, linear
tgggtctctc

SEQ ID NO: 668 - DNA, 10 bp; single-stranded, linear
gggtctctct

SEQ ID NO: 669 - DNA, 10 bp; single-stranded, linear
ggtctctctg

SEQ ID NO: 670 - DNA, 10 bp; single-stranded, linear
gtctctctgg

SEQ ID NO: 671 - DNA, 10 bp; single-stranded, linear
tctctctggt

SEQ ID NO: 672 - DNA, 10 bp; single-stranded, linear
ctctctggtt

SEQ ID NO: 673 - DNA, 10 bp; single-stranded, linear
tctctggtta

SEQ ID NO: 674 - DNA, 10 bp; single-stranded, linear
ctctggttag

SEQ ID NO: 675 - DNA, 10 bp; single-stranded, linear
tctctggtta

SEQ ID NO: 676 - DNA, 10 bp; single-stranded, linear
ctggttagac

SEQ ID NO: 677 - DNA, 10 bp; single-stranded, linear
tggttagacc

SEQ ID NO: 678 - DNA, 10 bp; single-stranded, linear
ggttagacca

SEQ ID NO: 679 - DNA, 10 bp; single-stranded, linear
gttagaccag

SEQ ID NO: 680 - DNA, 10 bp; single-stranded, linear
ttagaccaga

SEQ ID NO: 681 - DNA, 10 bp; single-stranded, linear
tagaccagat

SEQ ID NO: 682 - DNA, 10 bp; single-stranded, linear
agaccagatc

SEQ ID NO: 683 - DNA, 10 bp; single-stranded, linear
gaccagatct

SEQ ID NO: 684 - DNA, 10 bp; single-stranded, linear
accagatctg

SEQ ID NO: 685 - DNA, 10 bp; single-stranded, linear
ccagatctga

SEQ ID NO: 686 - DNA, 10 bp; single-stranded, linear
cagatctgag

SEQ ID NO: 687 - DNA, 10 bp; single-stranded, linear
agatctgagc

SEQ ID NO: 688 - DNA, 10 bp; single-stranded, linear
gatctgagcc

SEQ ID NO: 689 - DNA, 10 bp; single-stranded, linear
atctgagcct

SEQ ID NO: 690 - DNA, 10 bp; single-stranded, linear
tctgagcctg

SEQ ID NO: 691 - DNA, 10 bp; single-stranded, linear
ctgagcctgg

SEQ ID NO: 692 - DNA, 10 bp; single-stranded, linear
tgagcctggg

SEQ ID NO: 693 - DNA, 10 bp; single-stranded, linear
gagcctggga

SEQ ID NO: 694 - DNA, 10 bp; single-stranded, linear
agcctgggag

SEQ ID NO: 695 - DNA, 10 bp; single-stranded, linear
gcctgggagc

SEQ ID NO: 696 - DNA, 10 bp; single-stranded, linear
cctgggagct

SEQ ID NO: 697 - DNA, 10 bp; single-stranded, linear
ctgggagctc

SEQ ID NO: 698 - DNA, 10 bp; single-stranded, linear
tgggagctct

SEQ ID NO: 699 - DNA, 10 bp; single-stranded, linear
gggagctctc

SEQ ID NO: 700 - DNA, 10 bp; single-stranded, linear
ggagctctct

SEQ ID NO: 701 - DNA, 10 bp; single-stranded, linear
gagctctctg

SEQ ID NO: 702 - DNA, 10 bp; single-stranded, linear
agctctctgg

SEQ ID NO: 703 - DNA, 10 bp; single-stranded, linear
gctctctggc

SEQ ID NO: 704 - DNA, 10 bp; single-stranded, linear
ctctctggct

SEQ ID NO: 705 - DNA, 10 bp; single-stranded, linear
tctctggcta

SEQ ID NO: 706 - DNA, 10 bp; single-stranded, linear
ctctggctaa

SEQ ID NO: 707 - DNA, 10 bp; single-stranded, linear
tctggctaac

SEQ ID NO: 708 - DNA, 10 bp; single-stranded, linear
ctggctaact

SEQ ID NO: 709 - DNA, 10 bp; single-stranded, linear
tggctaacta

SEQ ID NO: 710 - DNA, 10 bp; single-stranded, linear
ggctaactag

SEQ ID NO: 711 - DNA, 10 bp; single-stranded, linear
gctaactagg

SEQ ID NO: 712 - DNA, 10 bp; single-stranded, linear
ctaactaggg

SEQ ID NO: 713 - DNA, 10 bp; single-stranded, linear
taactaggga

SEQ ID NO: 714 - DNA, 10 bp; single-stranded, linear
aactagggaa

SEQ ID NO: 715 - DNA, 10 bp; single-stranded, linear
actagggaac

SEQ ID NO: 716 - DNA, 10 bp; single-stranded, linear
ctagggaacc

SEQ ID NO: 717 - DNA, 10 bp; single-stranded, linear
tagggaaccc

SEQ ID NO: 718 - DNA, 10 bp; single-stranded, linear
agggaaccca

SEQ ID NO: 719 - DNA, 10 bp; single-stranded, linear
gggaacccac

SEQ ID NO: 720 - DNA, 10 bp; single-stranded, linear
ggaacccact

SEQ ID NO: 721 - DNA, 10 bp; single-stranded, linear
gaacccactg

SEQ ID NO: 722 - DNA, 10 bp; single-stranded, linear
aacccactgc

SEQ ID NO: 723 - DNA, 10 bp; single-stranded, linear
acccactgct

SEQ ID NO: 724 - DNA, 10 bp; single-stranded, linear
cccactgctt

SEQ ID NO: 725 - DNA, 10 bp; single-stranded, linear
ccactgctta

SEQ ID NO: 726 - DNA, 10 bp; single-stranded, linear
cactgcttaa

SEQ ID NO: 727 - DNA, 10 bp; single-stranded, linear
actgcttaag

SEQ ID NO: 728 - DNA, 10 bp; single-stranded, linear
ctgcttaagc

SEQ ID NO: 729 - DNA, 10 bp; single-stranded, linear
tgcttaagcc

SEQ ID NO: 730 - DNA, 10 bp; single-stranded, linear
gcttaagcct

SEQ ID NO: 731 - DNA, 10 bp; single-stranded, linear
cttaagcctc

SEQ ID NO: 732 - DNA, 10 bp; single-stranded, linear
ttaagcctca

SEQ ID NO: 733 - DNA, 10 bp; single-stranded, linear
taagcctcaa

SEQ ID NO: 734 - DNA, 10 bp; single-stranded, linear
aagcctcaat

SEQ ID NO: 735 - DNA, 10 bp; single-stranded, linear
agcctcaata

SEQ ID NO: 736 - DNA, 10 bp; single-stranded, linear
gcctcaataa

SEQ ID NO: 737 - DNA, 10 bp; single-stranded, linear
cctcaataaa

SEQ ID NO: 738 - DNA, 10 bp; single-stranded, linear
ctcaataaag

SEQ ID NO: 739 - DNA, 10 bp; single-stranded, linear
tcaataaagc

SEQ ID NO: 740 - DNA, 10 bp; single-stranded, linear
caataaagct

SEQ ID NO: 741 - DNA, 10 bp; single-stranded, linear
aataaagctt

SEQ ID NO: 742 - DNA, 10 bp; single-stranded, linear
ataaagcttg

SEQ ID NO: 743 - DNA, 10 bp; single-stranded, linear
taaagcttgc

SEQ ID NO: 744 - DNA, 10 bp; single-stranded, linear
aaagcttgcc

SEQ ID NO: 745 - DNA, 10 bp; single-stranded, linear
aagcttgcct

SEQ ID NO: 746 - DNA, 10 bp; single-stranded, linear
agcttgcctt

SEQ ID NO: 747 - DNA, 10 bp; single-stranded, linear
gcttgccttg

SEQ ID NO: 748 - DNA, 10 bp; single-stranded, linear
cttgccttga

SEQ ID NO: 749 - DNA, 10 bp; single-stranded, linear
ttgccttgag

SEQ ID NO: 750 - DNA, 10 bp; single-stranded, linear
tgccttgagt

SEQ ID NO: 751 - DNA, 10 bp; single-stranded, linear
gccttgagtg

SEQ ID NO: 752 - DNA, 10 bp; single-stranded, linear
ccttgagtgc

SEQ ID NO: 753 - DNA, 10 bp; single-stranded, linear
cttgagtgct

SEQ ID NO: 754 - DNA, 10 bp; single-stranded, linear
ttgagtgctt

SEQ ID NO: 755 - DNA, 10 bp; single-stranded, linear
tgagtgcttc

SEQ ID NO: 756 - DNA, 10 bp; single-stranded, linear
gagtgcttca

SEQ ID NO: 757 - DNA, 10 bp; single-stranded, linear
agtgcttcaa

SEQ ID NO: 758 - DNA, 10 bp; single-stranded, linear
gtgcttcaag

SEQ ID NO: 759 - DNA, 10 bp; single-stranded, linear
tgcttcaagt

SEQ ID NO: 760 - DNA, 10 bp; single-stranded, linear
gcttcaagta

SEQ ID NO: 761 - DNA, 10 bp; single-stranded, linear
cttcaagtag

SEQ ID NO: 762 - DNA, 10 bp; single-stranded, linear
ttcaagtagt

SEQ ID NO: 763 - DNA, 10 bp; single-stranded, linear
tcaagtagtg

SEQ ID NO: 764 - DNA, 10 bp; single-stranded, linear
caagtagtgt

SEQ ID NO: 765 - DNA, 10 bp; single-stranded, linear
aagtagtgtg

SEQ ID NO: 766 - DNA, 10 bp; single-stranded, linear
agtagtgtgt

SEQ ID NO: 767 - DNA, 10 bp; single-stranded, linear
gtagtgtgtg

SEQ ID NO: 768 - DNA, 10 bp; single-stranded, linear
tagtgtgtgc

SEQ ID NO: 769 - DNA, 10 bp; single-stranded, linear
agtgtgtgcc

SEQ ID NO: 770 - DNA, 10 bp; single-stranded, linear
gtgtgtgccc

SEQ ID NO: 771 - DNA, 10 bp; single-stranded, linear
tgtgtgcccg

SEQ ID NO: 772 - DNA, 10 bp; single-stranded, linear
gtgtgcccgt

SEQ ID NO: 773 - DNA, 10 bp; single-stranded, linear
tgtgcccgtc

SEQ ID NO: 774 - DNA, 10 bp; single-stranded, linear
gtgcccgtct

SEQ ID NO: 775 - DNA, 10 bp; single-stranded, linear
tgcccgtctg

SEQ ID NO: 776 - DNA, 10 bp; single-stranded, linear
gcccgtctgt

SEQ ID NO: 777 - DNA, 10 bp; single-stranded, linear
cccgtctgtt

SEQ ID NO: 778 - DNA, 10 bp; single-stranded, linear
ccgtctgttg

SEQ ID NO: 779 - DNA, 10 bp; single-stranded, linear
cgtctgttgt

SEQ ID NO: 780 - DNA, 10 bp; single-stranded, linear
gtctgttgtg

SEQ ID NO: 781 - DNA, 10 bp; single-stranded, linear
tctgttgtgt

SEQ ID NO: 782 - DNA, 10 bp; single-stranded, linear
ctgttgtgtg

SEQ ID NO: 783 - DNA, 10 bp; single-stranded, linear
tgttgtgtga

SEQ ID NO: 784 - DNA, 10 bp; single-stranded, linear
gttgtgtgac

SEQ ID NO: 785 - DNA, 10 bp; single-stranded, linear
ttgtgtgact

SEQ ID NO: 786 - DNA, 10 bp; single-stranded, linear
tgtgtgactc

SEQ ID NO: 787 - DNA, 10 bp; single-stranded, linear
gtgtgactct

SEQ ID NO: 788 - DNA, 10 bp; single-stranded, linear
tgtgtgactc

SEQ ID NO: 789 - DNA, 10 bp; single-stranded, linear
gtgtgactct

SEQ ID NO: 790 - DNA, 10 bp; single-stranded, linear
tgtgactctg

SEQ ID NO: 791 - DNA, 10 bp; single-stranded, linear
gtgactctgg

SEQ ID NO: 792 - DNA, 10 bp; single-stranded, linear
tgactctggt

SEQ ID NO: 793 - DNA, 10 bp; single-stranded, linear
gactctggta

SEQ ID NO: 794 - DNA, 10 bp; single-stranded, linear
actctggtaa

SEQ ID NO: 795 - DNA, 10 bp; single-stranded, linear
ctctggtaac

SEQ ID NO: 796 - DNA, 10 bp; single-stranded, linear
tctggtaact

SEQ ID NO: 797 - DNA, 10 bp; single-stranded, linear
ctggtaacta

SEQ ID NO: 798 - DNA, 10 bp; single-stranded, linear
tggtaactag

SEQ ID NO: 799 - DNA, 10 bp; single-stranded, linear
ggtaactaga

SEQ ID NO: 800 - DNA, 9207 bp; single-stranded, linear
tggaagggctaattcactcacggaaaagaccagttgaaccagaagaagatagaagaggccatgaagaagaaaacaacagattgttctgcttgctcagctggggactttccagaaggcgcggcctgagtgactaagccccgttggggactttccgaagaggcatgaagggactttccaaggcaggcgtggcctgggcgggactggggagtggcgagccctcagatgctgcatataagcagctgctttctgcctgtactgggtctctctggttagaccagatctgagcctgggagctctctggctagctagggaacccactgcttaagcctcaataaagcttgccttgagtgcttcaagtagtgtgtgcccgtctgttgtgtgactctggtatctagagatccctcagaccattttagtccgtgtggaaaatctctagcagtggcgcccgaacagggacttgaaagcgaaaggaaaaccagaggagctctctcgacgcaggactcggcttgctgaagcgcgcacggcaagaggcgaggggcggcgactggtgagtacgccgaaaattttgactagcggaggctagaaggagagagatgggtgcgagagcgtcaatattaagcgggggaaaattagatagatgggagaaaattcggttaaggccaggaggaaagaaaaagtataaattaaaacatatagtatgggcaagcagggagctagaacgattcgcagtcaatcctggcctgttggaaacatcagaaggctgtagacaaatactgggacagttacacccgtcccttcagacaggatcagaagaacttaaatcagtatataatgcagtagcagtcctctattgtgtgcatcaaaacatagacataaaggacaccaaggaagctttagaaaagatagaggaagagcaaaacaaatgtaagaaaaaagcacagcaagcagcagcacagcaagcagcagctggcacaggaaacagcaacccggtcagccaaaattaccctatagtacagaacatgcaggggcaaatggtacatcaggccatatcacctagaactttaaatgcatgggtaaaagtaatagaagagaaggctttcagcccagaggtaatacccatgttttcagcattatcagaaggagccaccccacaagatttaaacaccatgctaaacacagtggggggacatcaagcagctatgcaaatgttaaaagagaccatcaatgaggaagctgcagaatgggatagattacatccagcgcaggcagggcctgttgcaccaggccagatgagagacccaaggggaagtgacatagcaggaactactagtacccttcaggaacaaataggatggatgacaggtaatccagctatcccagtaggagaaatctataaaagatggataatcctgggattaaataaaatagtaaggatgtatagccctatcagcattctggacataaaacaaggaccaaaggaaccctttagagactatgtagaccggttctataaaactctaagagccgagcaagctacacaggaggtaaaaaattggatgacagaaaccttgttggtccaaaatgcaaacccagattgtaagactattttaaaagcattgggaccagcagctacactagaagaaatgatgacagcatgtcagggagtgggaggacccagccataaagcaagagttttggcagaagcaatgagccaagcaacaaatgcagctactgtaatgatgcagagaagcaattttagaaaccaaagaaagaatgttaagtgtttcaattgtggcaaagaagggcacatagccagaaattgcagggctcctaggaaaaggggctgttggaaatgtggaaaggaaggacaccaaatgaaagattgtactgagagacaggctaattttttagggaaaatctggccttcccacaaggggaggccagggaactttcttcagagcaggccagaaccaacagcccctctccagggcaggccggagccatcagccccgccagaagagagcttcaggtttggggaggagacaacaactccctctcagaagcaggagccgatagacagggacagggatctgtatcctttagcttccctcagatcactctttggcaacgacccctcgtcacaataaagataggggggcagctgaaggaagctctattagatacaggagcagatgatacagtattagaagacatgcatttgccaggaaaatggaaaccaaaaatgatagggggaattggaggttttatcaaagtaaaacaatatgatgaaattcttgtagaaatctgtggacataaagctataggtacagtattagtaggacctacacctgtcaacataattggaagaaatctgttgactcagattggttgcactttaaattttcccattagtcctattgaaactgtaccagtacaattaaagccaggaatggatggcccaaaggttaaacaatggccattgacagaagagaaaataaaagcattagtagaaatttgtacagaaatggaaaaggaaggaaagatttcaaaaattgggcctgaaaatccatacaatactccagtatttgccataaagaaaaaagatggtactaaatggagaaaattagtagatttcagagaccttaataagagaactcaagacttctgggaagttcaattaggaataccacatccctcaggattaaaaaagaaaaaatcagtaacagtactggatgtgggtgatgcatacttttcagttcccttagatgaaaacttcaggaagtatactgcatttaccatacctagtataaataatgagacaccagggattagatatcagtacaatgtgcttccacagggatggaaaggatcaccagcaatattccaaagtagcatgacaagaatcttagagccttttagaagacaaaatccagacatagttatctatcaatacatggatgacttgtatgtaggatctgatttagaaataggacagcatagaataaaaatagaggaactgagacaacatctgttgaagtggggatttaccacaccagacaaaaagcatcagaaagaacccccattcctttggatgggttatgaactccatcctgataaatggacagtgcaacctatagtactgccagaaaaagacagctggactgtcaatgacatacagaagttagtgggtaaattaaattgggcaagtcagatttacccaggaattaaagtaaggcaattatgtaaactccttaggggaaccaaagcactaacagaagtaataccactaacagaagaagcagagctagaactggcagaaaacagggaaattctaagagaaccagtacatggagtgtattatgacccatcaaaagacttaatagcagaaatacagaagcaggagcaaggccaatggacatatcaaatttatcaagatcaatttaaaaatctaaaaacaggaaagtatgcaagattgaggggtgcccacactaatgatgtaaaacaatttccagaggcagtgcaaaaaatagccacagaaagcatagtaatatggggaaagactcctaaatttagactacccatacaaaaagaaacatgggacgcatggtggacagagtattggcaagccacctggattcctgagtgggagtttgtcaatacccctcccctagtaaaattatggtaccagttagaaaaagaacccataataggagcagaaactttctatgtagatggggcagctaacagagagactaaattaggaaaagcaggatatgttactgacagaggaagacaaaaagttgtctccctaactgacacaacaaatcagaagactgagttacaagcaattcatctagctttgcaggattcaggattagaagtaaacatagtaacagactcacagtatgcattaggaatcattcaagcacaaccagataaaagtgaatcagaaatagtcaatcaaataatagagcaattaataaaaaaggaaaaggtctacctggcatgggtaccagcacacaaaggaattggagggaatgaacaagtagataaattagtcagtgctggaatcaggaaaatactatttttagatggaatagataaggcacaagaaggccatgagaaatatcacagtaattggagagcaatggctagtggttttaacctgccacctatagtagcaaaagaaatagtagccagctgtgataaatgtcagctaaaaggagaagccatgcatggacaagtagactgtagtccaggaatatggcaactagattgtacacatctagaaggaaaaattatcctggtagcagttcatgtagccagtggatatatagaagcagaagttattccagcagagacagggcaggaaacagcatactttatcttaaaattagcaggaaggtggccagtaaacacaatacatacagacaatggcggcaatttcatcagtaccacggttaaggccgcctgttggtgggcagggatcaagcaggaatttggcattccctacaatccccaaagccaaggagtagtggaatctatgaatagagaattaaagaaaattataggacaggtaagagatcaggctgaacatcttaagacagcagtacaaatggcagtattcatccacaattttaaaagaaaaggggggattgggggatacagtgcaggggaaagaatagtagacataatagcaacagacatacaaactaaagaattacaaaagcaaattacaaaaattcaaaattttcgggtttattacagggacagcagagatccactttggaaaggaccagcaaaacttctctggaaaggcgaaggggcagtagtaatacaagataatagtgacataaaagtagtgccaagaagaaaagtaaagatcattagggattatggaaaacagatggcaggtgatgattgtgtggcaagtagacaggatgaggattagaacatggaacagtttagtgaaacaccatatgtatgtttcaaagaaagctaagggatggatttatagacatcactatgaaaacactcatccaaaaataagctcagaagtacacatcccactaggggaagctagattggtaataacaacatattggggtctacatacaggagaaagagactggcatttgggtcagggagtctccatagaatggagggaaaggacatatagaacacaagtagaccccgaactagcagaccaactaattcatatacattactttgattgtttttcagaatctgccataagaagtgccatattaggatatagagttaggcataggtgtgaatatcaagcaggacataacaaggtaggatctctacagtacttggcactaacagcattaataacaccaaagaagataaagccacctttgcctagtgttgcgaaactgacagaggatagatggaacaagccccagaagaccaagggccacagaggcagccatacaatgaatggacactagaacttttagaggagcttaagaatgaagctgttaggcattttcctagggtatggctccatggcttagggcaacatatctatgaaacttatggggatacttgggaaggagtggaggccataacaagaactctgcaacaactgctgtttattcatttcagaattgggtgtcaacatagcagaataggcattattcgacagaggagagcaagaaatggagccagtagatcctagactagagccctggaagcatccaggaagtcagcctaagactgcgtgtaccacttgctattgtaaaaagtgctgctttcattgccaagtttgttttatgacaaaaggcttaggcatctcctatggcaggaagaagcggagacagcgacgaagagctcctcaagacagtcagactcatcaagcttatctatcaaagcagtaagtaatatatgtaatgcaacctttacaaatagtagcaatagtagcattagtagtagcaggaataatagcaatagttgtgtggaccatagtattcatagaatataagaaaatattaagacaaagaaaaatagacaggttgattgatagaataagagaaagagcagaagacagtggcaatgacagtgaaggggatcaggaagaattatcggcacttgtggacatggggcaccatgatccttgggatattaatgatctgtagagctgcaaacaatttgtgggtcacagtctattatggggtacctgtgtggagagaagcaaccaccactctattttgtgcatcagatgccaaggcatatgatgcagaggtacataatgtttgggccacacatgcctgtgtacccacagaccctaacccacaagaagtagaattgaaaaatgtgacagaaaattttaacatgtggaaaaataacatggtagaacagatgcatgaggatataatcagtttatgggatcaaagcctgaagccatgtgtaaaattaaccccactctgtgtttctttaaattgcactgatgctactaataccactaatagtaataccactagcagcagcgagaaaccgaaggggacaggggaaataaaaaactgctctttcaatatcaccacaagcataagagataaggtgcagaaacaatatgcacttttttatagccttgatgtagtaccaatggatgataatgataatagtacaagctataggttaataagttgtaacacctcaatcattacacaggcctgtccaaagatatcctttgagccaattcccatacattattgtgccccggctggttttgcgattctaaagtgtaaagataaaaggttcaatggaaaaggaccatgtacaagtgtcagcacagtacagtgtacacatggaattaggccagtagtatcaactcaactgttgttaaatggcagtctagcagaagaagaggtagtaattagatctgacaattttacgaacaatgctaaaaccataatagtacagctgagcaaatctgtagaaattacttgtgtaagacccaacaacaatacaagaaaaagtataagtatgggaccagggagagcattttatacaacagaaataataggagatataagacaagcatattgtaacattagtaaagcaaactggactgacactttagaacagatagctagaaaattaagagaacaatttgagaataaaacaatagtctttaagccatcctcaggaggggacccagaaattgtaacacagttttacagttttaattgtggaggggaatttttctactgtaattcaacacaactgtttaatggtacttggaatggtacttgggttaatggtacttggagtagtaataatacgactgatactgcaaatatcacactcccatgcagaataaaacaatttataaacatgtggcaggaagtaggaaaagcaatgtatgcccctcccatcaaaggacaaattaaatgtacatcaaatattacagggctgatattaacaagagatggtggtaacaataacaccacgaacgacaacgagaccgagaccttcagacctggaggaggagatatgagggacaattggagaagtgaattatataaatataaagtagtacaagttgaaccattaggagtagcacccaccaaggcaaagagaagagtggtgcaaagagaaaaaagagcagtgggaataggagctatgttccttgggttcttaggagcagcaggaagcactatgggcgcagcgtcagtgacgctgacggtacaagccagacaattattgtctggtatagtgcagcagcagaacaatctgctgagggctattgaggcgcaacagcatctgttgcaactcacagtctggggcatcaaacagctccaggcaagagtcctggctgtggaaagatacctaagggatcaacagctcctgggactttggggttgctctggaaaactcatttgcaccactactgtgccttggaacaatagctggagtaataaatctctggaaacaatttgggataacatgacctggatgcagtgggaaagagaaattgacaattacacaaacataatatacaccttaattgaagaatcgcagaaccaacaagaaaaaaatgaactagaattattggaattggataaatgggcaaatttgtggaattggtttagtatatcaaactggctatggtatataaaattattcataatggtagtaggaggcttggtaggtttaagaatagtttttactgtactttctatagttaatagagttaggcagggatactcaccattatcgtttcagacccacctcccaaccccgaagggacccgacaggccagaaggaatcgaagaagaaggtggagagagagacagaggcagctccactcgattagtgcacggattcttagcacttttctgggacgacctgaggagtctgtgcctcttcagctaccaccacttgagagacttactcttgattgtaacgaggattgtggaacttctgggacgcaggggatgggaagccctcaaatactggtggaatctcctgcagtattggaggcaggaactacagaagagtgctgttagcttgttcaatggcacggccatagcagtagctgaggggacagatagagttatagaagctttacgaagggcttatagagctattctccacatacctagaagaataagacagggcttagaaagggctttgctataaaatgggtggcaagtggtcagaaagtagtgtggttagaaggcatgtacctttaagacaaggcagctatagatcttagccgctttttaaaagaaaaggggggactggaagggctaattcactcacggaaaagaccagttgaaccagaagaagatagaagaggccatgaagaagaaaacaacagattgttctgcttgctcagctggggactttccagaaggcgcggcctgagtgactaagccccgttggggactttccgaagaggcatgaagggactttccaaggcaggcgtggcctgggcgggactggggagtggcgagccctcagatgctgcatataagcagctgctttctgcctgtactgggtctctctggttagaccagatctgagcctgggagctctctggctagctagggaacccactgcttaagcctcaataaagcttgccttgagtgcttcaagtagtgtgtgcccgtctgttgtgtgactctggtatctagagatccctcagaccattttagtccgtgtggaaaatctctagca

SEQ ID NO: 801 - protein, 17 aa
NTSLLHPVSLHGMDDPE

SEQ ID NO: 802 - DNA, 48 bp; single-stranded, linear
accagcttgttacaccctgtgagcctgcatggaatggatgaccctgag

SEQ ID NO: 803 - DNA, 10 bp; single-stranded, linear
accagcttgt

SEQ ID NO: 804 - DNA, 10 bp; single-stranded, linear
ccagcttgtt

SEQ ID NO: 805 - DNA, 10 bp; single-stranded, linear
cagcttgtta

SEQ ID NO: 806 - DNA, 10 bp; single-stranded, linear
agcttgttac

SEQ ID NO: 807 - DNA, 10 bp; single-stranded, linear
gcttgttaca

SEQ ID NO: 808 - DNA, 10 bp; single-stranded, linear
cttgttacac

SEQ ID NO: 809 - DNA, 10 bp; single-stranded, linear
ttgttacacc

SEQ ID NO: 810 - DNA, 10 bp; single-stranded, linear
tgttacaccc

SEQ ID NO: 811 - DNA, 10 bp; single-stranded, linear
gttacaccct

SEQ ID NO: 812 - DNA, 10 bp; single-stranded, linear
ttacaccctg

SEQ ID NO: 813 - DNA, 10 bp; single-stranded, linear
tacaccctgt

SEQ ID NO: 814 - DNA, 10 bp; single-stranded, linear
acaccctgtg

SEQ ID NO: 815 - DNA, 10 bp; single-stranded, linear
caccctgtga

SEQ ID NO: 816 - DNA, 10 bp; single-stranded, linear
accctgtgag

SEQ ID NO: 817 - DNA, 10 bp; single-stranded, linear
ccctgtgagc

SEQ ID NO: 818 - DNA, 10 bp; single-stranded, linear
cctgtgagcc

SEQ ID NO: 819 - DNA, 10 bp; single-stranded, linear
ctgtgagcct

SEQ ID NO: 820 - DNA, 10 bp; single-stranded, linear
tgtgagcctg

SEQ ID NO: 821 - DNA, 10 bp; single-stranded, linear
gtgagcctgc

SEQ ID NO: 822 - DNA, 10 bp; single-stranded, linear
tgagcctgca

SEQ ID NO: 823 - DNA, 10 bp; single-stranded, linear
gagcctgcat

SEQ ID NO: 824 - DNA, 10 bp; single-stranded, linear
agcctgcatg

SEQ ID NO: 825 - DNA, 10 bp; single-stranded, linear
gcctgcatgg

SEQ ID NO: 826 - DNA, 10 bp; single-stranded, linear
cctgcatgga

SEQ ID NO: 827 - DNA, 10 bp; single-stranded, linear
ctgcatggaa

SEQ ID NO: 828 - DNA, 10 bp; single-stranded, linear
tgcatggaat

SEQ ID NO: 829 - DNA, 10 bp; single-stranded, linear
gcatggaatg

SEQ ID NO: 830 - DNA, 10 bp; single-stranded, linear
catggaatgg

SEQ ID NO: 831 - DNA, 10 bp; single-stranded, linear
atggaatgga

SEQ ID NO: 832 - DNA, 10 bp; single-stranded, linear
tggaatggat

SEQ ID NO: 833 - DNA, 10 bp; single-stranded, linear
ggaatggatg

SEQ ID NO: 834 - DNA, 10 bp; single-stranded, linear
gaatggatga

SEQ ID NO: 835 - DNA, 10 bp; single-stranded, linear
aatggatgac

SEQ ID NO: 836 - DNA, 10 bp; single-stranded, linear
atggatgacc

SEQ ID NO: 837 - DNA, 10 bp; single-stranded, linear
tggatgaccc

SEQ ID NO: 838 - DNA, 10 bp; single-stranded, linear
ggatgaccct

SEQ ID NO: 839 - DNA, 10 bp; single-stranded, linear
gatgaccctg

SEQ ID NO: 840 - DNA, 10 bp; single-stranded, linear
atgaccctga

SEQ ID NO: 841 - DNA, 10 bp; single-stranded, linear
tgaccctgag